(19) United States
(12) Patent Application Publication
(10) Pub. No.: US 2022/0362308 A1
(43) Pub. Date: Nov. 17, 2022

(54) ORAL ADMINISTRATION OF GENETICALLY ENGINEERED BACTERIA

(71) Applicant: NOVOME BIOTECHNOLOGIES, INC., South San Francisco, CA (US)

(72) Inventors: Julie Huang, Berkeley, CA (US); Jonathan Kotula, Berkeley, CA (US)

(21) Appl. No.: 17/763,014

(22) PCT Filed: Sep. 24, 2020
(86) PCT No.: PCT/US2020/052582

§ 371 Date: Mar. 23, 2022

Related U.S. Application Data
(60) Provisional application No. 62/904,924, filed on Sep. 24, 2019.

(51) Int. Cl. A61K 35/74; A61K 35/17; A61P 35/00; A61K 9/00

(52) U.S. Cl. CPC A61K 35/74; A61K 35/17; A61P 35/00; A61K 9/0053

(57) ABSTRACT

Methods and compositions for administration of genetically engineered bacteria are provided. The methods include genetically engineering bacteria, e.g, gastrointestinal bacteria to include heterologous coding sequences for one or more tumor antigens, such as tumor-associated antigens, and administration, e.g, oral administration, of the genetically engineered microbes to a subject in need thereof.

### CROSS REFERENCE TO RELATED APPLICATIONS

This application claims the benefit of, and priority to, U.S. provisional patent application Ser. No. 62/904,924, filed Sep. 24, 2019, which is incorporated by reference herein in its entirety.

### TECHNICAL FIELD

The present disclosure relates to genetically engineered bacteria and uses thereof. More particularly, the present disclosure is directed to methods for producing bacteria that are engineered to express one or more genes encoding tumor-associated antigens, compositions comprising the genetically engineered bacteria, and administration, e.g., oral administration, of the compositions to a subject in need thereof.

### BACKGROUND

T cell recognition of tumor-associated antigens (TAAs) is critical for T cell priming in order to mount an effective anti-tumor response. TAA recognition is mediated by the binding of a T cell receptor to the peptide major histocompatibility complex (MHC) class I or class II, displayed on the surface of antigen-presenting cells or tumor cells. However, tumors downregulate the presentation of TAAs to T cells in order to evade the immune system. Dendritic cells (DCs) must be able to effectively engulf and process tumor cells to generate TAAs to activate cytotoxic T cells. Therapeutic cancer vaccines have been developed that include enriched populations of autologous DCs engineered ex vivo to include TAAs. See, e.g., Kantoff et al., N. Engl. J. Med. (2010) 363:411-422. However, to date, ex vivo therapies have practical limitations and elicit an immune response with limited scope.

Therapeutic cancer vaccines have also been developed that use bacteria in order to boost immune responses against TAAs. Bacteria naturally interact with DCs and have adjuvant properties that enhance a DC's ability to activate T cells. Engineered bacteria can enhance cytotoxic T cell priming so that a subsequent anti-tumor response can be mounted. These vaccines typically use live attenuated pathogens, such as attenuated Listeria monocytogenes and Salmonella enterica, as well as viral vectors, such as adeno-associated virus (AAV). These vectors encode genes for various TAAs and are administered intravenously. U.S. Pat. Nos. 9,878,024 and 9,764,013 describe bacterial immuno-oncology intravenous therapeutics using attenuated Listeria monocytogenes. However, patients in clinical trials for the Listeria-based therapy presented with gastrointestinal symptoms and tested positive for Listeria, prompting treatment with intravenous antibiotics. See, e.g., Macdonald, G., BioPharma-Reporter.com, Oct. 24, 2016.

Despite advances in the development of therapeutic cancer treatments, it is evident that challenges in developing an effective therapeutic cancer vaccine remain. Accordingly, new compositions and methods for enhancing immune responses to TAAs are highly desirable.

### SUMMARY

The present disclosure pertains to engineered bacteria for delivery to a subject in order to mount effective immune responses against one or more tumor antigens, such as TAAs, present on a tumor cell. The methods utilize engineered bacteria to deliver tumor antigens in order to enhance immune responses, e.g., T cell-mediated anti-tumor responses. In certain embodiments, the engineered bacteria are administered orally to provide an effective method for producing a robust immune response in vivo against a specific antigen presented by tumor cells in the subject in order to kill the tumor cells.

Accordingly, in one aspect, a method for enhancing an immune response to a selected tumor cell antigen is described. The method comprises: orally administering a genetically engineered, non-pathogenic, gut bacterium to a subject, wherein the subject comprises tumor cells expressing the selected tumor cell antigen, and further wherein the genetically engineered, non-pathogenic, gut bacterium comprises a heterologous polynucleotide encoding the tumor cell antigen operably linked to one or more control element(s), whereby said tumor cell antigen encoded by the polynucleotide can be expressed in the subject by the genetically engineered bacterium. Upon expression in the subject of the tumor cell antigen encoded by the polynucleotide, the immune response to the selected antigen expressed by the tumor cells is enhanced, as compared to the immune response against the tumor cell antigen expressed by the tumor cells in the absence of the genetically engineered, non-pathogenic, gut bacterium.

In another aspect, the disclosure provides a method of treating a cancer comprising cancerous cells that express a tumor cell antigen in a subject in need thereof. The method comprises orally administrating to the subject a genetically engineered, non-pathogenic, gut bacterium comprising a heterologous polynucleotide encoding the tumor cell antigen.

In another aspect, the disclosure provides a method of stimulating an immune response against a tumor cell antigen in a subject. The method comprises orally administrating to the subject a genetically engineered, non-pathogenic, gut bacterium comprising a heterologous polynucleotide encoding the tumor cell antigen.

In certain embodiments, the tumor cell antigen comprises a tumor-associated antigen.

In certain embodiments, the non-pathogenic, gut bacterium comprises a commensal or mutual gut bacterium.

In certain embodiments, the non-pathogenic, gut bacterium is selected from the group consisting of a Bacteroidetes, a Firmicutes, a Proteobacteria and an Actinobacteria bacterium, such as a Bacteroides spp., e.g., Bacteroides thetaiotaomicron.

In another aspect, a method for enhancing an immune response to a selected tumor-associated antigen is described. The method comprises: genetically engineering a Bacteroides bacterium, e.g., a Bacteroides thetaiotaomicron bacterium, to comprise a heterologous polynucleotide encoding the tumor-associated antigen operably linked to one or more control element(s), whereby said tumor-associated antigen encoded by the polynucleotide can be expressed in a subject by the genetically engineered Bacteroides bacterium; and orally administering the genetically engineered Bacteroides bacterium to the subject, wherein the subject comprises tumor cells expressing the selected tumor-associated antigen, and further wherein, upon expression in the subject of the tumor-associated antigen encoded by the polynucleotide, the immune response to the selected antigen expressed by the tumor cells is enhanced, as compared to the immune response against the tumor-associated antigen expressed by the tumor cells in the absence of the genetically engineered Bacteroides bacterium.

In another aspect, the disclosure provides a method for enhancing an immune response to a selected tumor-associated antigen in a subject comprising tumor cells expressing the tumor-associated antigen. The method comprises orally administering a genetically engineered Bacteroides bacterium, e.g., a genetically engineered Bacteroides thetaiotaomicron bacterium, to the subject, wherein the Bacteroides bacterium has been genetically engineered to comprise a heterologous polynucleotide encoding the tumor-associated antigen operably linked to one or more control element(s).

In certain embodiments of the above methods, the subject is a mammal, e.g., a human.

In additional embodiments of the above methods, the genetically engineered, non-pathogenic, gut bacterium is not attenuated.

In additional embodiments, an adoptive cell therapy such as, but not limited to, a CAR-T cell therapy, is administered to the subject prior to, concurrent with, and/or subsequent to, administering the genetically engineered, non-pathogenic, gut bacterium.

In further embodiments, an immune checkpoint inhibitor is administered to the subject prior to, concurrent with, and/or subsequent to, administering the genetically engineered, non-pathogenic, gut bacterium.

In another aspect, the disclosure provides a genetically engineered Bacteroides bacterium. The bacterium comprises a heterologous polynucleotide encoding a tumor cell antigen operably linked to one or more control element(s), wherein the tumor cell antigen encoded by the polynucleotide can be expressed in a subject by the genetically engineered Bacteroides bacterium, and wherein, upon expression in the subject of the tumor cell antigen encoded by the polynucleotide, an immune response to the tumor cell antigen in the subject is enhanced as compared to the immune response in the absence of the genetically engineered Bacteroides bacterium.

In another aspect, the disclosure provides a method for enhancing an immune response to a selected tumor cell antigen. The method comprises: orally administering a genetically engineered bacterium to a subject, wherein the subject comprises tumor cells expressing the selected tumor cell antigen, and further wherein the genetically engineered bacterium comprises a heterologous polynucleotide encoding the tumor cell antigen operably linked to one or more control element(s), whereby said tumor cell antigen encoded by the polynucleotide can be expressed in the subject by the genetically engineered bacterium. Upon expression in the subject of the tumor cell antigen encoded by the polynucleotide, the immune response to the selected antigen expressed by the tumor cells is enhanced, as compared to the immune response against the tumor cell antigen expressed by the tumor cells in the absence of the genetically engineered bacterium. In another aspect, the disclosure provides a method of treating a cancer comprising cancerous cells that express a tumor cell antigen in a subject in need thereof. The method comprises orally administrating to the subject a genetically engineered bacterium comprising a heterologous polynucleotide encoding the tumor cell antigen. In another aspect, the disclosure provides a method of stimulating an immune response against a tumor cell antigen in a subject. The method comprises orally administrating to the subject a genetically engineered bacterium comprising a heterologous polynucleotide encoding the tumor cell antigen. In certain embodiments the genetically engineered bacterium is an attenuated pathogenic bacteria. In certain embodiments the genetically engineered bacteria is a Listeria spp., e.g., Listeria monocytogenes.

In another aspect, the disclosure provides a method of treating a cancer comprising cancerous cells that express a tumor cell antigen in a subject in need thereof. The method comprises (i) orally administrating to the subject a genetically engineered, non-pathogenic, gut bacterium comprising a heterologous polynucleotide encoding the tumor cell antigen, and (ii) administrating to the subject a checkpoint inhibitor. In another aspect, the disclosure provides a method of stimulating an immune response against a tumor cell antigen in a subject. The method comprises (i) orally administrating to the subject a genetically engineered, non-pathogenic, gut bacterium comprising a heterologous polynucleotide encoding the tumor cell antigen, and (ii) administrating to the subject a checkpoint inhibitor.

These aspects and other embodiments of the present disclosure will readily occur to one of ordinary skill in the art in view of the disclosure herein.

### BRIEF DESCRIPTION OF THE FIGURES

The disclosure can be more completely understood with reference to the following drawings.

FIG. 1 is a diagram of plasmid pCB6655 used in the Examples.

FIG. 2 shows the DNA sequence (SEQ ID NO: 1) of plasmid pCB6655.

FIG. 3 shows tumor size at various days post-implantation with differing amounts of B16F10-OVA inoculum, as described in the Examples.

FIG. 4 shows tumor size at various days post-implantation with differing amounts of pre-activated OT-1 CD8+ cells, as described in the Examples.

FIG. 5 shows tumor size at various days post-implantation with differing amounts of non-activated OT-1 CD8+ cells, as described in the Examples.

FIG. 6 shows tumor volume of mice treated with OVA-expressing Listeria administered: intravenously at a dose of 1×10⁴ CFU (Lm IV low, G2), intravenously at a dose of 1×10⁵ CFU (Lm IV med, G3), intravenously at a dose of 1×10⁶ CFU (Lm IV high, G4), orally at a dose of 1×10⁸ CFU (Lm Oral low, G5), orally at a dose of 1×10⁹ CFU (Lm Oral med, G6), or orally at a dose of 1×10¹⁰ CFU (Lm Oral high, G7), in a B16-OVA murine tumor model.

FIG. 7 shows survival of mice treated with OVA-expressing Listeria administered: intravenously at a dose of 1×10⁴ (G2), intravenously at a dose of 1×10⁵ (G3), intravenously at a dose of 1×10⁶ (G4), orally at a dose of 1×10⁸ CFU (G5), orally at a dose of 1×10⁹ CFU (G6), or orally at a dose of 1×10¹⁰ CFU (G7), in a B16-OVA murine tumor model. P values were determined by log rank test.

FIG. 8 shows tumor volume of mice treated with: anti-PD-1 antibody (PD1); non-OVA-expressing Listeria (Lm parent); αPD1 in combination with Lm parent; OVA-expressing Listeria (Lm-OVA); or αPD1 in combination with Lm-OVA, in a B16-OVA murine tumor model.

FIG. 9 shows survival of mice treated with anti-PD-1 antibody (αPD1, G2); non-OVA-expressing Listeria (Lm parent, G3); αPD1 in combination with Lm parent (G4); OVA-expressing Listeria (Lm-OVA, G5); or αPD1 in combination with Lm-OVA (G6), in a B16-OVA murine tumor model. NS: not significant.

### DETAILED DESCRIPTION

It is to be understood that the terminology used herein is for the purpose of describing particular embodiments only, and is not intended to be limiting. As used in the present Specification and the appended claims, the singular forms “a,” “an,” and “the” include plural referents unless the context clearly dictates otherwise. Thus, for example, reference to “a gut microorganism” includes one or more such organisms; reference to “a cell” includes one or more cells; and the like.

Unless defined otherwise, all technical and scientific terms used herein have the same meaning as commonly understood by one of ordinary skill in the art to which the present disclosure pertains. Although other methods and materials similar, or equivalent, to those described herein can be used in the practice of the present disclosure, preferred materials and methods are described herein.

In view of the teachings of the present Specification, one of ordinary skill in the art can apply conventional techniques of immunology, biochemistry, chemistry, molecular biology, microbiology, cell biology, genomics, and recombinant polynucleotides, as taught, for example, by the following standard texts: Antibodies: A Laboratory Manual, Second edition, E. A. Greenfield, 2014, Cold Spring Harbor Laboratory Press, ISBN 978-1-936113-81-1; Culture of Animal Cells: A Manual of Basic Technique and Specialized Applications, 6th Edition, R. I. Freshney, 2010, Wiley-Blackwell, ISBN 978-0-470-52812-9; Transgenic Animal Technology, Third Edition: A Laboratory Handbook, 2014, C. A. Pinkert, Elsevier, ISBN 978-0124104907; The Laboratory Mouse, Second Edition, 2012, H. Hedrich, Academic Press, ISBN 978-0123820082; Manipulating the Mouse Embryo: A Laboratory Manual, 2013, R. Behringer, et al., Cold Spring Harbor Laboratory Press, ISBN 978-1936113019; PCR 2: A Practical Approach, 1995, M. J. McPherson, et al., TRL Press, ISBN 978-0199634248; Methods in Molecular Biology (Series), J. M. Walker, ISSN 1064-3745, Humana Press; RNA: A Laboratory Manual, 2010, D. C. Rio, et al., Cold Spring Harbor Laboratory Press, ISBN 978-0879698911; Methods in Enzymology (Series), Academic Press; Molecular Cloning: A Laboratory Manual (Fourth Edition), 2012, M. R. Green, et al., Cold Spring Harbor Laboratory Press, ISBN 978-1605500560; Bioconjugate Techniques, Third Edition, 2013, G. T. Hermanson, Academic Press, ISBN 978-0123822390; Methods in Plant Biochemistry and Molecular Biology, 1997, W. V. Dashek, CRC Press, ISBN 978-0849394805.

### Definitions

The “gastrointestinal (GI) tract,” also known as the “gut,” “digestive tract,” “digestional tract,” “GIT,” and “alimentary canal,” refers collectively to the mouth, esophagus, stomach, small intestine, large intestine, rectum, and anus.

The terms “gut microbiome,” “gut flora,” “gut microbiota,” and “GI microbiota” are used interchangeably herein and refer to the community of microorganisms that live in the GI tract of humans and other animals. Representative organisms that are part of the gut microbiome are described in detail herein.

The terms “GI bacteria,” “GI microorganism,” and “GI microbe” are used interchangeably herein and refer to microorganisms that can live in the gut of humans and other animals. The terms include microorganisms that are present in vivo in the GI tract or microorganisms that can live in the GI tract but that are isolated therefrom.

By a “non-pathogenic” bacterium is meant a bacterium that typically does not cause disease, harm, and/or death to a selected subject. The term includes non-disease causing bacteria that normally reside on and inside vertebrates as commensals. The term “non-pathogenic” includes microorganisms that are normally not harmful to the host in question, but that may have the potential of causing disease if they enter the body, multiply, and cause symptoms of infection, such as in immunocompromised individuals. Genes have also been identified that predispose disease and infection with non-pathogenic bacteria by a small number of persons. For the purposes of the present disclosure, these bacteria are also considered “non-pathogenic.” A number of bacterial strains that are non-pathogenic to humans and other mammals exist in the GI tract and are described further herein. The term “non-pathogenic” as used herein does not include a bacterium that has been intentionally attenuated, e.g., by reducing the virulence of a pathogenic bacterium. Nor does it include a pathogenic bacterium that has been inactivated, e.g., where the pathogen has been killed.

By “commensal” or “mutual” bacterium, in the context of the present disclosure, is meant a non-pathogenic bacterium that is normally found in the host in question. A “commensal” bacterium derives food or other benefits from another organism without necessarily hurting or helping it. Commensal bacteria are part of the normal flora in the mouth, skin, and gut microbiome. A “mutual” relationship is one in which both organisms benefit. A commensal or mutual organism may depend on its host for food, shelter, support, transport, or a combination of these factors. The host may receive a variety of benefits, including protection from infection, improved digestion, or other benefits (e.g., cleaner skin).

As used herein, the term “antigen” refers to a molecule that is recognized by antibodies, specific immunologically-competent cells, or both. An antigen may be derived from many types of substances, such as, but not limited to, molecules from organisms, such as, for example, proteins, subunits of proteins, nucleic acids, lipids, killed or inactivated whole cells or lysates, synthetic molecules, and a wide variety of other agents both biological and non-biological.

By a “tumor antigen” is meant an antigen that is expressed by a tumor or cancer cell. Unless indicated otherwise “tumor antigen” and “cancer antigen” are used interchangeably herein. Tumor antigens can be broadly categorized into aberrantly expressed self-antigens, mutated self-antigens, tumor-associated antigens (TAAs), and tumor-specific antigens (TSAs). TAAs are tumor antigens that are associated with malignant cell phenotypes and are relatively restricted to tumor cells, but are sometimes also expressed by normal cells. However, the expression of TAAs by malignant cells has unique features that contribute to their immunogenicity. In particular, TAA expression in tumor cells differs from that in normal tissues by the degree of expression in the tumor, alterations in protein structure in comparison with normal counterparts, or by aberrant subcellular localization within tumor cells. While TAAs are relatively restricted to tumor cells, TSAs are unique to tumor cells. TAAs and TSAs typically include portions of intracellular molecules and are expressed on the cell surface as part of the major histocompatibility complexes.

The terms “subject,” “individual,” or “patient” are used interchangeably herein and refer to any member of the phylum Chordata, including, without limitation, mammals such as humans and other primates, including non-human primates such as rhesus macaques, chimpanzees, and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like. The term does not denote a particular age or gender. Thus, the term includes adult, young, and newborn individuals as well as males and females.

The terms “effective amount” or “therapeutically effective amount” of a composition or agent refer to a sufficient amount of the composition or agent to provide the desired response. For example, in cancers, a desired response includes, but is not limited to, reducing tumor size; reducing the amount or frequency of symptoms associated with the cancer in question; inhibiting progression of the cancerous state, such as by reducing or eliminating metastasis; slowing the typical rate of progression of the particular cancer; and the like. The exact effective amount of the composition or agent required will vary from subject to subject, depending on the species, age, and general condition of the subject, the severity of the condition being treated, the particular agent administered, the mode of administration, and the like. An appropriate “effective” amount in any individual case may be determined by one of ordinary skill in the art using routine experimentation.

“Treatment” or “treating” a particular disorder includes: preventing the disorder, e.g., preventing the development of the disorder or causing the disorder to occur with less intensity in a subject that may be predisposed to the disorder but does not yet experience or display symptoms of the disorder; inhibiting the disorder, e.g., reducing the rate of development, arresting the development, or reversing the disease state; and/or relieving symptoms of the disorder e.g., decreasing the number of symptoms experienced by the subject.

The terms “engineered,” “genetically engineered,” “genetically modified,” “recombinant,” “modified,” and “non-naturally occurring” indicate intentional human manipulation of the genome of an organism. Methods of genetic modification include, for example, heterologous gene expression, gene or promoter insertion or deletion, nucleic acid mutation, altered gene expression or inactivation, enzyme engineering, directed evolution, knowledge-based design, random mutagenesis methods, gene shuffling, codon optimization, and the like. Methods for genetically engineering organisms are described in detail herein.

“Gene editing” or “genome editing,” as used herein, refers to the insertion, deletion, or replacement of a nucleotide sequence at a specific site in the genome of an organism or cell.

A “parental microorganism” is a microorganism used to generate a genetically engineered microorganism. The parental microorganism may be a naturally occurring microorganism (e.g., a wild-type microorganism) or a microorganism that has been previously modified (e.g., a mutant or recombinant microorganism). The microorganism may be engineered to express or overexpress one or more tumor antigens, such as TAAs, or metabolites that were not expressed in the parental microorganism. Similarly, microorganisms of the present disclosure may be engineered to contain one or more genes that were not contained by the parental microorganism.

The terms “wild-type,” “naturally occurring,” and “unmodified” are used herein to mean the typical (or most common) form, appearance, phenotype, or strain existing in nature; for example, the typical form of cells, organisms, characteristics, polynucleotides, proteins, macromolecular complexes, genes, RNAs, DNAs, or genomes as they occur in and can be isolated from a source in nature. The wild-type form, appearance, phenotype, or strain serve as the original parent before an intentional modification. Thus, mutant, variant, chimeric, engineered, and recombinant are not wild-type forms.

As used herein, the terms “nucleic acid,” “nucleotide sequence,” “oligonucleotide,” and “polynucleotide” are interchangeable. All refer to a polymeric form of nucleotides. The nucleotides may be deoxyribonucleotides (DNA) or ribonucleotides (RNA), or analogs thereof, and they may be of any length. Polynucleotides may perform any function and may have any secondary structure and three-dimensional structure. The terms encompass known analogs of natural nucleotides and nucleotides that are modified in the base, sugar, and/or phosphate moieties. Analogs of a particular nucleotide have the same base-pairing specificity (e.g., an analog of A base pairs with T). A polynucleotide may comprise one modified nucleotide or multiple modified nucleotides. Examples of modified nucleotides include methylated nucleotides and nucleotide analogs. Nucleotide structure may be modified before or after a polymer is assembled. Following polymerization, polynucleotides may be additionally modified via, for example, conjugation with a labeling component or target-binding component. A nucleotide sequence may incorporate non-nucleotide components. The terms also encompass nucleic acids comprising modified backbone residues or linkages, that (i) are synthetic, naturally occurring, and non-naturally occurring, and (ii) have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), and morpholino structures.

Polynucleotide sequences are displayed herein in the conventional 5′ to 3′ orientation.

As used herein, the term “complementarity” refers to the ability of a nucleic acid sequence to form hydrogen bond(s) with another nucleic acid sequence (e.g., through traditional Watson-Crick base pairing). A percent complementarity indicates the percentage of residues in a nucleic acid molecule that can form hydrogen bonds with a second nucleic acid sequence. When two polynucleotide sequences have 100% complementarity, the two sequences are perfectly complementary, i.e., all of a first polynucleotide's contiguous residues hydrogen bond with the same number of contiguous residues in a second polynucleotide.

As used herein, the term “sequence identity” generally refers to the percent identity of bases or amino acids determined by comparing a first polynucleotide or polypeptide to a second polynucleotide or polypeptide using algorithms having various weighting parameters. Sequence identity between two polypeptides or two polynucleotides can be determined using sequence alignment by various methods and computer programs (e.g., BLAST, CS-BLAST, FASTA, HIMMER, L-ALIGN, etc.), available through the worldwide web at sites including GENBANK (ncbi.nlm.nih.gov/genbank/) and EMBL-EBI (ebi.ac.uk.). Sequence identity between two polynucleotides or two polypeptide sequences is generally calculated using the standard default parameters of the various methods or computer programs. Generally, the various proteins for use herein will have at least about 75% or more sequence identity to the wild-type or naturally occurring sequence of the protein of interest, such as about 80%, such as about 85%, 90%, 91%, 92%, 93%, 94%, 95%, 96%, 97%, 98%, 99%, or complete identity.

As used herein, the term “recombination” refers to a process of exchange of genetic information between two polynucleotides.

The terms “vector” and “plasmid” are used interchangeably and as used herein refer to polynucleotide vehicles useful to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette. Vectors and plasmids include, but are not limited to, integrating vectors, prokaryotic plasmids, eukaryotic plasmids, plant synthetic chromosomes, episomes, viral vectors, cosmids, and artificial chromosomes.

As used herein the term “expression cassette” is a polynucleotide construct, generated recombinantly or synthetically, comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in an expression vector.

As used herein, the terms “regulatory sequences,” “regulatory elements,” and “control elements” are interchangeable and refer to polynucleotide sequences that are upstream (5′ non-coding sequences), within, or downstream (3′ non-translated sequences) of a polynucleotide target to be expressed. Regulatory sequences influence, for example, the timing of transcription, amount or level of transcription, RNA processing or stability, and/or translation of the related structural nucleotide sequence. Regulatory sequences may include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like.

As used herein the term “operably linked” refers to polynucleotide sequences or amino acid sequences placed into a functional relationship with one another. For instance, a promoter or enhancer is operably linked to a coding sequence if it regulates, or contributes to the modulation of, the transcription of the coding sequence. Operably linked DNA sequences encoding regulatory sequences are typically contiguous to the coding sequence. However, enhancers can function when separated from a promoter by up to several kilobases or more. Additionally, multicistronic constructs can include multiple coding sequences which use only one promoter by including a 2 A self-cleaving peptide, an IRES element, etc. Accordingly, some polynucleotide elements may be operably linked but not contiguous.

As used herein, the term “expression” refers to transcription of a polynucleotide from a DNA template, resulting in, for example, an mRNA or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene products.” Expression may include splicing the mRNA in a eukaryotic cell, if the polynucleotide is derived from genomic DNA.

As used herein, the term “amino acid” refers to natural and synthetic (unnatural) amino acids, including amino acid analogs, modified amino acids, peptidomimetics, glycine, and D or L optical isomers.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms may be used to refer to amino acid polymers that have been modified through, for example, acetylation, disulfide bond formation, glycosylation, lipidation, phosphorylation, cross-linking, and/or conjugation (e.g., with a labeling component or ligand). Polypeptide sequences are displayed herein in the conventional N-terminal to C-terminal orientation.

Polypeptides and polynucleotides can be made using routine techniques in the field of molecular biology (see, e.g., standard texts discussed herein). Furthermore, essentially any polypeptide or polynucleotide can be ordered from commercial sources.

The term “binding” as used herein includes a non-covalent interaction between macromolecules (e.g., between a protein and a polynucleotide, between a polynucleotide, and a polynucleotide, or between a protein and a protein). Such non-covalent interactions are also referred to as “associating” or “interacting” (e.g., when a first macromolecule interacts with a second macromolecule, the first macromolecule binds to second macromolecule in a non-covalent manner). Some portions of a binding interaction may be sequence-specific; however, all components of a binding interaction do not need to be sequence-specific, such as the contact of a protein with phosphate residues in a DNA backbone. Binding interactions can be characterized by a dissociation constant (Kd). “Affinity” refers to the strength of binding. An increased binding affinity is correlated with a lower Kd. An example of non-covalent binding is hydrogen bond formation between base pairs.

As used herein, the term “isolated” can refer to a nucleic acid, polypeptide, bacterium, and the like that, by the hand of a human, exists apart from its native environment and is therefore not a product of nature. Isolated means substantially pure. An isolated nucleic acid or polypeptide can exist in a purified form and/or can exist in a non-native environment such as, for example, in a recombinant cell.

As used herein, a “host cell” generally refers to a biological cell. A cell can be the basic structural, functional, and/or biological unit of a living organism. A cell can originate from any organism having one or more cells. Examples of host cells include, but are not limited to: a prokaryotic cell, eukaryotic cell, a bacterial cell, an archaeal cell, a cell of a single-cell eukaryotic organism, a protozoa cell, a cell from a plant, an algal cell, seaweeds, a fungal cell, an animal cell, a cell from an invertebrate animal, a cell from a vertebrate animal, a cell from a mammal (including humans), and the like. Further, a cell can be a stem cell or progenitor cell.

As used herein, with regard to bacteria, the term “species” refers to a taxonomic entity as conventionally defined by genomic sequence and phenotypic characteristics. A “strain” is a particular instance of a species that has been isolated and purified according to conventional microbiological techniques. The present disclosure encompasses derivatives of the disclosed bacterial strains. The term “derivative” includes daughter strains (progeny) or stains cultured (sub-cloned) from the original but modified in some way (including at the genetic level), without altering negatively a biological activity of the strain.

rRNA, 16S rDNA, 16S rRNA, 16S, 18S, 18S rRNA, and 18S rDNA refer to nucleic acids that are components of, or encode for, components of the ribosome. There are two subunits in the ribosome termed the small subunit (SSU) and large subunit (LSU). rDNA genes and their complementary RNA sequences are widely used for determination of the evolutionary relationships amount organisms as they are variable, yet sufficiently conserved to allow cross-organism molecular comparisons.

16S rDNA sequence (approximately 1542 nucleotides in length) of the 30S SSU can be used, in certain embodiments, for molecular-based taxonomic assignments of prokaryotes and the 18S rDNA sequence (approximately 1869 nucleotides in length) of 40S SSU may be used for eukaryotes. For example, 16S sequences may be used for phylogenetic reconstruction as they are general highly conserved but contain specific hypervariable regions that harbor sufficient nucleotide diversity to differentiate genera and species of most bacteria. Although 16S rDNA sequence data has been used to provide taxonomic classification, closely related bacterial strains that are classified within the same genus and species, may exhibit distinct biological phenotypes.

The identity of contemplated bacterial species or strains may be characterized by 16S rRNA or full genome sequence analysis. For example, in certain embodiments, contemplated bacterial strains may comprise a 16S rRNA or genomic sequence having a certain % identity to a reference sequence.

As used herein, the term “immune response” refers to a response by a cell of the immune system, such as a B cell, T cell (CD4+ or CD8+), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4+ T cell, CD8+ T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure. For example, it will be appreciated that all features described and depicted herein can be applicable to all aspects of the disclosure described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present disclosure remains operable. Moreover, two or more steps or actions may be conducted simultaneously.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

### Engineered Gut Bacteria

The present disclosure pertains to engineered bacteria for delivery, e.g., oral delivery, to a selected subject in order to mount effective immune responses against one or more tumor antigens, such as, but not limited to, TAAs present on a tumor cell. The methods utilize engineered bacteria, e.g., non-pathogenic bacteria, to deliver tumor antigens in vivo in order to enhance immune responses, e.g., T cell-mediated anti-tumor responses. The engineered bacteria can be administered orally to provide an effective method for producing robust immune responses against specific tumor antigens presented by tumor cells so that the cells are killed.

Without being bound by a particular theory or method, a representative mechanism of action and exemplary use of the technique of the present disclosure by which tumor cells are killed is presented below. In one embodiment, commensal gut bacteria that have been genetically engineered to carry a gene encoding one or more tumor antigens, such as one or more TAAs, are delivered orally to a patient with cancer. After oral administration, the engineered bacteria travel to the GI tract where antigen presenting cells (APCs) such as, but not limited to, dendritic cells (DCs), sample the engineered bacteria in the lumen of the intestine. The DCs engulf the bacteria and process proteins expressed by the bacteria, including the TAAs, into peptide antigens. DCs present these antigens on their MHC class I and II, which prime CD8+ and CD4+ T cells, respectively. The T cells that specifically recognize the TAAs on the MHC are activated and clonally expand to generate many T cell clones with the same specificity for the antigen. The activated T cells travel to the site of the tumor where, upon recognition of the antigen presented by the tumor cells, the activated T cells kill the tumor.

As explained herein, the methods typically include genetically engineering non-pathogenic, commensal, or mutual, gut bacteria. A number of naturally occurring, non-pathogenic, gut bacteria are known including, without limitation, bacteria in the phyla Bacteroidetes, Firmicutes, Proteobacteria, and Actinobacteria. Representative gut organisms include, without limitation, Lactobacillus spp., e.g., Lactobacillus reuteri, and Lactobacillus casei; Bifidobacterium spp., e.g., Bifidobacterium longum, and Bifdobacterium bifidum; Bacteroides spp., e.g., Bacteroides thetaiotaomicron and Bacteroides ovatus; Clostridium spp., e.g., Clostridium perfringens, Clostridium beijerinckii, and Clostridium cellulolyticum; Bacillus spp., e.g., Bacillus subtilus; Roseburia spp., e.g., Roseburia hominis and Roseburia intestinalis; Ruminococcus spp., e.g., Ruminococcus gnavus; Faecalibacterium spp., e.g., Faecalibacterium prausnitzii; Escherichia spp., e.g., E. coli; Corynebacterium spp., e.g., Corynebacterium glutamicum; Streptococcus spp., e.g., Streptococcus agalactiae; Eubacterium spp., e.g., Eubacterium eligens; and Peptococcus spp. Additional contemplated bacteria, for example, for use in a disclosed composition or method, include bacteria of genus Alistipes, Parabacteroides, Prevotella, Oscillibacter, Gemmiger, Barnesiella, Dialister, Parasutterella, Phascolarctobacterium, Propionibacterium, Sutterella, Blautia, Paraprevotella, Coprococcus, Odoribacter, Spiroplasma, Anaerostipes, or Akkermansia.

A contemplated bacterium, for example, for use in a disclosed composition or method, may be of the Bacteroides genus, i.e., may be a Bacteroides species bacterium. Exemplary Bacteroides species include B. acidifaciens, B. barnesiaes, B. caccae, B. caecicola, B. caecigallinarum, B. cellulosilyticus, B. cellulosolvens, B. clarus, B. coagulans, B. coprocola, B. coprophilus, B. coprosuis, B. distasonis, B. dorei, B. eggerthii, B. gracilis, B. faecichinchillae, B. faecis, B. finegoldii, B. fluxus, B. fragilis, B. galacturonicus, B. gallinaceum, B. gallinarum, B. goldsteinii, B. graminisolvens, B. helcogene, B. intestinalis, B. luti, B. massiliensis, B. melaninogenicus, B. nordii, B. oleiciplenus, B. oris, B. ovatus, B. paurosaccharolyticus, B. pectinophilus, B. plebeius, B. polypragmatus, B. propionicifaciens, B. putredinis, B. pyogenes, B. reticulotermitis, B. rodentium, B. salanitronis, B. salyersiae, B. sartorii, B. sediment B. stercoris, B. suis, B. tectus, B. thetaiotaomicron, B. uniformis, B. vulgatus, B. xylanisolvens, and B. xylanolyticusxylanolyticus. In certain embodiments, the bacterium is a Bacteroides vulgatus bacterium.

In other embodiments, a contemplated bacterium, for example, for use in a disclosed composition or method, is a pathogenic bacterium that has been attenuated, e.g., by reducing the virulence of the pathogenic bacterium. For example, a contemplated bacterium may be of the Listeria genus, i.e., may be a Listeria species bacterium. In certain embodiments, the Listeria species bacterium may have one or more virulence factors (e.g., LLO) knocked-out or be genetically engineered so that the expression of one or more virulence factors is otherwise reduced.

Disclosed bacteria may be genetically engineered in order to include genes encoding one or more tumor antigens. Tumor antigens for use in the present disclosure can be categorized in the following groups: nonmutated shared antigens, e.g., MAGE, BAGE, RAGE, NY-ESO, among others, which are expressed in testes and in multiple tumor cells; differentiation antigens, e.g., prostate-specific membrane antigen (PSMA) and prostate-specific antigen (PSA) in prostate carcinoma, Mart1/MelanA and tyrosinase present in many melanomas, and carcino embryonic antigen (CEA) present in a large percentage of colon cancers, which antigens are tissue-restricted and present in lineage-specific tumor cells; mutated oncogenes and tumor suppressor genes, e.g., mutated ras, rearranged bcr/abl, and mutated p53, which provide novel epitopes for immune recognition; unique idiotypes, e.g., immunoglobulin antigensin myeloma and B-cell myeloma, and T-cell receptor (TCR) expressed in CTCL; oncovirus-derived epitopes, e.g., the human papillomavirus-encoded E6 and E7 proteins and the Epstein Barr virus (EBV)-associated antigens present in primary brain lymphomas; and nonmutated oncofetal proteins such as CEA, α-fetoprotein, and survivin. See, e.g., Zitvogel et al., Advances in Immunol. (2004) 84:131-179, for a discussion of these antigens.

Non-limiting examples of particular tumor antigens include WTI, MUC1, LMP2, HPV E6 E7, HPV16 E7, EGFRvIII, HER-2/neu, idiotype, MAGE A3, P53 non-mutant NY-ESO-1, PSMA, GD-2, CEA, MelonA MART1, Ras-mutant, gp100, p53 mutant, proteinase 3/PR1, Bcr-able, tyrosinase, survivin, PSA, hTERT, Sarcoma translocation breakpoints, EphA2, PAP, ML-IAP, AFP, EpCAM, ERG (TMPRSS2 ETS fusion gene), NA17, PAX3, ALK, androgen receptor, cyclin B1, polysialic acid, MYCN, TRP-2, RhoC, GD3, fuosyl GM1, mesothelin, PSCA (prostate stem cell antigen), MAGE A1, sLe(a), Cyp1B1, PLAC1 (placenta-specific 1), CM3 ganglioside, BORIS (brother of regulator of imprinted sites), Tn, GloboH, ETV6-AML, NY-BR-1, PGS5, SART3, STn, carbonic anhydrase IX, PAX5, OY-TES1, sperm protein 17, LCK, HMWMAA, sperm fibrous shealth proteins, AKAP-4, SSX2, XAGE1, B7H3, legumain, Tie 2, Page4, VEGFR2, MAD-CT-1 (protamine 2), FAP, MAD-CT-2; PDGFR-beta, Fos-related antigen 1, and lung tumor-associated antigen (see, e.g., Cheever et al., Clin. Cancer Res. (2009) 15:5323-5337). Additional exemplary tumor antigens include adenosine A2a receptor (A2aR), A kinase anchor protein 4 (AKAP4), CA125, CAIX, CD19, CD20, CD22, CD30, CD33, CD52, CD73, CD137, CS1, estrogen receptor binding site associated antigen 9 (EBAG9), epidermal growth factor (EGF), epidermal growth factor receptor (EGFR), EGF-like module receptor 2 (EMR2), G antigen (GAGE), disialoganglioside GD2 (GD2), human epidermal growth factor receptor 2 (Her2), hepatocyte growth factor (HGF), human papillomavirus 16 (HPV-16), heat-shock protein 105 (HSP105), isocitrate dehydrogenase type 1 (IDH1), indoleamine-2,3-dioxygenase 1 (IDO1), IGF-1, IGF1R, IGG1K, lymphocyte antigen 6 complex K (LY6K), Matrix-metalloproteinase-16 (MMP16), melanotransferrin (MFI2), melanoma antigen C2 (MAGE-C2), melanoma antigen D4 (MAGE-D4), melanoma antigen recognized by T-cells 1 (Melan-A/MART-1), N-methyl-N′-nitroso-guanidine human osteosarcoma transforming gene (MET), mucin 4 (MUC4), mucin 16 (MUC16), programmed cell death receptor ligand 1 (PD-L1), phosphatidylserine, preferentially expressed antigen of melanoma (PRAME), protein tyrosine kinase 7 (PTK7, also known as colon carcinoma kinase 4 (CCK4)), receptor tyrosine kinase orphan receptor 1 (ROR1), scatter factor receptor kinase, sialyl-Tn, sperm-associated antigen 9 (SPAG-9), synovial sarcoma X-chromosome breakpoint 1 (SSX1), survivin, telomerase, vascular endothelial growth factor (VEGF) (e.g., VEGF-A), 5T4, Mesothelin, Glypican 3 (GPC3), Folate Receptor α (FRα), cMET, CD38, B Cell Maturation Antigen (BCMA), CD123, CLDN6, CLDN9, LRRC15, PRLR (Prolactin Receptor), RING finger protein 43 (RNF43), Uroplakin-1 B (UPK1 B), tumor necrosis factor superfamily member 9 (TNFSF9), tumor necrosis factor receptor superfamily member 21 (TNFSRF21), bone morphogenetic protein receptor type-1B (BMPR1B), Kringle domain-containing transmembrane protein 2 (KREMEN2), and Delta-like protein 3 (DLL3).

The protein and nucleotide sequences of these and other tumor antigens are known. See, e.g., the National Center for Biotechnology (NCBI) database, and Table 1 of U.S. Pat. No. 9,878,024.

In certain embodiments, a modified bacterium comprises SEQ ID NO: 1, or a nucleotide sequence having at least 80%, 85%, 86%, 87%, 88%, 89%, 90%, 91%, 92%, 93%, 94%, 95%, 96%, 97%, 98%, or 99% identity to SEQ ID NO: 1, or a functional fragment or variant thereof.

A disclosed bacterium may, e.g., have been modified to colonize the human gut with increased abundance, stability, predictability or ease of initial colonization relative to a similar or otherwise identical bacterium that has not been modified. For example, a contemplated bacterium may be modified to increase its ability to utilize a privileged nutrient as carbon source. A “privileged nutrient” is defined as a molecule or set of molecules that can be consumed to aid in the proliferation of a particular bacterial strain while providing proliferation assistance to no more than 1% of the other bacteria in the gut. Accordingly, in certain embodiments, a modified bacterium has the ability to consume the privileged nutrient to sustain its colonization and expand in the gut of a subject to a predictably high abundance, even in the absence of other carbon or energy sources, while most other bacteria in the gut of the subject do not. Exemplary privileged nutrients include, e.g., a marine polysaccharide, e.g., a porphyran.

For example, a bacterium may comprise one or more transgenes that increase its ability to utilize a privileged nutrient, e.g., a marine polysaccharide, e.g., a porphyran, as carbon source. In certain embodiments, a bacterium may comprise all or a portion of a polysaccharide utilization locus (PUL), a mobile genetic element that confers the ability to consume a carbohydrate, e.g., a privileged nutrient, upon a bacterium. An exemplary porphyran consumption PUL is the PUL from the porphyran-consuming Bacteroides strain NB001 depicted in SEQ ID NO: 6. Accordingly, in certain embodiments, a modified bacterium comprises SEQ ID NO: 6, or a functional fragment or variant thereof. In certain embodiments, a modified bacterium comprises a nucleotide sequence having at least 80%, 85%, 86%, 87%, 88%, 89%, 90%, 91%, 92%, 93%, 94%, 95%, 96%, 97%, 98%, or 99% identity to SEQ ID NO: 6, or a functional fragment or variant thereof.

Additional exemplary bacterial modifications to increase abundance in the gut of a subject, privileged nutrients, transgenes that increase the ability of a bacteria to utilize a privileged nutrient, PULs, and other methods and compositions for modulating the growth of a modified bacterium are described in International (PCT) Patent Publication No. WO2018112194.

In certain embodiments, a gene in a contemplated bacterium, e.g., a gene encoding a TAA, is operably linked to at least one constitutive promoter, e.g., a phage-derived promoter. Exemplary phage-derived promoters include those comprising the nucleotide sequence of SEQ ID NO: 2, SEQ ID NO: 3, SEQ ID NO: 4, or SEQ ID NO: 5, or a nucleotide sequence having at least 80%, 85%, 86%, 87%, 88%, 89%, 90%, 91%, 92%, 93%, 94%, 95%, 96%, 97%, 98%, or 99% identity to SEQ ID NO: 2, SEQ ID NO: 3, SEQ ID NO: 4, or SEQ ID NO: 5. Additional exemplary phage-derived promoters are described in International (PCT) Patent Publication No. WO2017184565.

Genetically engineered gut bacteria that can be used in the present disclosure may be produced by using any of several methods known in the art, such as, but not limited to, standard recombinant DNA technology. For example, the gene of interest can be delivered to a host bacterium using expression cassettes present in a vector. Expression cassettes typically comprise regulatory sequences functional in host cells into which they are introduced. Regulatory sequences are involved in one or more of the following: regulation of transcription, post-transcriptional regulation, and regulation of translation. Regulatory sequences operably linked to the components can include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like. Expression cassettes can be present in expression vectors and introduced into a wide variety of bacterial host cells.

Vectors useful for transforming bacteria include plasmids, viruses (including phages), and integratable nucleic acid fragments (i.e., fragments integratable into the host genome by homologous recombination). Vectors replicate and function independently of the host genome, or may, in some instances, integrate into the genome itself. Suitable replicating vectors will contain a replicon and control sequences derived from species compatible with the intended expression host cell. In some embodiments, a polynucleotide encoding the gene of interest is operably linked to an inducible promoter, a repressible promoter, or a constitutive promoter. One of ordinary skill in the art can determine the proper regulatory elements to use in a given vector for a given host cell. For a description of various components to be used in a representative commensal bacterium, Bacteroides thetaiotaomicron, see, e.g., the Examples herein, as well as Mimee et al., Cell Syst. (2015) 1:62-71; Lim et al., Cell (2017) 169:547-558. See, also, Wang et al., Microb. Cell Fact. (2018) 17:63 (CRISPR-Cas9 editing of Corynebacterium glutamicum); Wang et al., ACS Synth. Biol. (2016) 15:721-732 (CRISPR-Cas9 editing of Clostridium beijerinckii); Xu et al., Appl. Environ. Microbiol. (2015) (CRISPR-Cas9 editing of Clostridium cellulolyticum); Sun et al., Appl. Microbiol. Biotech. (2015) 99:5151-5162 (GP35-meditated recombineering of Bacillus subtilis). Expression vectors can also include polynucleotides encoding protein tags to aid in purification or selection (e.g., poly-His tags, hemagglutinin tags, fluorescent protein tags, bioluminescent tags, and nuclear localization tags). The coding sequences for such protein tags can be fused to the coding sequences for the tumor antigen or can be included in a separate expression cassette.

Prokaryotic vectors suitable for transforming bacteria are well known in the art. Typically, prokaryotic vectors comprise an origin of replication suitable for the target host cell (e.g., oriC derived from Escherichia coli, pUC derived from pBR322, pSC101 derived from Salmonella, 15 A origin derived from p15 A, and bacterial artificial chromosomes). Vectors can include a selectable marker (e.g., genes encoding resistance for ampicillin, chloramphenicol, gentamicin, and kanamycin). Zeocin™ (Life Technologies, Grand Island, N.Y.) can be used as a selection marker in bacteria, fungi (including yeast), plants, and mammalian cell lines. Accordingly, vectors can be designed that carry only one drug resistance gene for Zeocin for selection work in a number of organisms. Useful promoters are known for expression of proteins in prokaryotes, for example, T5, T7, Rhamnose (inducible), Arabinose (inducible), and PhoA (inducible). Furthermore, T7 promoters are widely used in vectors that also encode the T7 RNA polymerase. Prokaryotic vectors can also include ribosome binding sites of varying strength and secretion signals (e.g., mal, sec, tat, ompC, and pelB). Prokaryotic RNA polymerase transcription termination sequences are also well known (e.g., transcription termination sequences from Streptococcus pyogenes).

General methods for construction of expression vectors are known in the art. Expression vectors for most host cells are commercially available. There are several commercial software products designed to facilitate selection of appropriate vectors and construction thereof, such as bacterial plasmids for bacterial transformation and gene expression in bacterial cells. Methods of introducing plasmids into host cells are known in the art and include, for example conjugation, bacteriophage infection, electroporation, calcium phosphate precipitation, polyethyleneimine-mediated transfection, DEAE-dextran mediated transfection, protoplast fusion, lipofection, liposome-mediated transfection, particle gun technology, direct microinjection, and nanoparticle-mediated delivery.

In one embodiment, a conjugation method is used to transform bacteria. See, e.g., Lederberg et al., Science (1953) 118:169-175. The ability of bacteria to perform conjugation contributes to horizontal gene transfer and thus genome plasticity. In conjugations, plasmid DNA is transferred from one cell to another by a conjugative type IV secretion system (T4SS). See, e.g., Ilangovan et al., Trends Microbiol. (2015) 23:301-310. Conjugative T4SS is a multiprotein secretion apparatus that is found in both gram-negative and gram-positive bacteria. Although the T4SS DNA transfer process in gram-negative and gram-positive organisms is similar, there are also differences that account for the differing physiology between the two main types of bacteria. The conjugation process can occur in three distinct steps. In the first and second steps, DNA is processed and recruited to the T4SS. In the third step, the processed DNA is translocated from one cell to another through the T4SS, which is one type of membrane secretion apparatus. Initiation of conjugation requires the formation of a multiprotein-DNA complex, called the relaxosome, at the origin of transfer (oriT). The enzyme relaxase, together with other accessory proteins, plays a crucial role in guiding the DNA through the T4SS to the recipient cell (see, e.g., Ilangovan et al., Trends Microbiol. (2015) 23:301-310).

In gram-positive bacteria, there are distinct conjugative transfer mechanisms, which include the ability to transfer broad-host-range plasmids, e.g., pIP501, and the Enterococcus sex pheromone-responsive plasmid, pCF10 (see, e.g., Goessweiner-Mohr et al., Microbiol Spectr. (2014) 2:PLAS-0004-2013). Both plasmids mediate single-stranded DNA transfer in gram-positive bacteria. Alternatively, the conjugative transfer system found in Streptomyces mediates double-stranded DNA transfer.

In another embodiment of the present disclosure, the genetic material for use herein is cloned into a bacteriophage (phage) specific for the gut bacterium of interest. Phages for use herein typically include a proteinaceous capsid and tail structure, which together serve to deliver genetic information to the targeted host cell. A phage capable of delivering DNA of non-phage origin to a bacterial cell is referred to as a transducing particle (TP). One of ordinary skill in the art can readily design TPs of specific DNA content such that a particular bacterium can be transduced with the TP. See, e.g., Chung et al., J. Molec. Biol. (1990) 216:911-926 (1990); Chung et al., J. Molec. Biol. (1990) 216:927-938. The process of generating a TP normally involves transferring the DNA packaging-related sequence elements of the phage to a plasmid. The phage DNA packaging machinery recognizes the plasmid as self and loads the non-self DNA into the capsid. The TP can then be used to deliver the plasmid to a target cell population.

For example, phages specific for F. prausnitzii appear to be present in the human gut. See, e.g., Roux et al., PLoS One (2012) 7:e40418. One or more of these phages, or newly identified phages, can be isolated and used for TP production. A TP production methodology specific for F. prausnitzii, or other desired GI bacteria, can be combined with a genome engineering technology to enable the genetic manipulation of the target microorganism.

The bacterium of interest can also be engineered using gene editing methods that utilize programmable nucleases that enable targeted genetic modifications in a host cell genome by creating site-specific breaks at desired locations. Such nucleases include, but are not limited to, Clustered Regularly Interspaced Short Palindromic Repeats (CRISPR)-associated nucleases, zinc-finger nucleases (ZFNs), transcription activator-like effector nucleases (TALENs), and meganucleases. For reviews of these programmable nucleases see, e.g., Kim et al., Nature Reviews Genetics (2014) 15:321-334; Koonin et al., Curr Opin Microbiol. (2017) 37:67-78; Makarova et al., Cell (2017) 168:146; Makarova et al., Cell (2017) 168:328; Hsu et al., Cell (2014) 157:1262-1278; Jore, et al., Nature Struc. and Molec. Biol. (2011) 18:529-536; Urnov et al., Nature Reviews Genetics (2010) 11:636-646; Stoddard, B., Mobile DNA (2014) 5:7; Joung et al., Nature Reviews Molecular Cell Biology (2013) 14:49-55.

For example, a CRISPR guide polynucleotide can be used that preferentially targets a nucleic acid target sequence present in a genomic region in the organism to be modified. The CRISPR guide polynucleotide can be associated with a nucleic acid binding molecule, such as a DNA binding protein, that binds to and cleaves, the target sequence. A polynucleotide donor, e.g., a linear dsDNA donor, that contains the gene of interest and regions of homology to the targeted genetic region, or an expression cassette that includes the heterologous gene of interest, can be delivered for insertion into the cleavage site by homologous recombination. CRISPR methods are well known in the art and are described in, e.g., Koonin et al., Curr Opin Microbiol. (2017) 37:67-78; Makarova et al., Cell (2017) 168:146; Makarova et al., Cell (2017) 168:328; Hsu et al., Cell (2014) 157:1262-1278; Jore, et al., Nature Struc. and Molec. Biol. (2011) 18:529-536; Jinek, et al., Science (2012) 337:816-821; Briner et al., Molecular Cell (2014) 56:333-339; PCT Publication Nos. WO2013/176772 (published 28 Nov. 2013) and WO2014/150624 (published 25 Sep. 2014); and U.S. Pat. Nos. 9,580,701; 9,650,617; 9,688,972; 9,771,601; and 9,868,962.

In some cases, a genomic engineering procedure, such as lambda red recombineering, can be used to genetically modify the host cell genome. Lambda RED recombineering is based on the discovery and implementation of enzymes from the E. coli bacteriophage lambda and provides an efficient method of bacterial genome engineering (see, e.g., Court et al., Annual Review of Genetics (2002) 36:361-388). With lambda RED recombineering, cells are transformed by both a plasmid containing lambda RED recombination enzymes and linear double-stranded DNA (dsDNA) containing homology to the bacterial genome at the targeted genomic change. The lambda RED enzymes are exo, beta, and gam. Gam inhibits the endogenous recombination enzyme RecBCD that is also a highly potent and processive dsDNA exonuclease. Exo is a DNA exonuclease that generates single-stranded DNA (ssDNA) overhangs from the supplied linear dsDNA. Beta binds to ssDNA, and promotes strand invasion and homologous recombination (see, e.g., Court et al., Annual Review of Genetics (2002) 36:361-388; and Sawitzke et al., Methods Enzymol. (2013) 533: 157-177). Beta only requires 30-100 bases of homology for efficient recombination. Therefore, linear dsDNA for recombination can be generated by PCR with primers that contain homologous DNA.

This technology can be coupled with the use of programmable nucleases, as described herein. Typically, this requires the use of two plasmids and linear dsDNA. One plasmid encodes a programmable nuclease, and the other encodes a guide nucleic acid and the lambda RED enzymes. The linear dsDNA contains homology to the bacterial genome and the targeted genetic change. Each plasmid and the linear DNA are transformed into the bacteria sequentially. Other methods for genetically engineering bacteria include standard recombinant DNA techniques, well known in the art.

Bacteria that have been genetically engineered using a method as described herein, are then identified and purified using techniques well known in the art and described in the Examples. For example, integration of an expression vector into a host bacterium can be confirmed by PCR analysis. Successful transformation can be confirmed, e.g., by isolating individual clones and screening the target locus using Sanger sequencing or analyzing cleavage efficiency using a restriction digest-based assay, such as a T7 endonuclease assay or a Surveyor assay. High-throughput screening techniques can also be employed, including, but not limited to, flow cytometry techniques such as fluorescence-activated cell sorting (FACS)-based screening platforms, microfluidics-based screening platforms, emulsion/droplet-based analysis methods, and the like. These techniques are well known in the art (see, e.g., Wojcik et al., Int. J. Molec. Sci. (2015) 16:24918-24945.

The plasmids described above can also contain sequences coding for a selectable marker or an antibiotic resistance gene, such that bacteria including the plasmids can be identified and isolated.

Once isolated, the genetically engineered non-pathogenic, commensal bacteria can be orally delivered to an acceptable animal model for the particular cancer in question to test for efficacy. Such animal cancer models are known in the art and commercially available, e.g., a mouse syngeneic tumor model of a subcutaneous tumor consisting of the mouse melanoma cell line B16F10 expressing ovalbumin (B16-OVA), as described in the Examples.

Genetically engineered bacteria of the present disclosure can be used to treat various types of cancer. Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, B-cell lymphomas, e.g., large B-cell lymphomas, e.g., diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma). Additional exemplary cancers include melanomas, bone cancers, vaginal cancers, brain cancers, spinal cord cancers, oral cancers, parotid tumors, precursor B lymphoblastic leukemia, and B cell prolymphocytic leukemia.

Other cell proliferative disorders can also be treated, including precancerous conditions; hematologic disorders; and immune disorders, such as autoimmune disorders including, without limitation, Addison's disease, celiac disease, diabetes mellitus type 1, Grave's disease, Hashimoto's disease, inflammatory bowel disease, multiple sclerosis, psoriasis, rheumatoid arthritis, scleroderma, and systemic lupus erythematosus.

### Pharmaceutical Compositions

Once produced, bacteria that have been genetically engineered to include genes encoding tumor antigens, such as but not limited to, TAAs, can be formulated into compositions for delivery to the GI tract of the subject to be treated. Compositions include the genetically engineered bacteria and one or more pharmaceutically acceptable excipients. Typically, the compositions are formulated for oral administration. Alternatively, the compositions can be formulated as suppositories, aerosol, intranasal, and sustained release formulations. Methods of preparing such formulations are known, or will be apparent, to one of ordinary skill in the art and may be administered using routes of administration in accordance with any medically acceptable method known in the art. See, e.g., Remington's Pharmaceutical Sciences, Mack Publishing Company, Easton, Pa.

Oral vehicles include typical excipients, e.g., pharmaceutical grades of mannitol, lactose, starch, magnesium, stearate, sodium saccharin cellulose, magnesium carbonate, and the like. These oral compositions may be taken in the form of solutions, suspensions, tablets, pills, capsules, sustained release formulations, or powders, and contain from about 2% to about 95% total weight of the active ingredient per dose, e.g., about 5% to about 90%; about 10% to about 70%; about 20% to about 85%; about 30% to about 75%; or any percentage within these ranges of active ingredient per dose.

For suppositories, the vehicle composition will include traditional binders and carriers, such as, polyalkaline glycols, or triglycerides. Such suppositories may be formed from mixtures containing the active ingredient in the range of about 0.5% to about 10% (w/w), e.g., about 0.75% to about 5%; about 1% to about 2%; or any percentage within these ranges. The percentage will vary depending on the type of base material used and the locus of delivery.

An aerosol composition for inhalation can include the active ingredient in solution or dispersion in a propellant. Many propellants are known and include e.g., hydrocarbons such as n-propane, n-butane or isobutane; or halogen-substituted hydrocarbons. If the active ingredient is present in suspension in the propellant, the aerosol composition can also contain a lubricant and a surfactant. The aerosol composition can contain up to about 5% by weight of the active ingredient e.g., about 0.002% to about 5%; about 0.01% to about 3%; about 0.015% to about 2%; about 0.1% to about 2%; about 0.5% to about 2% or 0.5% to 1%, by weight of the active ingredient, based on the weight of the propellant.

Intranasal formulations will usually include vehicles that neither cause irritation to the nasal mucosa nor significantly disturb ciliary function. Diluents, such as water, aqueous saline, or other known substances, can be employed with the engineered bacteria. The nasal formulations may also contain preservatives and a surfactant may be present to enhance absorption of the active ingredients by the nasal mucosa.

Controlled or sustained release formulations are made by incorporating the active ingredients into carriers or vehicles such as liposomes; nonresorbable impermeable polymers such as ethylenevinyl acetate copolymers and Hytrel® copolymers (DuPont Co., Wilmington, Del.); swellable polymers e.g., hydrogels; or resorbable polymers, such as collagen and certain polyacids or polyesters, such as those used to make resorbable sutures.

Compositions of the present disclosure can also include an antimicrobial agent for preventing or deterring unwanted microbial growth. Antioxidants can also be present in the compositions. Antioxidants are used to prevent oxidation, thereby preventing the deterioration of the components of the preparation.

The components of the compositions of the present disclosure will vary depending on a number of factors. The compositions will include a therapeutically effective amount of the engineered bacteria as described below.

The amount of any individual excipient in the pharmaceutical composition will vary depending on the nature and function of the excipient and particular needs of the composition. Typically, the optimal amount of any individual excipient is determined through routine experimentation, e.g., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, such as from about 5% to about 98% by weight, from about 15% to about 95% by weight of the excipient, such as concentrations less than 30% by weight. The foregoing pharmaceutical excipients along with other excipients are described in, for example, Remington's Pharmaceutical Sciences, Mack Publishing Company, Easton, Pa.; Williams & Williams, The Physician's Desk Reference, Medical Economics, Montvale, N.J.; Kibbe, A. H., Handbook of Pharmaceutical Excipients, American Pharmaceutical Association, Washington, D.C.

The compositions herein may optionally include one or more additional agents, such as other medications used to treat a subject for the condition in question. For example, chemotherapeutics, anti-tumor immunotherapeutics, and checkpoint inhibitors as described herein.

The pharmaceutical compositions may be in unit doses, bulk packages (e.g., multi-dose packages), or subunit doses or in any convenient, appropriate packaging. For example, ampules with non-resilient, removable closures (e.g., sealed glass) or resilient stoppers are most conveniently used for liquid formulations. If the genetically engineered bacterial compositions are provided as a dry formulation (e.g., freeze dried or a dry powder), a vial with a resilient stopper can be used, so that the compositions may be easily resuspended by injecting fluid through the resilient stopper. A suitable set of physician and/or patient instructions relating to the use of the pharmaceutical compositions may be included, with information as to dosage and dosing schedule and other relevant information.

Contemplated bacteria may be used in disclosed compositions in any form, e.g., a stable form, as known to those skilled in the art, including in a lyophilized state (with optionally one or more appropriate cryoprotectants), frozen (e.g., in a standard or super-cooled freezer), spray dried, and/or freeze dried. A “stable” formulation or composition is one in which the biologically active material therein essentially retains its physical stability, chemical stability, and/or biological activity upon storage. Stability can be measured at a selected temperature and humidity conditions for a selected time period. Trend analysis can be used to estimate an expected shelf life before a material has actually been in storage for that time period. For live bacteria, for example, stability may be defined as the time it takes to lose 1 log of cfu/g dry formulation under predefined conditions of temperature, humidity and time period.

A bacterium disclosed herein may be combined with one or more cryoprotectants. Exemplary cryoprotectants include fructoligosaccharides (e.g., Raftilose©), trehalose, maltodextrin, sodium alginate, proline, glutamic acid, glycine (e.g., glycine betaine), mono-, di-, or polysaccharides (such as glucose, sucrose, maltose, lactose), polyols (such as mannitol, sorbitol, or glycerol), dextran, DMSO, methylcellulose, propylene glycol, polyvinylpyrrolidone, non-ionic surfactants such as Tween 80, and/or any combinations thereof

### Administration

At least one therapeutically effective cycle of treatment with the composition will be administered to a subject. By “therapeutically effective cycle of treatment” is intended a cycle of treatment that, when administered, brings about a positive therapeutic response, e.g., the individual undergoing treatment displays reduced tumor size; reduced amount or frequency of symptoms associated with the cancer in question; inhibited progression of the cancerous state, such as by a reduction or elimination of metastasis; slowed rate of progression of the particular cancer; and the like.

In certain embodiments, multiple therapeutically effective doses of compositions will be administered. As explained herein, the pharmaceutical compositions of the present disclosure are typically administered by oral delivery (including buccal and sublingual administration). The pharmaceutical preparation can be in the form of a liquid solution or suspension immediately prior to administration.

The actual dose to be administered will vary depending upon the age, weight, and general condition of the subject as well as the severity of the condition being treated, the judgment of the health care professional, and particular cells and compositions being administered. Therapeutically effective amounts can be determined by one of ordinary skill in the art, and will be adjusted to the particular requirements of each particular case. Generally, a therapeutically effective amount for the use of live bacteria will be measured as colony forming units (CFU). Typically, a dose will include from about 1×10⁵ to about 1×10¹⁴ colony-forming units (CFU) of the engineered bacteria e.g., 1×10⁶ to about 1×10¹¹; about 1×10⁷ to about 1×10¹⁰; about 1×10⁶ to about 1×10⁹, CFU of the genetically engineered bacteria; or any amount within the stated ranges. A therapeutically effective dose can be determined experimentally by repeated administration of increasing amounts of the composition in order to determine which amount produces a clinically desired endpoint.

Administration can be in a single bolus dose, or can be in two or more doses, such as one or more days apart. The amount of composition administered will depend on the potency of the specific composition, the tumor antigen that is being used to treat the patient, and the route of administration.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein, is administered in combination with one or more additional therapies, e.g., anti-tumor agents, cell therapies, checkpoint inhibitors, and the like. For example, various anti-cancer drugs are known and include, without limitation, alkylating agents (cisplatin, chlorambucil, procarbazine, carmustine etc.), antimetabolites (methotrexate, cytarabine, gemcitabine etc.), anti-microtubule agents (vinblastine, paclitaxel etc.), topoisomerase inhibitors (etoposide, doxorubicin etc.), and cytotoxic agents (bleomycin, mitomycin etc.). The appropriate chemotherapeutic drug to be used will depend on the type of cancer being treated and the tolerance the patient has to the therapeutic agent.

Cell therapies can also be used in conjunction with the genetically engineered bacteria described herein, and include, but are not limited to, bone marrow transplants, stem cell transplants, and adoptive cell therapies (ACTs). ACTs use genetically modified adoptive cells derived from either a specific patient returned to that patient (autologous cell therapy) or from a third-party donor (allogeneic cell therapy) to treat the patient. ACTs, include, but are not limited to, T cell therapies, CAR-T cell therapies, and natural killer (NK) cell therapies.

For example, T cells and NK cells can be modified to produce chimeric antigen receptors (CARs) on the T or NK cell surface (CAR-T cells and CAR-NK cells, respectively). These CAR-T cells recognize specific soluble antigens or antigens on a target cell surface, such as a tumor cell surface, or on cells in the tumor microenvironment. The CAR can comprise one or more extracellular ligand binding domains, a hinge region, a transmembrane region, and an intracellular signaling region. The extracellular ligand binding domain typically comprises a single-chain immunoglobulin variable fragment (scFv) or other ligand binding domain. The hinge region generally comprises a polypeptide hinge of variable length such as one or more amino acids, a CD8 alpha or an IgG4 region (or others), and combinations thereof. The transmembrane domain typically contains a transmembrane region derived from CD8 alpha, CD28, or other transmembrane proteins and combinations thereof. The intracellular signaling domain can consist of one or more intracellular signaling domains such as CD28, 4-1BB, CD3 zeta, OX40, or other intracellular signaling domains, and combinations thereof. When the extracellular ligand binding domain binds to a cognate ligand, the intracellular signaling domain of the CAR activates the lymphocyte. See, e.g., Brudno et al., Nature Rev. Clin. Oncol. (2018) 15:31-46; Maude et al., N. Engl. J. Med. (2014) 371:1507-1517; Sadelain et al., Cancer Disc. (2013) 3:388-398 (2018); U.S. Pat. Nos. 7,446,190 and 8,399,645 for descriptions of CAR-T cells, methods of making the same, and uses thereof. Examples of CAR-T approved therapies include e.g., the use of axicabtagene ciloleucel (Yescarta®; Kite Pharma, Inc., Foster City, Calif.); and tisagenlecleucel (Kymriah®; Novartis Pharmaceutical Corporation, Hanover, N.J.).

In certain embodiments, a method or composition described herein is administered in combination with a checkpoint inhibitor. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist.

In certain embodiments, the checkpoint inhibitor is a PD-1 or PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody based therapeutics. Exemplary treatment methods that employ PD-1/PD-L1 based immune checkpoint inhibition are described in U.S. Pat. Nos. 8,728,474 and 9,073,994, and EP Patent No. 1537878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), durvalumab (AstraZeneca), MEDI4736, avelumab, and BMS 936559 (Bristol Myers Squibb Co.).

In certain embodiments, a method or composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

These and other checkpoint inhibitors can be used to treat various cancers, such as, but not limited to melanoma; lung cancer; kidney cancer; bladder cancer; stomach cancer; head and neck cancer; lymphomas, such as Hodgkin lymphoma; and solid tumors.

If the agents and therapies described above are provided at the same time as the genetically engineered bacteria described herein, the engineered bacteria can be provided in the same or in a different composition. Thus, the genetically engineered bacteria and other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising genetically engineered organisms and a dose of a pharmaceutical composition comprising at least one other therapeutic agent, such as an anti-tumor agent, checkpoint inhibitor, and the like, which in combination comprises a therapeutically effective dose, according to a particular dosing regimen. Similarly, the genetically engineered microorganisms and therapeutic agents can be administered in at least one therapeutic dose. Administration of the separate pharmaceutical compositions can be performed simultaneously or at different times (e.g., sequentially, in either order, at the same time, or at different times), as long as the administration results in the therapeutic effect of the combination of these compositions.

Contemplated methods may further comprise administrating a privileged nutrient to the subject to support colonization of the bacterium. Exemplary privileged nutrients include marine polysaccharides, e.g., a porphyran. For example, a disclosed privileged nutrient may be administered to the subject prior to, at the same time as, or after a disclosed bacterium.

Contemplated methods may comprise administration of a disclosed bacterium or pharmaceutical composition to a subject every 12 hours, 24 hours, day, 2 days, 3 days, 4 days, 5 days, 6 days, 7 days, week, 2 weeks, 3 weeks, 4 weeks, month, 2 months, 3 months, 4 months, 5 months, or 6 months. In certain embodiments, the time between consecutive administrations of a disclosed bacterium or pharmaceutical composition to a subject is greater than 12 hours, 24 hours, 36 hours, 48 hours, 2 days, 3 days, 4 days, 5 days, 6 days, 7 days, 1 week, 2 weeks, 3 weeks, or 4 weeks.

### EXPERIMENTAL

Aspects of the present disclosure are further illustrated in the following Examples. Efforts have been made to ensure accuracy with respect to numbers used (e.g., amounts, concentrations, percent changes, etc.) but some experimental errors and deviations should be accounted for. Unless indicated otherwise, temperature is in degrees Centigrade and pressure is at or near atmospheric. It should be understood that these Examples, while indicating some embodiments of the present disclosure, are given by way of illustration only and are not intended to limit the scope of the disclosure.

### Example 1: Plasmid Construction for Preparing a Genetically Engineered Non-Pathogenic, Commensal Bacterium

A plasmid was constructed for generating a genetically engineered Bacteroides thetaiotaomicron strain that expressed ovalbumin (a model tumor-associated antigen). This and other conjugation plasmids can be used to transform other commensal strains of bacteria, in addition to B. thetaiotaomicron, such as other non-pathogenic, commensal bacteria as described herein. Additionally, other tumor antigen genes can be used.

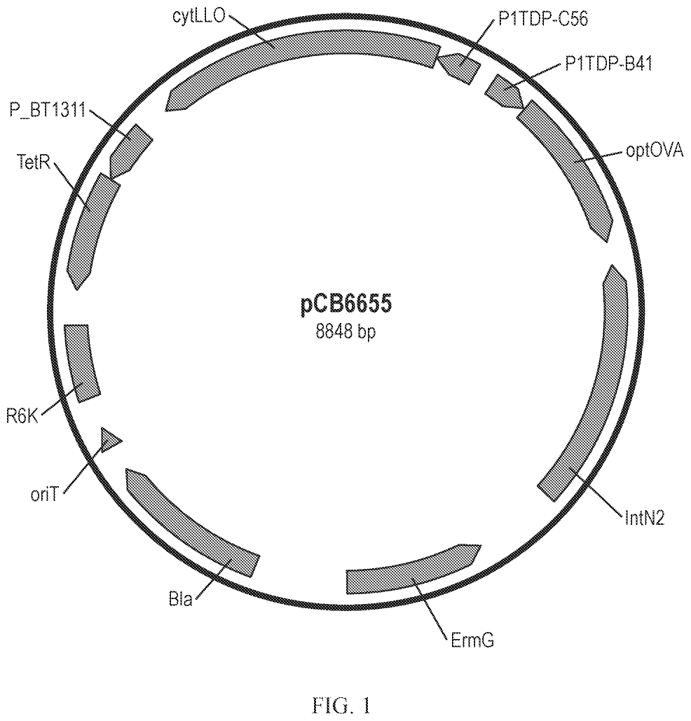

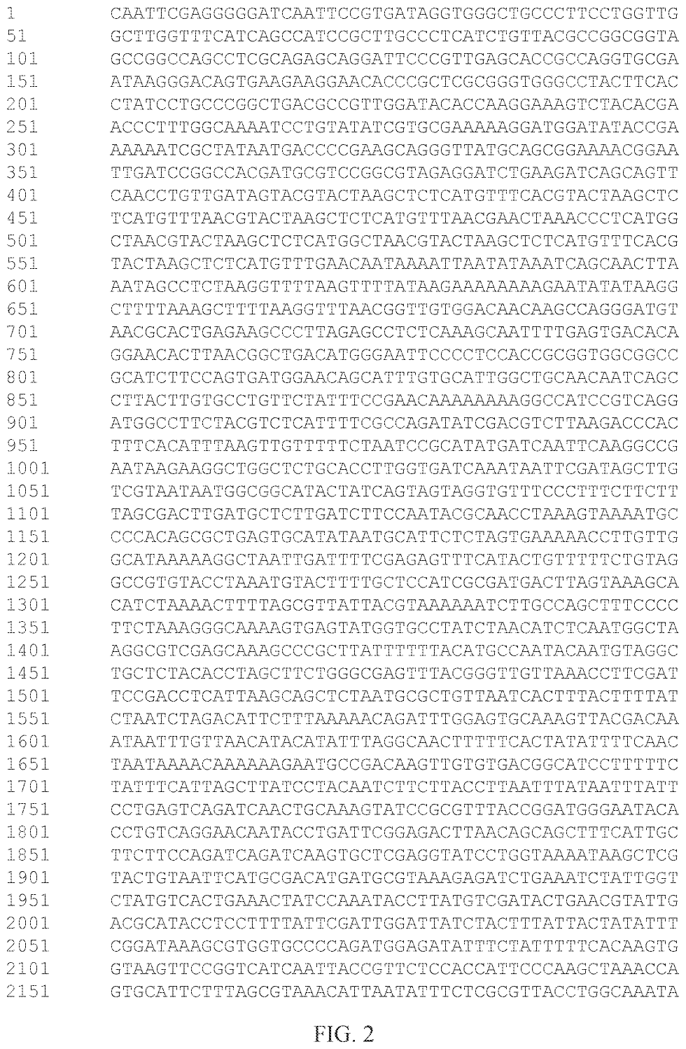

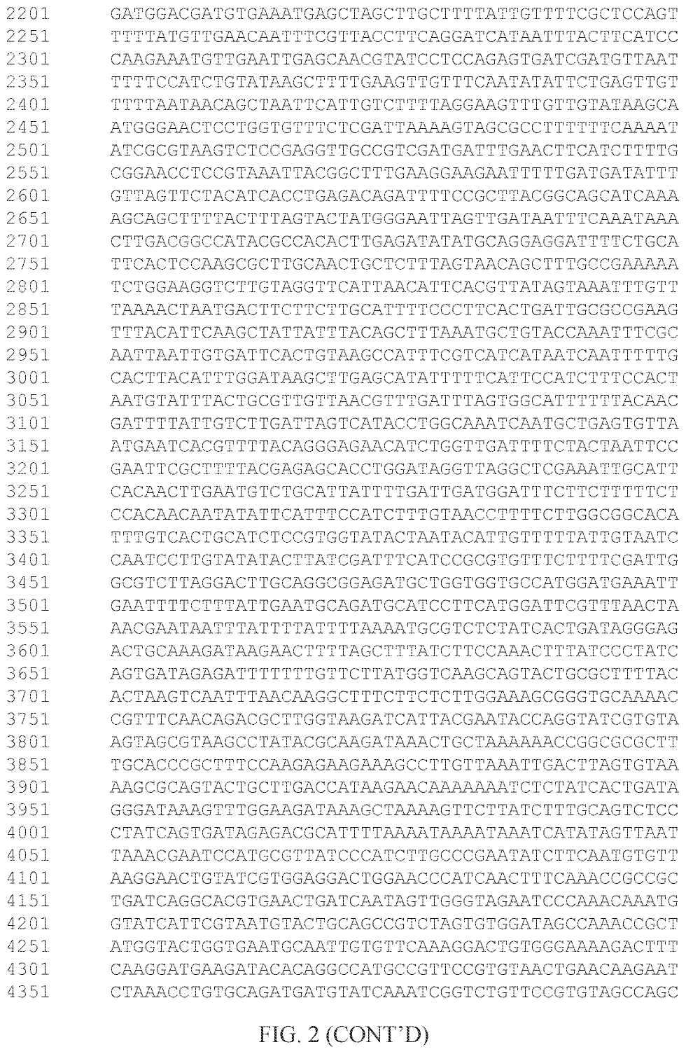

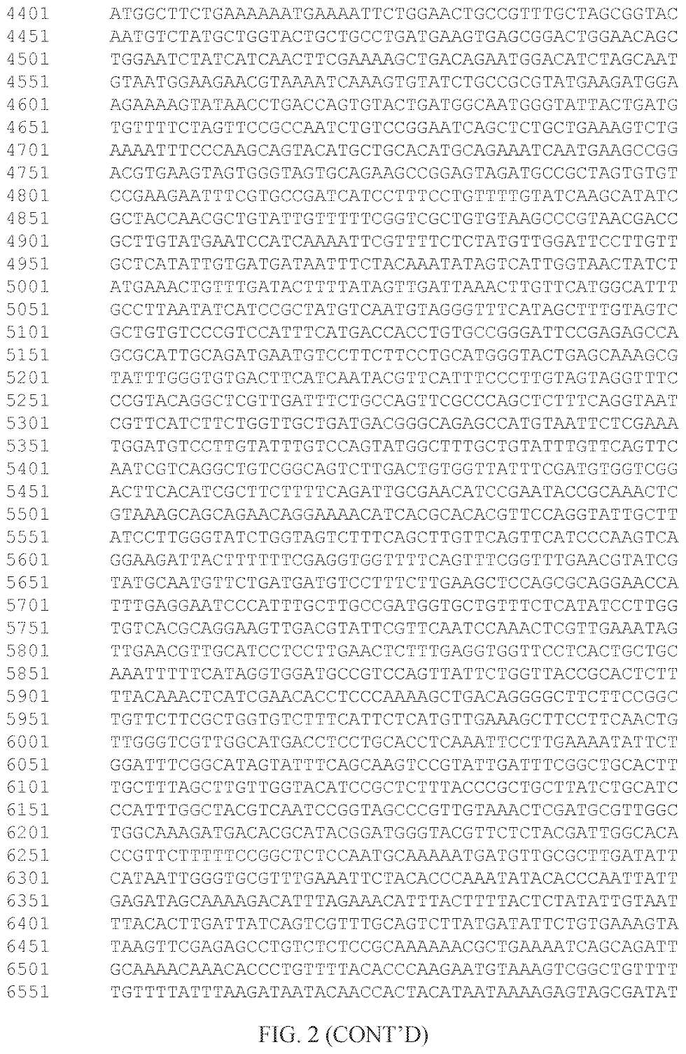

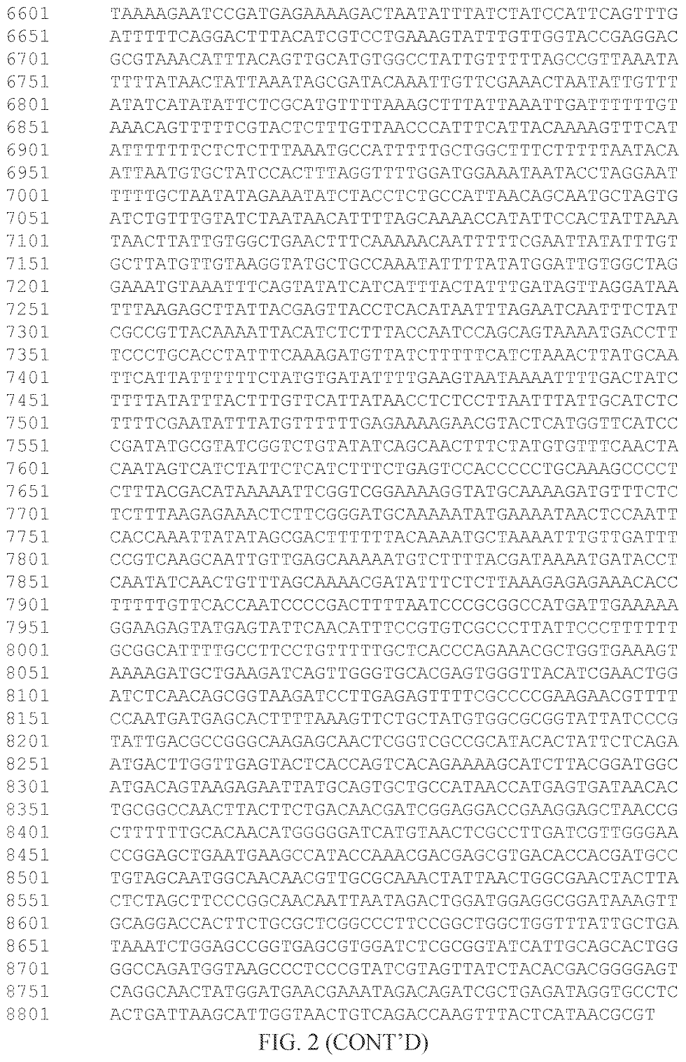

The following describes plasmid pCB6655 for use in genetically modifying bacteria. A diagram of the plasmid is shown in FIG. 1 and the sequence of plasmid pCB6655 is shown in FIG. 2. The Bacteroides integrase gene (IntN2; see, e.g., Wang et al., J. Bacteriol. (2000) 182:3559-3571) was included to catalyze the integration of the plasmid into one of the two attBT2 sites in the B. thetaiotaomicron genome (see, e.g., Wang et al., J. Bacteriol. (2000) 182:3559-3571). The native strong constitutive promoter, BT1311 (see, e.g., Mimee et al., Cell Syst. (2015) 1:62-71), was used to drive expression of the Tet repressor (TetR) to enable controlled gene expression. The Listeria monocytogenes endogenous hly gene sequence encoding listeriolysin O (LLO) (see, e.g., Moors et al., Infect. Immun. (1999) 67:131-139) that lacked the N-terminal secretion sequence signal (deletion of amino acids 2-24) (cytLLO) was cloned under a medium strength aTc-inducible promoter, P1T_{DP}-C56 (see, e.g., Lim et al., Cell (2017) 169:547-558). Expression of the ovalbumin (OVA) gene from Gallus, lacking the N-terminal secretion signal (deletion of amino acids 2-110) and codon-optimized for expression in B. thetaiotaomicron (optOVA), was driven by a strong aTc-inducible promoter, P1T_{DP}-B41 (see, e.g., Lim et al., Cell (2017) 169:547-558), in opposite orientation to cytLLO.

### Example 2: Preparation of a Genetically Engineered Non-Pathogenic, Commensal Bacterium

The following Example describes the generation of a human commensal bacteria that expresses a model tumor-associated antigen, ovalbumin.

The pCB6655 construct was conjugated into B. thetaiotaomicron using E. coli MFDpir strain (see, e.g., Ferrières et al., J. Bacteriol. (2010) 192:6418-6427). For mating, overnight cultures of B. thetaiotaomicron and E. coli MFDpir were diluted back and grown to an OD₆₀₀ of 0.2-0.3 and 0.5-0.7, respectively. B. thetaiotaomicron was added to E. coli MFDpir at a ratio of 5:1 (v/v). The mating mixture was pelleted, resuspended in 20 μl of brain-heart-infusion (BHI) media supplemented with hemin and L-cysteine (BHIS media), spotted onto a BHI agar plate and incubated aerobically at 37° C. overnight. Cells were then collected by scraping, resuspended in 1 ml BHIS, plated as serial dilutions on BHI agar plates containing 200 μg/ml gentamicin (Gm) and 25 μg/ml erythromycin (Erm), and incubated anaerobically at 37° C. for 2 days. Resultant colonies (B. theta-OVA) were re-isolated in BHI+Gm+Erm plates, and integration of the pCB6655 plasmid was confirmed by PCR analysis. The sequence was verified by whole-genome sequencing.

### Example 3: Production of a Murine Tumor Model

The following Example describes the generation of an in vivo murine tumor model using the murine melanoma cell line B16-OVA and ovalbumin-specific T cells derived from an OT-1 transgenic mouse.

### Ovalbumin-Specific OT-1 CD8+ T Cell Isolation and Activation

The evening before isolation of T cells, a 96-well round bottom tissue culture-treated plate was coated with 50 μl/well 1 μg/ml anti-CD3 (BD Biosciences, San Jose, Calif.; Catalog No. BDB553058). Spleens were harvested from OT-1 transgenic mice (C57BL/6-Tg(TcraTcrb)1100Mjb/J from The Jackson Laboratory, Bar Harbor, Me.). Spleens were then crushed over a 70 micron filter in 2% PBS with 1 mM EDTA to generate a single cell suspension. Cells were pelleted by centrifugation at 1500 rpm for 5 minutes at 4° C., and red blood cells were lysed with eBioscience™ RBC lysis buffer (ThermoFisher Scientific, Waltham, Mass.). The cells were washed once in 2% PBS with 1 mM EDTA, counted, and resuspended to 10×10⁸/ml. Isolation of total CD8+ T cells was performed using EasySep™ Mouse CD8+ T cell Isolation Kit (StemCell Technologies, Vancouver, BC, Canada; Catalog No. 19853) according to the manufacturer's protocol. Isolated CD8+ T cells were pelleted by centrifugation at 1500 rpm for 5 minutes at 4° C. and resuspended to 1×10⁶/ml in complete DMEM media (cDMEM, 10% heat-inactivated FBS, glutamax, sodium pyruvate, 1 mM HEPES, non-essential amino acids, 50 mM β-mercaptoethanol, penicillin/streptomycin). Each well coated with anti-CD3 of the 96-well plate was washed twice with 100 μl of PBS. 1×10⁵ cells were added to each well in 100 μl volume. An additional 100 μl of 4 μg/ml anti-CD28 (BD Biosciences, San Jose, Calif.; Catalog No. BDB553295) in cDMEM was added to each well for a final concentration of 2 μg/ml anti-CD28. Cells were grown at 37° C., 5% CO₂ for 3 days. On day 3 post-activation, every 3 wells of cells were transferred into a well of a 6-well plate with 3 ml of cDMEM with 20 ng/ml recombinant mouse IL2 (R&D Systems, Minneapolis, Minn.; Catalog No. 402ML020). T cells were collected and used for adoptive transfer on day 5 or 6 post activation.

### B16-OVA Murine Tumor Model

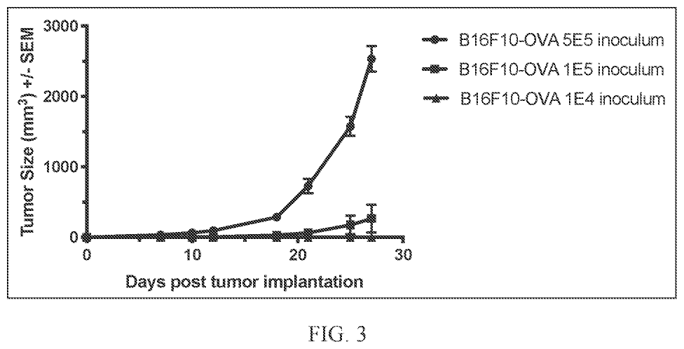

B16-OVA cells, a variant of the B16F10 murine melanoma cell line that overexpresses ovalbumin (see, e.g., Moore et al., Cell (1988) 54:777-785), were maintained in 10% FBS DMEM and 4 mg/ml geneticin. In the passage prior to harvesting cells for subcutaneous injections, cells were grown without geneticin. Cells were liberated via TrypLE™ (ThermoFisher Scientific, Waltham, Mass.), washed twice with pre-warmed PBS, counted, and resuspended to a final volume of 100 μl per dose. To determine the best inoculum to establish the subcutaneous tumor model, three groups of 10 animals/group of eight-week-old female C57BL/6 mice were implanted with 5×10⁵, 1×10⁵, and 4×10⁵ B16-OVA, respectively. B16-OVA was injected subcutaneously into the right flank of the mice. Tumor size was determined by caliper measurements and calculated as length×(width) 2/2. Body weight and tumor size were measured twice weekly until tumors reached 2000 mm³ (at approximately 21 days after implantation). Results from this study are shown in FIG. 3. Based on the results, subsequent experiments used 5×10⁵ inoculum.

### Example 4: Adoptive T Cell Transfer in B16-OVA Model

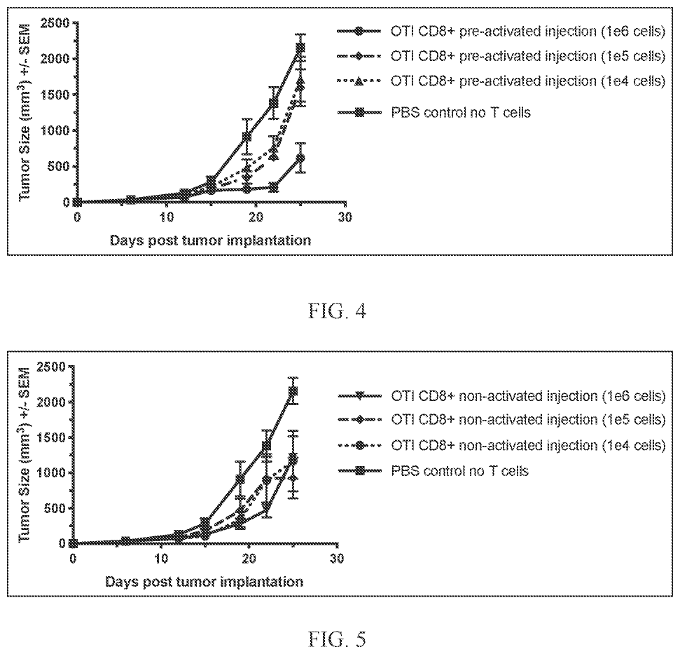

In this study, seven groups of 10 eight-week-old female C57BL/6 mice were implanted with 5×10⁵ B16-OVA, administered subcutaneously into the right flank. Seven days post tumor engraftment, 200 μl of non-activated or activated ovalbumin-specific OT-1 CD8+ T cells (C57BL/6-Tg(TcraTcrb)1100Mjb/J from The Jackson Laboratory, Bar Harbor, Me.) were adoptively transferred intravenously. See Table 1 for the study design. T cell inocula tested were 1×10⁶, 1×10⁵, or 1×10⁴ per mouse, as specified in Table 1. Non-activated T cells were isolated from fresh spleens as described above on the day of adoptive T cell transfer. Non-activated and activated T cells were washed twice with pre-warmed PBS before resuspending to the appropriate concentration for IV injection. Body weight and tumor size were measured twice weekly until tumors reached 2000 mm³ or twice the median survival for responders. Results from this study are shown in FIGS. 4 and 5. Based on the results, subsequent experiments use 1×10⁵ activated T cells inoculum as this inoculum showed partial tumor control.

TABLE 1
Experimental design of adoptive T
cell transfer in the B16-OVA model

| | No. of | | Pre- | Inoculum (No. |
|---|---|---|---|---|
| Group | animals/ | T cells for adoptive | activation of | of OTI cells |
| number | group | transfer | T cells | transferred) |
| 1 | 10 | OT-1 CD8+ | Yes | 1 × 10⁶ |
| 2 | 10 | OT-1 CD8+ | Yes | 1 × 10⁵ |
| 3 | 10 | OT-1 CD8+ | Yes | 1 × 10⁴ |
| 4 | 10 | OT-1 CD8+ | No | 1 × 10⁶ |
| 5 | 10 | OT-1 CD8+ | No | 1 × 10⁵ |
| 6 | 10 | OT-1 CD8+ | No | 1 × 10⁴ |
| 7 | 10 | PBS (vehicle control) | No | — |

### Example 5: Oral Delivery Versus Intravenous Delivery of Engineered Bacteria Expressing Ovalbumin in the B16-OVA Model

To compare the effectiveness of oral delivery with intravenous delivery of bacteria, the following experiment can be done. 5×10⁵ B16-OVA cells can be injected subcutaneously into the right flank of a mouse. Seven days post-tumor engraftment, 200 μl containing 1×10⁵ pre-activated OT-1 T cells can be transferred intravenously. Two days after T cell transfer, engineered bacteria expressing ovalbumin can be administered either intravenously (1×10⁴, 1×10⁵, or 1×10⁶ CFU/dose), or by oral gavage (1×10⁸, 1×10⁹, or 1×10¹⁰ CFU/dose). Listeria monocytogenes expressing ovalbumin (Lm-OVA) is an attenuated (deleted in the InlB and ActA virulence factor genes) pathogenic bacterium microbe (see, e.g., Wood et al., Front CellInfect. Microbiol. (2014) 4:51) that is administered through intravenous injection and used to deliver ovalbumin to enhance an anti-tumor response against B16-OVA. As such, one of the test conditions can be intravenous injection of Lm-OVA, which serves as a control and benchmark for an anti-tumor response against B16-OVA. Lm-OVA can also be administered orally to compare the two routes of delivery. B. theta-OVA, administered intravenously or orally, can serve as the commensal bacteria to be tested in delivery of ovalbumin to augment an anti-tumor response. Body weight and tumor size can be measured twice weekly until tumors reach 2000 mm³ or twice the median survival for responders.

Bacteria can be prepared by picking a colony from a BHI plate into LB or BHIS media for Lm-OVA and B. theta-OVA, respectively. Lm-OVA overnight cultures can be grown aerobically at 30° C. without shaking. The next morning, Lm-OVA can be subcultured at 1:10 dilution in LB media and grown aerobically at 37° C. with shaking. Cells can be pelleted when the OD₆₀₀ reaches log phase (0.5-1) and resuspended in 9% glycerol in PBS. Inocula can be generated and plated to determine exact CFU/dose. Inocula can be kept frozen at −80° C. until ready for use.

B. theta-OVA overnight cultures can be grown anaerobically at 37° C. without shaking. The next morning B. theta-OVA can be diluted 1:100 in BHIS and grown to stationary phase. The bacteria can then be pelleted and resuspended in 12% glycerol in PBS. Inocula can be generated and plated to determine exact CFU/dose. Inocula can be kept frozen at −80° C. until ready for use.

This model may also be used to test for the response of CD4+ T cells (using T cells derived from OT-II transgenic mice (B6.Cg-Tg(TcraTcrB)425Cbn/J from The Jackson Laboratory, Bar Harbor, Me.) to specific antigens of interest. Additionally, adoptive transfer of OT-1 CD8+ T cells may also be omitted if testing for the effect of the bacterial therapeutic in conjunction with the endogenous T cell response to B16-OVA.

### Example 6: Oral Delivery Versus Intravenous Delivery of Engineered Bacteria Expressing Ovalbumin in the B16-OVA Model

The following Example describes the anti-tumor efficacy of orally delivered Lm-OVA vs. intravenously delivered Lm-OVA.

Lm-OVA bacteria (as described in Example 5) were first generated by making overnight starter culture of the appropriate strain (BHI media+200 ug/ml streptomycin), inoculating into 1 L BHI subculture the next day, washing, resuspending in glycerol+PBS, dividing into single dose aliquots, and storing at −80° C. Expression of ovalbumin was verified from cultures by PCR and Western Blot, the latter using an antibody detecting the first 100 amino acids of ActA (which is fused to ovalbumin). Based on dilution plating to estimate cfu/mL, 1e11 cfu/mL inoculums were used to dose animals.

B16-OVA cells were prepared for tumor engraftment by thawing a vial of B16-OVA cells (3.6e6 cells), seeding in T175 flask along with G418 and IFNg, and passaging appropriately to confluency, seeding, trypsinizing, and growing until tumor engraftment at 6e5 tumor cells per mouse (8 week old, female C57BL/6 mice).

Tumors were engrafted on Day 0 to the following groups: no treatment control (G1), intravenous dose of 1×10⁴ CFU Lm-OVA (Lm IV low, G2), intravenous dose of 1×10⁵ CFU Lm-OVA (Lm IV med, G3), intravenous dose of 1×10⁶ CFU Lm-OVA (Lm IV high, G4), oral dose of 1×10⁸ CFU Lm-OVA (Lm Oral low, G5), oral dose of 1×10⁹ CFU Lm-OVA (Lm Oral med, G6), and oral dose of 1×10¹⁰ CFU Lm-OVA (Lm Oral high, G7). Lm-OVA intravenous groups were dosed on day 3. Lm-OVA oral groups were dosed on day 3 and continuously thereafter 2× per week. There were 12 mice per group. Tumor volume and body weight were measured twice weekly, with endpoints of tumor volume (2000 mm³) or median survival (2× median; tumor volume<2000 mm³).

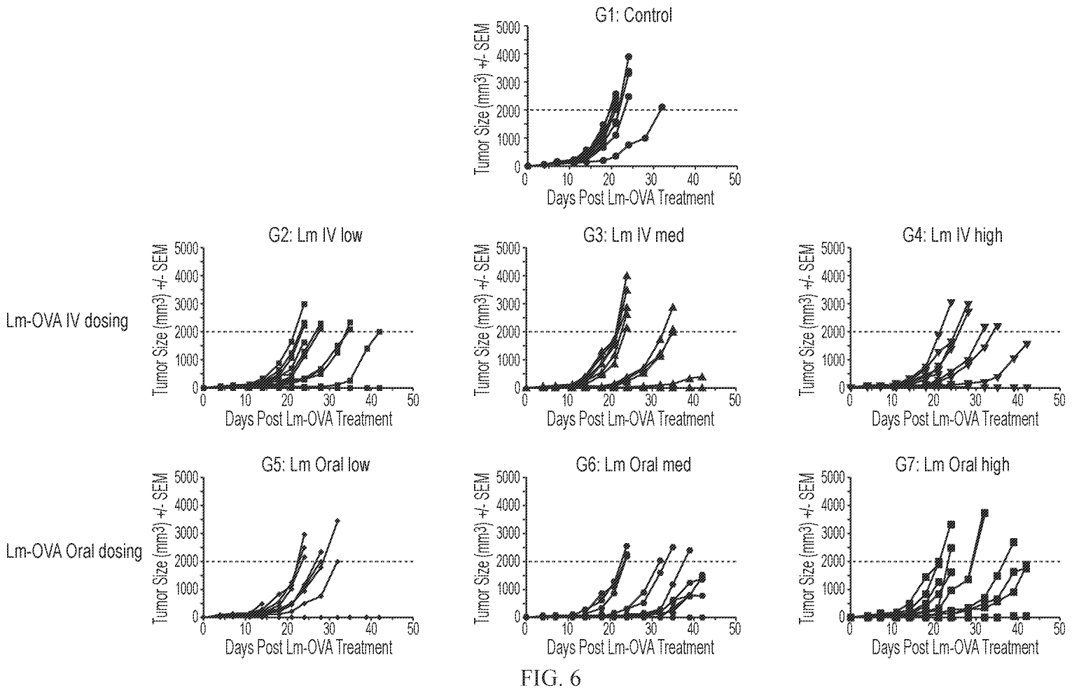

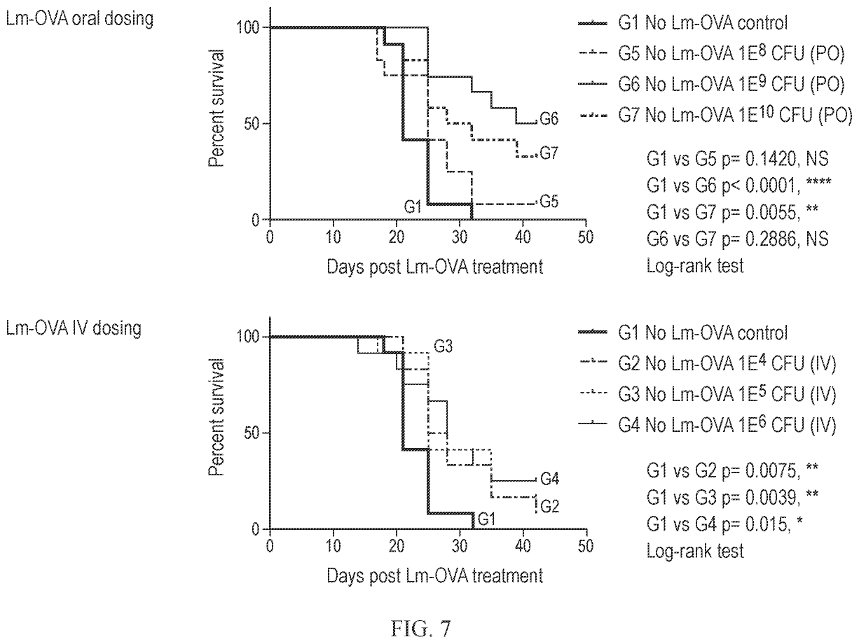

Results are shown in FIG. 6 (tumor growth curves) and FIG. 7 (Kaplan-Meier survival plots). Together, the results demonstrate that orally administered bacteria expressing OVA were more effective at controlling tumor growth and improving mouse survival than IV administered bacteria.

### Example 7: Oral Delivery of Engineered Bacteria Expressing Ovalbumin in Combination with Checkpoint Inhibitor in the B16-OVA Model

The following Example describes the anti-tumor efficacy of orally delivered Lm-OVA+anti-PD1 vs Lm-parent (non-OVA expressing)+anti-PD1 in a B16-OVA mouse model.

Lm-OVA (as described in Example 5) and Lm parental bacteria were first generated by making overnight starter culture of the appropriate strain (BHI media+200 ug/ml streptomycin), inoculating into 1 L BHI subculture the next day, washing, resuspending in glycerol+PBS, dividing into single dose aliquots, and storing at −80° C. Expression of ovalbumin was verified from cultures by PCR and Western Blot, the latter using an antibody detecting the first 100 amino acids of ActA (which is fused to ovalbumin). Based on dilution plating to estimate cfu/mL, 1×10¹¹ cfu/mL inoculums were used to dose animals.

B16-OVA cells were prepared for tumor engraftment by thawing a vial of B16-OVA cells (3.6e6 cells), seeding in T175 flask along with G418 and IFNg, and passaging appropriately to confluency, seeding, trypsinizing, and growing until tumor engraftment at 6e5 tumor cells per mouse (8 week old, female C57BL/6 mice).

Tumors were engrafted on Day 0 and randomized on Day 9 to the following groups: No treatment control (G1), aPD1 (G2), Lm parental (G3), aPD1+Lm parent (G4), Lm-OVA (G5), and aPD1+Lm-OVA (G6). Lm-OVA and Lm-parent groups were dosed PO on Day 10 (1×10¹⁰ cfu per mouse) and continuously thereafter 2× per week, and aPD1 (200 μg per mouse) was dosed IP on Days 11, 14, and 17. Tumor volume and body weight were measured twice weekly, with endpoints of tumor volume (2000 mm³) or median survival (2× median; tumor volume <2000 mm³).

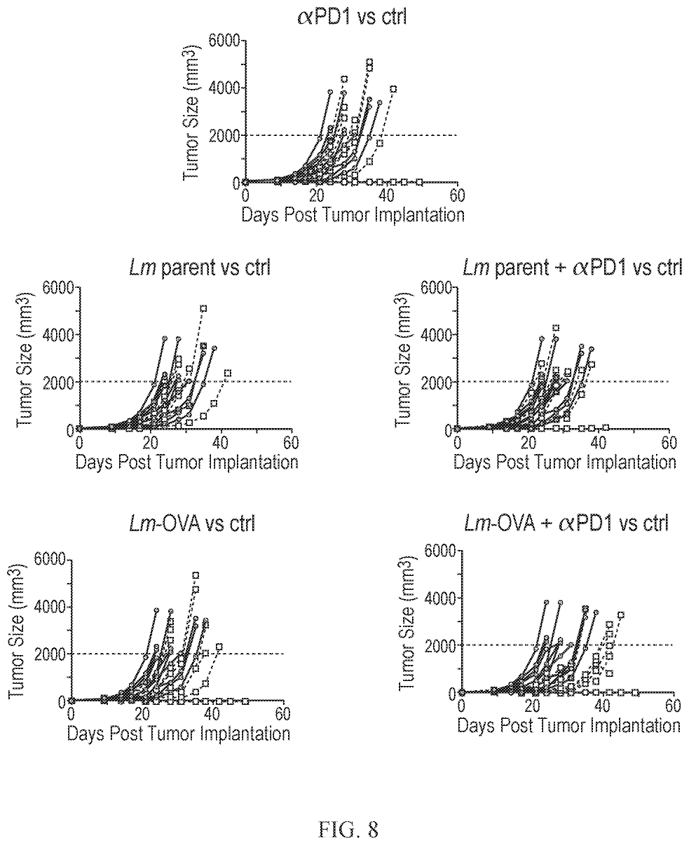

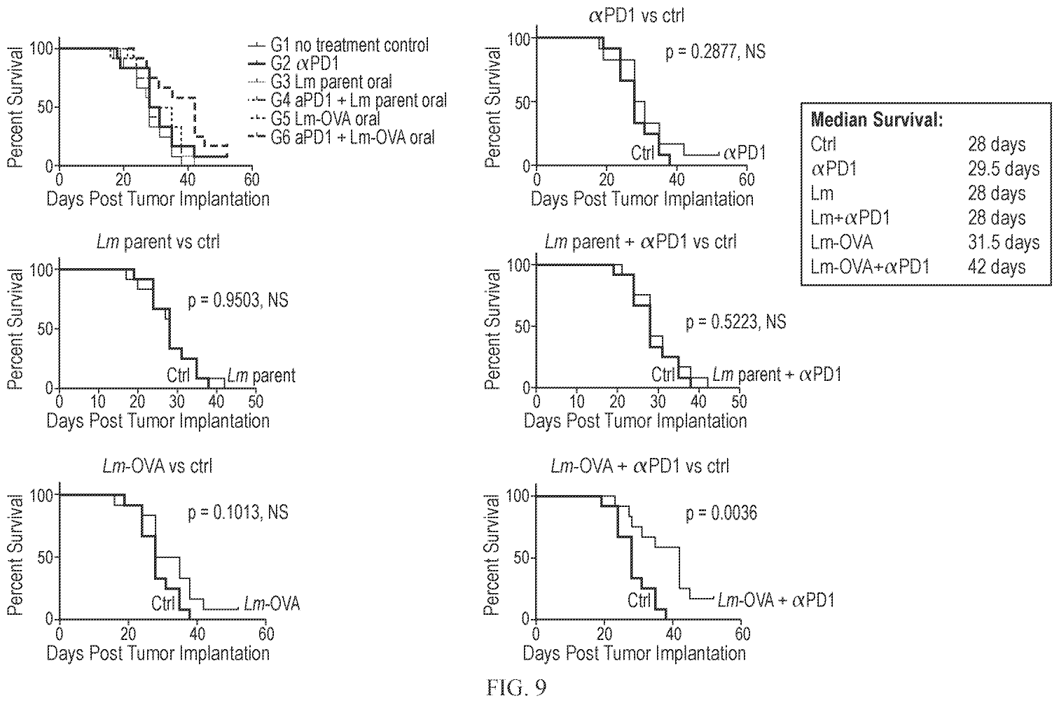

Results are shown in FIG. 7 (tumor growth curves) and FIG. 8 (Kaplan-Meier survival plots). Together, the results demonstrate that Lm-OVA+aPD1 was the most effective at tumor control.

### Example 8: Oral Delivery Versus Intravenous Delivery of Engineered Bacteria Expressing Ovalbumin in the B16-OVA Model

The following Example describes testing for the anti-tumor efficacy of orally delivered B. theta-OVA vs. intravenously delivered B. theta-OVA.

B16-OVA cells are prepared for tumor engraftment by thawing a vial of B16-OVA cells (3.6e6 cells), seeding in T175 flask along with G418 and IFNg, and passaging appropriately to confluency, seeding, trypsinizing, and growing until tumor engraftment at 6e5 tumor cells per mouse (8 week old, female C57BL/6 mice).

Tumors are engrafted on Day 0 to the following groups: no treatment control, intravenous dose of 1×10⁴ CFU B. theta-OVA (as described in Example 2), intravenous dose of 1×10⁵ CFU B. theta-OVA, intravenous dose of 1×10⁶ CFU B. theta-OVA, oral dose of 1×10⁸ CFU B. theta-OVA, oral dose of 1×10⁹ CFU B. theta-OVA, and oral dose of 1×10¹⁰B. theta-OVA. B. theta-OVA intravenous groups are dosed on day 3. B. theta-OVA oral groups are dosed on day 3 and continuously thereafter 2× per week. Tumor volume and body weight are measured twice weekly, with endpoints of tumor volume (2000 mm³) or median survival (2× median; tumor volume <2000 mm³).

### Example 9: Oral Delivery of Engineered Bacteria Expressing Ovalbumin in Combination with Checkpoint Inhibitor in the B16-OVA Model

The following Example describes testing for the anti-tumor efficacy of orally delivered B. theta-OVA (as described in Example 2)+anti-PD1 vs B. theta-parent (non-OVA expressing)+anti-PD1 in a B16-OVA mouse model.

B16-OVA cells are prepared for tumor engraftment by thawing a vial of B16-OVA cells (3.6e6 cells), seeding in T175 flask along with G418 and IFNg, and passaging appropriately to confluency, seeding, trypsinizing, and growing until tumor engraftment at 6e5 tumor cells per mouse (8 week old, female C57BL/6 mice).

Tumors are engrafted on Day 0 and randomized on Day 9 to the following groups: No treatment control (G1), aPD1 (G2), B. theta-parent (G3), aPD1+B. theta-parent (G4), B. theta-OVA (G5), and aPD1+B. theta-OVA (G6). B. theta-OVA and B. theta-parent groups are dosed PO on Day 10 (1×10¹⁰ cfu per mouse) and continuously thereafter 2× per week, and aPD1 (200 μg per mouse) is dosed IP on Days 11, 14, and 17. Tumor volume and body weight are measured twice weekly, with endpoints of tumor volume (2000 mm³) or median survival (2× median; tumor volume <2000 mm³).

While preferred embodiments of the present invention have been shown and described herein, it will be obvious to one of ordinary skill in the art that such embodiments are provided by way of example only. It is understood that obvious variations can be made without departing from the spirit and the scope of the compositions and methods set forth herein.

### INCORPORATION BY REFERENCE

The entire disclosure of each of the patent and scientific documents referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting on the disclosure described herein. Scope of the disclosure is thus indicated by the appended claims rather than by the foregoing description, and all changes that come within the meaning and range of equivalency of the claims are intended to be embraced therein.

SEQUENCE LISTING
SEQ ID NO: 1:
CAATTCGAGGGGGATCAATTCCGTGATAGGTGGGCTGCCCTTCCTGGTTGGCTTGGTTTCATCA
GCCATCCGCTTGCCCTCATCTGTTACGCCGGCGGTAGCCGGCCAGCCTCGCAGAGCAGGATTCC
CGTTGAGCACCGCCAGGTGCGAATAAGGGACAGTGAAGAAGGAACACCCGCTCGCGGGTGGGCC
TACTTCACCTATCCTGCCCGGCTGACGCCGTTGGATACACCAAGGAAAGTCTACACGAACCCTT
TGGCAAAATCCTGTATATCGTGCGAAAAAGGATGGATATACCGAAAAAATCGCTATAATGACCC
CGAAGCAGGGTTATGCAGCGGAAAACGGAATTGATCCGGCCACGATGCGTCCGGCGTAGAGGAT
CTGAAGATGAGGAGTTCAACCTGTTGATAGTACGTACTAAGCTCTCATGTTTCACGTACTAAGC
TCTCATGTTTAACGTACTAAGCTCTCATGTTTAACGAACTAAACCCTCATGGCTAACGTACTAA
GCTCTCATGGCTAACGTACTAAGCTCTCATGTTTCACGTACTAAGCTCTCATGTTTGAACAATA
AAATTAATATAAATCAGCAACTTAAATAGCCTCTAAGGTTTTAAGTTTTATAAGAAAAAAAAGA
ATATATAAGGCTTTTAAAGCTTTTAAGGTTTAACGGTTGTGGACAACAAGCCAGGGATGTAACG
CACTGAGAAGCCCTTAGAGCCTCTCAAAGCAATTTTGAGTGACACAGGAACACTTAACGGCTGA
CATGGGAATTCCCCTCCACCGCGGTGGCGGCCGCATCTTCCAGTGATGGAACAGCATTTGTGCA
TTGGCTGCAACAATCAGCCTTACTTGTGCCTGTTCTATTTCCGAACAAAAAAAAGGCCATCCGT
CAGGATGGCCTTCTACGTCTCATTTTCGCCAGATATCGACGTCTTAAGACCCACTTTCACATTT
AAGTTGTTTTTCTAATCCGCATATGATCAATTCAAGGCCGAATAAGAAGGCTGGCTCTGCACCT
TGGTGATCAAATAATTCGATAGCTTGTCGTAATAATGGCGGCATACTATCAGTAGTAGGTGTTT
CCCTTTCTTCTTTAGCGACTTGATGCTCTTGATCTTCCAATACGCAACCTAAAGTAAAATGCCC
CACAGCGCTGAGTGCATATAATGCATTCTCTAGTGAAAAACCTTGTTGGCATAAAAAGGCTAAT
TGATTTTCGAGAGTTTCATACTGTTTTTCTGTAGGCCGTGTACCTAAATGTACTTTTGCTCCAT
CGCGATGACTTAGTAAAGCACATCTAAAACTTTTAGCGTTATTACGTAAAAAATCTTGCCAGCT
TTCCCCTTCTAAAGGGCAAAAGTGAGTATGGTGCCTATCTAACATCTCAATGGCTAAGGCGTCG
AGCAAAGCCCGCTTATTTTTTACATGCCAATACAATGTAGGCTGCTCTACACCTAGCTTCTGGG
CGAGTTTACGGGTTGTTAAACCTTCGATTCCGACCTCATTAAGCAGCTCTAATGCGCTGTTAAT
CACTTTACTTTTATCTAATCTAGACATTCTTTAAAAACAGATTTGGAGTGCAAAGTTACGACAA
ATAATTTGTTAACATACATATTTAGGCAACTTTTTCACTATATTTTCAACTAATAAAACAAAAA
AGAATGCCGACAAGTTGTGTGACGGCATCCTTTTTCTATTTCATTAGCTTATCCTACAATCTTC
TTACCTTAATTTATAATTTATTCCTGAGTCAGATCAACTGCAAAGTATCCGCGTTTACCGGATG
GGAATACACCTGTCAGGAACAATACCTGATTCGGAGACTTAACAGCAGCTTTCATTGCTTCTTC
CAGATCAGATCAAGTGCTCGAGGTATCCTGGTAAAATAAGCTCGTACTGTAATTCATGCGACAT
GATGCGTAAAGAGATCTGAAATCTATTGGTCTATGTCACTGAAACTATCCAAATACCTTATGTC
GATACTGAACGTATTGACGCATACCTCCTTTTATTCGATTGGATTATCTACTTTATTACTATAT
TTCGGATAAAGCGTGGTGCCCCAGATGGAGATATTTCTATTTTTCACAAGTGGTAAGTTCCGGT
CATCAATTACCGTTCTCCACCATTCCCAAGCTAAACCAGTGCATTCTTTAGCGTAAACATTAAT
ATTTCTCGCGTTACCTGGCAAATAGATGGACGATGTGAAATGAGCTAGCTTGCTTTTATTGTTT
TCGCTCCAGTTTTTATGTTGAACAATTTCGTTACCTTCAGGATCATAATTTACTTCATCCCAAG
AAATGTTGAATTGAGCAACGTATCCTCCAGAGTGATCGATGTTAATTTTTCCATCTGTATAAGC
TTTTGAAGTTGTTTCAATATATTCTGAGTTGTTTTTAATAACAGCTAATTCATTGTCTTTTAGG
AAGTTTGTTGTATAAGCAATGGGAACTCCTGGTGTTTCTCGATTAAAAGTAGCGCCTTTTTTCA
AAATATCGCGTAAGTCTCCGAGGTTGCCGTCGATGATTTGAACTTCATCTTTTGCGGAACCTCC
GTAAATTACGGCTTTGAAGGAAGAATTTTTGATGATATTTGTTAGTTCTAGATCACCTGAGACA
GATTTTCCGCTTACGGCAGCATCAAAAGCAGCTTTTACTTTAGTACTATGGGAATTAGTTGATA
ATTTCAAATAAACTTGACGGCCATACGCCACACTTGAGATATATGCAGGAGGATTTTCTGCATT
CACTCCAAGCGCTTGCAACTGCTCTTTAGTAACAGCTTTGCCGAAAAATCTGGAAGGTCTTGTA
GGTTCATTAACATTCACGTTATAGTAAATTTGTTTAAAACTAATGACTTCTTCTTGCATTTTCC
CTTCACTGATTGCGCCGAAGTTTACATTCAAGCTATTATTTACAGCTTTAAATGCTGTACCAAA
TTTCGCAATTAATTGTGATTCACTGTAAGCCATTTCGTCATCATAATCAATTTTTGCACTTACA
TTTGGATAAGCTTGAGCATATTTTTCATTCCATCTTTCCACTAATGTATTTACTGCGTTGTTAA
CGTTTGATTTAGTGGCATTTTTTACAACGATTTTATTGTCTTGATTAGTCATACCTGGCAAATC
AATGCTGAGTGTTAATGAATCACGTTTTACAGGGAGAACATCTGGTTGATTTTCTACTAATTCC
GAATTCGCTTTTACGAGAGCACCTGGATAGGTTAGGCTCGAAATTGCATTCACAACTTGAATGT
CTGCATTATTTTGATTGATGGATTTCTTCTTTTTCTCCACAACAATATATTCATTTCCATCTTT
GTAACCTTTTCTTGGCGGCACATTTGTCACTGCATCTCCGTGGTATACTAATACATTGTTTTTA
TTGTAATCCAATCCTTGTATATACTTATCGATTTCATCCGCGTGTTTCTTTTCGATTGGCGTCT
TAGGACTTGCAGGCGGAGATGCTGGTGGTGCCATGGATGAAATTGAATTTTCTTTATTGAATGC
AGATGCATCCTTCATGGATTCGTTTAACTAAACGAATAATTTATTTTATTTTAAAATGCGTCTC
TATGAGTGATAGGGAGACTGCAAAGATAAGAACTTTTAGCTTTATCTTCCAAACTTTATCCCTA
TCAGTGATAGAGATTTTTTTGTTCTTATGGTCAAGCAGTACTGCGCTTTTACACTAAGTCAATT
TAACAAGGCTTTCTTCTCTTGGAAAGCGGGTGCAAAACCGTTTCAACAGACGCTTGGTAAGATC
ATTACGAATACCAGGTATCGTGTAAGTAGCGTAAGCCTATACGCAAGATAAACTGCTAAAAAAC
CGGCGCGCTTTGCACCCGCTTTCCAAGAGAAGAAAGCCTTGTTAAATTGACTTAGTGTAAAAGC
GCAGTACTGCTTGACCATAAGAACAAAAAAATCTCTATCACTGATAGGGATAAAGTTTGGAAGA
TAAAGCTAAAAGTTCTTATCTTTGCAGTCTCCCTATCAGTGATAGAGACGCATTTTAAAATAAA
ATAAATCATATAGTTAATTAAACGAATCCATGCGTTATCCCATCTTGCCCGAATATCTTCAATG
TGTTAAGGAACTGTATCGTGGAGGACTGGAACCCATCAACTTTCAAACCGCCGCTGATCAGGCA
CGTGAACTGATCAATAGTTGGGTAGAATCCCAAACAAATGGTATCATTCGTAATGTACTGCAGC
CGTCTAGTGTGGATAGCCAAACCGCTATGGTACTGGTGAATGCAATTGTGTTCAAAGGACTGTG
GGAAAAGACTTTCAAGGATGAAGATACACAGGCCATGCCGTTCCGTGTAACTGAACAAGAATCT
AAACCTGTGCAGATGATGTATCAAATCGGTCTGTTCCGTGTAGCCAGCATGGCTTCTGAAAAAA
TGAAAATTCTGGAACTGCCGTTTGCTAGCGGTACAATGTCTATGCTGGTACTGCTGCCTGATGA
AGTGAGCGGACTGGAACAGCTGGAATCTATCATCAACTTCGAAAAGCTGACAGAATGGACATCT
AGCAATGTAATGGAAGAACGTAAAATCAAAGTGTATCTGCCGCGTATGAAGATGGAAGAAAAGT
ATAACCTGACCAGTGTACTGATGGCAATGGGTATTACTGATGTGTTTTCTAGTTCCGCCAATCT
GTCCGGAATCAGCTCTGCTGAAAGTCTGAAAATTTCCCAAGCAGTACATGCTGCACATGCAGAA
ATCAATGAAGCCGGACGTGAAGTAGTGGGTAGTGCAGAAGCCGGAGTAGATGCCGCTAGTGTGT
CCGAAGAATTTCGTGCCGATCATCCTTTCCTGTTTTGTATCAAGCATATCGCTACCAACGCTGT
ATTGTTTTTCGGTCGCTGTGTAAGCCCGTAACGACCGCTTGTATGAATCCATCAAAATTCGTTT
TCTCTATGTTGGATTCCTTGTTGCTCATATTGTGATGATAATTTCTACAAATATAGTCATTGGT
AACTATCTATGAAACTGTTTGATACTTTTATAGTTGATTAAACTTGTTCATGGCATTTGCCTTA
ATATCATCCGCTATGTCAATGTAGGGTTTCATAGCTTTGTAGTCGCTGTGTCCCGTCCATTTCA
TGACCACCTGTGCCGGGATTCCGAGAGCCAGCGCATTGCAGATGAATGTCCTTCTTCCTGCATG
GGTACTGAGCAAAGCGTATTTGGGTGTGACTTCATCAATACGTTCATTTCCCTTGTAGTAGGTT
TCCCGTACAGGCTCGTTGATTTCTGCCAGTTCGCCCAGCTCTTTCAGGTAATCGTTCATCTTCT
GGTTGCTGATGACGGGCAGAGCCATGTAATTCTCGAAATGGATGTCCTTGTATTTGTCCAGTAT
GGCTTTGCTGTATTTGTTCAGTTCAATCGTCAGGCTGTCGGCAGTCTTGACTGTGGTTATTTCG
ATGTGGTCGGACTTCACATCGCTTCTTTTCAGATTGCGAACATCCGAATACCGCAAACTCGTAA
AGCAGCAGAACAGGAAAACATCACGCACACGTTCCAGGTATTGCTTATCCTTGGGTATCTGGTA
GTCTTTCAGCTTGTTCAGTTCATCCCAAGTCAGGAAGATTACTTTTTTCGAGGTGGTTTTCAGT
TTCGGTTTGAACGTATCGTATGCAATGTTCTGATGATGTCCTTTCTTGAAGCTCCAGCGCAGGA
ACCATTTGAGGAATCCCATTTGCTTGCCGATGGTGCTGTTTCTCATATCCTTGGTGTCACGCAG
GAAGTTGACGTATTCGTTCAATCCAAACTCGTTGAAATAGTTGAACGTTGCATCCTCCTTGAAC
TCTTTGAGGTGGTTCCTCACTGCTGCAAATTTTTCATAGGTGGATGCCGTCCAGTTATTCTGGT
TACCGCACTCTTTTACAAACTCATCGAACACCTCCCAAAAGCTGACAGGGGCTTCTTCCGGCTG
TTCTTCGCTGGTGTCTTTCATTCTCATGTTGAAAGCTTCCTTCAACTGTTGGGTCGTTGGCATG
ACCTCCTGCACCTCAAATTCCTTGAAAATATTCTGGATTTCGGCATAGTATTTCAGCAAGTCCG
TATTGATTTCGGCTGCACTTTGCTTTAGCTTGTTGGTACATCCGCTCTTTACCCGCTGCTTATC
TGCATCCCATTTGGCTACGTCAATCCGGTAGCCCGTTGTAAACTCGATGCGTTGGCTGGCAAAG
ATGACACGCATACGGATGGGTACGTTCTCTACGATTGGCACACCGTTCTTTTTCCGGCTCTCCA
ATGCAAAAATGATGTTGCGCTTGATATTCATAATTGGGTGCGTTTGAAATTCTACACCCAAATA
TAGACCCAATTATTGAGATAGCAAAAGACATTTAGAAACATTTACTTTTACTCTATATTGTAAT
TTACACTTGATTATCAGTCGTTTGCAGTCTTATGATATTCTGTGAAAGTATAAGTTCGAGAGCC
TGTCTCTCCGCAAAAAACGCTGAAAATCAGCAGATTGCAAAACAAACACCCTGTTTTAGACCCA
AGAATGTAAAGTCGGCTGTTTTTGTTTTATTTAAGATAATACAACCACTACATAATAAAAGAGT
AGCGATATTAAAAGAATCCGATGAGAAAAGACTAATATTTATCTATCCATTCAGTTTGATTTTT
CAGGACTTTACATCGTCCTGAAAGTATTTGTTGGTACCGAGGACGCGTAAACATTTACAGTTGC
ATGTGGCCTATTGTTTTTAGCCGTTAAATATTTTATAACTATTAAATAGCGATACAAATTGTTC
GAAACTAATATTGTTTATATCATATATTCTCGCATGTTTTAAAGCTTTATTAAATTGATTTTTT
GTAAACAGTTTTTCGTACTCTTTGTTAACCCATTTCATTACAAAAGTTTCATATTTTTTTCTCT
CTTTAAATGCCATTTTTGCTGGCTTTCTTTTTAATACAATTAATGTGCTATCCACTTTAGGTTT
TGGATGGAAATAATACCTAGGAATTTTTGCTAATATAGAAATATCTACCTCTGCCATTAACAGC
AATGCTAGTGATCTGTTTGTATCTAATAACATTTTAGCAAAACCATATTCCACTATTAAATAAC
TTATTGTGGCTGAACTTTCAAAAACAATTTTTCGAATTATATTTGTGCTTATGTTGTAAGGTAT
GCTGCCAAATATTTTATATGGATTGTGGCTAGGAAATGTAAATTTCAGTATATCATCATTTACT
ATTTGATAGTTAGGATAATTTAAGAGCTTATTACGAGTTACCTCACATAATTTAGAATCAATTT
CTATCGCCGTTACAAAATTACATCTCTTTACCAATCCAGCAGTAAAATGACCTTTCCCTGCACC
TATTTCAAAGATGTTATCTTTTTCATCTAAACTTATGCAATTCATTATTTTTTCTATGTGATAT
TTTGAAGTAATAAAATTTTGACTATCTTTTATATTTACTTTGTTCATTATAACCTCTCCTTAAT
TTATTGCATCTCTTTTCGAATATTTATGTTTTTTGAGAAAAGAACGTACTCATGGTTCATCCCG
ATATGCGTATCGGTCTGTATATCAGCAACTTTCTATGTGTTTCAACTACAATAGTCATCTATTC
TCATCTTTCTGAGTCCACCCCCTGCAAAGCCCCTCTTTACGACATAAAAATTCGGTCGGAAAAG
GTATGCAAAAGATGTTTCTCTCTTTAAGAGAAACTCTTCGGGATGCAAAAATATGAAAATAACT
CCAATTCACCAAATTATATAGCGACTTTTTTACAAAATGCTAAAATTTGTTGATTTCCGTCAAG
CAATTGTTGAGCAAAAATGTCTTTTACGATAAAATGATACCTCAATATCAACTGTTTAGCAAAA
CGATATTTCTCTTAAAGAGAGAAACACCTTTTTGTTCACCAATCCCCGACTTTTAATCCCGCGG
CCATGATTGAAAAAGGAAGAGTATGAGTATTCAACATTTCCGTGTCGCCCTTATTCCCTTTTTT
GCGGCATTTTGCCTTCCTGTTTTTGCTCACCCAGAAACGCTGGTGAAAGTAAAAGATGCTGAAG
ATCAGTTGGGTGCACGAGTGGGTTACATCGAACTGGATCTCAACAGCGGTAAGATCCTTGAGAG
TTTTCGCCCCGAAGAACGTTTTCCAATGATGAGCACTTTTAAAGTTCTGCTATGTGGCGCGGTA
TTATCCCGTATTGACGCCGGGCAAGAGCAACTCGGTCGCCGCATACACTATTCTCAGAATGACT
TGGTTGAGTACTCACCAGTCACAGAAAAGCATCTTACGGATGGCATGACAGTAAGAGAATTATG
CAGTGCTGCCATAACCATGAGTGATAACACTGCGGCCAACTTACTTCTGACAACGATCGGAGGA
CCGAAGGAGCTAACCGCTTTTTTGCACAACATGGGGGATCATGTAACTCGCCTTGATCGTTGGG
AACCGGAGCTGAATGAAGCCATACCAAACGACGAGCGTGACACCACGATGCCTGTAGCAATGGC
AACAACGTTGCGCAAACTATTAACTGGCGAACTACTTACTCTAGCTTCCCGGCAACAATTAATA
GACTGGATGGAGGCGGATAAAGTTGCAGGACCACTTCTGCGCTCGGCCCTTCCGGCTGGCTGGT
TTATTGCTGATAAATCTGGAGCCGGTGAGCGTGGATCTCGCGGTATCATTGCAGCACTGGGGCC
AGATGGTAAGCCCTCCCGTATCGTAGTTATCTACACGACGGGGAGTCAGGCAACTATGGATGAA
CGAAATAGACAGATCGCTGAGATAGGTGCCTCACTGATTAAGCATTGGTAACTGTCAGACCAAG
TTTACTCATAACGCGT
SEQ ID NO: 2:
GATAAAACGAAAGGCTCAGTCGAAAGACTGGGCCTTTCGTTTTACAATTGGGCTACCTTTTTTT
TGTTTTGTTTGCAATGGTTAATCTATTGTTAAAATTTAAAGTTTCACTTGAACTTTCAAATAAT
GTTCTTATATTTGCAGTGTCGAAAGAAACAAAGTAG
SEQ ID NO: 3:
GATAAAACGAAAGGCTCAGTCGAAAGACTGGGCCTTTCGTTTTACAATTGGGCTACCTTTTTTT
TGTTTTGTTTGCAATGGTTAATCTATTGTTGAAATTTAAAGTTTCACTTGAACTTTCAAATAAT
GTTCTTATATTTGCAGTGTCGAAAGAAACAAAGTAG
SEQ ID NO: 4:
GTTAA(n)₄₋₇GTTAA(n)₃₄₋₃₈TA(n)₂TTTG
SEQ ID NO: 5:
GTTAAnnnnnnnGTTAAnnnnnnnnnnnnnnnnnnnnnnnnnnnnnnnnnnnnnTAnnTTTG
SEQ ID NO: 6:
TAAGGATTGATTCGCTAGCTCAGCAGGTAGAGCACAACACTTTTAATGTTGGGGTCCTGGGTTC
GAGCCCCAGGCGGATCACTGAAACAAAAAGCAAAACAATGAAAACCGCTGATAATCAATCATTA
TCAGCGGTTTTTCTTTTTATCCATACTGCAAATTGAAGCAGAATACCGCATTTTACTGGAGGTG
AAATAGGTGGACTTAATTTCCACATAAAAACAAGTCCACCTGATTGGATTATATTTCACTGATT
CTCTGCGTTTTGCATAAAACAAACTCTTTTCAAAACATGTATTTTTAGACCATCAAAAAAAGAA
GAGTATGGCAATGCAAAGAAACTATTTTACGGTATTGTTTTTCCTGAAGAAATCAAAGCTGCTT
AAAAATGGAGAAGCACCAATCTGTATGCGTATCACAATAAACGGAAAACGTGCAGAGGTACAAA
TCAAGCGAAGTATAGATGTTACAAAATGGAATACGCAAAAAGAATGCGCGATTGGCAGGGAAAA
GAAGTATCAAGAAATAAACCACTATCTTGATACGATAAGAACTAAAATCCTTCAAATTCACCGT
GAACTTGAGCAGGACGGTAAACCTATTACAGCAGATATTATAAAAAATATCTATTATGGAGAAC
ACTCTACTCCCAAAATGCTGCTTGAAGTATTCCAGGAACACAATTCGGAATATCGGGAATTAAT
GAACAAGGAATATGCCGAAGGTACTGTACTTCGATACGAACGTACAGCAAGATATTTGAAGGAG
TTTATCAGTGAACAATATAAACTGGCTGATATTCCATTAAAATCAATCAACTATGAATTTATAA
CCAAATTCGAACATTTCATTAAAATACAGAAAAACTGTGCGCAAAATGCGACAGTGAAATATCT
GAAAAATTTAAAGAAAATCATCAAAACTGCATTGATAAAGAAGTGGATAACTGATGATCCGTTT
GCAGAAATACACTTCAAACAGACCAAGTGTAACCGTGAATTCTTAAACGAAATGGAACTTCGCA
AAATCATCAATAAAGATTTTGATATTCAACGATTACAAACCGTAAGGGACATATTCATCTTCTG
TTGTTTCACCGGTTTGGCTTTCACAGACGTAAAGAATCTGAAAAAGGAACACCTTGTACAGGCT
GATAATGGTGAATGGTGGATAAGAAAAGCAAGGGAAAAGACCGATAATATGTGCGACATTCCAT
TGTTGGATATACCAAGACTTATTTTAGAGAAATATCAGTCAAATCCAATCTGCAATGAAAAAGG
ATTATTACTTCCTGTTCCCAGCAACCAACGAATGAACAGTTATTTGAAAGAAATAGCTGATGTA
TGTGGTATTCAGAAGAATCTTTCCACACATATTGCAAGACATACATTTGCATCACTGGCTATTG
CAAATAAGGTTTCCTTGGAATCCATTGCCAAAATGTTAGGACACACGGACATTCGTACAACTCG
TATTTATGCCAAAATAATGAATTCTACCATTGCCAATGAAATGAAAGTACTGCAAAACAAGTTC
GCAATATAATTTTCAACCATTATTTCATTTCTTAGAGCAAATATCGCACTTTGCCACTGACTGT
GCAAGGCGGCCCTGTCGGGCTGGTTGGCGGAAAAAAATCATCCTCGCTTCGCTCCGGTATTTTT
TTCCGCCAAGCCTTGCACCGGTCATTGGCAAAGAACAGCCGGGCCAGTAAGAAATTGAAATACT
GGCTCCACGGAGCCGGTCATGTCTAATTTAAATAAAAGAATATGACTGAAGAAGTTGGAAAGAA
GGTATGTGAAGGTACAGTAGCAGACCTCATGAAGGACAAGACCGGAAAACAGACGGTTGTCACG
TTGACAAGAAAGAATGCTTACCGAGTGAAGAAAATCAGAGAACAAGGGACGGATGACGAAGCTG
TCCTTTTTCATTTCCGTGAACGCTGTACGGGAATGGGCTCCTATGTACACACAATCGAAGCGGC
AGACGGAGAAACAGAACTTCATCCGTCTGAATTTGAAAAATGGGAAGCTGTGGAATTCCTGTAT
CCCGGCTATCTGGAAGACCTGCTTGATGCTGCATACAACGCATACAGATGGAGTTCCTTCGAAC
CTGAAGCAAGGGCGGAAACAGACATCATGCAATATGAAAAACAACTTGTAGAGGATCTGAAACA
GATTCCGGAAGAAAAACAGAACGAGTATACCAGTGCATACCATAGCAAGTTCTCTGCCTTGCTG
GGCTGTCTCTCACGATGTGCCAGTCCGATGGTGACAGGGCCTGCCAAATTCAACTGCCAGCGCA
ACAACAAAGCCTTGGATGCATACCAGAACAGATTTGATGAATTTCATGATTGGCGTAACCGCTT
CAAGGCTGCCATGGAAAGGATGAAAGAGGCTGCCAAACCGGAAGAACAGAAGCAAGAGGAGGCA
TGGAACCGCCTGAAGCGTGACATTGCAAGCAGCGCACAGACCATTCATGATATTGATACCGGTA
AAGCAAGAGGATACAGCCGTGCCTTGTTTGTCAGCAGTATCCTTAATAAAGTAAGCACCTATGC
AGGAAAAGGAGAAGTGGAAATCGTACAGAAAGCGGTGGACTTCATTACAGACTTCAATGCACAA
TGCAAAAAACCGGTTATCACTCCGCGGAACCGTTTCTTCCAACTGCCGGAAATGGCACGCCAGG
CCAGACTGAAACTTCAGGAAATCAGAGAACGGGAAAACCGTGAACTGAAATTTGAAGGCGGAAC
GCTGGTATGGAACTATGAGGCAGACCGCCTGCAAATCCAGTTTGACAATATTCCGGATGACCAG
AGGCGCAAGGAACTGAAATCATACGGTTTCAAATGGTCGCCGAGATACCAGGCATGGCAACGGC
AACTTACACAGAATGCCGTATATGCAGTCAAAAGAGTGTTGAACCTTCAAAACCTATAAGACAT
GAAAGACCGATTGAAATATGTAATCGATTCCCGCTACTTCGACGGAACATGCCTGACAAGTATG
AGTGACGGATTCCATAATGACTATGGTGGGGAAACAATCGAAGAACTGCGCATACGGGAAAACA
ATCCCTATCTGAAAGCAGTAACACCTTCTGATATAGACAAGAAGCTGCGGCTATACAATCAGTC
CCTGTCCGAACCGTTCAAGGAAATCACTGAAGAAGAATACTATGACCTGCTGGATGTACTGCCA
CCCTTGCGCATGAGACAAAACTCGTTCTTTGTAGGAGAACCGTATTACGGAAATATGTACTCTT
TCTGCTTTACTCGTCAAGGAAGATATTTCAAGGGCCTACGCTCCGTACTTACTCCGCAATCCGA
ACTGGACAGTCAGATAGACCGTCACATGGAAATCATCAACCGGAAAGCCGTGATCTCAAAAGAG
GAAACAAGTAAAACGGTCACAACCGGAACCAGACTCATTCCCTATTATTTTTCACTGGACGGAA
AACAGCCCGTATTCATCTGCAACCTTGTCATCCAATCAGATTCCAGTCAAGCAAGGACGGACAT
GGCGAATACCCTGAAAAGTCTTCGCCGGAACCATTATCAGTTCTATAAAGGAAAAGGGCATTAC
GAAACTCCGGACGAACTGATAGACCATGTATCAGGAAAGAAGCTCACCCTTGTTTCCGACGGAC
ATTTCTTTCAATATCCTCCCGGCAGGGAATCCGCAACTTTCATCGGACACATCAAGGAGACATC
AGAGGAATTTCTTTTCCGGATCTATGACCGTGAATATTTCCTGTATCTTCTTAAAAGACTGAGG
ACCGTGAAAAAGGAATCGGCACAGGAACAAATAAATATCAAATCATAACATTCGGGGGAATGCG
GTAAAATGACTGCCGTATTCCCTCATAAAAACAATACAAGTATGAACAAATCAAACACTCTATA
CTGGAAAACAGCCACAGATCCGGCTGAACGCATTGAGGTCAGACTCGTCCTGAACAGTTATATC
GACAATGACAATCTGTATGTAGGACTTGAATCCCGGTCTAAGGAGAATCCGGAATGCTGGGAAT
CCTACACGGACATCACCGTCAACCTCAATTCTCTTCCCCCGTTCCATGCCTATGTGGACAACCG
GGACTGCAACAGACATGTGCATGATTTTCTGACCAGTAACAGAATAGCAGAACCTGCCGGATTT
GAATATCAGGGATTCAGAATGTTCCGCTTCAATCCTGACAGGTTGAAGGAACTCGCACCCGAAC
AGTTCAAGACAATCAGCGCCAAACTGCCACCACAGGATGACATGATAAAGGACATCATCTATCA
GGAAAGACGTTTCCCTTTGAGAACTGTTCAAGACATTCACGGAATATATCTTGTTTCAAGCAAG
GAACTGGAAGAATCTCTGATCGAAGGAGTACGGAACCTGGATGCTGCGGCATATGAACTGCTGG
ATGGCATCTGCCTGTTCTGCTCCACACAGGAACTGCGCTATCTTACGGATGCAGAACTGATAGA
AACAATCTACGCACAATAAAAAGGAGGAACAAATATGAAAACCGGAGACATTGTATTTCTGAGA
CGTCCCTATAAGGGATACCGTGCCGTCGAACTGATGGAAAGACTGGAATGCCGCTGGCTGGTCA
GGATTGTCGAGAGCGGTCTTGAACTGGAGGTATATGAAGATGAACTTATATCAGAATTTTAATA
CAGACAAAGTGTTATGGAAAAATATCAGTTTGCATTCCATTCGGAAATAATCGGCTATACCTCT
CCTCATATCGGTGAGGTCAGAAAAGCCATACACAGAAAAGTGGAAAAGGAAAAGTCTGCCGCCA
TAAAGAATGATATTGAGCTGCACATGTACAAAGTGCATGACGGCATACCGGTTCTCCTTAACAC
CTGCTACCTGTACGATGAAAAAGGATGTATGGTACACGGAAGTATCAAGGGAACCAAGGATTAT
CTGCTTGAGACATGGAGATACCATACAAACAGACATTCTAAAGGCATCAGTTCCACAAGAATCA
GGCCTTGCACGACAAGCAGGGCTTTTTCATTTGTATAACTCTTAAAATCAGAAATCATGAACCA
GACATTAGAACTTAGAGACTATATTCCACAGAATGTAAGCCTCTACTACGTGGACTACCGGGAT
GATCTTGATGAGCATGAAGACATCCAGGAGGAATGCATCCGTTCCAACAAAATGGAAAAACTCT
ATGAAAAGGCATACGAATGGTATGAGGAACAGGAAAGTTCAAACATGCACGACTATCTGGAGGA
GACAAGAAAGAATATGGAAACGGACAATTTAGCCGGAGAGTTTGAAGAGCATGAAGATGAAATC
AGGGAACTTATCTACGACCGGAACGATTCCGACCCGGTAAAGGATATGATACGCAACTCGTCCG
TCACTAATTTCTTCTATTCGCTCGGAGTGGAAATCAGCGGATATCTGACCGGTTGTTCACTGCG
GGGAGAATCAGTCGCCATGGCCTGCCATAAGGTACGTCGCGCACTGCATCTGAAAAAGGGGCAG
TTTGACGAGAAGATTGAAGAACTGGTAGAGAATGCCACATACGGTGGAGAACTGCGCATCTACT
TCAACGCCATGTTTGACAGGCTCATCAGCAAAGGCCCTGAGAACGATTTCAAGAGCATCCGTTT
CCACGGGAATGTAGTGGTGGTCATTGCCGACAGCCGGAACGGTTCCGGACATCATGTACGGATT
CCGCTGGACATCACTTTCCCTTTCCGAAGGGAGAACCTGTTTGTCGATTCACAGGTACACTATT
CCTATGCCAATGAAGTCTGCGGCATGACCAATGACTGGTGTGATTCCACAAAATGGGAAACAGG
CATGATACCTTTTACCGGATCTGTCCGAAAAAGCCGGATGGCTGAATACAAGAAACAGGAAGCC
GCTTATGAGCAGACATTCCGAGACGGGAAATGCACCTTCGGTGACATGAACTACAAACGCCACC
GTGACGTGCGGTATTCGAATGAATATCCTGCCGGATGCAGGTGCCCTCATTGCGGTACATTCTG
GATTGACTGAAAAAACATTTACCAACCAATAAATTCAAACGATATGAAAATCTGCTGTTCACAA
GAGCATTACGACAAGGTCGTACAGTATGCAAAATCAATCAATGACAAGACACTGGAAAACTGTC
TTGAACGTCTAAAACAATGGGAGAAGAACGAGAACCGTCCATGCGAAATCGAACTCTATTAGGA
TCATGCGCCGTATTCGTTCGGATTCTGCGAACGTTATCCGGACGGAAATACAGGCATTGTCGGA
GGACTGCTGTATCATGGAAATCCGGACGAATCCTTTGCCGTCACCATGGAACGTTTCCACGGAT
GGAGCATACATACCTGACATATATGCGACAGTCTGTATTGGGGAGCCTCATGCAATATGGGGTT
CCCTTTTTTTATGCCGCAGACATGATGACAGCATCCTCATTTCTTGCTGCAAAAATAGCTGTTT
GCCGCGCAACTCCCGCAAGGCGGCCCTGCCGGGCTGGTTGTCTGGAAAAAAATCATCCTCGCTT
CGCTCCGGTATTTTTTTCCGCCAAGCCTTGCAGGGATGCGGGCAAACAGACAACAGGGACAACA
AGAAATAAGAATGCCTGTACCTTAGAGGCAGACAATGTATAACAATAAATATCAGAAGTCATGA
TTACAGACCAGAAGACACAGAACAGGCTTCACGCGGATACCGGAACGGAACTGTTCTCCATCAG
ACAAAGGAAGGAAGCCGTCACAAGGATGCTGGACATTCTGAAAGAGACTCCGGAATACCTGCAG
GTTATGAACCATATACCGGCTTATGCCATGGATGACGATACGTCAGAATGGTGGAAATCGGAAG
AATCGGAAAATTTCATGAACTCACTCCTGGAAGTGATGGAAAGCTATACTCCGGACGGATACAG
GTTCGGACCGAAATCCGGCACGACTGACCTTTACGGCTACTGGGAAAGCAAGACCGGGCGGACA
ACCCTCTTCCATCTGCTTTTCAGTCTGGAAAGCGGATATGAATGGGGAAAAGGTCTTTCCCATG
AGAAAACGGACGCATTCTACAAGGAAATAAAAGAGAAATTTCATGGAGAAGGATTCGACACGGA
CAGAACCGGCTGTACATCACAGGCCATGTATCTTGTAAAAGGAAAAACACGCCTGTACGTGCAT
CCGATGGAAATAAGCGGCTACTGTGAAACACTGCATATTCCACAGATTACAGCCATACTGAAAA
AAGGAGGCCGTACATTCCGTCTTGTAAAGGATACGATAGCGGAAGAGGTGTATTCCTTCACCGA
TGAAGAAGAACTGGAATATTACCGTGCCAGATACGGAACGTGCATCCACCGGAATATACTGGAT
GCCTTCAGCAACCGCCACGCAGGGAAAGAGGACATACTTTCCATGATGGCATCACGGATAAATG
TGGCTACGACATCACATCTTTACGGTATCGGATATGATTCGCCTGCATACAGGTTTGTGCATGA
GGCATACGACAGACTGGTAAACAATGGAAAGCTGAAGGAGAATGTCCGGGAAATCGGTTGCTGC
AACATCATAATGGCCATTTCAAATACCAACGCAATATGAGACTGAATTACAATGACATGCTGCT
TCTGGCAATATGGGAATACAACAGGAGACAGGACGAGGATCTGACCCTGGAACTGTTTCAGGAA
ACATTCGGACAGGTTCCCGGCGCACATTTCCATGACAAATGGGTGCATTATTACAACAAGAACC
TGCTGATGATGGCCGCCTATTTCAGGGGTGAGGAAGAAAACGGCCAGAAATTCTGTGATATGAT
CACCCGACAGGTTGAACGCTATACACAAAACAGGAGGAGAACAGGATGAATACAAAGATACGAT
ATGACCTTGACAGTCTTGAACTGGCAAACGGTGACTTCGGGTATCCCATTACAGAAAAGGAAGT
ACGGAAAGTGAACCGTATGCTGGAACTGATGGAGAATGTCCGAAGCAGGCAGATGTGCCCGACA
GAAGGAGACTGCGTGGAATTTGTCTCACGTTCTGGTGACTATTTCGGAAAAGCTCATATAGAAC
GGATAACAGGAAAATATGCGGATATATGCCTGATACCGGAAACGGTATTCTGTTTTGATGACAT
GGGAAAAGCCGCCTATGATACCACCGGAAGTCCCTGGACGCAGGTCAATATCCGGAACATGAAA
CCCGCAGGTTCTGAAATCCGCATATTCAGAACATGGGGATTCGGGAAGCGCAGCAATACGGGCA
GTCTCAGGTTCGATGCTCCGGTCAGGAAATGGGAATACAGAGAACCGAATCCGTTATATGACGG
TTACACCACCCGTAACTGGTTCCGCTATCATATCATGAAACACCGGGACAGGGAAAGGACAGGC
GAATACACCTTCCGCAGCGATTCATTCACGCTGTACAGCCGGAGCGAGCTGGACGAGCTGGCCG
CAATCCTGAAAGGCAGACTCTACAAGGGAATCCTGCCTGACTCTCTTGTACTTTGGGGATACCG
CATGGATATTAAGGAAATATCACGTGAACAGTGGAACGGTATGGGACAGCACGGACAAATCCGC
ATGAAATTCATGGGATACGGTCCGGTCAGAATCCACACGGACAATGAAAACCATACCGTAACAG
TATACAGAATCAACGACATATTGTCTTCAACTATCAGAATTTTCATATTTTTTCAGTTCTTTTT
TTGTTTCTTCTATTAATATTTTAAGCCACTCCATGATTTGTATTGCATGTTCATGAACAGTTTC
ATTTTGGCTATCACTGTCGTGTAGTAGCCTTTGAAAATCACGTAAAATATTGTCTTTCCCAAGC
ATCTCCCATACAGGCATCATCCGGTGGATTATTTTTCTCATGGTCTCACGGTCGGTTATCCTGT
CAGCAGATTCCATCTCCTCCAGTTCTTTTTCAGATTCCATAACAACGAGAGAAAGCATATGACT
ATAATCATCCGTATTCTCCAGTAAACTGGAAAAATCGAATTCTCCGGAAACTGAAACTTGTGTA
CGAGATATAATGGTGGATAAAAAAGCAAGCAGTCCGTGGATATTGAACGGTTTATGAATACAGC
CTACAAATCCTTCTTTTTCATAAATTCCGGAATTTCCGTCACCACGGGCAGTCATGACTGCTAC
TGGAACAGTTCTAGAATTGCCGATGTCCGAATTGCGAAGCAATCTTAACAAACCGAATCCGTCA
GTATCAGGCATTTGTACATCTGTCAAGATCAAATCATATTCAGAATTTTCAAGAGCGGCCACTA
CTTCACGTGCATTCTTACAGGTTTTACAGGATATACCTTTGCGCCCGAGCATATCTTCCGCTAT
TTTCAGTTGTATAGGATCATCGTCCACTACAAGAACATTCTTAGGCAATATAGTTATTGTATTA
TGGTCCGATTTGTCTTCCTCAACTAACTCATCCGTTTCAGGCAAAGAAAGTTCCAGTCTGAACA
TGCTTCCTTTACCGAGTACACTTTCTACATCCATTTTTCCTTCCAAAACCTTAATTAATCCTTT
GGTAAGGAAAAGTCCCAAACCAAACCCTTCAGAATTGACATTCTGTGCGGCACGCTCAAATGGA
GCAAATATTCTTTTCAGTGTTTCCTCATCCATACCGATACCAGTATCCCTTATTTCAATACGAA
GTTTTCCTTCTGAATATTCTGAATGGAAATTGACGTTACCCCTGGAAGTAAACTTAATAGCGTT
TGTAAGTAGATTGGCTAAAACCTGTTCAAGTTTGTCCGCATCACCTTTTACTATTACATTTGAT
CCTTTATGTTCAGAATATAAAATCAGACCTTTTGAAGTCGCTTTACGAGAAAACTCATCTGAAA
TTCGTTGCAAGAAACGGTCAAGATAAAATGGTGTGTCGTTACGCAAATTACCGGCTTCATTGAT
TCGGTAAGCATCCATCAAATCATTAACCAGATGTAAAACGTGTCGACAAGAATGACGGATGTCA
TCTAAATATTTTTCGCGCTTCCTCTTTTCACGCGTTTCAGATACCAAATCTGCACAGTTATGGA
TATTAGCAAGTGGACCTCTAATATCATGAGAAACTGTCAGGATGATTTTCTTACGCATATCAAG
CAAATTCTCGTTTTCTTGAATAGCTTGTTGTAATTTAAATTTAATTATTTCTTCCTTACGTAAA
TCTGATTGTATAATTAAAAATGAAATTAATATTATAAAAACCGCAATACTCATCATTAGGATAA
ATAATCGAAAGGATTCTTGTTTGACTTCCGTTACCTCTAAGTTTCGTTCTATAAATGACAGCTG
TACCTGATTATCTAAAAAAGATACAAAATCATATAATTTTTGATTTAACAGCCTATTCTGCAAA
CGCAAGCTATCCACATAAGTTTCTATCTGATTGTTTCGCATATCTATGACAGAAACCAATCTAT
TATTAAAATTCTGTATTTCATTAGTTATATAGGGGACTTGTATCGTCTCCTTCTTTCCGAATAA
TCCGGCAATTCCTTTCTTTTTCTGAGTTATTGTCTTCACTTTTACTGTTTGAGTAGCTATTACA
GGCAATTCATTAGTAAGAATACTATCAGATTTATTCGCAAATTGGACTGCTTTCATTATTTGAA
ACAAGTGCATTTCTTTCGTTTTAAGCAATTCCCGTAAAGAATCAATTTGAACTGGACATAAAAA
ATCACAACTCCTTAATTTTATTTCAAGTAGAACACTATCTGTTTTAAAACGTTGATTATGAAAT
ATGTTATAATCAGACTCATCCCATACTATAACTGATTCGCCTAAAGTTGCCAACTTAGTAATAT
ACAAATGAACTTTATTAGTATTCTCATAAGCTTCATTAATTTGAATTATCAGATTCTCAAGTTC
TTTCAACCGGCAACGTTCATTTATCATTACAGTAACCATACTTAAGACTATAAATCCTGTAATA
AAATATCCAATAAATAGTCTTTTGCGTAATAATGAAGTCATCAGGAACATTCTATTGATTTATT
TGACATCATAATTCTATATATTTAACTAGTCATAGTATATATCATTCTCAAATATTTATTTCAA
ATTCAAGCAATAAAATAAAAAAACACTTCATATTACAACTGAACTCTTTTATGAAAAAGTTGAA
TATATGAAGTGTTTTTTTATTACGATATAAACTATAAAATCCTATTCTTCGGGAACTGGTGTAT
AAACCCTTATCCAGTCCACCAGGAAGGTGTGGTCTTCCACATTTTTCAGTTCCTCATCCGTAGG
GCTTAAACCTTTAACGGCTCTCCAGCTTTGGTCTTCCATATTTATTATGATGTCCATGTCTTTT
ACCAGACCTGTACCACCAGTGTAGTTGTTGGGGTCGATAATATCCTTGCCGCTTACGGTTCTGA
CAAGTTCTCCATCTACATAATATTCAAGTGTGAAAGGGTCTTTCCAGAACACTCCTACACGATG
AAAATCGTCGCGCCACAATGTTCCCTTGTCATCCTTATACCATGAGCCAAGATCTTTCGGCTGA
TAATCCTTGAATGGCTGGCGGATGAATATGTGATGGCTCAGGTGAAGTCTGTCGGCACCGTAAC
CTCCGCCGTCTCTGTCGCCGCCGTATGCTTCTATGATGTCGATTTCCTGAGTATCGTCAGGGCT
GAGCATCCATACATCGGATGCCATGGTTGAATTTGAAAGTTTTGCGTATGCCTCTACATAAACC
GGATACTTTACACGTGTCTTCGATGTGATACATCCCGTATAGGTTCCCGGCAGTTCCTTTGTGT
TGGGTCCGCTTACAACTTTCTTCATGGGGACATCTTCAGGACGGCTGGCTCTTATTTTAAGGTA
TCCGTCGGAAACGGAAACATGGTCTCTCTGCCATATTGTAGGAGCAGGTCCTGTCCAATGATTA
TGATAGAAATCGGTCCATTTGGCATAGAACTCTTTTCCTTTATCCTTTTCGTCGGCAACATAAT
TAAAGTCGTCCGACTGTGGATGGAGTTTCCACACCATACCGTCGCCGGCATCAGCGGGTACAGG
ATAGATATCCCACTCGTACGATTTATTATTGAAATCTTCTGCTGCACAGGCTATTTGCAGCGAT
GCTAAACAAATGGTAAACAGTTTTCTCATCGTGGTATCTTAGTTTAAGTTATAATAATTATTTT
CGTTCTTTTGATTCACCTTTAGCGGTATGTGTCTGCAATGTCCAGGTAGAAAATCTCATTATGC
TCTGATAGTCTGAACTGTTGTATATATGAGTAAGACCCCATCTCAATATTTCGGTAGGTTCTTT
TTCGGCATCTGCACTGCGGTTCAGGCCAATGGCGTGTGGCGCGCCTTTTACTACTGACATTATT
TCAAAGTTTATTCCGTCAGGCGACCACTGGAGTGTGTTCTTTTCAGGACCGTCGGTGGTGATAA
GTGAAGCTATACCTCCTTTGTAAGGCCATACGCAAACTTCATGCCCGCTGTTTGAAATAGGATT
ATATTCCGATTTCACATACGGACCCATAGGATTTTCCGCAATAGCCACTCCGTGTTTGATTTCA
CGGCCGCCCCATGTTATTTCTTCTCCCATACGTTCGCCTTTGTAGTACATATAGAACTTACCTT
TATAAGGTATTATACACGGGTCGTGTACCTTATGACTGTCGAAATCACCTTTCGACACTACCTT
GAATCTGTTATCCTCATCGCCTTCCCATTCGCCGGTATTAGAAGGTTCCAGTACAGGCTTGTCT
GTCTTGATCCACGGTCCTTCAGGGGAATCAGCACATGCCATACCGATAGTATTCTTTACACGGA
CTGTGTAAGGGGATTTTACCGCCTGATAGCAAAGATAATACTTTCCTTTCCATTCCATCACCTC
AGGAGTGAAGACTGAACGGTCGTCGTAAGCACCTTTTTCACCACGTTTCACTGCAATTCCCTGT
TCCTTCCATGTCCATCCGTCTTTTGATGTGGCATACCATATATCACATCTGTCCCATGGGAAAA
CCTTATCTTTCTCTATATCTCCAGCAAATCCTTGGGTAGGTCCATAGCTCTTTGAATACCATAC
ATAATATGTATTACCTATTTTCAGCATTGCACTCGGGTCTCTTCTTACTACGCCCTCTTCATAA
GCAAGATCACCTTTAAGTGGTTCCATCTTATACTCAAAGAACCATTTATTGTCGTGATTTTCCC
ATTTCATGGCACGTTTCATAGCTGCACTTAACTTATTTCCCTTAGGTATTCCCAATGAATCGGC
CTTACGCTCATCATAATTCTGAGTGTCGTCAACGGCAATAGTCTGTGTATTGCCTGTATTTCCG
CATGCTGCCAATAGCGACATCATGCCGGCTGCAAGAATAATTTTTCTCATACTAGACTTTATTT
TATATTAATTGTTAGTTTATTCGAGTGTAATTCACTTGTTTCTGCACTGATATTCAGTACCGAT
GATTTTTCTGTCGACTGAAGCATCAGCATACATCTTCCCTGATATGTCATAATATCCTTACTTT
GATATGGAGAAACGTTCTTCACGTTTCCATTGTCTATACCAAGCAGACGGTACTCTCCATCAAT
GTTGAACTTAAGCATCTGTTCTGTTGTCTTTACAGGATTACCTTTTTTGTCTGTTAGCTGAGCT
GTGACATGCAGAACATCCTTTCCATTTGCTGCGATACTTTGTTTGTCAACCGTCAGCAATATCG
AATGTTCTTTGCCTGAAGTCCTTATAGCTGTAGTGGTATTACCTAACTTATTTTTTCCTTTTGC
GGTAATAGTGCCAGGCTTGTACTGAACTGCCCATTTATAGATATGATCCTCAAAATCGTCTATA
TACTTCTTTCCCATCGACTTACCGTTAACGAAAAGTTCCACTTCATCACAATTGGAATATATCT
CTACTATTACCGAGTCACCTTTCTGATAATTCCAGTGAGAGTTTACATCATCCCAAACCCATAA
TTTTCTATCCCATTCATGTCCTTTCTTATCAGTAAATCCATCTTTTACATGGAGATACGAAGAT
TTGTCTGTAGTCTGTGAATATATAGCAATAAAAGGCTTGTCTGTCCACAATGATTTCATCATGT
CGTACGAAGGCTTCACATAGCCGCACATATCCAGGAGACCACATCCTATCGACTTTTGAGGCCA
TTTTGAAAGACGGCTTTCACTTTCTCCCAGATAATCGACTCCTGTCCATATAAACATACCCGGA
ACGAAATCCCTTTCAATCACCGCCTTCCATTCGTGCCACTGACCGAGATTTTCTGTACCCATTA
TAGGCTTGTCAGGATAATTCTTCTTAGCATAATCATACATCACGCGACGGTAGCTGAAGCCTGC
CACATCGAGCGCGTCGATATATCCTGACTCAAAGCTTATGGAAGGCAGGATGCAGTTGGCGGTA
ACTACACGTGTGGTGTCCATCTGGCGTGTCCATGCAGCTAATTTTTGCGCTGTACGGCCAATGT
CGTATGCATGTTTAGGCTGGATTTTCCACATTTCTCTGATTTTTTCTTTAGAGTATGGAGGCTG
ATTCCAGAAATAATTACCGTTGGAATCGGCACCGAAGAAACCTGTCGCCTCGCGGCATCCGGTA
TAAGTCCATTCTATTTCATTACCTATACTCCACTGGAAGATACAGGCATGATTACGGCTTCTCC
TCATTACGTTTTTCAAATCTCTTTCTGCCCATTCCTGGAAATGCTCGCAATAGCCATGCGTAGG
ATAGTCTTCTACAGTTTCCTTCATATTGAGTCTTTTATCTTTGGGATAATCCCACTCATCGAAG
AATTCTTCCTGAACCAGAAGACCTATCTCATCGCACAAAGACAGAAACTCTTCCGCTCCCGGAT
TGTGCGAGAGGCGGATGGCATTGCATCCTCCTTCCTTTAGGGTTTTCAGACGCCGGTACCACAC
ATCGCGTATCATTGCCGCGCCAACCATTCCGGCATCATGGTGCAGGCATACTCCTTTTATCTTC
ATGTTTTTCCCGTTAAGGAAGAAACCTTTGTCTGCATCAAAACGGAATGTCCGTATGCCGAACC
TGACAGTGTTTTCAGAAATTACTTCATCGCCATTCTTGATGCGTGTCTCGGCTGTATAGAGGAC
AGGTGTATCGACGCTCCACAAATCAGGCTGTTTAATCTCAGATACGATGTCGATAATTTTCTCC
TCACCAGCATTCAGTTTTATACTGAAGACCTCAAAGGCTGCGATATTGCCTTTATTATCCTTAT
ATACTACCTCAACAACTGCAGCTCTGGGTTCGGAGTAGCTGTTGCACACGGTAACCTGGTTGTT
TACTTTAGCATATTTATCAGTAACCACGGGAGTAGTGACAAATGTTCCCCAAACCGGAATATGC
AGTCTGTCGGTTACAATCATTTTCACATCCCTGTATATACCTGAACCGGTGTACCATCTGCTGT
CGGCATAATGGCTGTGGTCGACCCTTACAGTCATACGGTTATCCTCATTGGGATTGAGATAGTC
TGTGACATCAAAATAAAAAGGAGCATATCCCGAAGGATGATATCCAAGCTTTTTGCCATTTATC
CAATACTCAGAATTATTATATACTCCATCGAACACTATATAGCATTTCTGATTTGCACTGATTG
TTGTGGGAAATGATTTGCTATACCATCCTATTCCTCCCTGAAGGAAAGCTACACATCCTTCACC
CGAAATGGAATCGTAAGGTAAACCAACACTCCAGTCATGTGGCAGGTTCACTTTCTTCCATTCA
TCACCAGGGACATAAGAAGTATATGAATAATGAGCAGAATCTTTCAGTACGAATTTCCAATCTT
TATTGAAATCAACATTTGAATCAGATGCTGAAACCTTTAGGGTTGATAATAGGATTATTAAAGC
TAAAAGATTTTTATTTCTCATAATCTTAGGTTTTACATGTTTTTTGATGTCACAAAACTATATC
TTTCACTTATAATATATGAGGGGGATATTAATGTGATATAGGGTGGGAAATCAGAATTTTACAT
CTGCCCTGTATTCCACCGTCACCTACAACCTTGACAAAGGATGTTCCTTTCTTCCCTCTTATGG
TTCTCAGGACAAACAGACACTTTCCGTTATATGTCCTTACACTATTGTTTATGACGTTGATGTT
CAAATCTTCTATCGAAGGCGATCCATTGTCGAGTCCGGCAAGTTCAAGCTTGTCGTCGAGGATT
ATCCTCACATCCGAAGGTATATCGACTACTGTGTTTCCTTCTTTATCTTCAATGGATACTTCTA
CATGGATAAGGTCATAACCGTTGTCGGTAGCTGTTTTGCGGTCGCAGTTCAGTGCCAGACGGCA
CGGCTTGCCGCTTGTGGACAAAGTGTCTTTCGACAATATTCTGTCGCCGTCCTTGCCTACCGCA
AGGAGTGTTCCTTCCTTGTATGCCACCTTCCACATCAGTATATTATGCTCCATGAAATCGCTGC
GTTTCTTTGTTCCCAACGATTTGCCGTTCAGAAACAGTTCCACTTCTGGGGCGTTGGTATATAC
CTGCACCAGTATGTCCTCGTCCCTGCGGTACTTCCATTTATCGCGTGTGTCGTACCACTCCCAG
CGTCTGATCCATCCCGGGCGTGGAGTGTAGGTGAAACTTCCGTCAGTATCCATCTTGAACTCGC
TTTCCTTTTCAGGTATTGTTACAATATGGGTTTTCGGTGTGTCTTTCCACAGACATTCAAAGAA
ATGGCCACGCGCTGTCTTGTTGCCCACGAAATCGAAGAAAGAACAGTCTCCACCCCTTGCAGGC
CATGGGCCGTTCTCGCCAAGATAGTCGAATCCTGTCCACACGAAGATGCCCGCTATGTACTTCT
TGTCGGCCACGGCTGTCCATTCAAAGAGCTGACCAACATTCTCCGAACCGATAATAGGCTGATA
TGGATATAGCTTATGGTCGATTTCATAATATTTGTCTTTATAGTTATATCCCACTAGATCAAGA
ACGTCTGTATATCCGGAGAGACGCGAAACTGACGGAACAACGACTCCTGAAGAGACGGGACGGG
TAGTGTCCACATCCTTAACCCAACCGGCAAGGACAGCGGCTGTTTCAGCCAAATCGTCTTTTCC
TCCTGACAGACGGTTGAACTCTTTCAGTATAGACTTGTTGTCTGTTTCCGGGTCGCCCGTATGG
ATAAGACCCTTGAACCCTTTATTGTCTTTGCTCGATGCCCAGTAATATGGATAGGTCCATTCTA
TTTCATTGCCTATACTCCAGAGTATCACGCAAGGATGATTTCTGTCTCGCCTGATGAACGACTT
GAGGTCGTGCTCGGCATGCGTATCGAAGTATCTGGTATATCCTATTGATATGCTGTCGGGCGCA
TCTTCCTTAGCTCGCTCAGTAATCCACTTTTTCTTTGCCACCTTCCATTCGTCGATAAATTCAT
TCATTACAAGAAGTCCCAGACTGTCGCACATTTCCAGCAGACTTTCCGAATGCGGATTATGGGC
TGTACGTATGGCATTGCAGCCTATGGAACGAAGTTTCAGAAGGCGTCGCAACAGGGCATCATCG
TATGCGGCAACACCCATACATCCCAAGTCGTGGTGTATGTTCACTCCTTTTATTTTTACTGATT
TTCCGTTTAGAAGGAAGCCTTCATCCGCATCGAATTTAATGTCGCGGATACCAAATTTTGTTGT
TTTCTTATCCATCACATATCCGTCAGAAGCAATCAGAGTAGTATGAAGCTCATACATCGAAGGC
GTTTCAAGACTCCAGAGATGACAATTCTCCAGTTCAACAGATGCAGTGAACTCATTGAAATCGC
CTTTCAGGGCAACAAAATCATCGGAAACAGAAGCTATTGTCTTGCCGTCGTACACTACTTCGTG
CTTCACGGTGACTCCTTTTACACCTGTTCCAGCATTCTTCACCTCGCATACCACATTCACCATC
GAACGGTTGCCTACCTGTGGTGTGGTAACGAATATTCCGTCTGAAGGAATATAGAGCTCGTTTC
TTAGAATAAGACTCACATTCCTGTATATACCGGCACCGACATACCATCTGCTATCGGCATACGC
TCTTCTGTCAACGCAGACAGTTATTGTATTCATCGAACCTTTTGGTTTCAGATATTGAGTAAGT
TCATATTCAAATCCCACATATCCGTTAGGACGGAATCCCAACATATGCCCGTTTATCCAAACCT
TTGAGTTATTATATACACCTTCGAAATGAATGAACACTTTTTTCCCATTCATATCATCCGAGGT
GAGAAAATTCTTCATGTAAATCCCCACACCGCCAGACAGAAAACCATTGCTTCCGGCTGTCTGA
GTCTTGGTATATCCTTCGCTGATACTCCAGTCATGAGGCAGACACACATCCTCCCACTTTATAT
CTGGACTCAGGAACAAAGTGTCCTGAGGCACGAAACCTGCTGGTTTGCTGAATTTCCAATCGAA
GTTGAAATCCACTTTAGTGGAGGTTCCGGCATAACAGAATCCGGACAGAAAGATAGTTAAGACT
GTGATAATGTTTTTTATGGTCATATCGATTTTCAGATTAATATTAATGACAAAAATAATTTCAA
AAGTGTAAAAACAAAAAAACTCTCCATTTATATTTCAGATATCAACGGAGAGTTTCATCATTAA
AAAAAATAAAACATTTTATAAAGTTACTCCTTGCTTAAGGATAGCTATTTCCCGGTATCCCTTC
TTTTCGTTCAGTGCCTGCTTTCCGCTTGCCACTTCCACCACAAAGTCTATAAAACGTCTGCTTA
AAGATTCCATGCTTTCTCCCTCTACCAGAGTTCCGGCATTGAAATCAATCCACGTATGTTTCTG
TTCATAAAGCGGAGTGTTGGTCGAAACCTTCACGGTTGGAACGAATGTTCCGAACGGTGTTCCG
CGGCCTGTTGTGAACAGCACGATATGGCATCCGGCAGAAGCAAGAGCCGTACTTGCCACTAGGT
CGTTGCCTGGTGCGCTCAACAGGTTAAGTCCGTGTGTTGTGACACGGTCGCCATATTTCAGAAC
ATCCTCCACCATCGAGCTTCCCGACTTCTGTGTACATCCCAATGATTTCTCCTCAAGCGTGGAA
ATACCTCCCGCCTTGTTTCCCGGTGAAGGATTTTCATATATTGGCTGGTCGTTGCGGATGAAGT
AGTTCTTGAAGTCGTTTATCATGGCCACTGTGTCGTCGAATATCTCCTTCGTGCGGCAACGGTT
CATGAGCAGTGTCTCGGCTCCGAACATTTCAGGTACCTCCGTGAGGACTGTTGTCCCACCCTGG
GCAACAAGATAGTCAGAGAACACCCCAAGCATCGGATTGGCCGTGATACCGGACAGTCCATCAG
ACCCGCCGCACTTGAGTCCTATACGCAGTTTTGACAGGGGGACATCAGTCCGCTTGTCTTCCCT
GGCTATGGCATACATCTCACGGAGAAGTTTCATACCCTCTTCTATCTCATCATCTACTTTCTGA
GAAACAAGGAAACGGATCCTTTGGGTATCATAGTCACCTATAAACTCACGAAAGGCATCAGGCT
GGTTGTTCTCACAGCCAAGACCTACGACAAGGACAGCTCCGGCATTGGGATGAAGGACCATGTC
ACGCAATATCTTACGGGTGTTCTCATGGTCGTCACCCAACTGCGAGCATCCGTAGTTATGAGGG
AAAGATATAATGGAGTCAACCCCCTCGCAACCTGTTTCCTTGCGAAGCTGCTCGGCCAACTGGT
TTACTATTCCGTTCACGCAACCCACCGTAGGGATAATCCATATCTCATTACGTATGCCGGCTTC
TCCGTTAGCACGCAAATACCCTTTGAATGTATGGTTCTCGTTCGTGAATGTCTGTTTCTCGAAC
TTCGGAGTGTAAGTGTATGTACTCAGACCGGAAAGGTTCGTCTTGACGGTTTTCTCGTTCAGCA
GATGTCCTTTCCTGACTTCCTTTACAGCGTGCGATATGGGGAAACCGTATTTTATCACCATATC
ACCTTCTGCAAAATCCTTCAGGGCAATCTTATGACCGGCAGGTATATCCTCCATTAATTCTATG
GAATTGCCGTTCACCTCTATTACAGTCCCTTTGGACAATGGGTGCAGTGCCACAGCCACATTGT
CCGGAGGGTTTATCTGGATATATTGAGTCATAACAAACTAACATTTATAAATTGAAGAATACAG
GTAGAAGTATCAACCTACAAGGTCTTTTACTGTCTGAAGCATTCCTTCGCTCTGGATTTTGTTG
ATATAGTAAATTACACGGTCTGCCAGTCCCGAGATAGTATTAAGGTCTTCACCCCAAATGGAAG
TATCGGCGAGAACTGTCTTCACAAGATTTTCTACCGAGCCATCGTTCCACAAACTTGTAAGCAT
CGCCATGATTTCCTGTGCATCGTTAGGAACTATCTCTACACCATCGGCACGCTTTCCACCTTTG
TAGTATACTATGATGGCTGCAAGACCGAGTACAAGTCCTTCAGGAAGCACACCCTTACGTTTCA
GATATTCCTTCACTCCTGGAAGGTCGCGTGTGGCATACTTAGGGAATGAGTTAAGCATGATTGA
TGTTACCTGATGGTCTACGAAAGGATTATTGAAACGTTCCAGGACATCATCGGCAAACTTCTTG
AGTTCCTCTTTCGGCAGGTTGAGGGTCTCCATCAGCTCGTCGAACATCACACGTTTGATGAACT
TGCCTATCACCTCATGTTGGCATGCGTCTCTCACGATATTGACGCCCGAAAGGAATGCCACCGG
CGACAATACAGTGTGAGGACCGTTCAGCAGAGTAACCTTGCGTTCATGATAAGGCTCCTCCGAC
GGGACGAACAGAACGTTCAGTCCCGCCTTGTTTGCAGGAAATTCTTCGGCAACCGATTCCGGTG
CTTCGATAACCCACAGATGAAAAGCCTCGCCCTGTACAACTAAATTGTCATCAAAGTATAGTTT
AGTTTTTATGTTGTCTATGTCTTTACGAGGGAAACCCGGTACGATACGGTCCACCAGTGTGGCA
TATACACCACATGCAGTTTCAAACCATGACTTGAACTCTTCGCCAAGGTTCCACAATTCAATAT
ACTGATAGATTGTTTCCTTCAGTTTGTGACCGTTGAGGAAGATAAGCTCGCATGGGAAGATGAT
GAGTCCTTTCGACTTGTCACCGTTGAAATGTTTGAATCTGTGATAAAGCAACTGTGTCAGCTTG
CCCGGATAAGAGCTTGCAGGAGCATCCTCAAGCTTGCACGACGGATCGAAGTTGATACCGGCCT
CAGTAGTGTTCGAGATTACGAATCTCATATCAGGCTGTTCCGCCAGTGCCATGAAGTCATTATA
CTGGCTGTATGGATTCAGCGCGCGGCTGATGACATCAATCATTCTGAATGAGTTCACCACCTCG
CCATTGTTCAGTCCCTGAAGATTGACATGATACAGACAGTCCTGGGCATTGAGGGCATCAACCA
TACCTTTTTCTATAGGCTGCACCACAACAACACTGCTGTTGAAATCTGTCTTTTCATTCATATT
CGAGATAATCCAGTCGACAAACGCACGAAGGAAATTACCTTCGCCAAACTGTATGATACGTTCC
GGACGTACTGCCTTTACTGCAGTCTTACTATTTAAAGCTTTCATTGTAATGCCAAAAAATTAAA
ATTGATAAGATTAAAATTCAACCAACATTCTGAATACCTTACCTGGATTTTCCGACCATTTCTG
CAGAGCCTCGCCTGCCTCTTCAGGTTTCACTACGGCAGAGATAAGTTCGTTCATCGGGCAGTTG
CCATTCTGAAGATAATGTATCACGGCACGGAAATCCTCAGGCATTGCATTGCGCGAACCGCGTA
TGTCGAGTTCCTTCTGGACAAAATATTTTGTCTGGAAAGCCACTTCACTCTTGGCATAGCCGAT
ACATGCCACACGGCCTGTGAAACCTACAATGTCGATGGCAGTAACATATGTGATAGGACTACCC
ACAGCCTCTATCACCACATCAGCCATATAGCCGTCAGTAAGTTCCCTTACTCTTTCCACCACAT
TTTCAGTCTTCGAATTGATAACCATCGAAGCACCCAGGCGTTTTGCCAGTTCAAGCTTCTCATC
GTCAATATCCAATGCTATTACCCTTGCGCCACGAAGCGATGCTCTTACTATGGCGCCAAGTCCA
ATCATTCCGCAACCAATCACGGCCACAGTATCAATGTCAGTTACCTGAGCTCTCGACACGGCAT
GGAAACCTACGCTCATAGGCTCAATCAGCGCACATTCCTTATCCGAAAGACCGGCAGCCGGAAT
AACCTTTGTCCAAGGGAGGACAAGGAACTCCTGCATAGAACCGTTACGCTGAACACCCAAAGTC
TCGTTGTGTTCGCAGGCATTCACACGTCCGTTGCGGCATGAAGCACACTTTCCGCAGTTGGTAT
ATGGATTTACTGTCACGTTCATTCCCTTCTCGAAACCGACAGGAACGCCTTCGCCTATTTCCTC
TATCACAGCACCCACTTCATGTCCCGGGATGACAGGCATCTTCACCATAGGATTTCTTCCCAGG
TAAGTATTAAGGTCGGAACCACAGAATCCGACATATTTGATACGAAGTAAAATTTCTCCGGCTC
CAAGTGTTGGTTTAACTATATCAGCTACTTGAACCTTTCCGGCTTCAGTAATTTGTACAGCTTT
CATAATCTATGTATTTATTTAAATTTGTTATTGTATTATTTTGATGTTGCATTAATTCAATGTT
GTTTTTTCTCTATCTTATATCCTCTCCAGCCATAATATGCCGTAAAGAAGAAACATATCAGAGG
TATTACATATGCCACCTGATAGAAGTCCGCGTTATGATTCATCACAAATGCGGTGAACTGAGGG
ATGCACGCATTACCTATAATAGCCATCACAAGGAATGCCGAACCACTCTTTGTGTCCTCGCCAA
GGTCGCGTAGTGCAAGTGAGAACTGGGTTGGATACATTATCGACATGAAGAACGACACTGCAAG
CATGGCATAAAGTCCTGTCATACCACCGAACATGATAATTACTCCACACAGTATGATATTTACT
ATAGCGTATGTAAGCAGCATATCCTGAGGTCTGAATTTCGACATTAGCATAGTACCTATCCATC
TGCCGCCAAGGAAAGCCAGCATATACAGTCCGAAGAATGTGGTCGCCTCATCCTCCGACAGACC
TGCATACATGCAGCAGTAAACTAGGAACAGGCTGTTGATGGCTGTCTGCCCTCCGTTATAGAAG
AACTGTGCGATAACTCCCCATCTCAGGTGTTTGCGTTTCAACACTGCAAAATTGATAAGCTTGC
CCTTCTCGCCGTGCGATTCCTCCTTGTCAATATCAGGCAACTTATACAGTGCAAACACCACAGC
AAGAATAATCAGCAGGACTGCAAGAACCAGATAAGGCATCTTCATGGAGTCTGTCTCCATCTGA
ATAAATCCGTCCCAACCTCCGGGAAAGTCGGCAGGCAGAGTCTCGCGAGTATAGTTCTGTCCGG
TAAGTATAAGCTTACTCAGAAACATTGCGGATATGAAAGCACCAAGACCGTTGAACGACTGTGC
AAGATTCAGTCTTCTTGAAGCCGTATCGTGTGTACCCAGAGCTGTCACATACGGATTGGCAGCA
GTTTCGAGGAAGCACATTCCCGTTGCCATGATGAAGAAGATTACAAGATATGCCCAGTATTCCT
TTATCTCGGCTGCAGGGAAGAAAAGCAGACCACCGATGGCTGCAAGAATGAGACCGACAATTAT
ACCCGACTTATAGCTGAAACGTTTCATGAACATTGCTATCGGTATGGGAAACAGGAAGTAGGCC
AGCCAATAGGCAGCTTCAGTGAACGAGGCCTCAAAAGCATTCAGTTCACAGGTTTTCATCAACT
GCCTGATCATTGTAGGCAATAGATTAGTGCTGATAGCCCACATGAAGAACAAGCTGAATATCAG
TAAAAGCGGTATAAAATATTTGTTTTTCATTCTGACATGTTTTTAATATAAGGTAACTCAGGCA
GATTCTTGAAACCGTAAAAGGCTTTCGCGTTCTCGCCCAAGAAAAGTTTTTTGCTTCTCTCTTC
CAATTCTTTTGATTTAATCACAAAGTCGTACGACATCTTGTAGGTAATGGCTGTGATTGTGCGT
GGATAGTCGGAACCCCACATCAGTTTCTCGAAGCCAACAAGGTCGGCAGCTTCGTTGATGGCTC
TGACAGCGCTGCGGAACGGATAGAACTCGTCATTGAACAGCCAAGTGATACCGCCCGACTCAAT
CATCACATTCTTATGACGGGCAAGCATTATCTGCTTCTTCCAATCCGGTTTAGTCACCATACCG
AAATGCCCGATGGCAATCTTCAAGTACGGACATTCTGAAATGATTTCTTCCATCTCGCCCACCT
GGAGGTCTCCCTCTGCCATATCTATGGAAAGAATCACCCCCTTGTCTTCCATTAGATGAAACAT
CCTCATCATCTCGTCCGAGTTGAGCATCACCCTACCGTCCTTCAGTTGCAGGCGGTGTCCCGGA
ATCTTTATGGCCTTGAACCCTTTGTCTATAAGTTCAACCGCCTGGTTATAGAAACCCGGTTTTC
TGAATTCACACATACCACACACGAAGAACCTGTCCGGATATTTCGTCATCACCTCCATCAGATA
GTCATTCTGAATGCCGTCGATATACTCCTGTGTGACAACAGCCGCGCCAATCAGGGCATAATTC
ATATTAGCCAGGAAAACCTCAGCCGTGTTTCTTCCGTCAATCATAAAGGGGGGGGGAGCATTTG
TCTCACCTCCCCCATAAACAATGATTGACCGTTCTCTGTAGTCTTGATTTTCAGGCCATCTACT
TCAGTGTCCTGATAAAGCCACAGATGCGAATGGGCGTCAATTATTGTATAATCCATAGAAACAG
TATTTATGAATTTGCCCAACTTACTCTTTGCTGATCGCCTATTATCTCCTTAACCTTTTCCACA
AGGCTCCAGTCTATCGGTTCCTCAATGTATTTTATGTTCTGAAGCACAGACTCTGTTCTTGCCG
AGCTGAACAATGTTGTAGGTATTCTCGGATTGCTTACAGAGAACTGCACCGCAAGTTTCTCGAT
AGGGTATCCCTGTTCAGCACAATACTTGGCAGCCTTTGCACACACCTCAATCAATGGTTTTGGA
GCCGGATGCCATTCAGGAACACCTCTATGTGTGAGAAGTCCCATACCGAACGGCGAAGCGTTTA
TCACTCCCACACCATTTTCGTCAAAATAGTCGAGGAAGTCCACCAGCTTGTCGTCGTTCAATGA
ATAGTGACAGAAGTTAAGCACCGCCTCTACTGTACCCGGAGCGGCATGGTCGATAATCCATTTC
AGGTTTTCGAGCTGCAGGTCGGTGATACCCACGTGGCCCACCACGCCTTTCTTCTTCAGTTCCA
CCAGAGCAGGCAATGTCTCGTTCACCACCTGGTTCATATCCGAGAACTCAACGTCGTGAACGTT
GATAAGGTCGATATAGTCGATGTTCAGACGTTCCATACTTTCGTAAACACTCTCCTGAGCGCGT
TTGTCCGAGTAGTCCCACGTATTCACACCGTCCTTGCCATAGCGTCCCACCTTTGTAGAAAGGA
TGAACGATTCTCTTGGCAATTCCTTCAGAGCCTTACCCAATACGGTTTCGGCTTTATAATGTCC
GTAATATGGAGAAACATCAATAAAGTTCAGTCCGCGTTCCACTGCTGTAAAAACAGACTGTATA
GCGTCACTTTCTTTGATAGAATGAAAAACTCCGCCCAATGAAGATGCGCCATAACTCAATACAG
GAACCTTAAGTCCTGTCTTTCCCAATTCACGATATTCCATTTTTGATAAATAATTTAAAGGTTA
ATATTTTTTACTCTGTTTATTCTTATTCATACAGATAGAACATACGTTCCATCATCTTCCATTT
CTCGTCCGATGTGGCCCCCTCGGCACACTGCTGGAATTTGGCTACGTATTCTTCCCATTCGGCC
TGACGCGGCAGAGTGGCAAGCTTTGCCATAGCTGTATCCCAGTCAAAATCCAGAGGTGTTTCCA
CTATCATAAAGAGTTTTGACCCCAATATGTATATTTCCATTTCCAGGATTCCCACCTCGCGTAT
TCCGGCGCGTATCTCAGGCCATGCCTCTTCCTTACTGTGAGCCTTTCTGTAGGCTTCAATCAAT
TCCGGATTCTCACGCAGACTCAATGTCTGACAGTATCTCTTCACAGGCAGGGAATAACTTTTCA
CTTTATATCCTTCTGTCTTCATGATATTATTGATATTAATATGTTAGTATTAGATGTCACTGTC
TTTATCTTTTCGACGATGCTAAAGTATGAAGTATCCATCAAAACAATAGAGGAGATTTTCAAAA
AAGAAAGAGGGGATATTATACCCCCTCTTTTTCGACATTTTTACCCCTCATAAAGGAGATAAAA
AGTCACCCCAAACTCTATAAAAAATCAAAACAGATTGAACTGCATTCCTGTGTAGAAAAATCCC
TGGTTGGATTTCGGATTCCAATACGTCATCACCGTCAACGGGATTTCATATTCCATAATCCGAA
GTTTATAAATCACATTCAGGGACACCTGAGTAATTCCTGCCGATTCGGCATACATGGTTCTGTT
CACCATTTCCCCGCTTTCATTTCTTGAATTTCTCAATGCGAAAGCTGTTCCAATACCAGGACCG
ACCCTTAGCTTTTCGTTCTGATAGATGGTATAGCCCACATATACGAAACTGGAGTAGATGTTCT
TGCTGTTGTCCAGATCCCTGTCGCGACCGTAAACAAGTGTAGAGAAGCTCAACTCCAGCGGAAA
TTTCCTGTCGCCCGTATAATTGACCATGAGATCAACGAAACGTCCAGTTTCATCAGGCTTATAG
TTGAAGAACTCCTTATTATTATATGTAGCCCCGGGCGAGAAATTATATGTATCTATAGCCTTTA
TCTGAAACCTGCCATGAGTATATGCTATATACTGGCTCAGCTCCTTATAACTCCCCCTGGTGTT
CGATCCGCCAAGGAAACCGGCGGTAAACCTCCCCGATGGGTCGGAAACCGACAAATCGGACGAG
AGAATCAGTCCGTCGGCCACTTCAATGCCACGCCATAGAATCATGTTCTGTAGAGTAGTACTGA
AATGAAGCTGAGCCTGAACATTTGCTGACAAAAATATAAATACAGGAATTAACAGTCGCTTTTT
ATACTTACAGGTATCCAATGATAATATATGTATCATACTCAGAGCAGTAGAAAATCGGTTTTAA
ATTATTATTATGGATTTATTTGTCGAAATACTCTATAAGATTATAAACATTCCAGTTAATATCC
GACATGTATTTGGTCAATGATGTATAAGGTTTATAGTTATAATCGAGCATACCTTTATTGCAAT
CCTCATCATCCAGATACTTGAAGAAAACCCATCCTACACAATTCTTGGCTTCGAGCAGTCCCAA
GGTAAAATGCTGGTAAGCGAATCCACGGTTTTGCTGGTCGCGTACCACGAAACCAGCTCCACTT
GAATTGTCAAGCTTAGTATCCTCACCCTTGGTATAGAATTCCGTTACCATGAAAGGAGTACCGC
CCGCCTGGTTCTTCCAGCCATCCATGTAGCCTTTTTCAGGCGACCATTTACTATAATAATTTAT
GGAAATGACATCACAATATTTTCCCGCTGCCTTAATTATATAACTGTTGTATTTAGGAAGGCTG
TGCAGGCGTGAACCCAGATAAAGCAATTCAGGATCCTTCGATGCCTTAACCGCATTCTTTATGG
CAGAATAATATTTTTCCGCACAAATACCGGCAAACTCATTGTTCAGTTCATCCGTTACATCAGA
AACATTTGCACTCTTGTCCTTATCCGTCATAAACTTGGCGGCTGCAATATAAGCAGGATCCTGC
TTGTTTGAAATTTTCAGGAATCTGTCGAGCAGCCTGTTTCCCCATGTAGAGAAGTCTATCTCAT
TATCCGAGAAGAATCCCAACACATCCGGGTTGTTTCTGAACATGCCGAAAGCATCCGAATTGAG
ATACTCCTTGCACCATTCATCCCATCCATCATAAAACACAAGACCTATCTTAAGATTCACGTTC
TGCCCCGGATAGCTAATTCCCTTGCTATTCTTGAACTCTGCAAGGAATGAAAAGGAAGGAGCCT
GTGTCAGAGGACTTGAAGCCGATTTATTATAATCATTTACAGCCTTGTCGCCTTCTTCCTTACC
GAAAGCGCAGACACTATGAAATCCTATTTCAGAGAATTGTTTCTGCGACTTTGCCACCCAGTCA
TCTACTGAACTGTAAAGCTTGCCGAAAGCTGAGCTGTTGCCATCCATTCTGAATGAGGCGATAC
CCCTTACATAATATGGATAACCTTCGGGGTCGACTATCCAACTTCTTCCATTTGAGTTTTTCTC
AACCCTGAACCGTCCAGTAGCCTTGGATTTTTGCCCTTTTGCGTATGAGCCATATTTATTCACG
CTTTGCAAATACTCATCCTGTGTTTTTGTCTGCTGTTCATAACCAACCAGGTATGGCAATATCC
TTGTCTTTGCCTCTATAAAAGCCTTGTCAGGTTTTTCCGCATACTCGACAATTATCGGTTGATA
CTGCTTGGTGCTATTAGGATAGGTTTCAGCAGGACCGGGAACAGGCAGTTGCAGTTCTACATCA
TCATCGTCATTATCGCCTGCATTGTCGCCGGGAGTATTATAGTCCTCCACATTCCCCGGTTGTG
AGTAAATAACCTCAGGCGGAATATATGAGAACTCCTCCTGAGGGTCTTCACATGACAAAGCGAA
GAACGGAACACTCAAGCAAATGGTTTTAGTAATAATAGTAGAATATTTCATTGTTGCAAATATT
TAGTAAATTAATATAAATCCCATGTCCTGATTGTATCCCCCCATCGGTGGTCTATCGGGAACTC
CATTTCTCCCCATGCCTTAACAGAAGTCCAAGGTTGGTCGGCATCAGTCCAGAATGGGTCAGAG
GCAGGCAATCCCAACGGAAGGAATGCAAGTGTAGTCATATACAGGCTGCCATTGTTTGTATAAT
GATTCGAAATGCCAGTCTGATGTCCGCAGAATCCTATGGTGAGGAATCCGCCCTCATTGAAGTT
ATTGCCCGACTTGAACATACGTTTCATACACGCTGTCAGCGCACATCTCACCTGTGCTTTCGAT
ACTCCCGCCGGCAACTCATTATACCATGCTATAAGAGCCAGTGGCTGCATTGTTGCCATACGGT
AAGGTATAGAGCGTCCGAAAACAGGGAATGTTCCTTCAGGAGATATGAAACGCTCCAGAATCAT
GGCGAACCTCTGTGCCCTCATCAATGCCCTGTCATAGTACTTGCGATAGTCGAAACGTGTCCTC
ACGCCCGATTCCATTATTGCATGTATAGATTCGAGATACATAGGATGGAACACATAACTGCTAT
AATAATCGAATGCAAAGTGCTGTCCGTCTGCGTACCATCCGTCGCCTACATACCATTCCTCCAC
CTTGCGGAAAGTAGAATTTATACGATATGTATCCTGTCCGGCATCAATTTTGGCAAGGAAGCTT
TCAATGGTGGCCGAGAACAGCAGCCAGTTAGTGTAAGGAGGGTCAATGCGTCGGAGACCTTTGA
ACTCTTTTATGTAGCGTTCCTTTGTTGTCTGGTCCAGCGGTTTCCACAGCTGGTCGAACGCGCG
CAGGAAACTTTCCGCAATATAGGCAGCATCAACCAGTGCCTGACCATGACCGTTCCACAACAGA
TAATCCGGACTATTAGGGTCCACCGCATTTGCATAACTCTTCAATGCCCATTCTTTCAGTTGCT
TGCGCTGCTGTCCTTCTGCTGTATCATCGTCAGGCAGGCTCAACCATGGAGCTATACCGGCCAT
GAGACGTCCGAAAGTTTCCATATATGCAACCTTCTTGTTACGGTTATCCCAGTTTGGACTTACC
TCAAGAATCATATTTTTCTGCAGTTCCCCTTTCGCCATATTGCTCAACACAGGAGCAGCCATCC
TGTAAGCCATATCCGTCCAGTATTTTCTTGTCTCGTTGTTGTTTGCCTCGAGATAACGCACATA
CTCGCAAGCGGCAAGAAGGAATGCGCCTACCCCAAAGTTGGCAGTCGACTTGGCGTCAACCACC
TGTCCCGGAATAGCCTTTTCACCGATTGGCTGGACATAACCCACCGACCAGTCTTTCTGCAGTG
CAGTCTTGGTAAGATATTTCCATGCTTTCCCCACTACAGGCATAAATTCATCCTTGTCAAGATA
ACCGTTGTTTATCCCCCAAAGCATACCGTAAGTGAAGAAAGCGGTACCGCTTGTTTCCGGTCCC
GGAGCATGTTCCGGATCCATCATACTTCTTGTCCAGTAGCCCTCCGGCTGCTGCAGACATGCAA
CCGCCTTTGCCATACGCACAAACTTATCCTCGAAAAAAGACAGATGCTCATAACCCTCCGGCAG
GTCCTTCAGCACCTTTGCCAGAGCGGCAAGCACCCATCCGTCGCCTCTTGCCCAGAAATCCTTC
TTTCCGTTCAGACTCTTATGCTTGGGATAAACATATTTTGCGTCGCGATAATAGAGTCCTTCCT
CCTCATCATACATTATTGAGTCCGACGTACAAAGATATTCATACAGTTTCTTAAGATACCGGTG
ATTATGCGTAATCTTATACATCTTCGTCATTACCGGCATCACCATATAAAGTCCGTCGCTCCAC
CACCAGTAATCCTTACGCGGTGTGCTCATCTGGTACTCCATGACTTCGCGTGCACGCTTGATTT
TATAATTCTCCGGCATGACGTTATACAAGTCCGCATAAGTCTGGAAGCACACCTGATAATCGCC
GAACAGCACATAATCATCCTTTACCCCGTATTTATACTTCCATTCAGATTTGTTGTTGCTTTTC
GCACCCATCCACTGGTTATACTCAGCCCATGCCTCCGAATACTTTCTGTATTCTTCTTTCCCAG
TAAGGAAATAGGCTTCCATATTACCGGTGTGATATGCCGCATAATCCCAGAAAGACCTTGCTTC
GGGGGCATGATTTTTCTGCCAGGCATCGTTCACTTTTTCAATCATCTCCCTAACTTGCTGAGCC
TCAGTTTTTTTTTGCGAAGGAAAATGAAGGTAAAACAGCTATAAGGATGTATAACATCCAGTAG
TATCTATAACAGTTCATCTTTGTGATATTGTTTAGATTTTCTAAAACGAAATGGGGAAGAATAT
ATATTCCTCCCTCATTTCACGAATAATTGTATTATTATATTTATTTGTTAGGAGTCCATTCTGC
TCCGTTGTTGAAACCTTCTGTTGTAGAGTCAAAACTTGCATCTGCTCCTGTACTTGGTCTTTCT
GTAATTTCTTCAATCTTAAAAGAAGTGATTTTAGCGGTTCCAGTAGCATCAGTACCACCAGGGA
CATTAGTCTGTAGAGTTAAAATAACGTTCTCAAGAACCGGCCACACAAGTGAACCATCTGCTCT
TGAAGCTGGAGTTTCAGCAGAAGTAGAACTACTGATTGTGAATGTATTTGTATAGGTTCCACTT
CCGGTATTTCTTCCAATCCAGAATTTATATTTATCAGATGCTCCCAATCTGAATGTTGTTGCAC
AGTCGTTAGATGCGTATGTATAAGTAAATTTGTAAGTAGAACCATCACGGAATGACATTGATTT
AGTAACTGGGAATTGATTATCTGCTGGAACAATTTCCAATTCTCCACTTGCATTAATTTTTTCG
GCAACTCCTTCTGCAAGATATTCCTTAACTGCATCTATGTTAGCGAAGTTAAAATCAAAAGCAT
CTGCATGAGTCAAAGCAACATTGGCAGATTCAATCTTGATATTAGCATCTTCGTTGTTTTCGTT
TTTAGCAGTCAAAGCACTAACAGCATAATCAGTGTTATAACTTACGCTAATATTTGCGTCATTA
CTATAAATCTTATCACCAAGAATAAGAGTCATAGTAGTTCCATTCACAGAACCGGAAGCAACAG
GAATTGTTTTTCCTGCTACTGTTATGGTAAATGCTTTGTTAACAGCATCAGTGAATGTTCCAGA
AACTTCCTTATCGAGTGTAAGTTCAATTCGGTCATTACCTGTTGTCTGATCAGGAACAATTTCT
TTAGCTGAAGAAACGGCAACAGTAGTTTGTTTTTCCAAATCCACAGGAGGTTCACCGCCTCCTT
GATCATCATTCAATACTATCGTTACAATCTGTCCTTTAGTAACTATAAGGTTTTCACCACTGAA
GTTATAAGTTTTAGTACCAGAATTTCTTGTAAGTTCTAAAGTAAATCCATCGGTAAATGTCACC
GGAGCTACAACCATTGAGTATTCCTTGGCATTTTTATTTTGTTCATTAGGACCAACAAATGTTC
CCTCTTTAGCGGTTAGAGTTATAACATTAGAACCGGATTCCACTGTCAGGTTTGCTGAAGCATC
AATTTTTACGTTCCCTGCAATCTTTACATCACCACCAGCAGTAAGTTTAATACCTGTAAGGTCA
GTAAGATTATTTTTAAACTTAACCAATCCACAAGTATTCTGGAAAGTTAAAGATTTGTTATTAT
CTGTTGCAGTAGCATAAGATATATTTGCATTTGCATCGAATCCCCAAGCCGGAGCTGTCTGTTC
AGATGGCAGTGTAGTAGTTACGACACCTTCAAGACACACAGCTTCGGCATTATAAGGATAAAGA
GCTGTATATGAATTGTTAGGTGTAGCCTTACCTGTAAACGTTGTAACTGTGCTACCACCTGTAG
CGGTAGTAAACTTGTTATTTTCTTGGCCTGAAAAGATATTGATTGCATCTCCTGTTGTCCACCA
CACCGTTGTTCCATTCTGCAACGAACTACGGCTTGAAGGCGTACCGGCAACAAAAGTCATATCC
TGAGGACCACTGACTGCATTTACATTCGACAGTTCGTCTTTTGTACAAGACTGGAGCATTGCAA
TACTCATCAAAGCCGCTCCACAAAATAGCATCGTATTTTTCATGACATAAATTATTTGTTAAAC
AGTTTCAATAATAAAAAATCACATCACTTGTTATTCATATTCTTATTCTTTAGGATCAGGTTTC
CATTCAGTACCGTCATCTTCAAAATCATCATGACCGCCATCTACAATTCCGGGAGGTATTGATA
TTCGGCATACCGCACTTTTTATTCCATTACCCGTATCTACAGAAGCACCGATATTAGAATCTCT
GCCCCCGTCGATTGCCACGACCGTACATCTCATTTTATCGTCCGATGGTGTAATCATCAACACA
TCAGGGAAAGAAGTTCCCCAAACTATTGACTTGTAACCGGTATAAGGGAGATTATCCTTGGTTA
TATTAATACCCAACTCCACAGTGCCACTATATGGTAATTCTATATAACTGACAGGTTTGTTGTC
AGTCTGCCCATCCTTGAACACTACATATTCAATTTTTATCTCCTCAGCTGGTGTTCCATCACCT
CCACCTACGCCATCATCATCCTTATCACACGAGATTGCCGTAAACTGTATAAAAAGAAGTATGA
AAAGGTTGTATACTGACAGAATCCGTGGTTTTATATCAACCATAATAAAATGTTATTTAAGCGC
CAAACAAAATTTTCAATATTCAAAAGGCATAAGAGGAAACCCTGAATATGCCTTATTACCATGA
AAACAAATCAATCTACCTTTTTCAATCCGGAATCAGAAAAATATGTTATTTATTTAGAACATAT
TTTTCCGATTTGCCAGATTACAATCACAATAAATAAATCAACAACTAAATCTAATTACCTAATC
TTATAACTAAACCCTCAAACAATGTTATTTAACCTTTTCTATCTTGACATCATCAAGCAGGAAG
CATCCACCATTACCTGAACCCGGAACAGCTGTGAAACGATATACAAAACCATTTTCCTGCAATT
TGAATTTAACTGTTGTAAGATTGTAATTCTTACGGTCTTTCTTGACCTCAGCAGTGGCAATTTC
TTCCAGTTTCTTTGAATCCGGATTATAGTACTCAATCCTGAAGTTAGGTTTGTCACCCCAACTG
TATTTGGTATAAGCTGAAATCTGATATTCTGCTCCAGTTTCATAGCTGATGTTTACAGCCTGCC
ACATACCAACCTTCACCTCAACAGCATAGTTGCCTGAATGTGCCTTTTTCGCATCAACTATTTT
GTTATCTTTCTTTTCCCAGACATTCCATGATGTCAAGTCACCTGACTCAAAATCACCGTTCTTA
ATTTCCTGAGCGTATGCAGAAGTCATCATCATTCCGCAAGCCATCATTGCTAAAATTTCTTTTT
TCATTTTTTCTAAGGTTTTTAATTTAAGTATTATGTTGTATCTATTAAAATCACTCTTCTATTG
GAACCAACTTATAAGCCCTGACCCAGTCATAATAAGTAGTACTTTTGTCCTTATCCTTCAAGTC
CTCAGCTGTAGGTACTTGTTTTTCCCAATCGTATGTTTCAGTAACTATATGTATGAACATAGGT
CGGTCAAACGGAGTATCTGTATATTTTGTTGTAGGCTTGATAGTGTACATATACTTTCCGTCAT
AATAGAATTTCACGGTATTTGCATCCACCCACCAACAACCGTAAGTATGGAAATCTTCTGCCGA
TGGGTCCGTCATATACGAAACCACATCCGAACGTTTCGCCGTATTGTCAGTACGTTTGCCTCCT
TGTTCCTGATACCAATAGTGAGTATTACTGTTCATCTGCATATTCCATGTCTTGTTCCACGGAT
TATCAGGGTTGACACTTCTTATTATACCCATTGTTTCTATAATATCAAGTTCCTGACTGCTCCA
TGTCTTTATCTTCTTGCCGCCTTTCATTATTTCCTTCATTACCGGGCGGTTGGAAAGCCAAAAA
GTAGACGACATGGTAGTGAGCGAAGCCTTCATCCTTGTTTCATAATACCCATAATGTGCCTGGT
TCTTTGCAGAAGCAACCGCTCCACCGGCAAGACGATATTTATCGCCCGGCTTTCCATCAAGTCC
TTCTGTTGGCGACAAAACGGTATTGATTATACGAAGACAACCTTTCTTGACACTAACATTCTCT
GCCTTGAAAGTTGCAGGCGGCCGACCGTTAGTCCAATAAGGACTTTTAGCATGCCATTTAGCGG
CATTAAGACGTTTACCATTGAATTCATCAGTATAATCTTCGTTAACTACCCATTTATAACCCTC
AGGAGCCTCAGGCAAATTTTTTATATGCTCTTCAGCCAAAGAATATTCCTTATCATTTTTTAAT
GTATAAGATGACAGGAATAAAGATGCAGCAGATAAATACAATACTGTTTTTCTCATAAACTTTG
TCGTTTTAGATTTTTTGTTACACGACAAAAGTATATAAGTTTCATGAAAGCATTAAGGGGGATT
TACATCGTAAAAGGTGGGGTAAAATTCTACCACTCCCTGAAACACAATTATTTCACTCATGAAA
CCATGTGTTTTTACGATATATAAAACCCGACAGAAGAATAATACCGTATTACCGGCTAATTTAC
ATAAGAATAACTTTTCAAACCGCCATATACCCCACTTTACGTCCGTACCCTCAGTCCTCGACTC
CGGCAATATGTTTTCCATATCGAGATCTATGGTTTTCTGCCTCGGATTCAACCACTAACTGTCG
AGCATGTGGATTGCGTATCTGTCATAGAATCTCTTTCCGAACCATATTATCTCGTCTGTGCTAA
GTATGTTGTTCAGACGGATAATCTTTCCGGTATTTTACCACCTACTTCTCTTGCAAATCCTGAT
CTGATATAACCGGATACTCTCAATTCATTGATTTCCGACTTGTATACAGTCTGCGAAGAGGCAT
TGAAACTACTGCACAGACTGAACAGCAGCAGGGGAATAATTTAACTGATTTTAATAGTAGACAT
TCTGTGTTCATAATATTTCATTTTAATGATTACGTTTCTGACTTTCGTCTGATGCAAAATTATG
AGGTATCGGACGGGGTTGTATCTTTCAGTAAAAATCAGTAAAGTCTTGGCAAGGGGTAAAAAAC
TTAACATCTTGTATATAAATATATTACAAACAAGGTGCAAAGATTTTCAGTAAACGATGGCGAA
TACAGAACCTATATATTTACACGCCATAAAATGAAGAAAAAGCAGTAGGAAAAAAATGCGGGCA
AGTTCCGGATAAAATGTGGGCAAGTTTAAGGTAAAACTTGCCCGCATTTTAGATAGAATGCGAT
CGCATTTAAAACAAGTAAAAAACGAAGAAAAAAAATATGTGTTCTTCACAGAACACATATTTCA
AAAATAGGTATAAACACGCTAAACAATGTTAACAAAATCTATTTATAAAAAAAGCTCACATCAA
TAATATCTGCAACATTTTTACAATACTCCATAAATGAAGAGACCTTGGGATGATTTATACACAG
AGCTATCTGTGATGTAGGCGAAAAACGTCCTGTCCCGTCAAGAAACGCTGTAAGCTCAGATGGG
AGGAGTATACTGCCAATACCTGGATTTACGTCAGTCAGAACGACTGTATTTACAGCTTCCACCG
CTGACACATCAAGATAATCGAGTGCCGGAAGATCTGCGAAGTGCAATTTTCCTATCATATTGCC
GCCTTTGCTGCCCTGAAGAGAGACACTCTCCAATGAAGAACAACCGGATATATGGATTTCACTG
TCGAATATTGAAGTTTCGGAAACATCATCATTAAGTATAACAGAAGGAACAACTACCAATTGAA
GCGAACTGTTATTCTCCACCCTAAGTACTTTCAATGATGATGCGGAACTTAAATCCATTCCCAA
AGGTGTATCAATATTAGAGATTGAAAATACTGAAACTCCCGAAGACGGCTTGACATACGACATG
GAATAATGCTTGGACTTCACTCCCGAAATGTCTACTTTTCCTCTGAAACCGGGATTTGACAATA
TATACTCCACACCCTCAAGGTTAGCTGTCTGCGACAGGAAAATGAGGTCGTTCCCTTCGGTTAT
CCTCTTCGTGACATCAATCTCCAACGATGAGACAAACACCGACGGGAAGTTTCTGTAAAGATAT
GAACGGAGCAAAGGATCCGGTACTCTTCGGTTTACTGTATATTCAGTGTAATTTCCATCCTCGT
CCGACATCACGACAAGACATTTGTCCGTCATGGCTTTATAGAATGCAGGTATGACATCTGTATT
CCATTTTGCAAAGTAAGGAAGTTTCAGATTTGTAAGACTTTTACAAAGCACCGTAGTGCCATCG
GTTGAAATAAGATTGAGATACGAAGTTATGCCGTCATTGCCGCGTAAAGCTACCGATTTTATTC
CTTCGGGCAGGTCAGCAAAGTCGAATATAGAAAAACTGTTACACTCAAGATTGACATCTGCGAG
CGAAGGAAAACTCCTCAAACCGCTAATAGATGTAAGTTCGCATCTAGTCAAGTCCAAAGAAGTG
GTATTGAGAACTTGATTGTCACAAATCAGCTCTCCGTTTTCGCTGAAATTAAATCCTTTCCGGG
TCAAGACATCGCGTAACTTTGTATCAAAAGTCACTTCAGACACTTCAAAGTCGGAAATTTCTGT
TTCATCCTTACACGAGATTATTGTGAAACAGAGAACTATCAGTACATAAAAGCTAATAAAATTC
CTCATAACAATCAGTTTTGTGGTAATAAGACTATATTATCAATCCAAGCCGCGTCGTTCTGTCT
TTCGCACACAATGGCACACACTACTTTTTTCACTGTAGAATTAAAATCGAAAGATACGGCTTTA
TAATTGCCGGGAGAAGAAAATTCCTCTGTATATACCGTTCCTGTAGACATATCCTGTAGCATGA
CTTTCAACTTACATGCTCCTTCGGTCTTTACATCAGCAGAGAAGCGATAAGTCCTGCCACTCTC
CATGTCAACCCTCTGCATGAGTCCTGCATGACCAGATATACAGGCTACATTATTGCCTGCATTG
TCAGTCTGTACGCAAACCGTACCATAGTTACCCAATGGCTGCCATGCTGAAAGTCCTTCGCTGA
AGGTTCCATTCTGCAAGGTAGAGACAGTATATTTCTCAACCTGCAGTATCATGGACGATACGTG
ACCTCCTCCGTCGGAAAAGGTGATGTCGACATTATTATCGCCATTCTTCAGCAGCTGTATGTCG
AACGGTACTTCTATCATACCGAAAAATATATTGCGGTTGCTCTGGCCGTAGCCTTTCCAGTTGT
CGGGAACACTCACAGCGGTACCATTAATCTTTACCACCGGTTTCTTGGAAGCAGAGACAGGACG
GCCTATCGACATACGCAAGCTTGCTCTGCCCGAACCGGACTCGATTCCTGTGAAGGGGAACGAA
AGGGATGATCCGGCGGAAATCGGTTTCAGATACTCACTGCTGTAATATTTATTGCGGATTATGG
AGTTCGTGAATGCTGACGAAGACACATCTGCTACAAGGACTATGGTCTGATTTGGGACAATTGA
GATGCTTTCAGGCATGGACGGGACATTCTGTTCCGTATATTCTATACCTGCGTTATAATTGACA
TATAGAGAACGCTTTGTGACATTCGATACATCCTTCCAGCTATTCTTATTGTTCAGATATACAG
TCTGCGGGTTATCATCAAGATTATCAAGGGCGATATAGAGTCTGCCTCCATCCTTGAATGCCTG
TACCTGAATATCAGGATTACTGCTGGTTATATCAACACGTTCGCCTTTTACATTCTTCCAGAGT
TCGAAGAAATATTTTTTGTCATTAAGCCTCCATGTGGTATTCTTCAGATTCTGAGGATTGTCGG
GAATAAACAGTGCCGCACTATATGAAGTATAATTGTTTGCAGCGGTGATATGCCACTCAGCCTT
ATCTGAGACAAAAGGTATTGAGATAAACAAATTGTCCTGACGTTCCATCAGATTAAACAGAAAA
TGATTAAACGACGAAACACTCCGCACACTGCTTATGTCATCATAGCTGTCGTCGGGCTTGCTGT
TGTCAATACCTCCAAACTCGGAAATGGCAAGAGGCTTGACATGTCCGAACTTAATATAGGAATA
CGCCTCAACCATATCAAGAACTGCTTCGGAGTTACTTCCTGAACGTTTCGTATCGGTGCCGGTT
ACATTTATTCCATCATAAAGATGTACAGAGAATCCATCCATATATGCACCTGCCCGATCGATGA
ACATTTTCATGCGGGTGTTCCAGTAATTGAAGTTCCCATCCTCCCAGGCGGGGTAGGCTGCGGC
ATAGCCTATCACCTTCATCTTTCCGTTAAGACGCGGATTATTGTGTATATGTTTACCTATTGAA
GCATAAAAATCGACCATCAGTTCGCGCATAGCCTGTCCCTGAACGGTAAAACCGGCATCATTTG
CATGAACGAACGGTTCATTGAGGGGTTCAAAAAACTCAGGTACCAGCTCGCTGTTGGAATAATA
CTCAGCCGACCATGCACCTGCAGCCTGAACGTCTATGCCGCCCTGTATGTGCTGTACATAGGGA
TGCTCTGTGGCAATATATCTTTTTACGGAAATATTTCCGCTGTATGGTTTCATCTGAGGATATT
TGCCTACCTCATGCGTCTTGTTATACGCATACGAGTATGGTCCCCAGAACTTTCTTCCAAGACC
GACCTGATAGTCGGCAAGAAACTTGCCTACATCCTTATCATCATCGGAGGTGGAATGAATATTG
AAATATTTAGAACGGTCGAGTTCTGAAACACCGCTCAAAAAGCGACGGGTATTATAGTCGACAA
CCACCTCGTTCCTTTCCTGACAATAAATACCGGGAGGAACACCTAGGGTAAATGCCGATAACAG
AAAAATATATTTATAGCTCATAATTTCTTTCCTTTTAGACACAGAAACTTGTCAGTCCTGATGT
GGATACATTATTTTCTCACTTTCTTATCGTAGCGTTCAGTCTGAAGAATCATAGTAGCCACACG
GCCTCCATTATCCGGGAATGTTACTGACACCGAATTTTTTCCTTTTCTGATTAACCGGTAGTCG
AAAGGTATTTCTATCATACCGAAGAAATCGTCTCTGCCGGTCTGGTCATATCCTCTCCAATTGT
CGGGCATGTCGACTTTCTTGCCATTAACCATTATTTCAGGTTTCTTCGACATCTCGTGCTTCCT
GCCTATTGACATACGCAGAACAGCTCTTCCTGTACCCGGTTTCAGACCATCGAAATCAAACACA
ATTGGTTTTCCGGCTTCCACCGGCTGAAGATAAGTGTTGCTATAATATTTAGTACGAACTATTC
TGTTTGAATACTTTTTACGGATGATGTCGGCACACAATATTATTGTCTCATCTTTTATAATGTC
AATACTTTGAGGCATCGAGTTCAGCGTCTTTTCATCATAAACTATACCTTTATCGAAAATCATC
TTCAAAGAGCGCACAGAAACATTATCTACACCCTTCCAATTCAGTACGTTTTTCAAGTTTACCT
TATGTGTATAGTCATCAAGATTGTCGACAGCTATGTAAAGCCTGTCATCGTCCTTAAAAGCTGC
CACCTGTATGTCCGGATTGTCGGAAACAATATCTACACGTTCGCCTTTCACATCCTTCCATAAC
TTGAAGAAATATTTCTTGTCGTTCAGTTTCCATGCGGTATTCTTCAAGTCGTGAGGATTGTTGG
CAACAAATAAAGCAGCTCCGTATGGTTCGAAATTATATTGTTTCGTTATATGCCATTCGGCCTT
GTCAGAAACAAAGGGTATTGAGATGAGCATCTTGTCTTCGCGTTCAAGAAGATTGAACAGTATA
TGATTGAACGAAGCGACAGTTCGTACAGAGGCTATCGGATTATATCCTTTGGAAGTGTTGTCTA
TTCCTCCATATTCGGTTACGGCAAGAGGAAGAACTTTCCCCAAGCGGATGAACGAGTAGTTTTC
CATAAGGTCGAGAATAGCTTCGGAATTACTTCCCGAACGGCGGGAACTCTTGCCTACTATGTTT
ATTCCATCGTAAAGATGTACCGACAAGCCATCCATGTACTCCCCGGCACGGTCAATGAACATCT
TCATAGTATTATTCCAATGGTCGAAATCGCGCAACTCCATAGCCGGATATGCCGCGGCATATCC
AATGATTTTCATTTTTTTCAGACTTGGCTCAGCGTGAATATGCTTTCCTGTCTGTGCATAAAAA
TCTGCCATGAGCATCCTCATTTCCTGACCATGCATATTGAAACATTTGTCGCGTGCATGGACAA
AGGGTTCGTTAATGGGTTCGAAAAATTCAGGAACTGCCCCTTTCACATGCTTGGAATAGTATTC
GGCAGCCCATGCACCCGCCTTCACTGGGTCTATGCCCCATTGTATGGTACGCGCGTTGGCATGT
TCCGTAGCGACATATCGTTTTGTTTCCTTCAAATCAGTGTAGTTCAAAGGCTTTTCTGAAAAAG
GATATTCGCCAACCTTTTTTGTCTTGCCATATGAATAAGAGAACGGTCCCCAGAAAGAGCGGCC
GATTCCTACACCGTAATCTGCAAGAAATTTCCTGACATCTGGATCAGAATCTTTAGATGTGTGT
ATATTGAAATATTTACCTCTGTCAAGTGCCGATACATCATTCAGGTATCTCTGAGTGGCATAAT
CCACTGTGACAGTAGTGTTATAAGTCTTATTCTCGGAAGATGATAAAGGAAAAACCGAGAAAGA
CAAACACACAGACAAAGCTGTAAGAATTATGTTATTCATTGTATTATCAAAATTTAAAAGGCAG
AGAACACTCCGATAGTTCAATTAAAGTATTCCCTGCCATTAAGATTATCACTTCTGTTTAAACA
CTAATATCAGAAATCGGCCGGTTTGAGTACATCGTTCAGCACCACTTCATATTCAACTTCTGTT
CCGTCGTTTTCAGTAACAGTAAGATGGCCGTAACCGCCACTTGAGTTATTTTCTTTCTTACCTT
CAAACATGAACATTCTCTTCTTCGTCACTTCCTGTTCTTCTTTATCGCCTGTTTCAGGATTGAT
AACTTCTTCCTTTTCAGTATAGACTTCATTGAAAGAGAATGAGAGATGTTTTTCTGTATCCGAA
TTGATTTTCAGCCACTCGGGCAATTCCGAAGGAGCCTCAGACGACTCGGCAAAGAACTCGATCT
TATTCATTCTCAAAGTCTGATAGTCATTCTTCCAGGCAATGAGGTCGAACAATTCGCGATAAAC
AGAAAACTTGGAAACCTCGCCTGTCTTTCCTGTTTCCACATTCTTGAGATAGGTAAGTTCATAC
ACGGGAGTAGAATCAAGTTCGACCTCAGCCCATTTGTCATTATCACATGCTCCGAACAAAACCA
AAGCACATAAGAATGTAATTGTCTTATAAATTTTATCTATTAGCTTCATTGTTACTATAATTTA
TTATGGTCTTACTTCAATATATCCGAAAAATATATCGTCAAAATAAATATTATCCTTAAAGGCA
TTAAAGCGCATACTGAGCAATATATTGTCCATTTCAGCCTTTGAAGTCACAGTGGTTGTGGCCG
ACATCCATTTGCTGTCGGAGCCATTCACAATGCCGCACCATGGTCTATCGCTCTGCCATGTCAT
ATCTTCAGCTCCTTCTTTACCTGCCGGAACGAAATACGGACTCATACCCTTACCCTGTTTATAC
CCCGGTGTATAATATTTGTAGCTGAAAGTATATGTACCTTTACCACCAGTAAATGTCTTGGAGA
GTAATGCCCTGCATCGGTCAAATGCTTCGACAAACATACATTTTGCACTGTTGTTTATTCCATC
CTTCAGAGGATTGTCCACAACCTGTGAAGGAACTACAGGATGTGTTTTGGTATCGGCATCAATA
ACTTTCCAGTCGGCATATGTGTCAGAATTTTCAAAATCTTCATCCAGGAACGCACCAAAAGTAG
TCGCTACGTTTGGAGCTGTAGCCTTTATCTCAAGGTTCTGATATCCAACCAACGCTTCAGTTAA
AGTTCCTGTAAGGGTCAGTTCATCTGTGTTATAGATTTTCTCAACCAAAGTAAGAATCAGTTCA
TATCTGCTTTGCTTGTTTACTTCTGCTGCTGTGATGTTTACGCTACCCCTGACAGCTGACGGTC
TGTTATACGAGTTGGAGTAAGTAAGCTTTAGAGATGATGGATTTATCTCTTTATATCCAAACTC
AGAATTATCCAAATCTATAGCAATGTGTGTTTGGTCAATCTGACGGATGTTATAAGTAATAGGA
TCATCACTAGGTACTACTGTAATAGCCAAAGGCACAACAAGAGTTTTTGGCGAAGCTTTTGGAG
TGTACTTACCTTTACCCTCACTGGCAGAAGTTCTTTCTATTGTCATGGAAAGAAGCAATGGCTT
ATCGCTGAATTTCTTTGCAGTGAACTGGTATGGAGTGTCAAAACTGGTTAATTCGTCATTTACG
CCAGTATCCGCACATTTGAAAGTCCATTTGTTAGGCAATCCGTATGAATCGTCCTTAATATAGA
CAGACTTACCATATTCAAGTTCGTATTTTTCGTATTCGGGAGCTTCCTCAGTTCCACCGACTAT
TCCGGTCTTTATTTCCTGTGTACACTCCGGATCACTGTATACCTTTACGGCCGGTACGAGGTTA
GGATCATACACGCGGATATGGAAAGTTGTATCCATCACATATACATCACCCTCCTGCTTAGCAT
AACAATATTTTTTGATATATCCTCCGGTATTGTCGTCATACACCGAATATGGATATACAACCTG
TCTGCGGAAAGTATTGCACAAACGTACCGTATGGTCACCGGGTTTAGTGAAATACACATGTATG
GTTTTCAAATCGTTGGTATGAGGGATGGATTCATCAATCAGGTTTGTATAGTCTGTCTGTCCCC
ACTCCATCTTACCATTAAGGAACTTTGTACCATCATCCGACACAACCCACTGATGCGACAACAT
GCCTTGGGATAAGTCCATTATACTTATATAGTTATTAAGATTCAGCTGAATAGGTGAAACGTTT
TCCTGATCTGTACTCACATGCCAGGTACATTCAGCCACGTTATTCAACGGTTCAAACTCATCAT
CCTTACAAGATGTCAGAACCGAGATTAATGAAAGAGCAATATATAAAAATCTATTTTTCATCGT
ATTTATTTATTAATATCAGGATTTGATGTAATTTCTATATTTGGAATAGGCCAGTATGCCACTT
GCGGACCGTAGTTCAATGATGCTTGGAAATAATCCACAAAAGCGTTTCCTCTCTTTTCTGGCGG
CAGCTCATAAAATCTGTACTGCTTTCCAAAGTTGAATGCTGATACCAAAGCATTAGGGTCATCA
GGATTAGGCTTAAGATATTTGGTCTGAATCATACAGTACTTATATTCGTCGGATGCCAACTGAT
CAAACCTTTCCTTAGTTATATTCCAGCGTCTCAAATCAATGACACGTATGGCATGTCCTTCCAT
ACACAGTTCAAGAGGACGTTCCACATACATCAGATGATTCATTAGATCACTTGCAGCATATTCC
TTCTCATCGTATGTATATCTCTTGAATTCTCCCTGTTCCGATTTTCCGATAAGCACAACTCCAG
CACGGTGACGTACCTTGTTGATGGCATTGATAGCTGACTGAACATTTCCATCGCTTGCACCGCC
TTTAATCAGACATTCTGCATACATCAGATATATATCTGCCAAACGGATAAGACGATAGTTTATT
CCTGAGGCCATAGCAGGCTTAAATTCAGTTTCACTCTTACGTGTATCCCAATTTGATAATTTTC
TGAAATACGCTGAAGAGCCACGGTTGAATTTTGATACCTGTTGTGGGAGAGACTGATAATATAT
CAGACTTTCATCGCCGTTTATTGCAAGAGAGGCAGATGCACGCATGGAATAGCTTCTGAGGCGA
TATGCCTGACCGTCTTCCCATTTAAATTCCGGAACTATGTCATCGTAGCCGGTAATCTTATTGT
ATAAAACTTTATTATCTCCGACAGTTGAGACGAGTCGTTCGCGTACTCCAACATATTTTCCTGC
TGTTGCATCCCACGTATAAACGTACGTTCTGTTATATACGACACCCTGACGGTCCACCTGCGAG
CTGAAAGTTGTTCCCAACTGGTCGTATATAATATCCCTATGTTCAGGATCACCATAATTGTCGG
ACTGCATTTTTATCCAGTTACGTTCATCAAGTCTGTCCACCGGCTCTGTTTCGAATGCTTCAAC
AAGCCAAAAAGCAGGAACAGTGTTAAGCCAGGCATCGCCCAAGCCATTTACATTCATTCCCCAT
ATATTATATAAGGTAGACTCCGACCATGTACCGAATTCTGTATTATACTGTGTAGAATAGGAAA
CCTCGAGAATAGATTCCGAATTGAATTCATTGGCAGCAGTAAAATTATCGACTATGTCATCAAC
CAAAGCAAAACCTCCATTATCAATAATATCCTTAAAATATTCGGCAGCTTTATTATACTCTTTA
TCATAAAGGTAGCTTTTGCCTAATATTGCCTTTACAGCCCAAGAGGTGATACGTCCCAAATCGG
TTTTCTCCCATTTGTCATTCAAGCCAAGGTCAAGAGCTTTCTGTAAATCTTCTCTGTAATATTT
CTTGATTTCATCACTTGGTGTAACCTTTTTATAGTAATCTTCTTCTACCTCTGCAATTTCATTA
ATATAAGGAACATTACCATTATTGAATGAATTATTGAGATAAAAATAAAACAAGCCACGCAAAG
AATATGCCTGTGCCTCAATCTGAGCAAGCTTGGTTATTTGAGGTTCATCTGTAACATTTGGACG
GATTTTCTCTATACTGGCCAGAACCTGATTCGCACGGAACACACCAGTATACAGTGCAGACCAT
TTACCACGGACTGTTCCGTATGAATCATTAAAGGTTTGCTTATAGGCTTCGTTATCAAACTGCT
TTCTGTCCTTATTACCTTCAACTGCTATATCACTTCTACGGTTCTCATCGAGCGGATGATAAAT
ATTGGTATTTTTCAAAGCATTATATACAGCAGCCAGTCCTTTCTCGCAGTCGCCTATTGTTTTA
TAAAAATTCTGTGTTGTCAGCTGATGTATGTTTTCCTGCGTAAGGAAATCGTCGCATGAAACCA
ATGTCATGCCCGACATCAACAGACTGAATACTATTGTTTTATATCTGAAGTTCATATATTTATA
TTATTAAAAGTTAGAAATTAATCTGGAATCCGCCACGCATCTGGATACTTATAGGATATGTTCC
ATAGTCCAAACCACGACGTGACAATCCATTACTACCGACCTCAGGGTCGTATCCGTCGTATTTT
GTCAGTGTAAGAAGATTATCGGCTGCAACGTATAAACGGAACTTGCCCAATCCAAGCTTTGATA
CCCAACTCTTGGGGAATGAATATCCTAACATAATATTTTTAAGTCTGACAAATGAACCGTCCTC
AATCCACATATCAGTATGAGCACGATAGTTGTTATGCCCCTCTGTACGATAAGAAGGAATGGTA
GAGGTATAGTTGGTAGGGGTCCACATGTATATCAGTTCCTTATTGGTTCTTCTTTGATATGTAT
ATATCTTCGTACCGTTTATTATTTCATTTCCAACTGAAGCATACCAGTTCATAGAGAAATCGAA
GCCTCTATAGTCGGCCGAGAAGTTCAAACCAAGTTCATAATCCGGCATACCACTACCGGCATAA
ACACGGTCGTCATCATTAAGAACACCATCATTATTGGTATCGATATACATAAGGTCACCCATAC
GGGCACTTGACTGTAATTTCTGATATTCTGCAAGCTTCTGTTCAGTATTGATTACCCCTGCGGT
TGGCATAACAAAGAAAGCACCGGCTTCATATCCTTTCTTGATTGCAGTTACATAATCACTTCCT
GATGAAACAGGTTTACCGTCGGGGAAGAAATATAACTCATTTTTTCCTGCCATAGACACAATCT
CATTCACGTTTTTGGTAAATGTACCAGTCAAGCTGTAATTAACACCACGTATTTTGTTGCGGTG
AGTAAGTGAAAACTCAACACCACGGTTTTCCATATCTCCGGCATTCAATGTAACAGTTGAACTC
TGGCCCCCTCCATTTGACGGTGGCACGACCATCGGGAAAAGCATATTCTTCTTGTTACTCTTGT
ACAAATCAAGACCTAAGATAAGCTTGTTATTATATAAAGCCATGTCGATACCGGCATTAAGCTG
CTGGGTTGTTTCCCATTTCACATTCGGATTGGCAAATCCCAATTGGGTAAAACCATTTGCAAGA
ATTTCGGAAGTTCCGGTACCAAAAGTATAGTCGTAGTTTTTGTATATAGCTGGTGCGTATGAAT
AATCAGGGAAGTTCTGATTACCGGTAGTACCATAGCTGAATCTTAATTTTAACGAATTTACTAG
CCACCTGAATCTGTCGAAGAATGATTCCTCAGAAATATTCCATCCTACAGACAATGACGGGAAC
AATCCCCAACGATTTTCTTCGGAGAACTTAGATGAACCGTCGCGCCTGATACTGGCACTTGCCA
TGTATTTGTCTGCATAGCTATATTGTAGACGACCCAACATACCAACCATTGTACTGATACGGTC
CTGTCCCCACTGGCCACTGCCTGTACCCACAGTCATATCGGATGTTCCCGCATTTAGGTTCGGA
ATCTCGTTAGTAACCAAATCCATTATACTGGCATAGAACATCTCGTATGTATATTTCTCCATAC
TGAAAACTCCGGTAAATTTAATATCATGCTTTTTTATCTTCTTATTATAATTTACCATTGTTTC
CCAAGTGAGACTGGTATTCTTTGAATGAGTATCTTTTAATTGCGAACGGTAATTAGAGCTGGTT
ACCTTTTCGCCTTTCTGATTATATACCTCAAACTCAGGTCGAATTGAGACAGCTTTCTGATTGT
TATATCCAAAGCCCAAACGTGTGGAAACATTCAGTCCGGGAATTACATTATAAGCAAGATAAAA
ATTACCGTTAAATGATTCTGTGTCCTTATGATTTTCCTCTTTCAATCTTCCCAATGTATAACTT
ACGCCCTGTAAATCTGCAGGATCGCCAGCTGCATTTACTATACTTGCCTGTGGATAAATCTGAG
AACGAGTAGGCGAGTAGTCATAACATTCGTTCAATAACCCCCAAGCCGGAGATAACTGGTTTTC
TATCTTCATAGCGATGTTAGTGTTGATAGTCCATTTTCCGCGCTGAAAATGTGTATTCGAACGA
ATATTATATCTTTTGTAATCGGAATTTATCAACACACCTTTCTGGTCGAAATAGTTCGCGGTAA
GGTTATATGTCAAATCTTTCTTGCCGCCATTCGCAGTAACAGAATAATTCTGTATTGGTGCGTT
ATTATTGACTAGATATTCATATAAACTAGAGTTGTTGAAGAAATTCACAGGATATGTTTTCAGA
TTAGACCAGGCCAGGTCGTCTGTATTCTGGTTTCCTTCCATCATTCTGTTAGACATCACTTTTA
CAAATATACTCTCGTTGGCATCAAGCAAATGAATATTCGAAGTAATGTGCTGTAGACCATAATA
TCCGTCGACAGCTATCTTCATTTCTCCTTCCTTACCCTTCTTTGTGGTAATAAGGATAACACCG
GAAGCACCGCGAGTACCATAAATGGCAGCCGAAGCAGCATCCTTAAGAATATCTATACTTGCTA
TTTCGCTACTACTCAATCCCGGGTCGCCCTCGAACGGGACACCATCGACAACATATAAAGGAGA
ACTGTCGCCTGAGATAGAACTTAAACCACGAATCTGGATGTTGGATTTGGCTCCAGGCTCACCA
GAACTTGCCTGAACGTTAACTCCGGCAACCATACCCTGAAGAGCTGTACCCAAGTCGGAAGTAC
TGATCTTAGTAATCTCATCTGAGTTTACACGTGCCACTGCACCTGTCACCTCTTTTTTACGCAT
TGAGCCATAACCTACAACAACCACTTCATCCAACACTTTTGTGTCTTCCTGAAGCTTGATATTA
TAAATCTGACCATTCTTGATTGCAGCTTTTAGAGTTTTATACCCAACAAAACTGAACACTAAGT
TACCTTTAGTCGGTACCCCTTGAAGAACGAAATTAGCATCCATATCAGTAATAGTTCCAAGAGA
AGTACCTTCAACTTGAACAGCTGCGCCTATAACTTCAAGGTTATTGGCAGCATCAATCACCTTT
CCTTTAACTGTTATCTTCTGTGAATACATAGACAATGTATAGAAGATAAGCATCACGAACAACA
TGTACCTGCCATGGTACCATTTTTTCTGATTTCTCATTTGTAAAAATTTTAATTTAGCAATAGG
TTATGAAATTCCTTTTATAACTGACGCTAAATTATTTATTTATAATGGTACAAAAGGGGAGAAT
TATATATTTAAAAAGGGGGTAAAATTTTACCCCCACTTATATTAAGAATCCAAATCGGTCTGTA
TACTCTGTTCTTTGTACTGTTGCGGCAATACACCGAATTCTTTCTTGAAACATTCTCTGAAATA
CTTCAAATCATTGAACCCTACATCGTATGTCACCTCTGATACAGAATACCGTCCTGTCTTCAAC
AGTTCTGCCGCTCTCTTCATTCTTATTGAACGTACAAAAGCATTGGCTGTTACTCCCATAAGTG
CTTTCAGCTTCTTGTTCAGAACCAAGGCCGTCACGCCAAGACCTTTACATATATCCTCTATCTG
GAACGAAGAGTCTGTAATGTTGTCCTCTATTATCTTTACAAGTTTCTCAAGGAACTTATCGTCG
GTAGATGTAGTGCTTACCTCGGAAATCTTTATTGCCGGAACTTTCTTGTGTTGAAGAATCCGCT
TCCTGTTGGTTATAATGGAATTAAGCAGCTCTTTCATTATCTTGTTGTCGAAAGGTTTAGGGCA
ATAAGCATCTGCATGGAATTTATATCCGATGAAATAATCCTGCAATGTAGTCTTGGCTGAAAGC
AATACTAGAGGAATATGAGATGTCCTTACATCCTGCTTGATTCTCTCACACAGTTCCAGACCAT
TCATGCCCGGCATCATTATATCGGATAAAACAAGATCCGGTTGCAAATCTGGAATCATGTTCCA
TGCCATCTCCCCATCATGGGCTATCATTATCTTATACTTATCCGACAACAGTAATGACAACATA
TTAGATATATCCTTATTGTCATCAACAATCAATATAGCCGGAGATTCTCCGTCCACTTCTATGT
CTATCATCTCTTCATGCTCGCACGATTCACTTCTTAACACATCAGCAAACTTTTCATCCTCCCC
ACTGTTGGCAGAGATATTCTCCGTAACCATGTCCCCCTCAGTTATCATAGGAATTACAACATGG
AAAACAGTGCCTTTACCTTCCTCTGATACAAACGTAATATTTCCATTATGTATCTCTACAAGCC
GCTTGGTCAGAAACAGACCTATACCGGTACCTCCTTCAGCAGAGTTTTTATTCTGACTGTAGAA
ACGCTCGAAGAGGTGTGTTTTCAGGTTGTCGGATATTCCGTTTCCCGAGTCTGCCACAGAGATG
TTTATTTTGTTATCCTGTTCATTGACAGTAAACGATACAAATCCTCCGGCAGGAGTATGCTTAA
TGGCATTCGATACGAGATTATAGATTATCTGTTCCATAAGATGAGGGTCGAACAGAAAGCTTAT
ATCACTGCGTGAGACAGAATATTCCAGCCCTACACCTTTCTGTTTTGCCCAATACGTGAACTGC
TGAAATACTTCTTTTGAGAAAGACGAGAAGTTGCCATATTTGAGATTCAGACTAAGCATTCCTT
TCTCGCTCTTTGAGAAGTTCATCAGCTGGTTGACAAGACTTAACAGGAACTTACTGTTATGCTC
CATTGTCTGCAGCATGCCGGCAAGATACTTGTCGGACGAATACTTGCCCGATTCAATAATCATA
CTAAGTGGAGAATGAATAAGTGTGAGTGGTGTCCTCAATTCATGCGATATGTTGGTAAAAAATG
TAGTCTCCTTTTCAAGAAGTTCTTCAGTCTTGCGTTTTTCCATGTTTGCTATATATAGAGCATT
TCTGCGCTGCACCCGTGAGGTATAATACACCTTGAACCGGTATAAAGACAAGACAAGCAATATA
AAATAGAGTGTATAGGCATACCATGTACGCCAGAAAGGAGGGTTAATAATGACAGGTATGGAAA
GTTCATTCAAACTGTAGACTCCATCGCTATTCCTGACCCTCAGTCTGAACATATATTCGCCTGA
AGGAAGCTTTGTGTAGAAAGCCTCACGATGAAAAGCGGAGGTGGAAATCCATGAATCATCTACG
CCTTCGAGCATATATTCGTAACCAACCTTATAAGGACTTCTGTAATCCAGGGAGCTGAACTGGA
ATGAGAAAGTGTTTAAATTATAAGGCAATTCAATGTGCTCTGTAAAACTTACACTTTTGTCGAA
ATAAGCTGAATATGTGGAATCTGCCTCAACGCTGTGATTGAAGATTTTAAAATCAACGAGTGTA
GGACTACCGTTGAAATCTATCACATCAAAGTCATTAGGTCTAAAGACGTTAATTCCGTTTACGC
CACCGAATATCATTGTTCCATCCGTCATTACTCCAGCAGAAAGTTCCATAAATTCATAATCCTG
AAGACCATCGAAAATATCATAAGATCTTATTCTCTGTGTGTTGATATTCAACGAATTAATTCCT
TTATTGGTAGAAATCCATAATGTTCCATCCGTGCCATTAACAATTGATTTTATTGTATTGCTGC
TCAACCCGTCTGCAGAGCTAAAATTTTCAACGCAGGCATTATGGTTTTCATCCAAATCCACGAT
TTTCCTTAACCCACGTCCAAGTGTTCCATACCAGATATTATGATTCAAGTCTTCACATACAGGC
ACTATATAGTCGAGTTCATCAAGTCCCTTGACTGAGTTCAAAACAGGATTATCTATATACAAAT
CTGCAGATTCCAATACTTTAAGACCGAAGCTGGAAGCTACCCATATATTACCCTTATGATCTTT
AATGATGTTTCTTACTATCTTAAGTTCTTTATTGTCAGATGTTTTGATTTCCTTCATCACACCT
GTGGACAAATCATATCTGAAAAGACCTTTATTATATGTGCCAATCCACAAATATTTTCCATCGG
CAAGCATTGCGCGCACATTTCTCAAACCTGAGATCTTTTTATAATCATTATCAGAAGTGAAACT
GTAAATACCATCGTAGATCAGAGACACATACATGCAGTCGGTGTAGTTTGAGTATGCTGTTGAG
TATACTATCCTGTTTGCCGTGAAAGGAATAAGTCTGGCATTACCGGTAATGGAATTAAAATGAT
ATAGCCCTGAGCCTTCTGTGCCTAAATATATATCAGATTTGGCAAATGTATAAACGGACGATAT
ATGATCATTTCCTATTCCTCTGAATAAATCTATAGGTTTATTATTTTCGCGTATACTCATAAAG
CCACTCTTGAAAAATCCTATCCAAAGAATATCGTTTTTATCAAGAACTAGAGTTTGCGGATAGC
TGTAAGAATATGTAGCAATAACCTGTGGTTTTGACTCGATGGCATGCAATACATCAAAAGTCAA
CACATTCACAGTGCTTGTAGTGGCATAAAATAATCTTTTGTTTTTATATACCATTTTTCGTATA
TCACAGTTTTCCAACAGGGTACTTACCTTGCAGGTATGCTTGTCGTATAAACATAATTGATGAT
TTTCCAGATTTGAGTACAATATTTGAGAAGATGAGATGACTATGGCTGAAGCTATAGGGCATCC
CAATAGTTTGTTAAGCAGTAATTCATCTCCATCGACGTTACATTCGTACAGGCCGTCTTCGGAG
GAGAGCATTATCGTATTATCTATTTCTATGATGTCGGAAATGTATGGTAATTTTAATGTTGATC
TTAAGACAGTATTTATTTTGCCATTTTGAAAATCATAATTTACAAGGTATATACTTTCATCAGA
GGAATGAAACCAGACTCTGTCTTTAGAGTCGACAAGAATCTTATCGCAAGTGAAATTTTTATCA
ATACCGCTGTGACCAAGATTTAATGAAACGAATTCGTTCTTTACAGAATTGAACAGGAACACTC
CTCTATCGGCTGTACCTATCCACAGATTTCCATGTGAATCTTCGTCAATACATACTATCAGATT
ACTGTTAAGACCGTTTGACTGATATCCGTAAACCTTAAATTCATATCCGTCAAACCTGTTCAGT
CCGTCGTTCGTGGCCAACCATATAAAGCCTTTTGAGTCTTGATAAATACATTGCACATCATTTT
GGGAAAGTCCATCAAGAGTAGTGTACTTTCTTGTGACAAACTCATTGGATGCAAAGGATTTGCA
AACTATAATCAGAACTGATATTAAACTTAAGATTAATCTAAACATATAACTATTATTCTTTATA
TTTCATCAAGATTACAAAGTTATTGATTTTATCTAAAACATCAAGTATTTACAGTAGTTAATAG
ATAATTATAGATATTTTCCACTTTAGAATGCGTATCAAAATCAATCAAGAAAAAAATAAATCTT
TAACTTCATTTCATAGTATAAAACAAAAAAAGCATCGTACCATTACACTCAATAATAGATACGA
TGCCCGAAAGAAATTACAGTAACAGACTGTATTGGGATTGTTCTTAAAAAGACTTATCTGTATG
ACTTTATATATATGTCGAGTATTTCGGTATCCGACAGTTCATGAGGGTCCAGACTGAACAATGC
ACCCATGGCAGTTCGCGCATTATCAATCATCTTAGGGAAATCTTCCTTTACTATTCCCCAGTCG
CTAAGCTTCAAATCGCGGACATTGCATTCCTTCTGCATTCTCACCAAAGCATCTATAAAATGTT
CGGGATTAAGGTTCTTGCATCCGGTCATAACATCTGCCATGCGCATATATCTCTTTGTCCTGTC
ATAAATAAAAGTAGAGAAATAGGCCTCGCTTATAGCTATCAGGCCAACACCATGAGGAAGAGCG
GGATAGTATGCGCTGAGAGCGTGCTCGAGAGAATGTTCGGAAGTACAACTGGATGTGGATTCAA
CCATTCCCGCCAGCGTACTTGCCCAAGCCACCTTTGCCCTCGCTTTCAGGTTATTTCCATCCTT
CACCGCAACAGGTAAATATTTATACAGCAGTCTGATGGCCTCAAGAGCGAAAATATCACTTATT
GGGGTTGCACAATTGGCAATATAGCCTTCGGCTGCATGAAAGAATGCGTCGAATCCCTGATAGG
CAGTCAGATGTGGCGGAACTGAAACCATCAGTTCCGGGTCGATTATCGACAGACATGGGAAAGT
TAAAGTGGAGCCGATACCTATCTTTTCGTTTGTTTCCAGATTGGTTATGACAGTCCATGGGTCA
GCCTCGGTTCCGGTTCCGGCTGTTGTAGGAATGGCTATGATGGGCAATGCTTTGCTGTAAGGAA
GCCCCTTGCCGGTACCTCCTTCAACATATTCCCAATAATCGCCATCATTACATGCCATGATTGC
AATGGATTTGGCCGTATCTATCGAACTTCCGCCTCCCAAACCTATAATCATATCGCAATTTTCC
TCACGACAGATTGCCGTACCTTCCATTACATGGTCTTTTATTGGGTTAGGCAATATCTTGTCGT
ACACCACGGCATCAACATTATTTTCTTTCAGCAGACCAATCACCTTATCCAGATAACCATATTT
ACGCATTGATGTTCCGGATGAAATGACTATCAAAGCCTTTTTGCCGGGCAATGTCTCTGTTGAA
AGACGTTTAAGTTCGCCACATCCGAAGAGAATCTTCGTCGGAATATTATAACCAAAAACAAAAT
TATTGTCCATAAATATTATCAGTCAGTCAACTTACTATCTTAAAGCCTCATCAATCACTTTCTT
GAGTTCAGGATAAGCCTCATCTGTATCGCCCACCTGTTTTCTCAACTCACGCAGTTTCTTTTTC
ATGTCCTTAAGAACTTTGGCGTATTTAGGATTATCAGCCAGGTTTACCATTTCGTAAGGGTCGT
TCTTCACATCGTAGAGTTCGAAAGAAACCGGAGTAGGAACAATCTTGTGGCTGTTCTTCAACCA
TGACATTGATTTCTGTCCGTAACGTTTGTCGTCGTAATGACGGCCATAGAAAAGTATCAGCTTA
TAGTTTTCCGTGCGGATACCTATGTGTGCCGGAACGTCGTGATGAATCATGTGCATCCAGTATC
TGTAGTAAACAGCATCCTTCCAGTTTTCTGGCTTTTTGCCTTCGAACACAGAGGCAAAGCTCTT
TCCATCCATGTATGAAGGTTCTTTGCCACCGACCATCTCTATAAGAGTTGGAGCAAAATCAATG
TTGTTAATCATCAGGTCCGACTTGGCTCCCTTGTAAGGACATCTCGGGTCGCGGACTATGAAAG
GCATTCTTTGAGATTCTTCATACATCCATCTCTTATCCTGCAGATCGTGTTCGCCAAGCATCAT
ACCCTGGTCGCCTGTATATACGATAATGGTATTTTCCCAGAGTCCTTCCTTCTTGAGATAGTCG
AAAAGACGTTTCAGGTTGTCATCCACACCCTTTACGCAACGCAGATACGATTTCAGGTAATGCT
GGTAGGCAAGGTATGTATTCTCCATTTCATCACCTGTATTGCACTTATATTCCATTACATAATT
GCGGATTTCATGACGGCTTGAGACAGAAGTTCCGATGAAGTGACGAAGTGAATCGTTCTTGCCT
CTTGTGCCTTCGGAGCCCCATTTGTCTGTATCGAACAATGACAATGGAACAGGCACTTCCACAT
CGTCAAGATAATATTCATAGCGCGGTGCGTACTCGAACATATCGTGCGGTGCCTTGTAATGATG
CATCATGAAGAAAGGTTTGGACTTGTCGCGTCTGTTCTTCAACCAGTCAATAGCAAGGTTGGTC
ACGATATCCGAGGAGTAACCCATTTTCTTTATCTGGTTATTAGGCCATTTCTTGTCAGTTACGT
CACTTGTAAGGAAAATAGGGTCGAAGTATTCGCCCTGTCCGCCATGACCGTTGAATACAGAATA
ATAGTCGAAGTGCGACGGTTCGCATCCCAAATGCCATTTACCGATCATGGCAGTCTGATATCCC
ATATTATGGAACTCATCAACCAGATATTCCTGGTCCGGCTGAAGCACTTCATCCAAAGTGAGCA
CCTTGTTACGATGGGAATACTGTCCGGTCATGATACATGCACGGCTTGGGGTACTGATGGAGTT
TGTACAGAAACAGTTCTCGAAGAGCATACCGTCCCTTGCCAGTTCATCAATTGTAGGAGTAGGG
TTCAGTACTGCAAGACGACTTCCGTATGCGCCGATAGCCTGCGAAGTATGGTCGTCCGACATGA
TGTAGATGACATTCATCTGTTTCTGCTGTGCTGCGACACCAACACATACAGACAGGAATGGCAT
AACAGCCATTCCCTTCATTATATTATTTTTTAAATTCGTTTTCATAAGTCAGATTATCATTGAA
ATAGAACTTGCAAGACATATCATCGAATGATTTTACGTCCTTATTCTGCATTTTAACCCATTGT
TCTGATTTAGCCTTGACAGCGACCTGAGTTGAAACCTCATTACCGTCGACTACACTTTTAAGAG
TGACATTTGCATCCTCTGCATTATGGTTTGCCACACGTACAGTGATAAGGCATCCGTTATCAAC
CTTATCGTATAGCGGTTTGGAAACCACCGCCCCTTTAAGCTTAATCTTGAACACATGTGCATAT
TCAGTAGGTTTGTTCTTAGGGAAGTTTACTACAAGACCCTCGTCAGTCATCTTATAGTCAATCT
TCTCTGAGCTTCCAAGCATTTCAACCGACTCAATTTCCACGTTCTGGCAATACTTAGGAGCAAA
TGACTTGATAGTAACACTACCATCTGTCCAAGCCAGAGACACGGCATAGAGGTTATTGTCGCGT
GTAGTAAAGCGAATGTCGTCCGCTGTATATTCAGTTTTTGTATTGTCTGTCATATAACCTGCGG
TGCCTGCGTTATGTCCTTCGAAAGCAATCACCCATGGTCGTGAGCCATAAATAGCCTCACCGTT
AGTCTTCAACCATTTACCTATCTCGGCAAGTACGTTCTTCTGTTCGTCTGTAATAGTACCGTCG
GCCTTAGGACCTATATTCAGCAATAAGTTAGCGTTCTTGCTGACAATATCAACAAAGTCGTCGA
TGATATGGTCAGGACTCTTGTTTTCCTCGCCCACACAATAGCTCCACGATTTCTTGCCTACAGA
AGTATCAGTCTGCCATGGATATTCACGGATTCTGTCGCTCTTACCTCTTTCTATATCGAACACC
TGGATATTGTCGCCATATCCGAATTTAGTGTTAACCACAACTTCTTTATTCCAATCAAGAGCCG
AATTGTAATAATAAGCCATGAATTTATAGAAAGTAGGCTGGAACGGATATTTTCCCACAGTCCA
GTCGAACCATATCAATTCAGGCTGATATTTGTCGATAAGCTCGTATGTATGCATAAGGAACTGA
CGGCGTGAACGTTCGTTCGAGCCTTCATACTTACCACAATAAGGTGTCATACCCTGACCTTCGG
GCTCATGCAGTCTTTCGCCATACAGAGTGATTGTAGTGTCCTGAACATCAGAAGGAGTTTCCAT
TCCATATTCATAGAACCATGCATTCTCGCATCTGTGAGAAGAAAGTCCGAAACGCAGACCGGCT
TTCTTGGTAGCTTCCTTCAATTCGCCGATTATATCCCTTTTCGGTCCCATATCCACAGCATTCC
ACTTATTGAAAGTACTGCTGTACATGGCAAATCCGTCGTGATGCTCGGCCACCGGAACAATGTA
TTGTGCTCCAGATGATTTTACCACTGCCAGCCACTCGTCGGCATTGAAATTTTCGGCTTTGAAC
ATAGGGATGAAATCCTTATATCCGAATTTGGTCAAAGGACCGTAAGTCTGTACGTGATACTTAT
TAATAGGATGACCTTCCTTGTACATCCAGCGGGAATACCATTCACTGCCGTATGCAGGAACGGA
ATAAACTCCCGAGTGGATAAAGATACCGAACTTGGCATCCTTAAACCATTCAGGAATAGTGTAA
TTTTGAGCAATCGATGCCGAATCGGCCTTGAACACATCAGTACCTTTTAAAGATACAGTAGAAT
CTACATTAGGAGCGTATGTAGAATTGCACGACGCCAACAGGCTTAATGCCGCAACTCCTAAAAC
CGTTTTCATGGATTTCTTATTCATAATAATCTTATTACATTAAATAATGACATTAATTTTTTCT
GTAAGCAAAGATACACTTGAGTTCCATTTACAATAAATAATTTAATTACTATAGTAAGGGGTAA
AATATTTAGCACCTATTATTGAACAAATTTACCCCCTCTCATATATGATAATAAACTGCCAATA
TCGAATTACAAGTAAATATATATTTCAACAAAAAAGGTTTAGCCTATTATTACACAACAATTTC
ACCCTAAGAATAAAATATATATAGAGTAAATTTGCCAATATAACAAACTGTAAAAACAAATTTA
TGAAAAACTATTTGATTTACTTACTCGCAGCAGTATCGTGTACAACTGTAGCAGACCTAAATGC
TCAAGTCAGTACAAAAACAGGTAATGAAACCACAGAACTTACAATTCCGAAAAAGTTCTACAAG
GACAGCATTGATTTCAGCAATGCTCCGAAAAGACTTAACAACAAGTACCCTCTTTCCGACCAGA
AGAACGAAGGCGGATGGGTTCTAAACAAAAAGGCCTCTGACGAGTTCAAAGGAAAGAAGCTGAA
TGAGGAAAGATGGTTCCCGAACAACCCTAAATGGAAAGGAAGACAACCTACTTTCTTTGCAAAG
GAGAATACTACATTTGAAGACGGCTGTTGCGTGATGAGAACTTACAAGCCAGCAGGATCACTGC
CCGAAGGATATACTCACACTGCCGGTTTCCTGGTAAGCAAAGAACTTTTCCTTTACGGATATTT
CGAAGCAAGACTGAGACCAAACGACTCGCCATGGGTTTTCGGTTTCTGGATGTCGAACAATGAA
AGAAACTGGTGGACTGAAATAGACATTTGCGAGAACTGCCCCGGCAATCCTGCCAACAGACATG
ACCTGAACTCGAACGTGCATGTATTTAAAGCTCCAGCAGATAAGGGTGATATAAAGAAACATAT
CAACTTCCCTGCCAAATACTATATACCATTCGAATTGCAGAAAGACTTTCACGTATGGGGACTT
GACTGGAGCAAGGAATATATCCGACTATATATAGACGGAGTACTGTACAGAGAAATAGAGAACA
AGTACTGGCACCAGCCATTACGCATCAATCTTAACAACGAATCGAACAAATGGTTCGGAGCCTT
GCCGGACGACAACAATATGGATTCTGAATATCTGATAGATTATGTAAGGGTGTGGTACAAGAAA
TAAGAAATAACATAATCTGAAATTATAAAAGGCAGTCTTCATTATCAGTATGCTGATGATAAAG
TCTGCCTTTTTAACAAGAAGATAAAGATTTTAATCTGCCCTATCACTCATTTACTTCATCCGGA
TACTCTGTAAGCGAGTTTCCCGAATTGCTTATTTCAATAGAGCCGATAGGAAGATAATTGAACT
TCTTGCTCCATGCAGAGATACCATAATCTCTTCTAAGAATAGGCATCATGACCTCCTCGGCACG
TCCTGAGCGGACGAGGTCAAACCATCTGTCACCCTCGCATGCCAGTTCACAACGACGCTCATAC
CATAGAACATCAATTACGCTTTTAAATCTGTCAGGATACATCTGCATTAGCTTGTCAACATCAA
TATAACTTCCGTCGTCTGCATGAACATGCTTCTTTCTGAGTTCATTTATGTAATACTTCGCTTT
TGCTTCATCAGGATTAGTACCTCTGAGATATGCTTCGGCAAGCATCAGATACACTTCACCATAT
CTGATGACCCTTACGTTTCCAGGCTTGTTTAGATTGGGGTTTCCTATCATATCGTAATTTTTGA
AAGGAGGATATTTCTTCTGGGCATATCCCTGGAAATCAGGCCCGTAAGAGCCTGTCTCCCAAAC
AACTTTTTTTGATTCATCCTGAATATTGGCATTAGGTTTGGTTACAAGTTCATCGTAAGTAAAT
ATCGCCGCATCACGACGCACATGGTCATCCGGAAGGAAATAATCATACAATTCCTTAGTAGGCA
GACAAAAGCCATATCCATTATCATAATCAGGACTATTTTTCAACTGTCTCGGTCCGCAGAAAGT
CACCCACATAGCACCTTCGCCTGCATCAATATTACCCCAGTTTGTATTACCAGATTTGGTAGAG
GTCTGTATTTCAAATATAGATTCCTCGTTATTCTCCTGATGAGCCGCAAACAATTTAGAATAAT
CATCCGTCAGAGTATAATTACCACTTGAAATTACATCCTCCAATAAAGGTTTCGCTTTGTCAAA
AATCTTAGCATCATCGTTGCTCCAGTCAGCCCAATAAAGATAGACCTTGGCCAACAGGGCTTGA
GCCGCAGTCTTGGTAATACGTCCTTTCATTGTGTCCGGGAAATTATCCTTTAGAGAAGGGATAG
CTTCAAGAAGATCTTTCTCTATTGCTTTATTTACATTTTCGCGAGTATCTCTCGTAAACTTGAA
TCCTTCAGGATAAAGAGTCTCAAGACTGATAAAGCATGGACCATAATATCTCAACAATTCAAAA
TGATACCAAGCACGTAAGAACTTAGCTTCAGCTTTATAAACTTTAGCTTCCGGACTGTCATACT
CTGAATTTATTACAAGATTAGATCTATATATACCACGGTAACGAGTTTTCCACAAATTATCGGA
AATAGAATTGACACTCGTATTTGAATAATCCTCTATAGCCTGCATGTAAGGCTGATCCTGATCA
GAGCCACCACCAGTACGAGCATTATCCGAACGGATTTCACCCATAGGTACAATGGAAGCAAGTG
CATTACCCGAAGCACCACCTATGTGAGCTAACGGATCATAACAAGCAGTAAGCGCTTTGAACAT
CTGTTCATCGGTCCTATAAAAAGAACTTTCTGTTTCGGACATTATAGGAGCTGTATCCAGGAAA
CTGTCGCTGCAAGATGATGATGCAATAGCAGCAAACATGAGGACAAGAATATTATTATGTATTT
TCGACTTCATAATTTTCAATTTTAGAAATTAAGACTTAAACCAAATCTGAATGTACGGGCCTGA
GGGTAAGTACCATAGTCAATACCTGTGCTAAGAATATTGCCACCTGCCATATTTCCTACTTCAG
GATCCATAAACGGATAGCTGGTGAAAGTGGCAAGATTATCAATTGCTGCATAAATTCTTGCTTT
ATTCAGCATCAACTTGTTTATTAATTTAGTTGGGAATGAATAGCCTACCTCAAGTGAAGAAATC
TTTAAATGCGAACCATCATAAAGATAAAAATCGGATGGTTTGCCAAAGTTTCCATTAGGATCTT
TGGATGAAAGACGAGGCACTCCATTATCATCACCTTCTTTCCGCCATCTGTCAAGATAGAATGA
TGGAAGGTTGCTGCGTCCGTATGCTTCCTGTCGGTAAATATCAGAGAAGACTTTATATCCAGCT
TTTCCTGTTAAGAAGATTGTCATATCAATACCTCTCCAGTCGGCACCTAAATTCAAACCGAATG
TCCATTTTGGCCAAGGATTGCCACAATCGGTTCTATCTTCATCTGTAATCTGCCCATCGTTATT
TGTATCTTGCCATATAAAGTCACCCGGAACGGCATCAGGTTGTATCACTTTACCGTCTTTTGAT
TTATAGTTCTGTATCTGCTCTTCATTTTGGAATATTCCTAAGTTCTTATAAAGGCGGAAATAAC
CCATAGCATGACCTTCCTCCATACGCGTTAGATTAACAGATGTTCTCCAGCTACCACCATCAGT
ATATCCATTTACATTTCCTATCTTTACAACCTCATTTTTAAGATATGAGGCATTTGCGGAAATA
GAGAAGTTGATTTCGTTCCAATTTTTATTAAATGTCATCTGCATTTCCACACCCTGGTTTGTTA
TATTACCAAGGTTTCTAAAAGCTGCATTATTACCTCTAATGGCTTCAACTGTTGGCTGGAACAA
CAAATCCTTAGTAGTTTTTTTAAACCAGTCGAAACTTGCTCTAATCATACCATTATAGAATGTC
ATATCGGCACCAACATTAAATTGTTCAGAAGTTTCCCATTTCACGTCTGGATTAACAAGGTTAT
TAGGAGCAGATCCCACAGTGATGGCATTACCAAACGTGTAATTATAATTATTGCCAATAATAGA
AGTATAGGAGAATGGAGAAATTCGCTCATTTCCGTTCTGTCCCCAAGAGAATCTAAGTTTGAAG
ACATCAAAGTTCTTAATTTTCCAGAATTTCTCATTTGAAACATTCCAACCTAATGAAACGCCCG
GGAAAGTAGCATATCTGTTATTGGGACCGAAATTTGAAGACCCATCGCGTCTGACCACAACTTC
CGCCATATATTTTTCAGCATAATTATAGCTTAGACGAGCAAAATATGAGAACATACTATGTCTA
GGATTAGCACCGCCACTATTAGCTGATGTCATAACATCACCAGCATTAAGATACCAGTAATTCT
CATTGGTCATTGCTTCATTTGGATATTTATTTCGTGTTCCGGCCATAAACTCATAAACATCTCT
TGATGCAGAAGTACCTAACAGGACAGATGTAGAATGTTCACCAAAAGATTTTTTATATCGCAAT
GTATTCTCCCACTGCCAACTACTATTAGCATTTGTACTTTGTTCTACCCTAGAATTATCTTCTT
TAGATTCTGCAGAATGAAAAAACTTTGGTGCAAACATTCTTCCACGGAAATTCCGATGATTAAT
ACCAAAATCTGTGCGGAAAACAAGGTCTTTAATAAAAGTGATCTCAGCATAAACATTAGCAAAA
AATTGCTGGGTAATATTTTTATTCTTAGGTGCCTCATCCATAAATGCAATAGGGTTCCACATAC
GGCTATAAGGTACAGGAGAGACTCCATATCCGAAAGTATCGTTGCTATTCTCATCATAAACCGG
AGTAGTAGGATCAATATTATAGGCGTATGATATCGGATTATAACCATTGATACCGGTTGCCACT
CCACTATTCTCTATATATGCATAGTTGACGTTTGCACCTAGACTTAAGAAATCATTTATAGAAT
AGGAACTGTTCAGCCTTGTGCTGAATCGTTTGTAAAATGACGCATCTTCACCGATAATACCATT
CTGGTCTAGATAATTCAATGAAAGCAAGCTTGAACCCTTATCACTGCCAAAGTTAGCAGTAATG
TTATGCTCAGTAACAGGAGCTGTATTCAATATTTCATTAAACCAGTCTGTATTATAACCTGTTG
GAGCAGTAGGTACACCACCGGCAAGCGGCATATCATCATTGTCGGCAAACTCTTTCATCAGCAT
AATGTACTGTTCATCATTCAGCATGGTTGGTTTCTTTGCTACTGTAGAGAAACCATAGTAACCA
TCATAAGCAAGCGATGTCTTTCCTTTCTTTCCTTTCTTTGTGGTTATAAGGACTACACCATTAG
CGGCTCTGGCACCATAAATAGCAGCTGAAGTTGCATCCTTCAAGACTTCCATGCTTTCAATGTC
GTTGGGATTTACACTGTTCATGTCGTCCATAGGCAGTCCGTCAATTACAAAAAGAGGATTAGAG
TTTCCATTTGTACCAACACCACGAATTACCAGCTTCGGTGCTGTTCCTGGCTGACCGGAATTTG
TCACAACGTTCACACCACTAACCCTACCGCTCAATGCATTCACGGCATTTGCTGGTTTAGATTG
CAATAAATCATCGGAATCGATGCTAGTGATAGCACCTGTTAGAACACTTTTTTTCTTAACCTCA
TATCCTATTGCTACAACTTCCTCGAGTGCAATGGCAGATGTTTTTAATTGAACGTCTATCTTAG
ACTGACCTTTATACACTATATTCTGTGTATCATATCCTACGAAGCTATAAATCAATGTCGATTC
CATTGGTAGATTTTCCAAGATATAATTTCCGTCCAAATCAGAAATAATACCGTTTGTGGTACCT
TTAACTAAAATACTTGCACCTATCACAGGTAAACCATCGGAGTCTGTTATACAACCGGTAACTT
TCCCGTTCTGTGCATTTAATGGTAAACTGAACGTTATAAGAATCAGCATACACATTAATGATAG
TGTTCTGTTCATAATCTAGAGTTTTTTGTAATTAGTGTTTTTCTTAAAATAAAAAGTTTTGTTC
TATCAGTTGCGCGCTACTTACTGACACTTGCAAATATATATACTATGTAATATAACCAAAGGGG
GAAAATTTCATTTAAATAGGGGGGGGAAATAGATTAACTAAATATTTTAAGGAAAAATGGCTGT
TAGAATCCATTCCCAGACTCCAACAGCCATTTTATCACTAACAATCGCCTGTTAATCAATATAT
TTTTCTGCCCATTTCCTTAAGATTTGCATCCCTGCCCAGTGGAACAAAAGTAAATCCGTATGAA
TAGCTTCCCTTCAGAAGACGCTTGTCTATTGAAGGACGGGCTTTCAGACTCCAGCTATCTGTTC
CGCCCACTCCAGCCTGAACCAGGTCGATATTAAGAGTATTAGAATACAAGTCCTTTTCAAGTTC
ATTTATATGTTTAGCCTTATCAATCGCATTCTGCGACATCTCCCACACTGAAACAGATAGGGGT
TCATCGCCGACAATCATCACACCTGCCTTATCCGACTGCAAGGCAAACCATCTCACGTCACAAC
GGTTTCCGTTTTCCTGCGGCATTACATAGTCAAATCCCAGAGCGGACACCTTGCAGTTATATAT
AGACACCATTGCAGAGGCTTTTCTGTCGGAATAGTTTTCCCATGGGCCACGTCCATAATATGTC
ACATCCGACAAACGATTGGTACATTCGCATTGCAATCCTACGCGCAACATTTCTGATATTTCAG
GAGACTTCATCATTGAATAATGAACGCCTATTGTTCCGTCTGCTTTTACTTTATAATTCAAGGT
AAGTCTCAGTCTTTCATCTATAGCCTTTAGCACCTTAACCTCAAGATTGCCTTCCGATTTGCGT
ACATCTATAGAAACTGTCTTTAGCTTTAATGGAGCATCTTTCCAGAATGCAAACAGTCTATCGA
CCTTCCATCCTCGCCAGTCATTGTCTGTTGACGCTCTCCAGAAGTTTGGTTTCAGAGCAGATGT
GATGATACTTTCATTATCTATCTTATACTGACTGATATAACCATCACTGATATTCAGATAAAAG
TTCTTTCCCTTCACGCTGATGTCTTTCTTGTTATCTGAATCGATTTCCATATCCAATGTAGTAT
CAACGCATTCTACTATCTTTGGTAAAGAAAGATACTTAAACTGTTCCCAGGCAACCTCGTATCC
AGCTTTGGCATACAGATTGTCATTCTTGAGCCTGGCACTCAGGAATAACCAATATTCCGCACCG
TCATCGGCCTTGAAATTCTGAATAGGAAGTTTTAGTTTACAGCTCTCACCAGCTGGTGTTGTCG
GCACAATAATCTCACCTTCCTGCAATACACTGTCTTCGTCCTTCAATTGCCAAAAATAACGATA
CTCATCTGTTGAAAGGAAGAAGTTTCTGTTTTTTACAGTTATCTCTCCACTATAGACATTATCA
GTTGTAAATGATACAGGAGCAAACACGTACTTGCATTCCTCAGTAGCAGGTTTAATGGAGCGGT
CGGCACTGATAACACCATTTATACAGAAGTTTTGGTCGTTGTGCTCCCCTTTCTCATAGTCACC
ACCATAATTCCATGATTTCTTATTATATTTCCGTTCATTATCCAGCAATCCCTGGTCTATCCAG
TCCCAAATATATCCGCCGGCAAGCGCATCATGAGAACGTATTGCATCCCAGTATTCTTTCAGCC
CGCCGGTAGAGTTTCCCATAGAATGTGCATATTCACACATTATTATCGGACGGTTCATGACCGG
ATTCTTAGTCATTGCTATAAGCTCATCGACCATAGGATACATACGGCTAATGACATCGACGTAT
AAAGGATCATCGGGATTGGCATACACACAAAGCTCTTTCTTTGCCGGTTTGACATCTTCGTTCA
CATTAAAATCTATCTCACTAGTAACGATTGACGCTTCCTTACGTCCGATAGGTTTGTATAAAGG
ATTTTCCGGCTGTCCTTGCGCCCCCTCGTAATGAACAGGACGGGTTGGGTCATAATCTTTCAGC
CATCCTGACAGAGCTGCATGATTAGGGCCGCATCCAGACTCGTTGCCCAACGACCACATAAACA
CAGAAGGATGGTTCCTGTCTCTCACAGCCATTCTTACCACTCTCTCCATGAACGAGTTAGCCCA
CTCAGGCCTATTGGACAGATACCCCCTTTGATGATGAGTTTCAAGATTAGCCTCATCCATTACG
TATATACCATACTTATCGCACAGTTCATAGAAATAAGGGTCGTTAGGATAGTGCGATGTACGGA
CTGTATTGAAGTTATAACGCTTCATAAGCAGAACGTCTTCGAGCATCTCATCACGTGTAACGGT
CTTACCTCCGGTCTCGCTATGGTCATGGCGGTTTACACCAATGAGTTTAATAGGAGTGTCATTC
ACCAGAATCTGATTACCTGTTATTTTAATATCCCTGAACCCTACCTTATTACTTCTCGCATCCA
CCACGTTGCCCTTTTTGTCTGTGAGCTTTATAACCAAAGTGTATAGATAAGGGTGTTCCGAATT
CCATAGTTTTGGCTTAGAAACAATTCCCTCCATCATTCCGTAATAAACATTATCACGCTGAGGA
TAAGGTTCGTTCACCACATAATCGGCAGTAACGGTAATGTCTTTTCCAAACACCGGTTTCCCAT
CGGCATCATATAATTGGGCTGACAGATTCCATCCCTTCAAATCATCCATATTCTGATTTGTTAT
TTCCGGACGGATCTGTAACCGTGCTATATTCTTCCGGAAATCGATGCGTGTCCTTACTCCATAA
TCATATATTGCCACCTGCGGAATGGACATGATATATACTTCACGATGGATACCAGCCATTCGCC
AGTGGTCGGCATCTTCCATATAACTTCCGTCGGTCCACTTATACACTTGCACCGCCAGTTTATT
CTCCCCCTTCTTAACGTATTCGGTAATATCAAATTCAGTAGGCAGACAACTGTCTTCGGAATAT
CCCACCTTCTGTCCGTTTATCCATACATTAAATCCCGAATAGACGCCTCCGAAATGGAGTATAA
TCCTGTCGCTCTTCCACTTGTCAGGAACAACAAACTCCTTGATATAACACCCCGTCTGATTATT
CCTGTCAATATATGGCGGACGAGCAGGGAAAGGATAAATAGTATTTGTATATATAGGATAGCCA
TATCCCTGCATCTCCCAACATGAAGGAACAGGAATAGTTTTCCATGATGATGAATTGTACTCCA
CTTTATAAAAACCGGCGGGAGCCAATGCCATATCCTCGGAAAAGTTAAACTTCCATTGGCCGTT
CAACGACATATACTCCGATTTCTCTCTGTCTCCATCCAAAGCCCAATCCACTCTCCGGAAAGAA
TAAGTAGTACTGCGGGAAGGCAAACGGTTAATTCCGTTTATGGTCTGATCCTGCCATACATTCT
GATTGTTTCTCCACTGATTGGCACCGTTGTCCGATGCAGACAGAAATTGCATCATGAAAAATAA
CACAGAAAATGAAAAAATAGATTTTAAGTTCAAGTTCATAAATTCGCATTTTAAGTTTCTATGC
AAATATATAAGTATAACGAACAATGAATAGGGGGTATTTCTATCTATATAGAGTGGTATTTTTA
CATATGAGCTAAAACTTAAAAAAAACTGTCAGTATTACTATGCTATGTAGCACTCTATATGAAA
ATATTATATATTCCCAAGTCAAAAGCCTTTTCAAACAATTTTTATATATTCTCATCCTATCCCT
TCCATCAAAGATAAATTCCAATCCTGATTTGCCAGCCGCATTTATTCCTTTTTTCAGGAGAATT
TTCTTTATGGCTATCGCCATGAAAATTCACCTGAAAAAGAATGCGGCGGCAAACGGATTAGAAT
TAAAGAAAAGATTACAGGGATTAACTGCGACCGACGTGACGCATAGCCGTAATTCAAAGGCGGC
TATCCTTATATTCCATATATGACCTCACAAATACTGTGAAAATCCACTTTCCCCAATAACAAAA
CATAGCCTGCCATATCAACACCCAAAATAAGACAGGGATTTCAACTCCCTCCGATCTGCATAGT
CTGGTGGCTTCGCTATGCTTTTACTCCTACATCCATTTTTTTTCTTTCTTTTTTCCTCTGTTCC
CGTTCTTTCCTATCCTTCGTGTGACATTTGATGACACCTGATGACATCTAATGTCATCTATTTG
TAAATCAATTGTTTACTCAATTTATCATCTTACATTTGGACTGTGAAACAAATCAAGTAGTCAC
TCAAAACAAAAGATTATGGCACAAGAAAACAGTCCTGACAAGGAAAAAAGGCAAGGCCGGACAA
AGAAACCCGAAAAGCCTTATGTGGAACAAATTGACGAGCTTCTGCTGGTACATAACAAGAATGA
CCCAAAGGAAGGTTTGGGAGTAATCAGCAAGATGGACGAGAAAGGCAATTATCAGACGGTTACA
CCGGAAGAGAAGAATGAGAACTCATTCCTGAAATTCGACAAGAATTCGAGTATTCTCGAAAACT
TCATCAAGAATTTCTGGAGCCAGCTGAAGGAGCCTACGCATTTCAGGCTTATCCGTATGACCTT
CAATGATTACAAACAGAACAAACAGGCTCTCAAGGACCTGGCCGAAGGCAAGAAGACAGACGCG
GTAAAGGAGTTTCTGAAACGCTATGAAATCAGACCGAAAGTAAACAATCAGAAAAACAGTCAAA
CAAAAGAGGAGGAAACAACAATGGCAAAGAAGCAGGAACAGACAACGCAGGCTCAGCCTGAACA
GGTATCACAGGTGGAAGCTGCCGCACAGGGGCGCGAACAGCAGGAACCGCAACGCCAGCAGACA
CCCACGTACCGCTACAACGAGAACATGATTAATTGGGAGGAACTGGGTAAGTTCGGTATATCCA
AAGAAATGCTGGAGCAGTCCGGACAGCTTGACAGCATGTTGAAAGGATACAAGACCAACAGAAC
CATGCCGCTGACACTCAACATTCCTGGGGTACTGACCGCAAAACTTGATGCACGCCTTTCGTTC
ATATCCAACGGCGGGCAGGTCATGCTGGGCATCCACGGTATCAGAAAGGAACCTGAACTGGACC
GTCCTTATTTCGGACATATCTTCACGGAAGAGGACAAGAAAAACCTGCGTGAAAGTGGAAACAT
GGGACGCGTGGCTGACCTTAACCTGCGTGGCAACACGACAGAGCCGTGTCTGATTTCCATCGAC
AAGAATACCAACGAACTGGTAGCCGTACGGCAGGAGCATGTCTATATCCCGAATGAAATCAAAG
GGATAACCTTGACTCCGGACGAAATCCAGAAACTGAAAAACGGAGAACAGATATTCGTAGAGGG
AATGAAGTCCAATCAAGGTAAAGAGTTTAATGCCAATCTGCAATATAGTGCGGAAAGAAGAGGC
ATCGAATTTATCTTCCCGAAAGACCAGGCTTTCAACCAGCAGACGCTTGGCGGTGTACCGCTTT
CCCCCATGCAGCTCAAAGCGTTGAACGAAGGACACACCATCCTTGTAGAGGATATGAAACGAAA
GAACGGCGAACTGTTTTCTTCCTTTGTTACCATGGACAAGGTTACAGGCGGGCTCCAATATACG
CGCCACAATCCGGAAACGGGAGAAATCTACATACCAAAGGAAATCTGTTCGGTACAGCTCACAC
CGGAGGACAAGGAAGCGTTACGCAAAGGGCAGCCCATCTATCTTGAGAACATGATCAACCGTAA
AGGTGAGGAATTCTCGTCATTCGTCAAGCTGGACCTGGCAAGCGGAAGACCACAGTATTCCAGA
ACTCCGGACGGTTTCAACGAACGACAGGCACCAGCCATCCCGGCTGAGGTTTACGGACACCTGC
TTTCGGCACAGGAAAGAGCTAATCTTCAGGACGGAAAGGCTATCCTCGTAACGGGTATGAAAGG
TCCCAACGGCAAACCGTTCGATTCCTATCTGAAAGTAAACGCAAACACCGGACAGCTGCAATAT
TTCCAGGAAAATCCGGATGTGCGCCGCAATACTTCACAGCGTGCTTCACAGACTGACAATACCC
AGCAGCAGGAACAGAAGAAGGGAGCAAAACAGGCTGTCTGACCTGAACGGGATTCAAATCATTC
AAATCATCAATTACTAAAAAAGGAAAGAACATGAACAAGACCAATCATCATATCTACAAGACTG
AACAAATCGACTGGGAGAAACTGGAATCGGTAGGTATCAGCAGATCGCAAATTGAAAAGGACGG
AAACATGGACCTGCTCCTTCAGGGAGAGGAAACCAATGTCATGTCCATTAAAATCAAGACTCCT
GTATTTTCACTGACCATGGACGCCACACTCAGTCTGATTGAAGACGAGAATGGAAATCCGGTCA
TCAGCGTAAACGGTATCAACCCTTCAGGTGAATAAATAAGAAACCATAATGTATCATCTCTCTT
TCCATACGGACTTACCGTATGGAAAGAGATAAAAACAGAATTTATCATGATTGCCATATTAACA
GACAAACCAAGTGTAGGAAAAGAAATCGGAAGAATCATCGGTGCAACCAAAGTAAGAAACGGAT
ATGTGGAAGGAAACGGCTACATGGTTACATGGACTTTCGGGAACATGCTGTCACTGGCCATGCC
GAAGGACTACGGAACCCAGAAGCTGGAACGGAATGACTTTCCTTTCATCCCGTCCGAATTCGAA
CTGATGGTACGGCATACACGCACCGAGAACGGATGGATACCGGACATTGATGCCGTGCTCCAGC
TTAAAGTAATCGAGAGAGTGTTTCAGGCATGCGATACCATCATTGCGGCTACCGATGCCAGCCG
TGACGGGGAAATGACATTCCGCTATGTCTATCAATACCTGAACTGTACACTGCCTTGCTTCCGT
CTGTGGATTTCCTCTCTTACCGACGAGTCTGTGCGTAAAGGCATGGAAAACCTGAAGCCGGACA
GTTGCTACGACAGCCTGTTCCTTGCTGCCGACAGCCGCAACAAGGCGGACTGGATTCTCGGAAT
CAACGCCAGCTATGCCATGTGCAAGGCGACGGGCCTTGGCAACAATTCTCTCGGACGGGTACAG
ACACCGGTACTGGCTACCATCAGCAGACGCTACCGTGAAAGGGAGAACCATATTTCATCGGACA
GCTGGCCCATCTACATCAGCCTGCAAAAGGACGGCATCCTTTTCAAGATGCGCCGCACACAGGA
TCTTCCCGACAAAGAATCCGCTACAATGTTTTTCCAGGACTGCAAGCTGGCACATCAGGCACAG
ATTACAGGTATCAGCCACAGCGTTAAGGAAATACTTCCACCGGACCTGCTTGACCTGACACAAC
TTCAGAAGGAAGCGAACATCCGCTATGGTTTTACCGCATCAGAGGTGTATGACATCGCCCAGTC
TCTTTATGAAAAGAAACTGATTTCCTATCCGCGGACTTCCAGCCGTTATCTGACGGAGGATGTG
TTTGACTCGCTTCCACCAATCATGGCGCGTCTGCTTTCATGGGAGCTGTTCCCTGCAGCTAAAG
GAACTGGAGGTATTGACATATCCAATTTGTCCCGCCACGTAATAAGCGCAGAAAAAGCCAATGT
ACATCATGCCATCATCATTACAGGTATCCGTCCCGGAAATCTGTCCGAAAAGGAAATACAGGTT
TACAGACTTGTAGCCGGAAGGATGCTTGAAACATTCATGGCTCCATGCCGCATAGAAACGACAA
ATGTTGAAGCGGTTTGTGCGGCACAGCATTTCAAGGCCGAACAAACAAGAATCATTGAAGCCGG
CTGGCATGATGTGTTTATGCGTTCCGACATGGTTCCAAAATCAGGATATTCTGTCAATGAACTC
CCCGAAGTGGAGAAAAGTGATACTCTGAATGTATGCGGATGCAACATGGTACACAAGAAACAGC
TGCCGGTAAATCCGTTCACGGATGCAGAACTGGTGGAATACATGGAACAGAACGGACTGGGTAC
AGTATCCTCACGTACCAATATCATCCGTAGACTGGTTAACCGTAAGTATATCCGTTATTCAGGG
AAATATATCGTTCCGACCCCGAAAGGCATGTTCACCTACGAAACCATCCGTGGAAAGAAAATTG
CGGATACTTCACTCACCGCAGACTGGGAAAAACAGCTGGCCGGACTTGAAAGCGGAATGATAAC
CGGACAGGACTTCCTGAACAGGATCAGGACTCTCGCCAAGGAAATGACTGATGACATTTTCAAC
ACCTATTCCACAAAAGAAGAATAACATCTATACCTAATCAACCAAGAGAATGCAGGCCGGAAGG
TCTGCATTTTTTTGTATCCGTACAGAAAAGAATCTGTTTTTCCGCTTTTAAGCGGCAAAGGTCT
TGGATTGCCTGCCTTTTGCCGCAAGGCTGCCCTCATGGGCTTGGCTGGACAGGAAAAAATCATC
CTCGCTGCGCTCCGGTATTTTTTCCTGCCAGGCCTTGCGCAAAAAGGCAATCCAAGAGGCCGGA
GGCCTATAAAATCGGGAAAACACATCCCGATGGGATTATTCATTCATAAAATTAAGGATTATGA
AACTACAGATTATCAGAAAGATCGGCAGACATGCAACAGCGATATTCCTGATTACCGGAATATG
TCTGCTGACAAGTAAAGGGATTGTCCCTACTGGGATGATTACGCTGCTGTTGCTTGCAGGAGGG
TTCATCGGTTTTCTGTTCAGGATACTGGTCATTATTTTCAAGATTCTTATTCTTCTGTTCATTG
TAGGATTATTTGTCGCATAACCCAAAATATAAATATACATATATGGAAACAGTTGCTATAACCT
CACAAGCTCCTGTCATGCCGGCTGTATGGCCACAGAACGAACATATCAGACCGGTTAAAAGACG
TCTGCCCAATACAGTTGATGAACCTAAAAATATCGGCTACTATCTGGAATCGCTACGTGATATT
TCCAGCAATCCGGACAGAGAGAATATTCTGAAAGAATTCTTCAAGGAAACTTATGTATAACCAT
AAAATTTTTCAATTATGTTTTTTCAATCAATTTATCAGATGATTACAGCAGGTAGGGATCTGAA
TATCAATATCCGTAAAGTGGACAACAGCCTGAGCGTAGCAGTCATGCCAAGGCGGAACAGCCTG
AAAGAGGATACGCGACAGAACATGGTGCCACTGATCGTGAACGGAACACCGGCAGAACTGGATA
TGGGCTTCCTGCAGACCATACTCCAACCGATACAGAAGGTACAGGGACTGCTTGTCAATGCGGA
AAATTTCGAGAAACAGGCAGAAAAGGCTACATCACAGGCCAAATCATCCAAGGCTCCAACAATA
CCGGCCGAATCAAAGGAAGCCAGGGAAAAACGGGAAAAGATGGAAAAGCTCCTCAAGAAGGCTG
ATGAAGCAACCGCCGCAAAAAGGTACTCCGAAGCAATGACATGGCTGAAACAGGCACGGGTACT
GGCTCCTACAGAAAAACAGAAGGATATTGACGAAAAGATGCAGGAAGTACAGAAACAGGCTAGT
GCAGGAAGCCTGTTCGGTATGGCAGAGGAACCGGCGCCGGTAATTCCCCAACCACAAGGCTATA
TGAACGGTCAGTCACAACCAGGTATGCAAACAAGCATATTCCCGGAGCAACAGACCCATACTAT
GAATCCTGAACCTGTCATGCAGCCTGCTCCACAGCAGGTATCACAACAAATTCCACAAGGAATA
CCTCAACCGGCATATGGAACGAACGGGACATATAACCCACCTGCTCCAAACAGCCCGATAGTAA
AAGGAGCAGACATACCGCAAGGCGCAACAATGCATCCTTACCCACAGCAGCCATACTACCAGCA
AGAGGCGACTCCTTATCCAACACAACAGCCACAGCAACCGACAAACGGACATATACCGAATGGG
GCTGCGCAAGTACAGAATGGAAACGGACGGGAATACCAGACTGCATCGGCTACACATGAGACAT
TCTGCTTCGATCCGGAAGACGAGAATGACAGGGAACTTCTAAGAGAGGACCCGTATGCGGAATA
TCCGGATTTTCCGGCTGAGTACCGAATGAAGGACGAGGCACAGGTAGAAATGGTATACTGCTGA
TATACACAATAAACGATTTGTAAAACCAATAAACTATAAACAATATGGCACTGGAAATTAAAGG
AATGAAAAGAGTATTCAAGATGAAGAAGAACAATGAGGAAATCGTACTGGATGATCCGAACGTA
AACATGTCTCCGGCTGAAGTGATGGACTTCTATTCCATGAATTATCCGGAACTGACAACCGCGA
CCGTACACGGACCGGAAATCGAAGACGACCGGGCGGTATATGAATTCAAGACCACTATCGGAGT
AAAAGGGTAAGAGCATGAAAAAAGGACAACGTAAAGACAAGAAACCATGTACACAACTTACGGA
ACGGGCTTTGGAAAATTTAGCCAGACTTATCATATCGGAACTCGAAAATACGGACATAAGCCGG
GGCATCAGGAACAGAAAGAAAAGAAGACTCCCTCCCGCAGAAAGCCTCATGGTTTTCTGAACAC
GAGAATACCTTCCATCGCTCCCGATCTGTATGTTGAGAATGACAGGGATGTAACGGTAAATGTC
ACCACCAAAGAGAATCTTGATTTCCTGTACCGTTCAGCCATGAAGTATGCGCAGCTCCTGGATG
TGGAGCTGCCATACCATCCTACAGGCAGGACTTCCACAAGAGAGAAAATATGCCTGCTATATAA
TGCACTGGATTCCATAGTATCTCATCATGTAAATCTGGAACTTATTGGTGACAGGCTCCAGTTC
TGCATCTACCATTTCCATGAATGGCCGGATTATACGCTTTTCTTTATGCCGATAGACTTTACGG
AAAGGCTGCACGGTGAAATTAAAAAGATTACACTGGAGTTCATCAGAAAGTTCATCAAATATCA
CAGGATGATGGATATAACCGATACCCCTTATTTTGAGATGTCGGAAGTCTGTATCGATTATGTG
GACTTTGAACAGCTCGATGAGGAAGAGAAAAAGGATTTGTACAGAAAGGAAAAGCTTTTCAGGT
CATATGAGAAAGGGAGAATCCACAGGAAGCTGTGCCGGATGCACTCCAGGGCTTTCTGTAGGAA
TCTGGAAGAACATATCCGCAACTGTACTCCTTCCAGCGATAAGGAAAGAAGACTTTTGGAACTG
ATTACCGAAGGGCTGTCCCTGATTGCAAAGGACAGCCCTTATATCTTGAATTATGATTATGATT
TTGCAAGCGAAAAGGAACGGGATTTCGAGCCGCCACCGCTCGAATATCAGATTCTGCTTACATA
TTCCATCACGGATACGGTTACCAAAGACATGGAAAGCTGTTTCAGTACTGACTGTCAGGAAACA
TATAACCAGACTCCCGTATCATTTACCTTCATCACGCCGGAAACAGAGGAACTTTTCAAGCCGG
ACAACTATCCGGAACGGTTTGAGAAATGGTTTGAGAAATTTGTAGAACATGTTACCTATAATTT
ATAAACATCATGAATGAACTGACCAAAAATATGCAAAAAATGATGGTACCGAAGGCTGCAATCA
TAGCCTACAAGTATGAAGACAGAAGAAATCTTGATACCAGGTACTTTATAGAATTACGTCCAAT
CAGAAAAAGCGGACAGATGGGGGCAGGTATCCCCGTCACATACGAATTCATGAATACCCTGCTG
GAATCCTATACGGAAGAAATGAGCGGGATACCGGCAGGCAGAGTCCCTGAAAACATGCTGGCCT
GCAATCCGAGAAAAGGACAGGAAGAATATATCTGGTACAATCCGCCCGGAAAAAGACAGATGTT
CTTTCACAAGGATCTCAATATACAGGACGGCATGTTCAATCTGCCGGGAATTATCTACCAAGTA
AAAAACGGAAACATGGACGTGTTCGCTTTCAAGGGGAAACGTCCGGTGGAGACGACTCCGCTGT
TCCGTGCCCCGTTCTTCAACGTGACCGGATCAAGTGTCTGCCTTGGCAACAGTTCTCTGGAAAA
GCCACAGAACCCGACTTTCCTTTCCCTGCTGGAATACTGGGAAAAACGGTTCTGGCTGACTGAA
TTCTCCCATCTGGGAGGAAATGTCAATCCTACCGTTTCAAATCTTGTCATCGTCACCGAAAATA
TAAGAAACAATCCGTTCGACATGAACGAACTCAAGCCCATGAATAAAAAACTTAAAGACATACT
TCCATGAAAAAGATACATTTTACCGACCGCTACCTGCTCAATCCACGTCATCCGGTAACGGTAT
TCGTCATCGGAGCTGGAGGTACCGGCTCACAAGTGATAACCAATCTGGCACGCATGAGCATGGC
ACTTCAGGCATTAGGTCATCCGGGACTGCATGTCACCGTATTCGATCCCGATACGGTTAGCCAG
GCCAATATAGGACGCCAGCTTTTCAGTGAGACGGAACTGGGACTGAACAAGGCCGTATCACTTG
TCACACGCATCAACCGTTTCTTCGGATACGCATGGACTGCCGAACCGAAATGTTTCCCAACGAA
GAAATTTTCAGGATATGATACAGCCAACATATTTATCACCTGCACTGACAATATACGTTCACGT
CTTGAGATTTGGAAATTTCTAAAGAAAACTCGTAAAGAGAACTTCAATGACTATTTGGTTCCTA
TATATTGGATGGATTTTGGGAACAGCCAGACAAAGGGACAGGTCATCATCGGGACGGTACGTGA
GAAAGTTCTCCAACCTTCTTCACAAGAATATATTCCCATGCCTAAAATGAATGTCATCACCGAG
GAAGTGGACTATGCGAAAATCAAGGAAAAAGAATCAGGACCAAGCTGTTCTCTGGCGGAAGCCC
TGGAAAAACAGGATTTGTTCATTAACTCCACACTGGCACATATCGGATGTGACATATTATGGAG
AATGTTCAAGGAAGGAAAGACACTGTATCGCGGTGCCTATGTCAATCTGGATACATTGAAAATG
ACCGCAATCCCGGTGTAATGACAGAAGTGACCGTATCATCTTTCCATCAGAATACGGTCACTTA
TTCTATTTGCTACTTATTATTTACTACGTTCTTACCACGCTGGAGCAGGAAACTCTGTATCTCT
GAGGCGAGATAGAATGATTTCCCGTTCTTTTCCACCGAGTAATATTTAATCTTGCCCTCTTGCC
TGTAACGTGCCAAAGTTCTTTGTGACACACCAAGGAGTTCTGCCAGATCCACATTATCAAGCAG
TCTGTCTCCATTCATACATTCTTTCAGACGATTCATCTGGTCCAGTTTCTTTTCAATGCGGGCA
AATCCCTCTACCATTGTTCCTATAAGTCTTTCGAGTATCTCATTATCTATATATGACATAATTC
CAATGTTATTAAGTGAATAAATCGATACTCTCTTCGTGCGCACTCTAAGAGTATGTAGTTATAG
TAGTGAAAATAGTATGCCTGAATCTAAGACAAAGATCAACAAGCTTATTAGGCGCTGATAATCA
GGCGTATAATTTTTTCTACTTAATATTTAGTGTAAACCAAAAGTGTAAACTATGTAATACAGAA
TTGGGAACGGGTTAACACAGCCACCAACAATGACATCTGATGCTACCTGACGACACCTAATGAC
AACATTTTGTATCATATACATATTCAAAATACATTTGTACAAACTCAACTTTTTTGGATATGGA
AATCATTGGAATTGAAACAGCTACATATGAAAAGACATTAAAGGAAATTGAAAACTTCCTTGAT
ACCATTGATAAATTGATTACAGCTTCTTCACAGAAAACAATAGGGGAATGGTTGGATAACCAAG
AAGTTTGCCTGATCCTCAAAATTTCTCCAAGAACATTACAGAATCTTAGAGATACAGACCAAAT
CTCTTATTCTCAAATTGGGAAAAAGATTTATTATAAAAAAGAAGATATTCAGAAGTTCATTGAA
AAACACAACAGAAAATTATGAGCAAGGTAATTACCCAAGATAATGAGCAAGTTATTCAGATATA
CAATAGGTTAAAAGATACGCTAACAAGACTCGAAGATATTCTGAAGAATAACAACCCAACACTT
AATGGGCATAGATATATGAATGATGCAGAATTGGCTAATTACCTTAAAGTATCAAGACGCACTT
TACAAGAATATAGAAATAATGGAATCTTATCTTATTATCAGATTGGAGGTAAAATTCTATATCG
GGAATCTGATATAGAAGAACTTCTTGAGAAAAACAGACAGGAAGCATTCCGTTAAACATTTCTT
GGAATTTTCGTTGATTTTCAAAGCAAAAATCAGTATCTTTGCAATACTGACAAAGAGTTGTATA
TCAGTGCAGAACAAAGAAGTTCAATCGAGGTGAAATAGGTGGACTAAATGACAAACAACAAGAT
AAGTAATTGATTATTAGCGATAAAAAATATAAGGTTCCGCCCCCAGGCGGATCACTGAAAACAA
AAGAGAAAT

1. A method for enhancing an immune response to a selected tumor cell antigen, the method comprising:
orally administering a genetically engineered Bacteroides bacterium to a subject, wherein the subject comprises tumor cells expressing the selected tumor cell antigen, and further wherein the genetically engineered Bacteroides bacterium comprises a heterologous polynucleotide encoding the tumor cell antigen operably linked to control elements, whereby the tumor cell antigen encoded by the polynucleotide can be expressed in the subject by the genetically engineered Bacteroides bacterium,
wherein, upon expression in the subject of the tumor cell antigen encoded by the polynucleotide, the immune response to the selected antigen expressed by the tumor cells is enhanced, as compared to the immune response against the tumor cell antigen expressed by the tumor cells in the absence of the genetically engineered Bacteroides bacterium.

2. A method of treating a cancer comprising cancerous cells that express a tumor cell antigen in a subject in need thereof, the method comprising orally administrating to the subject a genetically engineered Bacteroides bacterium comprising a heterologous polynucleotide encoding the tumor cell antigen.

3. A method of stimulating an immune response against a tumor cell antigen in a subject, the method comprising orally administrating to the subject a genetically engineered Bacteroides bacterium comprising a heterologous polynucleotide encoding the tumor cell antigen.

4. The method of any one of claims 1-3, wherein the tumor cell antigen comprises a tumor-associated antigen.

5. The method of any one of claims 1-4, wherein the Bacteroides bacterium is a Bacteroides thetaiotaomicron bacterium.

6. The method of any one of claims 1-5, wherein the genetically engineered Bacteroides bacterium is not attenuated.

7. The method of any one of claims 1-6, wherein the subject is a human.

8. The method of any one of claims 1-7, wherein an adoptive cell therapy is administered to the subject prior to, concurrent with, and/or subsequent to, administering the genetically engineered Bacteroides bacterium.

9. The method of claim 8, wherein the adoptive cell therapy comprises a CAR-T cell therapy.

10. The method of any one of claims 1-9, wherein an immune checkpoint inhibitor is administered to the subject prior to, concurrent with, and/or subsequent to, administering the genetically engineered Bacteroides bacterium.

11. A method for enhancing an immune response to a selected tumor-associated antigen, the method comprising:
genetically engineering a Bacteroides thetaiotaomicron bacterium to comprise a heterologous polynucleotide encoding the tumor-associated antigen operably linked to control elements, whereby the tumor-associated antigen encoded by the polynucleotide can be expressed in a subject by the genetically engineered Bacteroides thetaiotaomicron; and
orally administering the genetically engineered Bacteroides thetaiotaomicron bacterium to the subject, wherein the subject comprises tumor cells expressing the selected tumor-associated antigen, and further wherein, upon expression in the subject of the tumor-associated antigen encoded by the polynucleotide, the immune response to the selected antigen expressed by the tumor cells is enhanced, as compared to the immune response against the tumor-associated antigen expressed by the tumor cells in the absence of the genetically engineered Bacteroides thetaiotaomicron bacterium.

12. A method for enhancing an immune response to a tumor-associated antigen in a subject comprising tumor cells expressing the tumor-associated antigen, the method comprising orally administering a genetically engineered Bacteroides thetaiotaomicron bacterium to the subject, wherein the Bacteroides thetaiotaomicron bacterium has been genetically engineered to comprise a heterologous polynucleotide encoding the tumor-associated antigen operably linked to control elements.

13. The method of claim 11 or 12, wherein the subject is a human.

14. The method of any one of claims 11-13, wherein an adoptive cell therapy is administered to the subject prior to, concurrent with, and/or subsequent to, administering the genetically engineered Bacteroides thetaiotaomicron.

15. The method of claim 14, wherein the adoptive cell therapy comprises a CAR-T cell therapy.

16. The method of any one of claims 11-15, wherein an immune checkpoint inhibitor is administered to the subject prior to, concurrent with, and/or subsequent to, administering the genetically engineered Bacteroides thetaiotaomicron.

17. A genetically engineered Bacteroides bacterium comprising a heterologous polynucleotide encoding a tumor cell antigen operably linked to control elements, wherein the tumor cell antigen encoded by the polynucleotide can be expressed in a subject by the genetically engineered Bacteroides bacterium, and wherein, upon expression in the subject of the tumor cell antigen encoded by the polynucleotide, an immune response to the tumor cell antigen in the subject is enhanced as compared to the immune response in the absence of the genetically engineered Bacteroides bacterium.

18. A method for enhancing an immune response to a selected tumor cell antigen, the method comprising:
orally administering a genetically engineered Listeria bacterium to a subject, wherein the subject comprises tumor cells expressing the selected tumor cell antigen, and further wherein the genetically engineered Listeria bacterium comprises a heterologous polynucleotide encoding the tumor cell antigen operably linked to control elements, whereby the tumor cell antigen encoded by the polynucleotide can be expressed in the subject by the genetically engineered Listeria bacterium,
wherein, upon expression in the subject of the tumor cell antigen encoded by the polynucleotide, the immune response to the selected antigen expressed by the tumor cells is enhanced, as compared to the immune response against the tumor cell antigen expressed by the tumor cells in the absence of the genetically engineered Listeria bacterium.

19. A method of treating a cancer comprising cancerous cells that express a tumor cell antigen in a subject in need thereof, the method comprising orally administrating to the subject a genetically engineered Listeria bacterium comprising a heterologous polynucleotide encoding the tumor cell antigen.

20. A method of stimulating an immune response against a tumor cell antigen in a subject, the method comprising orally administrating to the subject a genetically engineered Listeria bacterium comprising a heterologous polynucleotide encoding the tumor cell antigen.

21. A method of treating a cancer comprising cancerous cells that express a tumor cell antigen in a subject in need thereof, the method comprising (i) orally administrating to the subject a genetically engineered, non-pathogenic, gut bacterium comprising a heterologous polynucleotide encoding the tumor cell antigen, and (ii) administrating to the subject a checkpoint inhibitor.

22. A method of stimulating an immune response against a tumor cell antigen in a subject, the method comprising (i) orally administrating to the subject a genetically engineered, non-pathogenic, gut bacterium comprising a heterologous polynucleotide encoding the tumor cell antigen, and (ii) administrating to the subject a checkpoint inhibitor.


SEQUENCE LISTING (6 sequences)

SEQ ID NO: 1 - DNA, 8848 bp, Artificial Sequence
  Synthetic sequence
caattcgagggggatcaattccgtgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaacggaattgatccggccacgatgcgtccggcgtagaggatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggaattcccctccaccgcggtggcggccgcatcttccagtgatggaacagcatttgtgcattggctgcaacaatcagccttacttgtgcctgttctatttccgaacaaaaaaaaggccatccgtcaggatggccttctacgtctcattttcgccagatatcgacgtcttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacattctttaaaaacagatttggagtgcaaagttacgacaaataatttgttaacatacatatttaggcaactttttcactatattttcaactaataaaacaaaaaagaatgccgacaagttgtgtgacggcatcctttttctatttcattagcttatcctacaatcttcttaccttaatttataatttattcctgagtcagatcaactgcaaagtatccgcgtttaccggatgggaatacacctgtcaggaacaatacctgattcggagacttaacagcagctttcattgcttcttccagatcagatcaagtgctcgaggtatcctggtaaaataagctcgtactgtaattcatgcgacatgatgcgtaaagagatctgaaatctattggtctatgtcactgaaactatccaaataccttatgtcgatactgaacgtattgacgcatacctccttttattcgattggattatctactttattactatatttcggataaagcgtggtgccccagatggagatatttctatttttcacaagtggtaagttccggtcatcaattaccgttctccaccattcccaagctaaaccagtgcattctttagcgtaaacattaatatttctcgcgttacctggcaaatagatggacgatgtgaaatgagctagcttgcttttattgttttcgctccagtttttatgttgaacaatttcgttaccttcaggatcataatttacttcatcccaagaaatgttgaattgagcaacgtatcctccagagtgatcgatgttaatttttccatctgtataagcttttgaagttgtttcaatatattctgagttgtttttaataacagctaattcattgtcttttaggaagtttgttgtataagcaatgggaactcctggtgtttctcgattaaaagtagcgccttttttcaaaatatcgcgtaagtctccgaggttgccgtcgatgatttgaacttcatcttttgcggaacctccgtaaattacggctttgaaggaagaatttttgatgatatttgttagttctacatcacctgagacagattttccgcttacggcagcatcaaaagcagcttttactttagtactatgggaattagttgataatttcaaataaacttgacggccatacgccacacttgagatatatgcaggaggattttctgcattcactccaagcgcttgcaactgctctttagtaacagctttgccgaaaaatctggaaggtcttgtaggttcattaacattcacgttatagtaaatttgtttaaaactaatgacttcttcttgcattttcccttcactgattgcgccgaagtttacattcaagctattatttacagctttaaatgctgtaccaaatttcgcaattaattgtgattcactgtaagccatttcgtcatcataatcaatttttgcacttacatttggataagcttgagcatatttttcattccatctttccactaatgtatttactgcgttgttaacgtttgatttagtggcattttttacaacgattttattgtcttgattagtcatacctggcaaatcaatgctgagtgttaatgaatcacgttttacagggagaacatctggttgattttctactaattccgaattcgcttttacgagagcacctggataggttaggctcgaaattgcattcacaacttgaatgtctgcattattttgattgatggatttcttctttttctccacaacaatatattcatttccatctttgtaaccttttcttggcggcacatttgtcactgcatctccgtggtatactaatacattgtttttattgtaatccaatccttgtatatacttatcgatttcatccgcgtgtttcttttcgattggcgtcttaggacttgcaggcggagatgctggtggtgccatggatgaaattgaattttctttattgaatgcagatgcatccttcatggattcgtttaactaaacgaataatttattttattttaaaatgcgtctctatcactgatagggagactgcaaagataagaacttttagctttatcttccaaactttatccctatcagtgatagagatttttttgttcttatggtcaagcagtactgcgcttttacactaagtcaatttaacaaggctttcttctcttggaaagcgggtgcaaaaccgtttcaacagacgcttggtaagatcattacgaataccaggtatcgtgtaagtagcgtaagcctatacgcaagataaactgctaaaaaaccggcgcgctttgcacccgctttccaagagaagaaagccttgttaaattgacttagtgtaaaagcgcagtactgcttgaccataagaacaaaaaaatctctatcactgatagggataaagtttggaagataaagctaaaagttcttatctttgcagtctccctatcagtgatagagacgcattttaaaataaaataaatcatatagttaattaaacgaatccatgcgttatcccatcttgcccgaatatcttcaatgtgttaaggaactgtatcgtggaggactggaacccatcaactttcaaaccgccgctgatcaggcacgtgaactgatcaatagttgggtagaatcccaaacaaatggtatcattcgtaatgtactgcagccgtctagtgtggatagccaaaccgctatggtactggtgaatgcaattgtgttcaaaggactgtgggaaaagactttcaaggatgaagatacacaggccatgccgttccgtgtaactgaacaagaatctaaacctgtgcagatgatgtatcaaatcggtctgttccgtgtagccagcatggcttctgaaaaaatgaaaattctggaactgccgtttgctagcggtacaatgtctatgctggtactgctgcctgatgaagtgagcggactggaacagctggaatctatcatcaacttcgaaaagctgacagaatggacatctagcaatgtaatggaagaacgtaaaatcaaagtgtatctgccgcgtatgaagatggaagaaaagtataacctgaccagtgtactgatggcaatgggtattactgatgtgttttctagttccgccaatctgtccggaatcagctctgctgaaagtctgaaaatttcccaagcagtacatgctgcacatgcagaaatcaatgaagccggacgtgaagtagtgggtagtgcagaagccggagtagatgccgctagtgtgtccgaagaatttcgtgccgatcatcctttcctgttttgtatcaagcatatcgctaccaacgctgtattgtttttcggtcgctgtgtaagcccgtaacgaccgcttgtatgaatccatcaaaattcgttttctctatgttggattccttgttgctcatattgtgatgataatttctacaaatatagtcattggtaactatctatgaaactgtttgatacttttatagttgattaaacttgttcatggcatttgccttaatatcatccgctatgtcaatgtagggtttcatagctttgtagtcgctgtgtcccgtccatttcatgaccacctgtgccgggattccgagagccagcgcattgcagatgaatgtccttcttcctgcatgggtactgagcaaagcgtatttgggtgtgacttcatcaatacgttcatttcccttgtagtaggtttcccgtacaggctcgttgatttctgccagttcgcccagctctttcaggtaatcgttcatcttctggttgctgatgacgggcagagccatgtaattctcgaaatggatgtccttgtatttgtccagtatggctttgctgtatttgttcagttcaatcgtcaggctgtcggcagtcttgactgtggttatttcgatgtggtcggacttcacatcgcttcttttcagattgcgaacatccgaataccgcaaactcgtaaagcagcagaacaggaaaacatcacgcacacgttccaggtattgcttatccttgggtatctggtagtctttcagcttgttcagttcatcccaagtcaggaagattacttttttcgaggtggttttcagtttcggtttgaacgtatcgtatgcaatgttctgatgatgtcctttcttgaagctccagcgcaggaaccatttgaggaatcccatttgcttgccgatggtgctgtttctcatatccttggtgtcacgcaggaagttgacgtattcgttcaatccaaactcgttgaaatagttgaacgttgcatcctccttgaactctttgaggtggttcctcactgctgcaaatttttcataggtggatgccgtccagttattctggttaccgcactcttttacaaactcatcgaacacctcccaaaagctgacaggggcttcttccggctgttcttcgctggtgtctttcattctcatgttgaaagcttccttcaactgttgggtcgttggcatgacctcctgcacctcaaattccttgaaaatattctggatttcggcatagtatttcagcaagtccgtattgatttcggctgcactttgctttagcttgttggtacatccgctctttacccgctgcttatctgcatcccatttggctacgtcaatccggtagcccgttgtaaactcgatgcgttggctggcaaagatgacacgcatacggatgggtacgttctctacgattggcacaccgttctttttccggctctccaatgcaaaaatgatgttgcgcttgatattcataattgggtgcgtttgaaattctacacccaaatatacacccaattattgagatagcaaaagacatttagaaacatttacttttactctatattgtaatttacacttgattatcagtcgtttgcagtcttatgatattctgtgaaagtataagttcgagagcctgtctctccgcaaaaaacgctgaaaatcagcagattgcaaaacaaacaccctgttttacacccaagaatgtaaagtcggctgtttttgttttatttaagataatacaaccactacataataaaagagtagcgatattaaaagaatccgatgagaaaagactaatatttatctatccattcagtttgatttttcaggactttacatcgtcctgaaagtatttgttggtaccgaggacgcgtaaacatttacagttgcatgtggcctattgtttttagccgttaaatattttataactattaaatagcgatacaaattgttcgaaactaatattgtttatatcatatattctcgcatgttttaaagctttattaaattgattttttgtaaacagtttttcgtactctttgttaacccatttcattacaaaagtttcatatttttttctctctttaaatgccatttttgctggctttctttttaatacaattaatgtgctatccactttaggttttggatggaaataatacctaggaatttttgctaatatagaaatatctacctctgccattaacagcaatgctagtgatctgtttgtatctaataacattttagcaaaaccatattccactattaaataacttattgtggctgaactttcaaaaacaatttttcgaattatatttgtgcttatgttgtaaggtatgctgccaaatattttatatggattgtggctaggaaatgtaaatttcagtatatcatcatttactatttgatagttaggataatttaagagcttattacgagttacctcacataatttagaatcaatttctatcgccgttacaaaattacatctctttaccaatccagcagtaaaatgacctttccctgcacctatttcaaagatgttatctttttcatctaaacttatgcaattcattattttttctatgtgatattttgaagtaataaaattttgactatcttttatatttactttgttcattataacctctccttaatttattgcatctcttttcgaatatttatgttttttgagaaaagaacgtactcatggttcatcccgatatgcgtatcggtctgtatatcagcaactttctatgtgtttcaactacaatagtcatctattctcatctttctgagtccaccccctgcaaagcccctctttacgacataaaaattcggtcggaaaaggtatgcaaaagatgtttctctctttaagagaaactcttcgggatgcaaaaatatgaaaataactccaattcaccaaattatatagcgacttttttacaaaatgctaaaatttgttgatttccgtcaagcaattgttgagcaaaaatgtcttttacgataaaatgatacctcaatatcaactgtttagcaaaacgatatttctcttaaagagagaaacacctttttgttcaccaatccccgacttttaatcccgcggccatgattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggatctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcataacgcgt

SEQ ID NO: 2 - DNA, 164 bp, Artificial Sequence
  Description of Artificial Sequence Synthetic polynucleotide
gataaaacgaaaggctcagtcgaaagactgggcctttcgttttacaattgggctaccttttttttgttttgtttgcaatggttaatctattgttaaaatttaaagtttcacttgaactttcaaataatgttcttatatttgcagtgtcgaaagaaacaaagtag

SEQ ID NO: 3 - DNA, 164 bp, Artificial Sequence
  Description of Artificial Sequence Synthetic polynucleotide
gataaaacgaaaggctcagtcgaaagactgggcctttcgttttacaattgggctaccttttttttgttttgtttgcaatggttaatctattgttgaaatttaaagtttcacttgaactttcaaataatgttcttatatttgcagtgtcgaaagaaacaaagtag

SEQ ID NO: 4 - DNA, 63 bp, Artificial Sequence
  Description of Artificial Sequence Synthetic oligonucleotide
  modified_base (6)..(12): a, c, t, g, unknown or other
  misc_feature (6)..(12): This region  may encompass 4-7 nucleotides
  modified_base (18)..(55): a, c, t, g, unknown or other
  misc_feature (18)..(55): This region may encompass 34-38 nucleotides
  modified_base (58)..(59): a, c, t, g, unknown or other
  misc_feature (58)..(59): n is a, c, g, or t
gttaannnnnnngttaannnnnnnnnnnnnnnnnnnnnnnnnnnnnnnnnnnnnntanntttg

SEQ ID NO: 5 - DNA, 62 bp, Artificial Sequence
  Description of Artificial Sequence Synthetic oligonucleotide
  modified_base (6)..(12): a, c, t, g, unknown or other
  misc_feature (6)..(12): n is a, c, g, or t
  modified_base (18)..(54): a, c, t, g, unknown or other
  misc_feature (18)..(54): n is a, c, g, or t
  modified_base (57)..(58): a, c, t, g, unknown or other
  misc_feature (57)..(58): n is a, c, g, or t
gttaannnnnnngttaannnnnnnnnnnnnnnnnnnnnnnnnnnnnnnnnnnnntanntttg

SEQ ID NO: 6 - DNA, 78665 bp, Bacteroides ovatus
taaggattgattcgctagctcagcaggtagagcacaacacttttaatgttggggtcctgggttcgagccccaggcggatcactgaaacaaaaagcaaaacaatgaaaaccgctgataatcaatcattatcagcggtttttctttttatccatactgcaaattgaagcagaataccgcattttactggaggtgaaataggtggacttaatttccacataaaaacaagtccacctgattggattatatttcactgattctctgcgttttgcataaaacaaactcttttcaaaacatgtatttttacaccatcaaaaaaagaagagtatggcaatgcaaagaaactattttacggtattgtttttcctgaagaaatcaaagctgcttaaaaatggagaagcaccaatctgtatgcgtatcacaataaacggaaaacgtgcagaggtacaaatcaagcgaagtatagatgttacaaaatggaatacgcaaaaagaatgcgcgattggcagggaaaagaagtatcaagaaataaaccactatcttgatacgataagaactaaaatccttcaaattcaccgtgaacttgagcaggacggtaaacctattacagcagatattataaaaaatatctattatggagaacactctactcccaaaatgctgcttgaagtattccaggaacacaattcggaatatcgggaattaatgaacaaggaatatgccgaaggtactgtacttcgatacgaacgtacagcaagatatttgaaggagtttatcagtgaacaatataaactggctgatattccattaaaatcaatcaactatgaatttataaccaaattcgaacatttcattaaaatacagaaaaactgtgcgcaaaatgcgacagtgaaatatctgaaaaatttaaagaaaatcatcaaaactgcattgataaagaagtggataactgatgatccgtttgcagaaatacacttcaaacagaccaagtgtaaccgtgaattcttaaacgaaatggaacttcgcaaaatcatcaataaagattttgatattcaacgattacaaaccgtaagggacatattcatcttctgttgtttcaccggtttggctttcacagacgtaaagaatctgaaaaaggaacaccttgtacaggctgataatggtgaatggtggataagaaaagcaagggaaaagaccgataatatgtgcgacattccattgttggatataccaagacttattttagagaaatatcagtcaaatccaatctgcaatgaaaaaggattattacttcctgttcccagcaaccaacgaatgaacagttatttgaaagaaatagctgatgtatgtggtattcagaagaatctttccacacatattgcaagacatacatttgcatcactggctattgcaaataaggtttccttggaatccattgccaaaatgttaggacacacggacattcgtacaactcgtatttatgccaaaataatgaattctaccattgccaatgaaatgaaagtactgcaaaacaagttcgcaatataattttcaaccattatttcatttcttacagcaaatatcgcactttgccactgactgtgcaaggcggccctgtcgggctggttggcggaaaaaaatcatcctcgcttcgctccggtatttttttccgccaagccttgcaccggtcattggcaaagaacagccgggccagtaagaaattgaaatactggctccacggagccggtcatgtctaatttaaataaaagaatatgactgaagaagttggaaagaaggtatgtgaaggtacagtagcagacctcatgaaggacaagaccggaaaacagacggttgtcacgttgacaagaaagaatgcttaccgagtgaagaaaatcagagaacaagggacggatgacgaagctgtcctttttcatttccgtgaacgctgtacgggaatgggctcctatgtacacacaatcgaagcggcagacggagaaacagaacttcatccgtctgaatttgaaaaatgggaagctgtggaattcctgtatcccggctatctggaagacctgcttgatgctgcatacaacgcatacagatggagttccttcgaacctgaagcaagggcggaaacagacatcatgcaatatgaaaaacaacttgtagaggatctgaaacagattccggaagaaaaacagaacgagtataccagtgcataccatagcaagttctctgccttgctgggctgtctctcacgatgtgccagtccgatggtgacagggcctgccaaattcaactgccagcgcaacaacaaagccttggatgcataccagaacagatttgatgaatttcatgattggcgtaaccgcttcaaggctgccatggaaaggatgaaagaggctgccaaaccggaagaacagaagcaagaggaggcatggaaccgcctgaagcgtgacattgcaagcagcgcacagaccattcatgatattgataccggtaaagcaagaggatacagccgtgccttgtttgtcagcagtatccttaataaagtaagcacctatgcaggaaaaggagaagtggaaatcgtacagaaagcggtggacttcattacagacttcaatgcacaatgcaaaaaaccggttatcactccgcggaaccgtttcttccaactgccggaaatggcacgccaggccagactgaaacttcaggaaatcagagaacgggaaaaccgtgaactgaaatttgaaggcggaacgctggtatggaactatgaggcagaccgcctgcaaatccagtttgacaatattccggatgaccagaggcgcaaggaactgaaatcatacggtttcaaatggtcgccgagataccaggcatggcaacggcaacttacacagaatgccgtatatgcagtcaaaagagtgttgaaccttcaaaacctataagacatgaaagaccgattgaaatatgtaatcgattcccgctacttcgacggaacatgcctgacaagtatgagtgacggattccataatgactatggtggggaaacaatcgaagaactgcgcatacgggaaaacaatccctatctgaaagcagtaacaccttctgatatagacaagaagctgcggctatacaatcagtccctgtccgaaccgttcaaggaaatcactgaagaagaatactatgacctgctggatgtactgccacccttgcgcatgagacaaaactcgttctttgtaggagaaccgtattacggaaatatgtactctttctgctttactcgtcaaggaagatatttcaagggcctacgctccgtacttactccgcaatccgaactggacagtcagatagaccgtcacatggaaatcatcaaccggaaagccgtgatctcaaaagaggaaacaagtaaaacggtcacaaccggaaccagactcattccctattatttttcactggacggaaaacagcccgtattcatctgcaaccttgtcatccaatcagattccagtcaagcaaggacggacatggcgaataccctgaaaagtcttcgccggaaccattatcagttctataaaggaaaagggcattacgaaactccggacgaactgatagaccatgtatcaggaaagaagctcacccttgtttccgacggacatttctttcaatatcctcccggcagggaatccgcaactttcatcggacacatcaaggagacatcagaggaatttcttttccggatctatgaccgtgaatatttcctgtatcttcttaaaagactgaggaccgtgaaaaaggaatcggcacaggaacaaataaatatcaaatcataacattcgggggaatgcggtaaaatgactgccgtattccctcataaaaacaatacaagtatgaacaaatcaaacactctatactggaaaacagccacagatccggctgaacgcattgaggtcagactcgtcctgaacagttatatcgacaatgacaatctgtatgtaggacttgaatcccggtctaaggagaatccggaatgctgggaatcctacacggacatcaccgtcaacctcaattctcttcccccgttccatgcctatgtggacaaccgggactgcaacagacatgtgcatgattttctgaccagtaacagaatagcagaacctgccggatttgaatatcagggattcagaatgttccgcttcaatcctgacaggttgaaggaactcgcacccgaacagttcaagacaatcagcgccaaactgccaccacaggatgacatgataaaggacatcatctatcaggaaagacgtttccctttgagaactgttcaagacattcacggaatatatcttgtttcaagcaaggaactggaagaatctctgatcgaaggagtacggaacctggatgctgcggcatatgaactgctggatggcatctgcctgttctgctccacacaggaactgcgctatcttacggatgcagaactgatagaaacaatctacgcacaataaaaaggaggaacaaatatgaaaaccggagacattgtatttctgagacgtccctataagggataccgtgccgtcgaactgatggaaagactggaatgccgctggctggtcaggattgtcgagagcggtcttgaactggaggtatatgaagatgaacttatatcagaattttaatacagacaaagtgttatggaaaaatatcagtttgcattccattcggaaataatcggctatacctctcctcatatcggtgaggtcagaaaagccatacacagaaaagtggaaaaggaaaagtctgccgccataaagaatgatattgagctgcacatgtacaaagtgcatgacggcataccggttctccttaacacctgctacctgtacgatgaaaaaggatgtatggtacacggaagtatcaagggaaccaaggattatctgcttgagacatggagataccatacaaacagacattctaaaggcatcagttccacaagaatcaggccttgcacgacaagcagggctttttcatttgtataactcttaaaatcagaaatcatgaaccagacattacaacttacagactatattccacagaatgtaagcctctactacgtggactaccgggatgatcttgatgagcatgaagacatccaggaggaatgcatccgttccaacaaaatggaaaaactctatgaaaaggcatacgaatggtatgaggaacaggaaagttcaaacatgcacgactatctggaggagacaagaaagaatatggaaacggacaatttagccggagagtttgaagagcatgaagatgaaatcagggaacttatctacgaccggaacgattccgacccggtaaaggatatgatacgcaactcgtccgtcactaatttcttctattcgctcggagtggaaatcagcggatatctgaccggttgttcactgcggggagaatcagtcgccatggcctgccataaggtacgtcgcgcactgcatctgaaaaaggggcagtttgacgagaagattgaagaactggtagagaatgccacatacggtggagaactgcgcatctacttcaacgccatgtttgacaggctcatcagcaaaggccctgagaacgatttcaagagcatccgtttccacgggaatgtagtggtggtcattgccgacagccggaacggttccggacatcatgtacggattccgctggacatcactttccctttccgaagggagaacctgtttgtcgattcacaggtacactattcctatgccaatgaagtctgcggcatgaccaatgactggtgtgattccacaaaatgggaaacaggcatgataccttttaccggatctgtccgaaaaagccggatggctgaatacaagaaacaggaagccgcttatgagcagacattccgagacgggaaatgcaccttcggtgacatgaactacaaacgccaccgtgacgtgcggtattcgaatgaatatcctgccggatgcaggtgccctcattgcggtacattctggattgactgaaaaaacatttaccaaccaataaattcaaacgatatgaaaatctgctgttcacaagagcattacgacaaggtcgtacagtatgcaaaatcaatcaatgacaagacactggaaaactgtcttgaacgtctaaaacaatgggagaagaacgagaaccgtccatgcgaaatcgaactctattacgatcatgcgccgtattcgttcggattctgcgaacgttatccggacggaaatacaggcattgtcggaggactgctgtatcatggaaatccggacgaatcctttgccgtcaccatggaacgtttccacggatggagcatacatacctgacatatatgcgacagtctgtattggggagcctcatgcaatatggggttccctttttttatgccgcagacatgatgacagcatcctcatttcttgctgcaaaaatagctgtttgccgcgcaactcccgcaaggcggccctgccgggctggttgtctggaaaaaaatcatcctcgcttcgctccggtatttttttccgccaagccttgcagggatgcgggcaaacagacaacagggacaacaagaaataagaatgcctgtaccttacaggcagacaatgtataacaataaatatcagaagtcatgattacagaccagaagacacagaacaggcttcacgcggataccggaacggaactgttctccatcagacaaaggaaggaagccgtcacaaggatgctggacattctgaaagagactccggaatacctgcaggttatgaaccatataccggcttatgccatggatgacgatacgtcagaatggtggaaatcggaagaatcggaaaatttcatgaactcactcctggaagtgatggaaagctatactccggacggatacaggttcggaccgaaatccggcacgactgacctttacggctactgggaaagcaagaccgggcggacaaccctcttccatctgcttttcagtctggaaagcggatatgaatggggaaaaggtctttcccatgagaaaacggacgcattctacaaggaaataaaagagaaatttcatggagaaggattcgacacggacagaaccggctgtacatcacaggccatgtatcttgtaaaaggaaaaacacgcctgtacgtgcatccgatggaaataagcggctactgtgaaacactgcatattccacagattacagccatactgaaaaaaggaggccgtacattccgtcttgtaaaggatacgatagcggaagaggtgtattccttcaccgatgaagaagaactggaatattaccgtgccagatacggaacgtgcatccaccggaatatactggatgccttcagcaaccgccacgcagggaaagaggacatactttccatgatggcatcacggataaatgtggctacgacatcacatctttacggtatcggatatgattcgcctgcatacaggtttgtgcatgaggcatacgacagactggtaaacaatggaaagctgaaggagaatgtccgggaaatcggttgctgcaacatcataatggccatttcaaataccaacgcaatatgagactgaattacaatgacatgctgcttctggcaatatgggaatacaacaggagacaggacgaggatctgaccctggaactgtttcaggaaacattcggacaggttcccggcgcacatttccatgacaaatgggtgcattattacaacaagaacctgctgatgatggccgcctatttcaggggtgaggaagaaaacggccagaaattctgtgatatgatcacccgacaggttgaacgctatacacaaaacaggaggagaacaggatgaatacaaagatacgatatgaccttgacagtcttgaactggcaaacggtgacttcgggtatcccattacagaaaaggaagtacggaaagtgaaccgtatgctggaactgatggagaatgtccgaagcaggcagatgtgcccgacagaaggagactgcgtggaatttgtctcacgttctggtgactatttcggaaaagctcatatagaacggataacaggaaaatatgcggatatatgcctgataccggaaacggtattctgttttgatgacatgggaaaagccgcctatgataccaccggaagtccctggacgcaggtcaatatccggaacatgaaacccgcaggttctgaaatccgcatattcagaacatggggattcgggaagcgcagcaatacgggcagtctcaggttcgatgctccggtcaggaaatgggaatacagagaaccgaatccgttatatgacggttacaccacccgtaactggttccgctatcatatcatgaaacaccgggacagggaaaggacaggcgaatacaccttccgcagcgattcattcacgctgtacagccggagcgagctggacgagctggccgcaatcctgaaaggcagactctacaagggaatcctgcctgactctcttgtactttggggataccgcatggatattaaggaaatatcacgtgaacagtggaacggtatgggacagcacggacaaatccgcatgaaattcatgggatacggtccggtcagaatccacacggacaatgaaaaccataccgtaacagtatacagaatcaacgacatattgtcttcaactatcagaattttcatattttttcagttctttttttgtttcttctattaatattttaagccactccatgatttgtattgcatgttcatgaacagtttcattttggctatcactgtcgtgtagtagcctttgaaaatcacgtaaaatattgtctttcccaagcatctcccatacaggcatcatccggtggattatttttctcatggtctcacggtcggttatcctgtcagcagattccatctcctccagttctttttcagattccataacaacgagagaaagcatatgactataatcatccgtattctccagtaaactggaaaaatcgaattctccggaaactgaaacttgtgtacgagatataatggtggataaaaaagcaagcagtccgtggatattgaacggtttatgaatacagcctacaaatccttctttttcataaattccggaatttccgtcaccacgggcagtcatgactgctactggaacagttctagaattgccgatgtccgaattgcgaagcaatcttaacaaaccgaatccgtcagtatcaggcatttgtacatctgtcaagatcaaatcatattcagaattttcaagagcggccactacttcacgtgcattcttacaggttttacaggatatacctttgcgcccgagcatatcttccgctattttcagttgtataggatcatcgtccactacaagaacattcttaggcaatatagttattgtattatggtccgatttgtcttcctcaactaactcatccgtttcaggcaaagaaagttccagtctgaacatgcttcctttaccgagtacactttctacatccatttttccttccaaaaccttaattaatcctttggtaaggaaaagtcccaaaccaaacccttcagaattgacattctgtgcggcacgctcaaatggagcaaatattcttttcagtgtttcctcatccataccgataccagtatcccttatttcaatacgaagttttccttctgaatattctgaatggaaattgacgttacccctggaagtaaacttaatagcgtttgtaagtagattggctaaaacctgttcaagtttgtccgcatcaccttttactattacatttgatcctttatgttcagaatataaaatcagaccttttgaagtcgctttacgagaaaactcatctgaaattcgttgcaagaaacggtcaagataaaatggtgtgtcgttacgcaaattaccggcttcattgattcggtaagcatccatcaaatcattaaccagatgtaaaacgtgtcgacaagaatgacggatgtcatctaaatatttttcgcgcttcctcttttcacgcgtttcagataccaaatctgcacagttatggatattaccaagtggacctctaatatcatgagaaactgtcaggatgattttcttacgcatatcaagcaaattctcgttttcttgaatagcttgttgtaatttaaatttaattatttcttccttacgtaaatctgattgtataattaaaaatgaaattaatattataaaaaccgcaatactcatcattacgataaataatcgaaaggattcttgtttgacttccgttacctctaagtttcgttctataaatgacagctgtacctgattatctaaaaaagatacaaaatcatataatttttgatttaacagcctattctgcaaacgcaagctatccacataagtttctatctgattgtttcgcatatctatgacagaaaccaatctattattaaaattctgtatttcattagttatataggggacttgtatcgtctccttctttccgaataatccggcaattcctttctttttctgagttattgtcttcacttttactgtttgagtagctattacaggcaattcattagtaagaatactatcagatttattcgcaaattggactgctttcattatttgaaacaagtgcatttctttcgttttaagcaattcccgtaaagaatcaatttgaactggacataaaaaatcacaactccttaattttatttcaagtagaacactatctgttttaaaacgttgattatgaaatatgttataatcagactcatcccatactataactgattcgcctaaagttgccaacttagtaatatacaaatgaactttattagtattctcataagcttcattaatttgaattatcagattctcaagttctttcaaccggcaacgttcatttatcattacagtaaccatacttaagactataaatcctgtaataaaatatccaataaatagtcttttgcgtaataatgaagtcatcaggaacattctattgatttatttgacatcataattctatatatttaactagtcatagtatatatcattctcaaatatttatttcaaattcaagcaataaaataaaaaaacacttcatattacaactgaactcttttatgaaaaagttgaatatatgaagtgtttttttattacgatataaactataaaatcctattcttcgggaactggtgtataaacccttatccagtccaccaggaaggtgtggtcttccacatttttcagttcctcatccgtagggcttaaacctttaacggctctccagctttggtcttccatatttattatgatgtccatgtcttttaccagacctgtaccaccagtgtagttgttggggtcgataatatccttgccgcttacggttctgacaagttctccatctacataatattcaagtgtgaaagggtctttccagaacactcctacacgatgaaaatcgtcgcgccacaatgttcccttgtcatccttataccatgagccaagatctttcggctgataatccttgaatggctggcggatgaatatgtgatggctcaggtgaagtctgtcggcaccgtaacctccgccgtctctgtcgccgccgtatgcttctatgatgtcgatttcctgagtatcgtcagggctgagcatccatacatcggatgccatggttgaatttgaaagttttgcgtatgcctctacataaaccggatactttacacgtgtcttcgatgtgatacatcccgtataggttcccggcagttcctttgtgttgggtccgcttacaactttcttcatggggacatcttcaggacggctggctcttattttaaggtatccgtcggaaacggaaacatggtctctctgccatattgtaggagcaggtcctgtccaatgattatgatagaaatcggtccatttggcatagaactcttttcctttatccttttcgtcggcaacataattaaagtcgtccgactgtggatggagtttccacaccataccgtcgccggcatcagcgggtacaggatagatatcccactcgtacgatttattattgaaatcttctgctgcacaggctatttgcagcgatgctaaacaaatggtaaacagttttctcatcgtggtatcttagtttaagttataataattattttcgttcttttgattcacctttagcggtatgtgtctgcaatgtccaggtagaaaatctcattatgctctgatagtctgaactgttgtatatatgagtaagaccccatctcaatatttcggtaggttctttttcggcatctgcactgcggttcaggccaatggcgtgtggcgcgccttttactactgacattatttcaaagtttattccgtcaggcgaccactggagtgtgttcttttcaggaccgtcggtggtgataagtgaagctatacctcctttgtaaggccatacgcaaacttcatgcccgctgtttgaaataggattatattccgatttcacatacggacccataggattttccgcaatagccactccgtgtttgatttcacggccgccccatgttatttcttctcccatacgttcgcctttgtagtacatatagaacttacctttataaggtattatacacgggtcgtgtaccttatgactgtcgaaatcacctttcgacactaccttgaatctgttatcctcatcgccttcccattcgccggtattagaaggttccagtacaggcttgtctgtcttgatccacggtccttcaggggaatcagcacatgccataccgatagtattctttacacggactgtgtaaggggattttaccgcctgatagcaaagataatactttcctttccattccatcacctcaggagtgaagactgaacggtcgtcgtaagcacctttttcaccacgtttcactgcaattccctgttccttccatgtccatccgtcttttgatgtggcataccatatatcacatctgtcccatgggaaaaccttatctttctctatatctccagcaaatccttgggtaggtccatagctctttgaataccatacataatatgtattacctattttcagcattgcactcgggtctcttcttactacgccctcttcataagcaagatcacctttaagtggttccatcttatactcaaagaaccatttattgtcgtgattttcccatttcatggcacgtttcatagctgcacttaacttatttcccttaggtattcccaatgaatcggccttacgctcatcataattctgagtgtcgtcaacggcaatagtctgtgtattgcctgtatttccgcatgctgccaatagcgacatcatgccggctgcaagaataatttttctcatactagactttattttatattaattgttagtttattcgagtgtaattcacttgtttctgcactgatattcagtaccgatgatttttctgtcgactgaagcatcagcatacatcttccctgatatgtcataatatccttactttgatatggagaaacgttcttcacgtttccattgtctataccaagcagacggtactctccatcaatgttgaacttaagcatctgttctgttgtctttacaggattaccttttttgtctgttagctgagctgtgacatgcagaacatcctttccatttgctgcgatactttgtttgtcaaccgtcagcaatatcgaatgttctttgcctgaagtccttatagctgtagtggtattacctaacttattttttccttttgcggtaatagtgccaggcttgtactgaactgcccatttatagatatgatcctcaaaatcgtctatatacttctttcccatcgacttaccgttaacgaaaagttccacttcatcacaattggaatatatctctactattaccgagtcacctttctgataattccagtgagagtttacatcatcccaaacccataattttctatcccattcatgtcctttcttatcagtaaatccatcttttacatggagatacgaagatttgtctgtagtctgtgaatatatagcaataaaaggcttgtctgtccacaatgatttcatcatgtcgtacgaaggcttcacatagccgcacatatccaggagaccacatcctatcgacttttgaggccattttgaaagacggctttcactttctcccagataatcgactcctgtccatataaacatacccggaacgaaatccctttcaatcaccgccttccattcgtgccactgaccgagattttctgtacccattataggcttgtcaggataattcttcttagcataatcatacatcacgcgacggtagctgaagcctgccacatcgagcgcgtcgatatatcctgactcaaagcttatggaaggcaggatgcagttggcggtaactacacgtgtggtgtccatctggcgtgtccatgcagctaatttttgcgctgtacggccaatgtcgtatgcatgtttaggctggattttccacatttctctgattttttctttagagtatggaggctgattccagaaataattaccgttggaatcggcaccgaagaaacctgtcgcctcgcggcatccggtataagtccattctatttcattacctatactccactggaagatacaggcatgattacggcttctcctcattacgtttttcaaatctctttctgcccattcctggaaatgctcgcaatagccatgcgtaggatagtcttctacagtttccttcatattgagtcttttatctttgggataatcccactcatcgaagaattcttcctgaaccagaagacctatctcatcgcacaaagacagaaactcttccgctcccggattgtgcgagaggcggatggcattgcatcctccttcctttagggttttcagacgccggtaccacacatcgcgtatcattgccgcgccaaccattccggcatcatggtgcaggcatactccttttatcttcatgtttttcccgttaaggaagaaacctttgtctgcatcaaaacggaatgtccgtatgccgaacctgacagtgttttcagaaattacttcatcgccattcttgatgcgtgtctcggctgtatagaggacaggtgtatcgacgctccacaaatcaggctgtttaatctcagatacgatgtcgataattttctcctcaccagcattcagttttatactgaagacctcaaaggctgcgatattgcctttattatccttatatactacctcaacaactgcagctctgggttcggagtagctgttgcacacggtaacctggttgtttactttagcatatttatcagtaaccacgggagtagtgacaaatgttccccaaaccggaatatgcagtctgtcggttacaatcattttcacatccctgtatatacctgaaccggtgtaccatctgctgtcggcataatggctgtggtcgacccttacagtcatacggttatcctcattgggattgagatagtctgtgacatcaaaataaaaaggagcatatcccgaaggatgatatccaagctttttgccatttatccaatactcagaattattatatactccatcgaacactatatagcatttctgatttgcactgattgttgtgggaaatgatttgctataccatcctattcctccctgaaggaaagctacacatccttcacccgaaatggaatcgtaaggtaaaccaacactccagtcatgtggcaggttcactttcttccattcatcaccagggacataagaagtatatgaataatgagcagaatctttcagtacgaatttccaatctttattgaaatcaacatttgaatcagatgctgaaacctttagggttgataataggattattaaagctaaaagatttttatttctcataatcttaggttttacatgttttttgatgtcacaaaactatatctttcacttataatatatgagggggatattaatgtgatatagggtgggaaatcagaattttacatctgccctgtattccaccgtcacctacaaccttgacaaaggatgttcctttcttccctcttatggttctcaggacaaacagacactttccgttatatgtccttacactattgtttatgacgttgatgttcaaatcttctatcgaaggcgatccattgtcgagtccggcaagttcaagcttgtcgtcgaggattatcctcacatccgaaggtatatcgactactgtgtttccttctttatcttcaatggatacttctacatggataaggtcataaccgttgtcggtagctgttttgcggtcgcagttcagtgccagacggcacggcttgccgcttgtggacaaagtgtctttcgacaatattctgtcgccgtccttgcctaccgcaaggagtgttccttccttgtatgccaccttccacatcagtatattatgctccatgaaatcgctgcgtttctttgttcccaacgatttgccgttcagaaacagttccacttctggggcgttggtatatacctgcaccagtatgtcctcgtccctgcggtacttccatttatcgcgtgtgtcgtaccactcccagcgtctgatccatcccgggcgtggagtgtaggtgaaacttccgtcagtatccatcttgaactcgctttccttttcaggtattgttacaatatgggttttcggtgtgtctttccacagacattcaaagaaatggccacgcgctgtcttgttgcccacgaaatcgaagaaagaacagtctccaccccttgcaggccatgggccgttctcgccaagatagtcgaatcctgtccacacgaagatgcccgctatgtacttcttgtcggccacggctgtccattcaaagagctgaccaacattctccgaaccgataataggctgatatggatatagcttatggtcgatttcataatatttgtctttatagttatatcccactacatcaagaacgtctgtatatccggagagacgcgaaactgacggaacaacgactcctgaagagacgggacgggtagtgtccacatccttaacccaaccggcaaggacagcggctgtttcagccaaatcgtcttttcctcctgacagacggttgaactctttcagtatagacttgttgtctgtttccgggtcgcccgtatggataagacccttgaaccctttattgtctttgctcgatgcccagtaatatggataggtccattctatttcattgcctatactccagagtatcacgcaaggatgatttctgtctcgcctgatgaacgacttgaggtcgtgctcggcatgcgtatcgaagtatctggtatatcctattgatatgctgtcgggcgcatcttccttagctcgctcagtaatccactttttctttgccaccttccattcgtcgataaattcattcattacaagaagtcccagactgtcgcacatttccagcagactttccgaatgcggattatgggctgtacgtatggcattgcagcctatggaacgaagtttcagaaggcgtcgcaacagggcatcatcgtatgcggcaacacccatacatcccaagtcgtggtgtatgttcactccttttatttttactgattttccgtttagaaggaagccttcatccgcatcgaatttaatgtcgcggataccaaattttgttgttttcttatccatcacatatccgtcagaagcaatcagagtagtatgaagctcatacatcgaaggcgtttcaagactccagagatgacaattctccagttcaacagatgcagtgaactcattgaaatcgcctttcagggcaacaaaatcatcggaaacagaagctattgtcttgccgtcgtacactacttcgtgcttcacggtgactccttttacacctgttccagcattcttcacctcgcataccacattcaccatcgaacggttgcctacctgtggtgtggtaacgaatattccgtctgaaggaatatagagctcgtttcttagaataagactcacattcctgtatataccggcaccgacataccatctgctatcggcatacgctcttctgtcaacgcagacagttattgtattcatcgaaccttttggtttcagatattgagtaagttcatattcaaatcccacatatccgttaggacggaatcccaacatatgcccgtttatccaaacctttgagttattatatacaccttcgaaatgaatgaacacttttttcccattcatatcatccgaggtgagaaaattcttcatgtaaatccccacaccgccagacagaaaaccattgcttccggctgtctgagtcttggtatatccttcgctgatactccagtcatgaggcagacacacatcctcccactttatatctggactcaggaacaaagtgtcctgaggcacgaaacctgctggtttgctgaatttccaatcgaagttgaaatccactttagtggaggttccggcataacagaatccggacagaaagatagttaagactgtgataatgttttttatggtcatatcgattttcagattaatattaatgacaaaaataatttcaaaagtgtaaaaacaaaaaaactctccatttatatttcagatatcaacggagagtttcatcattaaaaaaaataaaacattttataaagttactccttgcttaaggatagctatttcccggtatcccttcttttcgttcagtgcctgctttccgcttgccacttccaccacaaagtctataaaacgtctgcttaaagattccatgctttctccctctaccagagttccggcattgaaatcaatccacgtatgtttctgttcataaagcggagtgttggtcgaaaccttcacggttggaacgaatgttccgaacggtgttccgcggcctgttgtgaacagcacgatatggcatccggcagaagcaagagccgtacttgccactaggtcgttgcctggtgcgctcaacaggttaagtccgtgtgttgtgacacggtcgccatatttcagaacatcctccaccatcgagcttcccgacttctgtgtacatcccaatgatttctcctcaagcgtggaaatacctcccgccttgtttcccggtgaaggattttcatatattggctggtcgttgcggatgaagtagttcttgaagtcgtttatcatggccactgtgtcgtcgaatatctccttcgtgcggcaacggttcatgagcagtgtctcggctccgaacatttcaggtacctccgtgaggactgttgtcccaccctgggcaacaagatagtcagagaacaccccaagcatcggattggccgtgataccggacagtccatcagacccgccgcacttgagtcctatacgcagttttgacagggggacatcagtccgcttgtcttccctggctatggcatacatctcacggagaagtttcataccctcttctatctcatcatctactttctgagaaacaaggaaacggatcctttgggtatcatagtcacctataaactcacgaaaggcatcaggctggttgttctcacagccaagacctacgacaaggacagctccggcattgggatgaaggaccatgtcacgcaatatcttacgggtgttctcatggtcgtcacccaactgcgagcatccgtagttatgagggaaagatataatggagtcaaccccctcgcaacctgtttccttgcgaagctgctcggccaactggtttactattccgttcacgcaacccaccgtagggataatccatatctcattacgtatgccggcttctccgttagcacgcaaataccctttgaatgtatggttctcgttcgtgaatgtctgtttctcgaacttcggagtgtaagtgtatgtactcagaccggaaaggttcgtcttgacggttttctcgttcagcagatgtcctttcctgacttcctttacagcgtgcgatatggggaaaccgtattttatcaccatatcaccttctgcaaaatccttcagggcaatcttatgaccggcaggtatatcctccattaattctatggaattgccgttcacctctattacagtccctttggacaatgggtgcagtgccacagccacattgtccgcagggtttatctggatatattcagtcataacaaactaacatttataaattgaagaatacaggtagaagtatcaacctacaaggtcttttactgtctgaagcattccttcgctctggattttgttgatatagtaaattacacggtctgccagtcccgagatagtattaaggtcttcaccccaaatggaagtatcggcgagaactgtcttcacaagattttctaccgagccatcgttccacaaacttgtaagcatcgccatgatttcctgtgcatcgttaggaactatctctacaccatcggcacgctttccacctttgtagtatactatgatggctgcaagaccgagtacaagtccttcaggaagcacacccttacgtttcagatattccttcactcctggaaggtcgcgtgtggcatacttagggaatgagttaagcatgattgatgttacctgatggtctacgaaaggattattgaaacgttccaggacatcatcggcaaacttcttgagttcctctttcggcaggttgagggtctccatcagctcgtcgaacatcacacgtttgatgaacttgcctatcacctcatgttggcatgcgtctctcacgatattgacgcccgaaaggaatgccaccggcgacaatacagtgtgaggaccgttcagcagagtaaccttgcgttcatgataaggctcctccgacgggacgaacagaacgttcagtcccgccttgtttgcaggaaattcttcggcaaccgattccggtgcttcgataacccacagatgaaaagcctcgccctgtacaactaaattgtcatcaaagtatagtttagtttttatgttgtctatgtctttacgagggaaacccggtacgatacggtccaccagtgtggcatatacaccacatgcagtttcaaaccatgacttgaactcttcgccaaggttccacaattcaatatactgatagattgtttccttcagtttgtgaccgttgaggaagataagctcgcatgggaagatgatgagtcctttcgacttgtcaccgttgaaatgtttgaatctgtgataaagcaactgtgtcagcttgcccggataagagcttgcaggagcatcctcaagcttgcacgacggatcgaagttgataccggcctcagtagtgttcgagattacgaatctcatatcaggctgttccgccagtgccatgaagtcattatactggctgtatggattcagcgcgcggctgatgacatcaatcattctgaatgagttcaccacctcgccattgttcagtccctgaagattgacatgatacagacagtcctgggcattgagggcatcaaccatacctttttctataggctgcaccacaacaacactgctgttgaaatctgtcttttcattcatattcgagataatccagtcgacaaacgcacgaaggaaattaccttcgccaaactgtatgatacgttccggacgtactgcctttactgcagtcttactatttaaagctttcattgtaatgccaaaaaattaaaattgataagattaaaattcaaccaacattctgaataccttacctggattttccgaccatttctgcagagcctcgcctgcctcttcaggtttcactacggcagagataagttcgttcatcgggcagttgccattctgaagataatgtatcacggcacggaaatcctcaggcattgcattgcgcgaaccgcgtatgtcgagttccttctggacaaaatattttgtctggaaagccacttcactcttggcatagccgatacatgccacacggcctgtgaaacctacaatgtcgatggcagtaacatatgtgataggactacccacagcctctatcaccacatcagccatatagccgtcagtaagttcccttactctttccaccacattttcagtcttcgaattgataaccatcgaagcacccaggcgttttgccagttcaagcttctcatcgtcaatatccaatgctattacccttgcgccacgaagcgatgctcttactatggcgccaagtccaatcattccgcaaccaatcacggccacagtatcaatgtcagttacctgagctctcgacacggcatggaaacctacgctcataggctcaatcagcgcacattccttatccgaaagaccggcagccggaataacctttgtccaagggaggacaaggaactcctgcatagaaccgttacgctgaacacccaaagtctcgttgtgttcgcaggcattcacacgtccgttgcggcatgaagcacactttccgcagttggtatatggatttactgtcacgttcattcccttctcgaaaccgacaggaacgccttcgcctatttcctctatcacagcacccacttcatgtcccgggatgacaggcatcttcaccataggatttcttcccaggtaagtattaaggtcggaaccacagaatccgacatatttgatacgaagtaaaatttctccggctccaagtgttggtttaactatatcagctacttgaacctttccggcttcagtaatttgtacagctttcataatctatgtatttatttaaatttgttattgtattattttgatgttgcattaattcaatgttgttttttctctatcttatatcctctccagccataatatgccgtaaagaagaaacatatcagaggtattacatatgccacctgatagaagtccgcgttatgattcatcacaaatgcggtgaactgagggatgcacgcattacctataatagccatcacaaggaatgccgaaccactctttgtgtcctcgccaaggtcgcgtagtgcaagtgagaactgggttggatacattatcgacatgaagaacgacactgcaagcatggcataaagtcctgtcataccaccgaacatgataattactccacacagtatgatatttactatagcgtatgtaagcagcatatcctgaggtctgaatttcgacattagcatagtacctatccatctgccgccaaggaaagccagcatatacagtccgaagaatgtggtcgcctcatcctccgacagacctgcatacatgcagcagtaaactaggaacaggctgttgatggctgtctgccctccgttatagaagaactgtgcgataactccccatctcaggtgtttgcgtttcaacactgcaaaattgataagcttgcccttctcgccgtgcgattcctccttgtcaatatcaggcaacttatacagtgcaaacaccacagcaagaataatcagcaggactgcaagaaccagataaggcatcttcatggagtctgtctccatctgaataaatccgtcccaacctccgggaaagtcggcaggcagagtctcgcgagtatagttctgtccggtaagtataagcttactcagaaacattgcggatatgaaagcaccaagaccgttgaacgactgtgcaagattcagtcttcttgaagccgtatcgtgtgtacccagagctgtcacatacggattggcagcagtttcgaggaagcacattcccgttgccatgatgaagaagattacaagatatgcccagtattcctttatctcggctgcagggaagaaaagcagaccaccgatggctgcaagaatgagaccgacaattatacccgacttatagctgaaacgtttcatgaacattgctatcggtatgggaaacaggaagtaggccagccaataggcagcttcagtgaacgaggcctcaaaagcattcagttcacaggttttcatcaactgcctgatcattgtaggcaatagattactgctgatagcccacatgaagaacaagctgaatatcagtaaaagcggtataaaatatttgtttttcattctgacatgtttttaatataaggtaactcaggcagattcttgaaaccgtaaaaggctttcgcgttctcgcccaagaaaagttttttgcttctctcttccaattcttttgatttaatcacaaagtcgtacgacatcttgtaggtaatggctgtgattgtgcgtggatagtcggaaccccacatcagtttctcgaagccaacaaggtcggcagcttcgttgatggctctgacagcgctgcggaacggatagaactcgtcattgaacagccaagtgataccgcccgactcaatcatcacattcttatgacgggcaagcattatctgcttcttccaatccggtttagtcaccataccgaaatgcccgatggcaatcttcaagtacggacattctgaaatgatttcttccatctcgcccacctggaggtctccctctgccatatctatggaaagaatcacccccttgtcttccattagatgaaacatcctcatcatctcgtccgagttgagcatcaccctaccgtccttcagttgcaggcggtgtcccggaatctttatggccttgaaccctttgtctataagttcaaccgcctggttatagaaacccggttttctgaattcacacataccacacacgaagaacctgtccggatatttcgtcatcacctccatcagatagtcattctgaatgccgtcgatatactcctgtgtgacaacagccgcgccaatcagggcataattcatattagccaggaaaacctcagccgtgtttcttccgtcaatcataaagggggggggagcatttgtctcacctcccccataaacaatgattgaccgttctctgtagtcttgattttcaggccatctacttcagtgtcctgataaagccacagatgcgaatgggcgtcaattattgtataatccatagaaacagtatttatgaatttgcccaacttactctttgctgatcgcctattatctccttaaccttttccacaaggctccagtctatcggttcctcaatgtattttatgttctgaagcacagactctgttcttgccgagctgaacaatgttgtaggtattctcggattgcttacagagaactgcaccgcaagtttctcgatagggtatccctgttcagcacaatacttggcagcctttgcacacacctcaatcaatggttttggagccggatgccattcaggaacacctctatgtgtgagaagtcccataccgaacggcgaagcgtttatcactcccacaccattttcgtcaaaatagtcgaggaagtccaccagcttgtcgtcgttcaatgaatagtgacagaagttaagcaccgcctctactgtacccggagcggcatggtcgataatccatttcaggttttcgagctgcaggtcggtgatacccacgtggcccaccacgcctttcttcttcagttccaccagagcaggcaatgtctcgttcaccacctggttcatatccgagaactcaacgtcgtgaacgttgataaggtcgatatagtcgatgttcagacgttccatactttcgtaaacactctcctgagcgcgtttgtccgagtagtcccacgtattcacaccgtccttgccatagcgtcccacctttgtagaaaggatgaacgattctcttggcaattccttcagagccttacccaatacggtttcggctttataatgtccgtaatatggagaaacatcaataaagttcagtccgcgttccactgctgtaaaaacagactgtatagcgtcactttctttgatagaatgaaaaactccgcccaatgaagatgcgccataactcaatacaggaaccttaagtcctgtctttcccaattcacgatattccatttttgataaataatttaaaggttaatattttttactctgtttattcttattcatacagatagaacatacgttccatcatcttccatttctcgtccgatgtggccccctcggcacactgctggaatttggctacgtattcttcccattcggcctgacgcggcagagtggcaagctttgccatagctgtatcccagtcaaaatccagaggtgtttccactatcataaagagttttgaccccaatatgtatatttccatttccaggattcccacctcgcgtattccggcgcgtatctcaggccatgcctcttccttactgtgagcctttctgtaggcttcaatcaattccggattctcacgcagactcaatgtctgacagtatctcttcacaggcagggaataacttttcactttatatccttctgtcttcatgatattattgatattaatatgttagtattacatgtcactgtctttatcttttcgacgatgctaaagtatgaagtatccatcaaaacaatagaggagattttcaaaaaagaaagaggggatattataccccctctttttcgacatttttacccctcataaaggagataaaaagtcaccccaaactctataaaaaatcaaaacagattgaactgcattcctgtgtagaaaaatccctggttggatttcggattccaatacgtcatcaccgtcaacgggatttcatattccataatccgaagtttataaatcacattcagggacacctgagtaattcctgccgattcggcatacatggttctgttcaccatttccccgctttcatttcttgaatttctcaatgcgaaagctgttccaataccaggaccgacccttagcttttcgttctgatagatggtatagcccacatatacgaaactggagtagatgttcttgctgttgtccagatccctgtcgcgaccgtaaacaagtgtagagaagctcaactccagcggaaatttcctgtcgcccgtataattgaccatgagatcaacgaaacgtccagtttcatcaggcttatagttgaagaactccttattattatatgtagccccgggcgagaaattatatgtatctatagcctttatctgaaacctgccatgagtatatgctatatactggctcagctccttataactccccctggtgttcgatccgccaaggaaaccggcggtaaacctccccgatgggtcggaaaccgacaaatcggacgagagaatcagtccgtcggccacttcaatgccacgccatagaatcatgttctgtagagtagtactgaaatgaagctgagcctgaacatttgctgacaaaaatataaatacaggaattaacagtcgctttttatacttacaggtatccaatgataatatatgtatcatactcagagcagtagaaaatcggttttaaattattattatggatttatttgtcgaaatactctataagattataaacattccagttaatatccgacatgtatttggtcaatgatgtataaggtttatagttataatcgagcatacctttattgcaatcctcatcatccagatacttgaagaaaacccatcctacacaattcttggcttcgagcagtcccaaggtaaaatgctggtaagcgaatccacggttttgctggtcgcgtaccacgaaaccagctccacttgaattgtcaagcttagtatcctcacccttggtatagaattccgttaccatgaaaggagtaccgcccgcctggttcttccagccatccatgtagcctttttcaggcgaccatttactataataatttatggaaatgacatcacaatattttcccgctgccttaattatataactgttgtatttaggaaggctgtgcaggcgtgaacccagataaagcaattcaggatccttcgatgccttaaccgcattctttatggcagaataatatttttccgcacaaataccggcaaactcattgttcagttcatccgttacatcagaaacatttgcactcttgtccttatccgtcataaacttggcggctgcaatataagcaggatcctgcttgtttgaaattttcaggaatctgtcgagcagcctgtttccccatgtagagaagtctatctcattatccgagaagaatcccaacacatccgggttgtttctgaacatgccgaaagcatccgaattgagatactccttgcaccattcatcccatccatcataaaacacaagacctatcttaagattcacgttctgccccggatagctaattcccttgctattcttgaactctgcaaggaatgaaaaggaaggagcctgtgtcagaggacttgaagccgatttattataatcatttacagccttgtcgccttcttccttaccgaaagcgcagacactatgaaatcctatttcagagaattgtttctgcgactttgccacccagtcatctactgaactgtaaagcttgccgaaagctgagctgttgccatccattctgaatgaggcgataccccttacataatatggataaccttcggggtcgactatccaacttcttccatttgagtttttctcaaccctgaaccgtccagtagccttggatttttgcccttttgcgtatgagccatatttattcacgctttgcaaatactcatcctgtgtttttgtctgctgttcataaccaaccaggtatggcaatatccttgtctttgcctctataaaagccttgtcaggtttttccgcatactcgacaattatcggttgatactgcttggtgctattaggataggtttcagcaggaccgggaacaggcagttgcagttctacatcatcatcgtcattatcgcctgcattgtcgccgggagtattatagtcctccacattccccggttgtgagtaaataacctcaggcggaatatatgagaactcctcctgagggtcttcacatgacaaagcgaagaacggaacactcaagcaaatggttttagtaataatagtagaatatttcattgttgcaaatatttagtaaattaatataaatcccatgtcctgattgtatccccccatcggtggtctatcgggaactccatttctccccatgccttaacagaagtccaaggttggtcggcatcagtccagaatgggtcagaggcaggcaatcccaacggaaggaatgcaagtgtagtcatatacaggctgccattgtttgtataatgattcgaaatgccagtctgatgtccgcagaatcctatggtgaggaatccgccctcattgaagttattgcccgacttgaacatacgtttcatacacgctgtcagcgcacatctcacctgtgctttcgatactcccgccggcaactcattataccatgctataagagccagtggctgcattgttgccatacggtaaggtatagagcgtccgaaaacagggaatgttccttcaggagatatgaaacgctccagaatcatggcgaacctctgtgccctcatcaatgccctgtcatagtacttgcgatagtcgaaacgtgtcctcacgcccgattccattattgcatgtatagattcgagatacataggatggaacacataactgctataataatcgaatgcaaagtgctgtccgtctgcgtaccatccgtcgcctacataccattcctccaccttgcggaaagtagaatttatacgatatgtatcctgtccggcatcaattttggcaaggaagctttcaatggtggccgagaacagcagccagttagtgtaaggagggtcaatgcgtcggagacctttgaactcttttatgtagcgttcctttgttgtctggtccagcggtttccacagctggtcgaacgcgcgcaggaaactttccgcaatataggcagcatcaaccagtgcctgaccatgaccgttccacaacagataatccggactattagggtccaccgcatttgcataactcttcaatgcccattctttcagttgcttgcgctgctgtccttctgctgtatcatcgtcaggcaggctcaaccatggagctataccggccatgagacgtccgaaagtttccatatatgcaaccttcttgttacggttatcccagtttggacttacctcaagaatcatatttttctgcagttcccctttcgccatattgctcaacacaggagcagccatcctgtaagccatatccgtccagtattttcttgtctcgttgttgtttgcctcgagataacgcacatactcgcaagcggcaagaaggaatgcgcctaccccaaagttggcagtcgacttggcgtcaaccacctgtcccggaatagccttttcaccgattggctggacataacccaccgaccagtctttctgcagtgcagtcttggtaagatatttccatgctttccccactacaggcataaattcatccttgtcaagataaccgttgtttatcccccaaagcataccgtaagtgaagaaagcggtaccgcttgtttccggtcccggagcatgttccggatccatcatacttcttgtccagtagccctccggctgctgcagacatgcaaccgcctttgccatacgcacaaacttatcctcgaaaaaagacagatgctcataaccctccggcaggtccttcagcacctttgccagagcggcaagcacccatccgtcgcctcttgcccagaaatccttctttccgttcagactcttatgcttgggataaacatattttgcgtcgcgataatagagtccttcctcctcatcatacattattgagtccgacgtacaaagatattcatacagtttcttaagataccggtgattatgcgtaatcttatacatcttcgtcattaccggcatcaccatataaagtccgtcgctccaccaccagtaatccttacgcggtgtgctcatctggtactccatgacttcgcgtgcacgcttgattttataattctccggcatgacgttatacaagtccgcataagtctggaagcacacctgataatcgccgaacagcacataatcatcctttaccccgtatttatacttccattcagatttgttgttgcttttcgcacccatccactggttatactcagcccatgcctccgaatactttctgtattcttctttcccagtaaggaaataggcttccatattaccggtgtgatatgccgcataatcccagaaagaccttgcttcgggggcatgatttttctgccaggcatcgttcactttttcaatcatctccctaacttgctgagcctcagtttttttttgcgaaggaaaatgaaggtaaaacagctataaggatgtataacatccagtagtatctataacagttcatctttgtgatattgtttacattttctaaaacgaaatggggaagaatatatattcctccctcatttcacgaataattgtattattatatttatttgttaggagtccattctgctccgttgttgaaaccttctgttgtagagtcaaaacttgcatctgctcctgtacttggtctttctgtaatttcttcaatcttaaaagaagtgattttagcggttccagtagcatcagtaccaccagggacattagtctgtacagttaaaataacgttctcaagaaccggccacacaagtgaaccatctgctcttgaagctggagtttcagcagaagtagaactactgattgtgaatgtatttgtataggttccacttccggtatttcttccaatccagaatttatatttatcagatgctcccaatctgaatgttgttgcacagtcgttagatgcgtatgtataagtaaatttgtaagtacaaccatcacggaatgacattgatttagtaactgggaattgattatctgctggaacaatttccaattctccacttgcattaattttttcggcaactccttctgcaagatattccttaactgcatctatgttagcgaagttaaaatcaaaagcatctgcatgagtcaaagcaacattggcagattcaatcttgatattagcatcttcgttgttttcgtttttagcagtcaaagcactaacagcataatcagtgttataacttacgctaatatttgcgtcattactataaatcttatcaccaagaataagagtcatagtagttccattcacagaaccggaagcaacaggaattgtttttcctgctactgttatggtaaatgctttgttaacagcatcagtgaatgttccagaaacttccttatcgagtgtaagttcaattcggtcattacctgttgtctgatcaggaacaatttctttagctgaagaaacggcaacagtagtttgtttttccaaatccacaggaggttcaccgcctccttgatcatcattcaatactatcgttacaatctgtcctttagtaactataaggttttcaccactgaagttataagttttagtaccagaatttcttgtaagttctaaagtaaatccatcggtaaatgtcaccggagctacaaccattgagtattccttggcatttttattttgttcattaggaccaacaaatgttccctctttagcggttagagttataacattagaaccggattccactgtcaggtttgctgaagcatcaatttttacgttccctgcaatctttacatcaccaccagcagtaagtttaatacctgtaaggtcagtaagattatttttaaacttaaccaatccacaagtattctggaaagttaaagatttgttattatctgttgcagtagcataagatatatttgcatttgcatcgaatccccaagccggagctgtctgttcagatggcagtgtagtagttacgacaccttcaagacacacagcttcggcattataaggataaagagctgtatatgaattgttaggtgtagccttacctgtaaacgttgtaactgtgctaccacctgtagcggtagtaaacttgttattttcttggcctgaaaagatattgattgcatctcctgttgtccaccacaccgttgttccattctgcaacgaactacggcttgaaggcgtaccggcaacaaaagtcatatcctgaggaccactgactgcatttacattcgacagttcgtcttttgtacaagactggagcattgcaatactcatcaaagccgctccacaaaatagcatcgtatttttcatgacataaattatttgttaaacagtttcaataataaaaaatcacatcacttgttattcatattcttattctttaggatcaggtttccattcagtaccgtcatcttcaaaatcatcatgaccgccatctacaattccgggaggtattgatattcggcataccgcactttttattccattacccgtatctacagaagcaccgatattagaatctctgcccccgtcgattgccacgaccgtacatctcattttatcgtccgatggtgtaatcatcaacacatcagggaaagaagttccccaaactattgacttgtaaccggtataagggagattatccttggttatattaatacccaactccacagtgccactatatggtaattctatataactgacaggtttgttgtcagtctgcccatccttgaacactacatattcaatttttatctcctcagctggtgttccatcacctccacctacgccatcatcatccttatcacacgagattgccgtaaactgtataaaaagaagtatgaaaaggttgtatactgacagaatccgtggttttatatcaaccataataaaatgttatttaagcgccaaacaaaattttcaatattcaaaaggcataagaggaaaccctgaatatgccttattaccatgaaaacaaatcaatctacctttttcaatccggaatcagaaaaatatgttatttatttagaacatatttttccgatttgccagattacaatcacaataaataaatcaacaactaaatctaattacctaatcttataactaaaccctcaaacaatgttatttaaccttttctatcttgacatcatcaagcaggaagcatccaccattacctgaacccggaacagctgtgaaacgatatacaaaaccattttcctgcaatttgaatttaactgttgtaagattgtaattcttacggtctttcttgacctcagcagtggcaatttcttccagtttctttgaatccggattatagtactcaatcctgaagttaggtttgtcaccccaactgtatttggtataagctgaaatctgatattctgctccagtttcatagctgatgtttacagcctgccacataccaaccttcacctcaacagcatagttgcctgaatgtgcctttttcgcatcaactattttgttatctttcttttcccagacattccatgatgtcaagtcacctgactcaaaatcaccgttcttaatttcctgagcgtatgcagaagtcatcatcattccgcaagccatcattgctaaaatttcttttttcattttttctaaggtttttaatttaagtattatgttgtatctattaaaatcactcttctattggaaccaacttataagccctgacccagtcataataagtagtacttttgtccttatccttcaagtcctcagctgtaggtacttgtttttcccaatcgtatgtttcagtaactatatgtatgaacataggtcggtcaaacggagtatctgtatattttgttgtaggcttgatagtgtacatatactttccgtcataatagaatttcacggtatttgcatccacccaccaacaaccgtaagtatggaaatcttctgccgatgggtccgtcatatacgaaaccacatccgaacgtttcgccgtattgtcagtacgtttgcctccttgttcctgataccaatagtgagtattactgttcatctgcatattccatgtcttgttccacggattatcagggttgacacttcttattatacccattgtttctataatatcaagttcctgactgctccatgtctttatcttcttgccgcctttcattatttccttcattaccgggcggttggaaagccaaaaagtagacgacatggtagtgagcgaagccttcatccttgtttcataatacccataatgtgcctggttctttgcagaagcaaccgctccaccggcaagacgatatttatcgcccggctttccatcaagtccttctgttggcgacaaaacggtattgattatacgaagacaacctttcttgacactaacattctctgccttgaaagttgcaggcggccgaccgttagtccaataaggacttttagcatgccatttagcggcattaagacgtttaccattgaattcatcagtataatcttcgttaactacccatttataaccctcaggagcctcaggcaaattttttatatgctcttcagccaaagaatattccttatcattttttaatgtataagatgacaggaataaagatgcagcagataaatacaatactgtttttctcataaactttgtcgttttagattttttgttacacgacaaaagtatataagtttcatgaaagcattaagggggatttacatcgtaaaaggtggggtaaaattctaccactccctgaaacacaattatttcactcatgaaaccatgtgtttttacgatatataaaacccgacagaagaataataccgtattaccggctaatttacataagaataacttttcaaaccgccatataccccactttacgtccgtaccctcagtcctcgactccggcaatatgttttccatatcgagatctatggttttctgcctcggattcaaccactaactgtcgagcatgtggattgcgtatctgtcatagaatctctttccgaaccatattatctcgtctgtgctaagtatgttgttcagacggataatctttccggtattttaccacctacttctcttgcaaatcctgatctgatataaccggatactctcaattcattgatttccgacttgtatacagtctgcgaagaggcattgaaactactgcacagactgaacagcagcaggggaataatttaactgattttaatagtagacattctgtgttcataatatttcattttaatgattacgtttctgactttcgtctgatgcaaaattatgaggtatcggacggggttgtatctttcagtaaaaatcagtaaagtcttggcaaggggtaaaaaacttaacatcttgtatataaatatattacaaacaaggtgcaaagattttcagtaaacgatggcgaatacagaacctatatatttacacgccataaaatgaagaaaaagcagtaggaaaaaaatgcgggcaagttccggataaaatgtgggcaagtttaaggtaaaacttgcccgcattttagatagaatgcgatcgcatttaaaacaagtaaaaaacgaagaaaaaaaatatgtgttcttcacagaacacatatttcaaaaataggtataaacacgctaaacaatgttaacaaaatctatttataaaaaaagctcacatcaataatatctgcaacatttttacaatactccataaatgaagagaccttgggatgatttatacacagagctatctgtgatgtaggcgaaaaacgtcctgtcccgtcaagaaacgctgtaagctcagatgggaggagtatactgccaatacctggatttacgtcagtcagaacgactgtatttacagcttccaccgctgacacatcaagataatcgagtgccggaagatctgcgaagtgcaattttcctatcatattgccgcctttgctgccctgaagagagacactctccaatgaagaacaaccggatatatggatttcactgtcgaatattgaagtttcggaaacatcatcattaagtataacagaaggaacaactaccaattgaagcgaactgttattctccaccctaagtactttcaatgatgatgcggaacttaaatccattcccaaaggtgtatcaatattagagattgaaaatactgaaactcccgaagacggcttgacatacgacatggaataatgcttggacttcactcccgaaatgtctacttttcctctgaaaccgggatttgacaatatatactccacaccctcaaggttagctgtctgcgacaggaaaatgaggtcgttcccttcggttatcctcttcgtgacatcaatctccaacgatgagacaaacaccgacgggaagtttctgtaaagatatgaacggagcaaaggatccggtactcttcggtttactgtatattcagtgtaatttccatcctcgtccgacatcacgacaagacatttgtccgtcatggctttatagaatgcaggtatgacatctgtattccattttgcaaagtaaggaagtttcagatttgtaagacttttacaaagcaccgtagtgccatcggttgaaataagattgagatacgaagttatgccgtcattgccgcgtaaagctaccgattttattccttcgggcaggtcagcaaagtcgaatatagaaaaactgttacactcaagattgacatctgcgagcgaaggaaaactcctcaaaccgctaatagatgtaagttcgcatctactcaagtccaaagaagtggtattgagaacttgattgtcacaaatcagctctccgttttcgctgaaattaaatcctttccgggtcaagacatcgcgtaactttgtatcaaaagtcacttcagacacttcaaagtcggaaatttctgtttcatccttacacgagattattgtgaaacagagaactatcagtacataaaagctaataaaattcctcataacaatcagttttgtggtaataagactatattatcaatccaagccgcgtcgttctgtctttcgcacacaatggcacacactacttttttcactgtagaattaaaatcgaaagatacggctttataattgccgggagaagaaaattcctctgtatataccgttcctgtagacatatcctgtagcatgactttcaacttacatgctccttcggtctttacatcagcagagaagcgataagtcctgccactctccatgtcaaccctctgcatgagtcctgcatgaccagatatacaggctacattattgcctgcattgtcagtctgtacgcaaaccgtaccatagttacccaatggctgccatgctgaaagtccttcgctgaaggttccattctgcaaggtagagacagtatatttctcaacctgcagtatcatggacgatacgtgacctcctccgtcggaaaaggtgatgtcgacattattatcgccattcttcagcagctgtatgtcgaacggtacttctatcataccgaaaaatatattgcggttgctctggccgtagcctttccagttgtcgggaacactcacagcggtaccattaatctttaccaccggtttcttggaagcagagacaggacggcctatcgacatacgcaagcttgctctgcccgaaccggactcgattcctgtgaaggggaacgaaagggatgatccggcggaaatcggtttcagatactcactgctgtaatatttattgcggattatggagttcgtgaatgctgacgaagacacatctgctacaaggactatggtctgatttgggacaattgagatgctttcaggcatggacgggacattctgttccgtatattctatacctgcgttataattgacatatagagaacgctttgtgacattcgatacatccttccagctattcttattgttcagatatacagtctgcgggttatcatcaagattatcaagggcgatatagagtctgcctccatccttgaatgcctgtacctgaatatcaggattactgctggttatatcaacacgttcgccttttacattcttccagagttcgaagaaatattttttgtcattaagcctccatgtggtattcttcagattctgaggattgtcgggaataaacagtgccgcactatatgaagtataattgtttgcagcggtgatatgccactcagccttatctgagacaaaaggtattgagataaacaaattgtcctgacgttccatcagattaaacagaaaatgattaaacgacgaaacactccgcacactgcttatgtcatcatagctgtcgtcgggcttgctgttgtcaatacctccaaactcggaaatggcaagaggcttgacatgtccgaacttaatataggaatacgcctcaaccatatcaagaactgcttcggagttacttcctgaacgtttcgtatcggtgccggttacatttattccatcataaagatgtacagagaatccatccatatatgcacctgcccgatcgatgaacattttcatgcgggtgttccagtaattgaagttcccatcctcccaggcggggtaggctgcggcatagcctatcaccttcatctttccgttaagacgcggattattgtgtatatgtttacctattgaagcataaaaatcgaccatcagttcgcgcatagcctgtccctgaacggtaaaaccggcatcatttgcatgaacgaacggttcattgaggggttcaaaaaactcaggtaccagctcgctgttggaataatactcagccgaccatgcacctgcagcctgaacgtctatgccgccctgtatgtgctgtacatagggatgctctgtggcaatatatctttttacggaaatatttccgctgtatggtttcatctgaggatatttgcctacctcatgcgtcttgttatacgcatacgagtatggtccccagaactttcttccaagaccgacctgatagtcggcaagaaacttgcctacatccttatcatcatcggaggtggaatgaatattgaaatatttagaacggtcgagttctgaaacaccgctcaaaaagcgacgggtattatagtcgacaaccacctcgttcctttcctgacaataaataccgggaggaacacctagggtaaatgccgataacagaaaaatatatttatagctcataatttctttccttttagacacagaaacttgtcagtcctgatgtggatacattattttctcactttcttatcgtagcgttcagtctgaagaatcatagtagccacacggcctccattatccgggaatgttactgacaccgaattttttccttttctgattaaccggtagtcgaaaggtatttctatcataccgaagaaatcgtctctgccggtctggtcatatcctctccaattgtcgggcatgtcgactttcttgccattaaccattatttcaggtttcttcgacatctcgtgcttcctgcctattgacatacgcagaacagctcttcctgtacccggtttcagaccatcgaaatcaaacacaattggttttccggcttccaccggctgaagataagtgttgctataatatttagtacgaactattctgtttgaatactttttacggatgatgtcggcacacaatattattgtctcatcttttataatgtcaatactttgaggcatcgagttcagcgtcttttcatcataaactatacctttatcgaaaatcatcttcaaagagcgcacagaaacattatctacacccttccaattcagtacgtttttcaagtttaccttatgtgtatagtcatcaagattgtcgacagctatgtaaagcctgtcatcgtccttaaaagctgccacctgtatgtccggattgtcggaaacaatatctacacgttcgcctttcacatccttccataacttgaagaaatatttcttgtcgttcagtttccatgcggtattcttcaagtcgtgaggattgttggcaacaaataaagcagctccgtatggttcgaaattatattgtttcgttatatgccattcggccttgtcagaaacaaagggtattgagatgagcatcttgtcttcgcgttcaagaagattgaacagtatatgattgaacgaagcgacagttcgtacagaggctatcggattatatcctttggaagtgttgtctattcctccatattcggttacggcaagaggaagaactttccccaagcggatgaacgagtagttttccataaggtcgagaatagcttcggaattacttcccgaacggcgggaactcttgcctactatgtttattccatcgtaaagatgtaccgacaagccatccatgtactccccggcacggtcaatgaacatcttcatagtattattccaatggtcgaaatcgcgcaactccatagccggatatgccgcggcatatccaatgattttcatttttttcagacttggctcagcgtgaatatgctttcctgtctgtgcataaaaatctgccatgagcatcctcatttcctgaccatgcatattgaaacatttgtcgcgtgcatggacaaagggttcgttaatgggttcgaaaaattcaggaactgcccctttcacatgcttggaatagtattcggcagcccatgcacccgccttcactgggtctatgccccattgtatggtacgcgcgttggcatgttccgtagcgacatatcgttttgtttccttcaaatcagtgtagttcaaaggcttttctgaaaaaggatattcgccaaccttttttgtcttgccatatgaataagagaacggtccccagaaagagcggccgattcctacaccgtaatctgcaagaaatttcctgacatctggatcagaatctttagatgtgtgtatattgaaatatttacctctgtcaagtgccgatacatcattcaggtatctctgagtggcataatccactgtgacagtagtgttataagtcttattctcggaagatgataaaggaaaaaccgagaaagacaaacacacagacaaagctgtaagaattatgttattcattgtattatcaaaatttaaaaggcagagaacactccgatagttcaattaaagtattccctgccattaagattatcacttctgtttaaacactaatatcagaaatcggccggtttgagtacatcgttcagcaccacttcatattcaacttctgttccgtcgttttcagtaacagtaagatggccgtaaccgccacttgagttattttctttcttaccttcaaacatgaacattctcttcttcgtcacttcctgttcttctttatcgcctgtttcaggattgataacttcttccttttcagtatagacttcattgaaagagaatgagagatgtttttctgtatccgaattgattttcagccactcgggcaattccgaaggagcctcagacgactcggcaaagaactcgatcttattcattctcaaagtctgatagtcattcttccaggcaatgaggtcgaacaattcgcgataaacagaaaacttggaaacctcgcctgtctttcctgtttccacattcttgagataggtaagttcatacacgggagtagaatcaagttcgacctcagcccatttgtcattatcacatgctccgaacaaaaccaaagcacataagaatgtaattgtcttataaattttatctattagcttcattgttactataatttattatggtcttacttcaatatatccgaaaaatatatcgtcaaaataaatattatccttaaaggcattaaagcgcatactgagcaatatattgtccatttcagcctttgaagtcacagtggttgtggccgacatccatttgctgtcggagccattcacaatgccgcaccatggtctatcgctctgccatgtcatatcttcagctccttctttacctgccggaacgaaatacggactcatacccttaccctgtttataccccggtgtataatatttgtagctgaaagtatatgtacctttaccaccagtaaatgtcttggagagtaatgccctgcatcggtcaaatgcttcgacaaacatacattttgcactgttgtttattccatccttcagaggattgtccacaacctgtgaaggaactacaggatgtgttttggtatcggcatcaataactttccagtcggcatatgtgtcagaattttcaaaatcttcatccaggaacgcaccaaaagtagtcgctacgtttggagctgtagcctttatctcaaggttctgatatccaaccaacgcttcagttaaagttcctgtaagggtcagttcatctgtgttatagattttctcaaccaaagtaagaatcagttcatatctgctttgcttgtttacttctgctgctgtgatgtttacgctacccctgacagctgacggtctgttatacgagttggagtaagtaagctttagagatgatggatttatctctttatatccaaactcagaattatccaaatctatagcaatgtgtgtttggtcaatctgacggatgttataagtaataggatcatcactaggtactactgtaatagccaaaggcacaacaagagtttttggcgaagcttttggagtgtacttacctttaccctcactggcagaagttctttctattgtcatggaaagaagcaatggcttatcgctgaatttctttgcagtgaactggtatggagtgtcaaaactggttaattcgtcatttacgccagtatccgcacatttgaaagtccatttgttaggcaatccgtatgaatcgtccttaatatagacagacttaccatattcaagttcgtatttttcgtattcgggagcttcctcagttccaccgactattccggtctttatttcctgtgtacactccggatcactgtatacctttacggccggtacgaggttaggatcatacacgcggatatggaaagttgtatccatcacatatacatcaccctcctgcttagcataacaatattttttgatatatcctccggtattgtcgtcatacaccgaatatggatatacaacctgtctgcggaaagtattgcacaaacgtaccgtatggtcaccgggtttagtgaaatacacatgtatggttttcaaatcgttggtatgagggatggattcatcaatcaggtttgtatagtctgtctgtccccactccatcttaccattaaggaactttgtaccatcatccgacacaacccactgatgcgacaacatgccttgggataagtccattatacttatatagttattaagattcagctgaataggtgaaacgttttcctgatctgtactcacatgccaggtacattcagccacgttattcaacggttcaaactcatcatccttacaagatgtcagaaccgagattaatgaaagagcaatatataaaaatctatttttcatcgtatttatttattaatatcaggatttgatgtaatttctatatttggaataggccagtatgccacttgcggaccgtagttcaatgatgcttggaaataatccacaaaagcgtttcctctcttttctggcggcagctcataaaatctgtactgctttccaaagttgaatgctgataccaaagcattagggtcatcaggattaggcttaagatatttggtctgaatcatacagtacttatattcgtcggatgccaactgatcaaacctttccttagttatattccagcgtctcaaatcaatgacacgtatggcatgtccttccatacacagttcaagaggacgttccacatacatcagatgattcattacatcacttgcagcatattccttctcatcgtatgtatatctcttgaattctccctgttccgattttccgataagcacaactccagcacggtgacgtaccttgttgatggcattgatagctgactgaacatttccatcgcttgcaccgcctttaatcagacattctgcatacatcagatatatatctgccaaacggataagacgatagtttattcctgaggccatagcaggcttaaattcagtttcactcttacgtgtatcccaatttgataattttctgaaatacgctgaagagccacggttgaattttgatacctgttgtgggagagactgataatatatcagactttcatcgccgtttattgcaagagaggcagatgcacgcatggaatagcttctgaggcgatatgcctgaccgtcttcccatttaaattccggaactatgtcatcgtagccggtaatcttattgtataaaactttattatctccgacagttgagacgagtcgttcgcgtactccaacatattttcctgctgttgcatcccacgtataaacgtacgttctgttatatacgacaccctgacggtccacctgcgagctgaaagttgttcccaactggtcgtatataatatccctatgttcaggatcaccataattgtcggactgcatttttatccagttacgttcatcaagtctgtccaccggctctgtttcgaatgcttcaacaagccaaaaagcaggaacagtgttaagccaggcatcgcccaagccatttacattcattccccatatattatataaggtagactccgaccatgtaccgaattctgtattatactgtgtagaataggaaacctcgagaatagattccgaattgaattcattggcagcagtaaaattatcgactatgtcatcaaccaaagcaaaacctccattatcaataatatccttaaaatattcggcagctttattatactctttatcataaaggtagcttttgcctaatattgcctttacagcccaagaggtgatacgtcccaaatcggttttctcccatttgtcattcaagccaaggtcaagagctttctgtaaatcttctctgtaatatttcttgatttcatcacttggtgtaacctttttatagtaatcttcttctacctctgcaatttcattaatataaggaacattaccattattgaatgaattattgagataaaaataaaacaagccacgcaaagaatatgcctgtgcctcaatctgagcaagcttggttatttgaggttcatctgtaacatttggacggattttctctatactggccagaacctgattcgcacggaacacaccagtatacagtgcagaccatttaccacggactgttccgtatgaatcattaaaggtttgcttataggcttcgttatcaaactgctttctgtccttattaccttcaactgctatatcacttctacggttctcatcgagcggatgataaatattggtatttttcaaagcattatatacagcagccagtcctttctcgcagtcgcctattgttttataaaaattctgtgttgtcagctgatgtatgttttcctgcgtaaggaaatcgtcgcatgaaaccaatgtcatgcccgacatcaacagactgaatactattgttttatatctgaagttcatatatttatattattaaaagttagaaattaatctggaatccgccacgcatctggatacttataggatatgttccatagtccaaaccacgacgtgacaatccattactaccgacctcagggtcgtatccgtcgtattttgtcagtgtaagaagattatcggctgcaacgtataaacggaacttgcccaatccaagctttgatacccaactcttggggaatgaatatcctaacataatatttttaagtctgacaaatgaaccgtcctcaatccacatatcagtatgagcacgatagttgttatgcccctctgtacgataagaaggaatggtagaggtatagttggtaggggtccacatgtatatcagttccttattggttcttctttgatatgtatatatcttcgtaccgtttattatttcatttccaactgaagcataccagttcatagagaaatcgaagcctctatagtcggccgagaagttcaaaccaagttcataatccggcataccactaccggcataaacacggtcgtcatcattaagaacaccatcattattggtatcgatatacataaggtcacccatacgggcacttgactgtaatttctgatattctgcaagcttctgttcagtattgattacccctgcggttggcataacaaagaaagcaccggcttcatatcctttcttgattgcagttacataatcacttcctgatgaaacaggtttaccgtcggggaagaaatataactcattttttcctgccatagacacaatctcattcacgtttttggtaaatgtaccagtcaagctgtaattaacaccacgtattttgttgcggtgagtaagtgaaaactcaacaccacggttttccatatctccggcattcaatgtaacagttgaactctggccccctccatttgacggtggcacgaccatcgggaaaagcatattcttcttgttactcttgtacaaatcaagacctaagataagcttgttattatataaagccatgtcgataccggcattaagctgctgggttgtttcccatttcacattcggattggcaaatcccaattgggtaaaaccatttgcaagaatttcggaagttccggtaccaaaagtatagtcgtagtttttgtatatagctggtgcgtatgaataatcagggaagttctgattaccggtagtaccatagctgaatcttaattttaacgaatttactagccacctgaatctgtcgaagaatgattcctcagaaatattccatcctacagacaatgacgggaacaatccccaacgattttcttcggagaacttagatgaaccgtcgcgcctgatactggcacttgccatgtatttgtctgcatagctatattgtagacgacccaacataccaaccattgtactgatacggtcctgtccccactggccactgcctgtacccacagtcatatcggatgttcccgcatttaggttcggaatctcgttagtaaccaaatccattatactggcatagaacatctcgtatgtatatttctccatactgaaaactccggtaaatttaatatcatgcttttttatcttcttattataatttaccattgtttcccaagtgagactggtattctttgaatgagtatcttttaattgcgaacggtaattagagctggttaccttttcgcctttctgattatatacctcaaactcaggtcgaattgagacagctttctgattgttatatccaaagcccaaacgtgtggaaacattcagtccgggaattacattataagcaagataaaaattaccgttaaatgattctgtgtccttatgattttcctctttcaatcttcccaatgtataacttacgccctgtaaatctgcaggatcgccagctgcatttactatacttgcctgtggataaatctgagaacgagtaggcgagtagtcataacattcgttcaataacccccaagccggagataactggttttctatcttcatagcgatgttagtgttgatagtccattttccgcgctgaaaatgtgtattcgaacgaatattatatcttttgtaatcggaatttatcaacacacctttctggtcgaaatagttcgcggtaaggttatatgtcaaatctttcttgccgccattcgcagtaacagaataattctgtattggtgcgttattattgactacatattcatataaactagagttgttgaagaaattcacaggatatgttttcagattagaccaggccaggtcgtctgtattctggtttccttccatcattctgttagacatcacttttacaaatatactctcgttggcatcaagcaaatgaatattcgaagtaatgtgctgtacaccataatatccgtcgacagctatcttcatttctccttccttacccttctttgtggtaataaggataacaccggaagcaccgcgagtaccataaatggcagccgaagcagcatccttaagaatatctatacttgctatttcgctactactcaatcccgggtcgccctcgaacgggacaccatcgacaacatataaaggagaactgtcgcctgagatagaacttaaaccacgaatctggatgttggatttggctccaggctcaccagaacttgcctgaacgttaactccggcaaccataccctgaagagctgtacccaagtcggaagtactgatcttagtaatctcatctgagtttacacgtgccactgcacctgtcacctcttttttacgcattgagccataacctacaacaaccacttcatccaacacttttgtgtcttcctgaagcttgatattataaatctgaccattcttgattgcagcttttacagttttatacccaacaaaactgaacactaagttacctttagtcggtaccccttgaagaacgaaattaccatccatatcagtaatagttccaagagaagtaccttcaacttgaacagctgcgcctataacttcaaggttattggcagcatcaatcacctttcctttaactgttatcttctgtgaatacatagacaatgtatagaagataagcatcacgaacaacatgtacctgccatggtaccattttttctgatttctcatttgtaaaaattttaatttagcaataggttatgaaattccttttataactgacgctaaattatttatttataatggtacaaaaggggagaattatatatttaaaaagggggtaaaattttacccccacttatattaagaatccaaatcggtctgtatactctgttctttgtactgttgcggcaatacaccgaattctttcttgaaacattctctgaaatacttcaaatcattgaaccctacatcgtatgtcacctctgatacagaataccgtcctgtcttcaacagttctgccgctctcttcattcttattgaacgtacaaaagcattggctgttactcccataagtgctttcagcttcttgttcagaaccaaggccgtcacgccaagacctttacatatatcctctatctggaacgaagagtctgtaatgttgtcctctattatctttacaagtttctcaaggaacttatcgtcggtagatgtagtgcttacctcggaaatctttattgccggaactttcttgtgttgaagaatccgcttcctgttggttataatggaattaagcagctctttcattatcttgttgtcgaaaggtttagggcaataagcatctgcatggaatttatatccgatgaaataatcctgcaatgtagtcttggctgaaagcaatactacaggaatatgagatgtccttacatcctgcttgattctctcacacagttccagaccattcatgcccggcatcattatatcggataaaacaagatccggttgcaaatctggaatcatgttccatgccatctccccatcatgggctatcattatcttatacttatccgacaacagtaatgacaacatattacatatatccttattgtcatcaacaatcaatatagccggagattctccgtccacttctatgtctatcatctcttcatgctcgcacgattcacttcttaacacatcagcaaacttttcatcctccccactgttggcagagatattctccgtaaccatgtccccctcagttatcataggaattacaacatggaaaacagtgcctttaccttcctctgatacaaacgtaatatttccattatgtatctctacaagccgcttggtcagaaacagacctataccggtacctccttcagcagagtttttattctgactgtagaaacgctcgaagaggtgtgttttcaggttgtcggatattccgtttcccgagtctgccacagagatgtttattttgttatcctgttcattgacagtaaacgatacaaatcctccggcaggagtatgcttaatggcattcgatacgagattatagattatctgttccataagatgagggtcgaacagaaagcttatatcactgcgtgagacagaatattccagccctacacctttctgttttgcccaatacgtgaactgctgaaatacttcttttgagaaagacgagaagttgccatatttgagattcagactaagcattcctttctcgctctttgagaagttcatcagctggttgacaagacttaacaggaacttactgttatgctccattgtctgcagcatgccggcaagatacttgtcggacgaatacttgcccgattcaataatcatactaagtggagaatgaataagtgtgagtggtgtcctcaattcatgcgatatgttggtaaaaaatgtagtctccttttcaagaagttcttcagtcttgcgtttttccatgtttgctatatatagagcatttctgcgctgcacccgtgaggtataatacaccttgaaccggtataaagacaagacaagcaatataaaatagagtgtataggcataccatgtacgccagaaaggagggttaataatgacaggtatggaaagttcattcaaactgtagactccatcgctattcctgaccctcagtctgaacatatattcgcctgaaggaagctttgtgtagaaagcctcacgatgaaaagcggaggtggaaatccatgaatcatctacgccttcgagcatatattcgtaaccaaccttataaggacttctgtaatccagggagctgaactggaatgagaaagtgtttaaattataaggcaattcaatgtgctctgtaaaacttacacttttgtcgaaataagctgaatatgtggaatctgcctcaacgctgtgattgaagattttaaaatcaacgagtgtaggactaccgttgaaatctatcacatcaaagtcattaggtctaaagacgttaattccgtttacgccaccgaatatcattgttccatccgtcattactccagcagaaagttccataaattcataatcctgaagaccatcgaaaatatcataagatcttattctctgtgtgttgatattcaacgaattaattcctttattggtagaaatccataatgttccatccgtgccattaacaattgattttattgtattgctgctcaacccgtctgcagagctaaaattttcaacgcaggcattatggttttcatccaaatccacgattttccttaacccacgtccaagtgttccataccagatattatgattcaagtcttcacatacaggcactatatagtcgagttcatcaagtcccttgactgagttcaaaacaggattatctatatacaaatctgcagattccaatactttaagaccgaagctggaagctacccatatattacccttatgatctttaatgatgtttcttactatcttaagttctttattgtcagatgttttgatttccttcatcacacctgtggacaaatcatatctgaaaagacctttattatatgtgccaatccacaaatattttccatcggcaagcattgcgcgcacatttctcaaacctgagatctttttataatcattatcagaagtgaaactgtaaataccatcgtacatcagagacacatacatgcagtcggtgtagtttgagtatgctgttgagtatactatcctgtttgccgtgaaaggaataagtctggcattaccggtaatggaattaaaatgatatagccctgagccttctgtgcctaaatatatatcagatttggcaaatgtataaacggacgatatatgatcatttcctattcctctgaataaatctataggtttattattttcgcgtatactcataaagccactcttgaaaaatcctatccaaagaatatcgtttttatcaagaactacagtttgcggatagctgtaagaatatgtagcaataacctgtggttttgactcgatggcatgcaatacatcaaaagtcaacacattcacagtgcttgtagtggcataaaataatcttttgtttttatataccatttttcgtatatcacagttttccaacagggtacttaccttgcaggtatgcttgtcgtataaacataattgatgattttccagatttgagtacaatatttgagaagatgagatgactatggctgaagctatagggcatcccaatagtttgttaagcagtaattcatctccatcgacgttacattcgtacaggccgtcttcggaggagagcattatcgtattatctatttctatgatgtcggaaatgtatggtaattttaatgttgatcttaagacagtatttattttgccattttgaaaatcataatttacaaggtatatactttcatcagaggaatgaaaccagactctgtctttagagtcgacaagaatcttatcgcaagtgaaatttttatcaataccgctgtgaccaagatttaatgaaacgaattcgttctttacagaattgaacaggaacactcctctatcggctgtacctatccacagatttccatgtgaatcttcgtcaatacatactatcagattactgttaagaccgtttgactgatatccgtaaaccttaaattcatatccgtcaaacctgttcagtccgtcgttcgtggccaaccatataaagccttttgagtcttgataaatacattgcacatcattttgggaaagtccatcaagagtagtgtactttcttgtgacaaactcattggatgcaaaggatttgcaaactataatcagaactgatattaaacttaagattaatctaaacatataactattattctttatatttcatcaagattacaaagttattgattttatctaaaacatcaagtatttacagtagttaatagataattatagatattttccactttagaatgcgtatcaaaatcaatcaagaaaaaaataaatctttaacttcatttcatagtataaaacaaaaaaagcatcgtaccattacactcaataatagatacgatgcccgaaagaaattacagtaacagactgtattgggattgttcttaaaaagacttatctgtatgactttatatatatgtcgagtatttcggtatccgacagttcatgagggtccagactgaacaatgcacccatggcagttcgcgcattatcaatcatcttagggaaatcttcctttactattccccagtcgctaagcttcaaatcgcggacattgcattccttctgcattctcaccaaagcatctataaaatgttcgggattaaggttcttgcatccggtcataacatctgccatgcgcatatatctctttgtcctgtcataaataaaagtagagaaataggcctcgcttatagctatcaggccaacaccatgaggaagagcgggatagtatgcgctgagagcgtgctcgagagaatgttcggaagtacaactggatgtggattcaaccattcccgccagcgtacttgcccaagccacctttgccctcgctttcaggttatttccatccttcaccgcaacaggtaaatatttatacagcagtctgatggcctcaagagcgaaaatatcacttattggggttgcacaattggcaatatagccttcggctgcatgaaagaatgcgtcgaatccctgataggcagtcagatgtggcggaactgaaaccatcagttccgggtcgattatcgacagacatgggaaagttaaagtggagccgatacctatcttttcgtttgtttccagattggttatgacagtccatgggtcagcctcggttccggttccggctgttgtaggaatggctatgatgggcaatgctttgctgtaaggaagccccttgccggtacctccttcaacatattcccaataatcgccatcattacatgccatgattgcaatggatttggccgtatctatcgaacttccgcctcccaaacctataatcatatcgcaattttcctcacgacagattgccgtaccttccattacatggtcttttattgggttaggcaatatcttgtcgtacaccacggcatcaacattattttctttcagcagaccaatcaccttatccagataaccatatttacgcattgatgttccggatgaaatgactatcaaagcctttttgccgggcaatgtctctgttgaaagacgtttaagttcgccacatccgaagagaatcttcgtcggaatattataaccaaaaacaaaattattgtccataaatattatcagtcagtcaacttactatcttaaagcctcatcaatcactttcttgagttcaggataagcctcatctgtatcgcccacctgttttctcaactcacgcagtttctttttcatgtccttaagaactttggcgtatttaggattatcagccaggtttaccatttcgtaagggtcgttcttcacatcgtagagttcgaaagaaaccggagtaggaacaatcttgtggctgttcttcaaccatgacattgatttctgtccgtaacgtttgtcgtcgtaatgacggccatagaaaagtatcagcttatagttttccgtgcggatacctatgtgtgccggaacgtcgtgatgaatcatgtgcatccagtatctgtagtaaacagcatccttccagttttctggctttttgccttcgaacacagaggcaaagctctttccatccatgtatgaaggttctttgccaccgaccatctctataagagttggagcaaaatcaatgttgttaatcatcaggtccgacttggctcccttgtaaggacatctcgggtcgcggactatgaaaggcattctttgagattcttcatacatccatctcttatcctgcagatcgtgttcgccaagcatcataccctggtcgcctgtatatacgataatggtattttcccagagtccttccttcttgagatagtcgaaaagacgtttcaggttgtcatccacaccctttacgcaacgcagatacgatttcaggtaatgctggtaggcaaggtatgtattctccatttcatcacctgtattgcacttatattccattacataattgcggatttcatgacggcttgagacagaagttccgatgaagtgacgaagtgaatcgttcttgcctcttgtgccttcggagccccatttgtctgtatcgaacaatgacaatggaacaggcacttccacatcgtcaagataatattcatagcgcggtgcgtactcgaacatatcgtgcggtgccttgtaatgatgcatcatgaagaaaggtttggacttgtcgcgtctgttcttcaaccagtcaatagcaaggttggtcacgatatccgaggagtaacccattttctttatctggttattaggccatttcttgtcagttacgtcacttgtaaggaaaatagggtcgaagtattcgccctgtccgccatgaccgttgaatacagaataatagtcgaagtgcgacggttcgcatcccaaatgccatttaccgatcatggcagtctgatatcccatattatggaactcatcaaccagatattcctggtccggctgaagcacttcatccaaagtgagcaccttgttacgatgggaatactgtccggtcatgatacatgcacggcttggggtactgatggagtttgtacagaaacagttctcgaagagcataccgtcccttgccagttcatcaattgtaggagtagggttcagtactgcaagacgacttccgtatgcgccgatagcctgcgaagtatggtcgtccgacatgatgtagatgacattcatctgtttctgctgtgctgcgacaccaacacatacagacaggaatggcataacagccattcccttcattatattattttttaaattcgttttcataagtcagattatcattgaaatagaacttgcaagacatatcatcgaatgattttacgtccttattctgcattttaacccattgttctgatttagccttgacagcgacctgagttgaaacctcattaccgtcgactacacttttaagagtgacatttgcatcctctgcattatggtttgccacacgtacagtgataaggcatccgttatcaaccttatcgtatagcggtttggaaaccaccgcccctttaagcttaatcttgaacacatgtgcatattcagtaggtttgttcttagggaagtttactacaagaccctcgtcagtcatcttatagtcaatcttctctgagcttccaagcatttcaaccgactcaatttccacgttctggcaatacttaggagcaaatgacttgatagtaacactaccatctgtccaagccagagacacggcatagaggttattgtcgcgtgtagtaaagcgaatgtcgtccgctgtatattcagtttttgtattgtctgtcatataacctgcggtgcctgcgttatgtccttcgaaagcaatcacccatggtcgtgagccataaatagcctcaccgttagtcttcaaccatttacctatctcggcaagtacgttcttctgttcgtctgtaatagtaccgtcggccttaggacctatattcagcaataagttaccgttcttgctgacaatatcaacaaagtcgtcgatgatatggtcaggactcttgttttcctcgcccacacaatagctccacgatttcttgcctacagaagtatcagtctgccatggatattcacggattctgtcgctcttacctctttctatatcgaacacctggatattgtcgccatatccgaatttagtgttaaccacaacttctttattccaatcaagagccgaattgtaataataagccatgaatttatagaaagtaggctggaacggatattttcccacagtccagtcgaaccatatcaattcaggctgatatttgtcgataagctcgtatgtatgcataaggaactgacggcgtgaacgttcgttcgagccttcatacttaccacaataaggtgtcataccctgaccttcgggctcatgcagtctttcgccatacagagtgattgtagtgtcctgaacatcagaaggagtttccattccatattcatagaaccatgcattctcgcatctgtgagaagaaagtccgaaacgcagaccggctttcttggtagcttccttcaattcgccgattatatcccttttcggtcccatatccacagcattccacttattgaaagtactgctgtacatggcaaatccgtcgtgatgctcggccaccggaacaatgtattgtgctccagatgattttaccactgccagccactcgtcggcattgaaattttcggctttgaacatagggatgaaatccttatatccgaatttggtcaaaggaccgtaagtctgtacgtgatacttattaataggatgaccttccttgtacatccagcgggaataccattcactgccgtatgcaggaacggaataaactccccagtggataaagataccgaacttggcatccttaaaccattcaggaatagtgtaattttgagcaatcgatgccgaatcggccttgaacacatcagtaccttttaaagatacagtagaatctacattaggagcgtatgtagaattgcacgacgccaacaggcttaatgccgcaactcctaaaaccgttttcatggatttcttattcataataatcttattacattaaataatgacattaattttttctgtaagcaaagatacacttgagttccatttacaataaataatttaattactatagtaaggggtaaaatatttaccacctattattgaacaaatttaccccctctcatatatgataataaactgccaatatcgaattacaagtaaatatatatttcaacaaaaaaggtttagcctattattacacaacaatttcaccctaagaataaaatatatatagagtaaatttgccaatataacaaactgtaaaaacaaatttatgaaaaactatttgatttacttactcgcagcagtatcgtgtacaactgtagcagacctaaatgctcaagtcagtacaaaaacaggtaatgaaaccacagaacttacaattccgaaaaagttctacaaggacagcattgatttcagcaatgctccgaaaagacttaacaacaagtaccctctttccgaccagaagaacgaaggcggatgggttctaaacaaaaaggcctctgacgagttcaaaggaaagaagctgaatgaggaaagatggttcccgaacaaccctaaatggaaaggaagacaacctactttctttgcaaaggagaatactacatttgaagacggctgttgcgtgatgagaacttacaagccagcaggatcactgcccgaaggatatactcacactgccggtttcctggtaagcaaagaacttttcctttacggatatttcgaagcaagactgagaccaaacgactcgccatgggttttcggtttctggatgtcgaacaatgaaagaaactggtggactgaaatagacatttgcgagaactgccccggcaatcctgccaacagacatgacctgaactcgaacgtgcatgtatttaaagctccagcagataagggtgatataaagaaacatatcaacttccctgccaaatactatataccattcgaattgcagaaagactttcacgtatggggacttgactggagcaaggaatatatccgactatatatagacggagtactgtacagagaaatagagaacaagtactggcaccagccattacgcatcaatcttaacaacgaatcgaacaaatggttcggagccttgccggacgacaacaatatggattctgaatatctgatagattatgtaagggtgtggtacaagaaataagaaataacataatctgaaattataaaaggcagtcttcattatcagtatgctgatgataaagtctgcctttttaacaagaagataaagattttaatctgccctatcactcatttacttcatccggatactctgtaagcgagtttcccgaattgcttatttcaatagagccgataggaagataattgaacttcttgctccatgcagagataccataatctcttctaagaataggcatcatgacctcctcggcacgtcctgagcggacgaggtcaaaccatctgtcaccctcgcatgccagttcacaacgacgctcataccatagaacatcaattacgcttttaaatctgtcaggatacatctgcattagcttgtcaacatcaatataacttccgtcgtctgcatgaacatgcttctttctgagttcatttatgtaatacttcgcttttgcttcatcaggattagtacctctgagatatgcttcggcaagcatcagatacacttcaccatatctgatgacccttacgtttccaggcttgtttagattggggtttcctatcatatcgtaatttttgaaaggaggatatttcttctgggcatatccctggaaatcaggcccgtaagagcctgtctcccaaacaactttttttgattcatcctgaatattggcattaggtttggttacaagttcatcgtaagtaaatatcgccgcatcacgacgcacatggtcatccggaaggaaataatcatacaattccttagtaggcagacaaaagccatatccattatcataatcaggactatttttcaactgtctcggtccgcagaaagtcacccacatagcaccttcgcctgcatcaatattaccccagtttgtattaccagatttggtagaggtctgtatttcaaatatagattcctcgttattctcctgatgagccgcaaacaatttagaataatcatccgtcagagtataattaccacttgaaattacatcctccaataaaggtttcgctttgtcaaaaatcttagcatcatcgttgctccagtcagcccaataaagatagaccttggccaacagggcttgagccgcagtcttggtaatacgtcctttcattgtgtccgggaaattatcctttagagaagggatagcttcaagaagatctttctctattgctttatttacattttcgcgagtatctctcgtaaacttgaatccttcaggataaagagtctcaagactgataaagcatggaccataatatctcaacaattcaaaatgataccaagcacgtaagaacttagcttcagctttataaactttagcttccggactgtcatactctgaatttattacaagattacatctatatataccacggtaacgagttttccacaaattatcggaaatagaattgacactcgtatttgaataatcctctatagcctgcatgtaaggctgatcctgatcagagccaccaccagtacgagcattatccgaacggatttcacccataggtacaatggaagcaagtgcattacccgaagcaccacctatgtgagctaacggatcataacaagcagtaagcgctttgaacatctgttcatcggtcctataaaaagaactttctgtttcggacattataggagctgtatccaggaaactgtcgctgcaagatgatgatgcaatagcagcaaacatgaggacaagaatattattatgtattttcgacttcataattttcaattttagaaattaagacttaaaccaaatctgaatgtacgggcctgagggtaagtaccatagtcaatacctgtgctaagaatattgccacctgccatatttcctacttcaggatccataaacggatagctggtgaaagtggcaagattatcaattgctgcataaattcttgctttattcagcatcaacttgtttattaatttagttgggaatgaatagcctacctcaagtgaagaaatctttaaatgcgaaccatcataaagataaaaatcggatggtttgccaaagtttccattaggatctttggatgaaagacgaggcactccattatcatcaccttctttccgccatctgtcaagatagaatgatggaaggttgctgcgtccgtatgcttcctgtcggtaaatatcagagaagactttatatccagcttttcctgttaagaagattgtcatatcaatacctctccagtcggcacctaaattcaaaccgaatgtccattttggccaaggattgccacaatcggttctatcttcatctgtaatctgcccatcgttatttgtatcttgccatataaagtcacccggaacggcatcaggttgtatcactttaccgtcttttgatttatagttctgtatctgctcttcattttggaatattcctaagttcttataaaggcggaaataacccatagcatgaccttcctccatacgcgttacattaacagatgttctccagctaccaccatcagtatatccatttacatttcctatctttacaacctcatttttaagatatgaggcatttgcggaaatagagaagttgatttcgttccaatttttattaaatgtcatctgcatttccacaccctggtttgttatattaccaaggtttctaaaagctgcattattacctctaatggcttcaactgttggctggaacaacaaatccttagtactttttttaaaccagtcgaaacttgctctaatcataccattatagaatgtcatatcggcaccaacattaaattgttcagaagtttcccatttcacgtctggattaacaaggttattaggagcagatcccacagtgatggcattaccaaacgtgtaattataattattgccaataatagaagtataggagaatggagaaattcgctcatttccgttctgtccccaagagaatctaagtttgaagacatcaaagttcttaattttccagaatttctcatttgaaacattccaacctaatgaaacgcccgggaaagtagcatatctgttattgggaccgaaatttgaagacccatcgcgtctgaccacaacttccgccatatatttttcagcataattatagcttagacgagcaaaatatgagaacatactatgtctaggattagcaccgccactattagctgatgtcataacatcaccagcattaagataccagtaattctcattggtcattgcttcatttggatatttatttcgtgttccggccataaactcataaacatctcttgatgcagaagtacctaacaggacagatgtagaatgttcaccaaaagattttttatatcgcaatgtattctcccactgccaactactattagcatttgtactttgttctaccctagaattatcttctttacattctgcagaatgaaaaaactttggtgcaaacattcttccacggaaattccgatgattaataccaaaatctgtgcggaaaacaaggtctttaataaaagtgatctcagcataaacattaccaaaaaattgctgggtaatatttttattcttaggtgcctcatccataaatgcaatagggttccacatacggctataaggtacaggagagactccatatccgaaagtatcgttgctattctcatcataaaccggagtagtaggatcaatattataggcgtatgatatcggattataaccattgataccggttgccactccactattctctatatatgcatagttgacgtttgcacctacacttaagaaatcatttatagaataggaactgttcagccttgtgctgaatcgtttgtaaaatgacgcatcttcaccgataataccattctggtctagataattcaatgaaagcaagcttgaacccttatcactgccaaagttagcagtaatgttatgctcagtaacaggagctgtattcaatatttcattaaaccagtctgtattataacctgttggagcagtaggtacaccaccggcaagcggcatatcatcattgtcggcaaactctttcatcagcataatgtactgttcatcattcagcatggttggtttctttgctactgtagagaaaccatagtaaccatcataagcaagcgatgtctttcctttctttcctttctttgtggttataaggactacaccattagcggctctggcaccataaatagcagctgaagttgcatccttcaagacttccatgctttcaatgtcgttgggatttacactgttcatgtcgtccataggcagtccgtcaattacaaaaagaggattagagtttccatttgtaccaacaccacgaattaccagcttcggtgctgttcctggctgaccggaatttgtcacaacgttcacaccactaaccctaccgctcaatgcattcacggcatttgctggtttagattgcaataaatcatcggaatcgatgctactgatagcacctgttacaacactttttttcttaacctcatatcctattgctacaacttcctcgagtgcaatggcagatgtttttaattgaacgtctatcttagactgacctttatacactatattctgtgtatcatatcctacgaagctataaatcaatgtcgattccattggtacattttccaagatataatttccgtccaaatcagaaataataccgtttgtggtacctttaactaaaatacttgcacctatcacaggtaaaccatcggagtctgttatacaaccggtaactttcccgttctgtgcatttaatggtaaactgaacgttataagaatcagcatacacattaatgatagtgttctgttcataatctagagttttttgtaattagtgtttttcttaaaataaaaagttttgttctatcagttgcgcgctacttactgacacttgcaaatatatatactatgtaatataaccaaagggggaaaatttcatttaaataggggggggaaatagattaactaaatattttaaggaaaaatggctgttagaatccattcccagactccaacagccattttatcactaacaatcgcctgttaatcaatatatttttctgcccatttccttaagatttgcatccctgcccagtggaacaaaagtaaatccgtatgaatagcttcccttcagaagacgcttgtctattgaaggacgggctttcagactccagctatctgttccgcccactccagcctgaaccaggtcgatattaagagtattagaatacaagtccttttcaagttcatttatatgtttagccttatcaatcgcattctgcgacatctcccacactgaaacagataggggttcatcgccgacaatcatcacacctgccttatccgactgcaaggcaaaccatctcacgtcacaacggtttccgttttcctgcggcattacatagtcaaatcccagagcggacaccttgcagttatatatagacaccattgcagaggcttttctgtcggaatagttttcccatgggccacgtccataatatgtcacatccgacaaacgattggtacattcgcattgcaatcctacgcgcaacatttctgatatttcaggagacttcatcattgaataatgaacgcctattgttccgtctgcttttactttataattcaaggtaagtctcagtctttcatctatagcctttagcaccttaacctcaagattgccttccgatttgcgtacatctatagaaactgtctttagctttaatggagcatctttccagaatgcaaacagtctatcgaccttccatcctcgccagtcattgtctgttgacgctctccagaagtttggtttcagagcagatgtgatgatactttcattatctatcttatactgactgatataaccatcactgatattcagataaaagttctttcccttcacgctgatgtctttcttgttatctgaatcgatttccatatccaatgtagtatcaacgcattctactatctttggtaaagaaagatacttaaactgttcccaggcaacctcgtatccagctttggcatacagattgtcattcttgagcctggcactcaggaataaccaatattccgcaccgtcatcggccttgaaattctgaataggaagttttagtttacagctctcaccagctggtgttgtcggcacaataatctcaccttcctgcaatacactgtcttcgtccttcaattgccaaaaataacgatactcatctgttgaaaggaagaagtttctgttttttacagttatctctccactatagacattatcagttgtaaatgatacaggagcaaacacgtacttgcattcctcagtagcaggtttaatggagcggtcggcactgataacaccatttatacagaagttttggtcgttgtgctcccctttctcatagtcaccaccataattccatgatttcttattatatttccgttcattatccagcaatccctggtctatccagtcccaaatatatccgccggcaagcgcatcatgagaacgtattgcatcccagtattctttcagcccgccggtagagtttcccatagaatgtgcatattcacacattattatcggacggttcatgaccggattcttagtcattgctataagctcatcgaccataggatacatacggctaatgacatcgacgtataaaggatcatcgggattggcatacacacaaagctctttctttgccggtttgacatcttcgttcacattaaaatctatctcactagtaacgattgacgcttccttacgtccgataggtttgtataaaggattttccggctgtccttgcgccccctcgtaatgaacaggacgggttgggtcataatctttcagccatcctgacagagctgcatgattagggccgcatccagactcgttgcccaacgaccacataaacacagaaggatggttcctgtctctcacagccattcttaccactctctccatgaacgagttagcccactcaggcctattggacagataccccctttgatgatgagtttcaagattagcctcatccattacgtatataccatacttatcgcacagttcatagaaataagggtcgttaggatagtgcgatgtacggactgtattgaagttataacgcttcataagcagaacgtcttcgagcatctcatcacgtgtaacggtcttacctccggtctcgctatggtcatggcggtttacaccaatgagtttaataggagtgtcattcaccagaatctgattacctgttattttaatatccctgaaccctaccttattacttctcgcatccaccacgttgccctttttgtctgtgagctttataaccaaagtgtatagataagggtgttccgaattccatagttttggcttagaaacaattccctccatcattccgtaataaacattatcacgctgaggataaggttcgttcaccacataatcggcagtaacggtaatgtcttttccaaacaccggtttcccatcggcatcatataattgggctgacagattccatcccttcaaatcatccatattctgatttgttatttccggacggatctgtaaccgtgctatattcttccggaaatcgatgcgtgtccttactccataatcatatattgccacctgcggaatggacatgatatatacttcacgatggataccagccattcgccagtggtcggcatcttccatataacttccgtcggtccacttatacacttgcaccgccagtttattctcccccttcttaacgtattcggtaatatcaaattcagtaggcagacaactgtcttcggaatatcccaccttctgtccgtttatccatacattaaatcccgaatagacgcctccgaaatggagtataatcctgtcgctcttccacttgtcaggaacaacaaactccttgatataacaccccgtctgattattcctgtcaatatatggcggacgagcagggaaaggataaatagtatttgtatatataggatagccatatccctgcatctcccaacatgaaggaacaggaatagttttccatgatgatgaattgtactccactttataaaaaccggcgggagccaatgccatatcctcggaaaagttaaacttccattggccgttcaacgacatatactccgatttctctctgtctccatccaaagcccaatccactctccggaaagaataagtagtactgcgggaaggcaaacggttaattccgtttatggtctgatcctgccatacattctgattgtttctccactgattggcaccgttgtccgatgcagacagaaattgcatcatgaaaaataacacagaaaatgaaaaaatagattttaagttcaagttcataaattcgcattttaagtttctatgcaaatatataagtataacgaacaatgaatagggggtatttctatctatatagagtggtatttttacatatgagctaaaacttaaaaaaaactgtcagtattactatgctatgtagcactctatatgaaaatattatatattcccaagtcaaaagccttttcaaacaatttttatatattctcatcctatcccttccatcaaagataaattccaatcctgatttgccagccgcatttattccttttttcaggagaattttctttatggctatcgccatgaaaattcacctgaaaaagaatgcggcggcaaacggattagaattaaagaaaagattacagggattaactgcgaccgacgtgacgcatagccgtaattcaaaggcggctatccttatattccatatatgacctcacaaatactgtgaaaatccactttccccaataacaaaacatagcctgccatatcaacacccaaaataagacagggatttcaactccctccgatctgcatagtctggtggcttcgctatgcttttactcctacatccattttttttctttcttttttcctctgttcccgttctttcctatccttcgtgtgacatttgatgacacctgatgacatctaatgtcatctatttgtaaatcaattgtttactcaatttatcatcttacatttggactgtgaaacaaatcaagtagtcactcaaaacaaaagattatggcacaagaaaacagtcctgacaaggaaaaaaggcaaggccggacaaagaaacccgaaaagccttatgtggaacaaattgacgagcttctgctggtacataacaagaatgacccaaaggaaggtttgggagtaatcagcaagatggacgagaaaggcaattatcagacggttacaccggaagagaagaatgagaactcattcctgaaattcgacaagaattcgagtattctcgaaaacttcatcaagaatttctggagccagctgaaggagcctacgcatttcaggcttatccgtatgaccttcaatgattacaaacagaacaaacaggctctcaaggacctggccgaaggcaagaagacagacgcggtaaaggagtttctgaaacgctatgaaatcagaccgaaagtaaacaatcagaaaaacagtcaaacaaaagaggaggaaacaacaatggcaaagaagcaggaacagacaacgcaggctcagcctgaacaggtatcacaggtggaagctgccgcacaggggcgcgaacagcaggaaccgcaacgccagcagacacccacgtaccgctacaacgagaacatgattaattgggaggaactgggtaagttcggtatatccaaagaaatgctggagcagtccggacagcttgacagcatgttgaaaggatacaagaccaacagaaccatgccgctgacactcaacattcctggggtactgaccgcaaaacttgatgcacgcctttcgttcatatccaacggcgggcaggtcatgctgggcatccacggtatcagaaaggaacctgaactggaccgtccttatttcggacatatcttcacggaagaggacaagaaaaacctgcgtgaaagtggaaacatgggacgcgtggctgaccttaacctgcgtggcaacacgacagagccgtgtctgatttccatcgacaagaataccaacgaactggtagccgtacggcaggagcatgtctatatcccgaatgaaatcaaagggataaccttgactccggacgaaatccagaaactgaaaaacggagaacagatattcgtagagggaatgaagtccaatcaaggtaaagagtttaatgccaatctgcaatatagtgcggaaagaagaggcatcgaatttatcttcccgaaagaccaggctttcaaccagcagacgcttggcggtgtaccgctttcccccatgcagctcaaagcgttgaacgaaggacacaccatccttgtagaggatatgaaacgaaagaacggcgaactgttttcttcctttgttaccatggacaaggttacaggcgggctccaatatacgcgccacaatccggaaacgggagaaatctacataccaaaggaaatctgttcggtacagctcacaccggaggacaaggaagcgttacgcaaagggcagcccatctatcttgagaacatgatcaaccgtaaaggtgaggaattctcgtcattcgtcaagctggacctggcaagcggaagaccacagtattccagaactccggacggtttcaacgaacgacaggcaccagccatcccggctgaggtttacggacacctgctttcggcacaggaaagagctaatcttcaggacggaaaggctatcctcgtaacgggtatgaaaggtcccaacggcaaaccgttcgattcctatctgaaagtaaacgcaaacaccggacagctgcaatatttccaggaaaatccggatgtgcgccgcaatacttcacagcgtgcttcacagactgacaatacccagcagcaggaacagaagaagggagcaaaacaggctgtctgacctgaacgggattcaaatcattcaaatcatcaattactaaaaaaggaaagaacatgaacaagaccaatcatcatatctacaagactgaacaaatcgactgggagaaactggaatcggtaggtatcagcagatcgcaaattgaaaaggacggaaacatggacctgctccttcagggagaggaaaccaatgtcatgtccattaaaatcaagactcctgtattttcactgaccatggacgccacactcagtctgattgaagacgagaatggaaatccggtcatcagcgtaaacggtatcaacccttcaggtgaataaataagaaaccataatgtatcatctctctttccatacggacttaccgtatggaaagagataaaaacagaatttatcatgattgccatattaacagacaaaccaagtgtaggaaaagaaatcggaagaatcatcggtgcaaccaaagtaagaaacggatatgtggaaggaaacggctacatggttacatggactttcgggaacatgctgtcactggccatgccgaaggactacggaacccagaagctggaacggaatgactttcctttcatcccgtccgaattcgaactgatggtacggcatacacgcaccgagaacggatggataccggacattgatgccgtgctccagcttaaagtaatcgagagagtgtttcaggcatgcgataccatcattgcggctaccgatgccagccgtgacggggaaatgacattccgctatgtctatcaatacctgaactgtacactgccttgcttccgtctgtggatttcctctcttaccgacgagtctgtgcgtaaaggcatggaaaacctgaagccggacagttgctacgacagcctgttccttgctgccgacagccgcaacaaggcggactggattctcggaatcaacgccagctatgccatgtgcaaggcgacgggccttggcaacaattctctcggacgggtacagacaccggtactggctaccatcagcagacgctaccgtgaaagggagaaccatatttcatcggacagctggcccatctacatcagcctgcaaaaggacggcatccttttcaagatgcgccgcacacaggatcttcccgacaaagaatccgctacaatgtttttccaggactgcaagctggcacatcaggcacagattacaggtatcagccacagcgttaaggaaatacttccaccggacctgcttgacctgacacaacttcagaaggaagcgaacatccgctatggttttaccgcatcagaggtgtatgacatcgcccagtctctttatgaaaagaaactgatttcctatccgcggacttccagccgttatctgacggaggatgtgtttgactcgcttccaccaatcatggcgcgtctgctttcatgggagctgttccctgcagctaaaggaactggaggtattgacatatccaatttgtcccgccacgtaataagcgcagaaaaagccaatgtacatcatgccatcatcattacaggtatccgtcccggaaatctgtccgaaaaggaaatacaggtttacagacttgtagccggaaggatgcttgaaacattcatggctccatgccgcatagaaacgacaaatgttgaagcggtttgtgcggcacagcatttcaaggccgaacaaacaagaatcattgaagccggctggcatgatgtgtttatgcgttccgacatggttccaaaatcaggatattctgtcaatgaactccccgaagtggagaaaagtgatactctgaatgtatgcggatgcaacatggtacacaagaaacagctgccggtaaatccgttcacggatgcagaactggtggaatacatggaacagaacggactgggtacagtatcctcacgtaccaatatcatccgtacactggttaaccgtaagtatatccgttattcagggaaatatatcgttccgaccccgaaaggcatgttcacctacgaaaccatccgtggaaagaaaattgcggatacttcactcaccgcagactgggaaaaacagctggccggacttgaaagcggaatgataaccggacaggacttcctgaacaggatcaggactctcgccaaggaaatgactgatgacattttcaacacctattccacaaaagaagaataacatctatacctaatcaaccaagagaatgcaggccggaaggtctgcatttttttgtatccgtacagaaaagaatctgtttttccgcttttaagcggcaaaggtcttggattgcctgccttttgccgcaaggctgccctcatgggcttggctggacaggaaaaaatcatcctcgctgcgctccggtattttttcctgccaggccttgcgcaaaaaggcaatccaagaggccggaggcctataaaatcgggaaaacacatcccgatgggattattcattcataaaattaaggattatgaaactacagattatcagaaagatcggcagacatgcaacagcgatattcctgattaccggaatatgtctgctgacaagtaaagggattgtccctactgggatgattacgctgctgttgcttgcaggagggttcatcggttttctgttcaggatactggtcattattttcaagattcttattcttctgttcattgtaggattatttgtcgcataacccaaaatataaatatacatatatggaaacagttgctataacctcacaagctcctgtcatgccggctgtatggccacagaacgaacatatcagaccggttaaaagacgtctgcccaatacagttgatgaacctaaaaatatcggctactatctggaatcgctacgtgatatttccagcaatccggacagagagaatattctgaaagaattcttcaaggaaacttatgtataaccataaaatttttcaattatgttttttcaatcaatttatcagatgattacagcaggtacggatctgaatatcaatatccgtaaagtggacaacagcctgagcgtagcagtcatgccaaggcggaacagcctgaaagaggatacgcgacagaacatggtgccactgatcgtgaacggaacaccggcagaactggatatgggcttcctgcagaccatactccaaccgatacagaaggtacagggactgcttgtcaatgcggaaaatttcgagaaacaggcagaaaaggctacatcacaggccaaatcatccaaggctccaacaataccggccgaatcaaaggaagccagggaaaaacgggaaaagatggaaaagctcctcaagaaggctgatgaagcaaccgccgcaaaaaggtactccgaagcaatgacatggctgaaacaggcacgggtactggctcctacagaaaaacagaaggatattgacgaaaagatgcaggaagtacagaaacaggctagtgcaggaagcctgttcggtatggcagaggaaccggcgccggtaattccccaaccacaaggctatatgaacggtcagtcacaaccaggtatgcaaacaagcatattcccggagcaacagacccatactatgaatcctgaacctgtcatgcagcctgctccacagcaggtatcacaacaaattccacaaggaatacctcaaccggcatatggaacgaacgggacatataacccacctgctccaaacagcccgatagtaaaaggagcagacataccgcaaggcgcaacaatgcatccttacccacagcagccatactaccagcaagaggcgactccttatccaacacaacagccacagcaaccgacaaacggacatataccgaatggggctgcgcaagtacagaatggaaacggacgggaataccagactgcatcggctacacatgagacattctgcttcgatccggaagacgagaatgacagggaacttctaagagaggacccgtatgcggaatatccggattttccggctgagtaccgaatgaaggacgaggcacaggtagaaatggtatactgctgatatacacaataaacgatttgtaaaaccaataaactataaacaatatggcactggaaattaaaggaatgaaaagagtattcaagatgaagaagaacaatcaggaaatcgtactggatgatccgaacgtaaacatgtctccggctgaagtgatggacttctattccatgaattatccggaactgacaaccgcgaccgtacacggaccggaaatcgaagacgaccgggcggtatatgaattcaagaccactatcggagtaaaagggtaagagcatgaaaaaaggacaacgtaaagacaagaaaccatgtacacaacttacggaacgggctttggaaaatttagccagacttatcatatcggaactcgaaaatacggacataagccggggcatcaggaacagaaagaaaagaagactccctcccgcagaaagcctcatggttttctgaacacgagaataccttccatcgctcccgatctgtatgttgagaatgacagggatgtaacggtaaatgtcaccaccaaagagaatcttgatttcctgtaccgttcagccatgaagtatgcgcagctcctggatgtggagctgccataccatcctacaggcaggacttccacaagagagaaaatatgcctgctatataatgcactggattccatagtatctcatcatgtaaatctggaacttattggtgacaggctccagttctgcatctaccatttccatgaatggccggattatacgcttttctttatgccgatagactttacggaaaggctgcacggtgaaattaaaaagattacactggagttcatcagaaagttcatcaaatatcacaggatgatggatataaccgataccccttattttgagatgtcggaagtctgtatcgattatgtggactttgaacagctcgatgaggaagagaaaaaggatttgtacagaaaggaaaagcttttcaggtcatatgagaaagggagaatccacaggaagctgtgccggatgcactccagggctttctgtaggaatctggaagaacatatccgcaactgtactccttccagcgataaggaaagaagacttttggaactgattaccgaagggctgtccctgattgcaaaggacagcccttatatcttgaattatgattatgattttgcaagcgaaaaggaacgggatttcgagccgccaccgctcgaatatcagattctgcttacatattccatcacggatacggttaccaaagacatggaaagctgtttcagtactgactgtcaggaaacatataaccagactcccgtatcatttaccttcatcacgccggaaacagaggaacttttcaagccggacaactatccggaacggtttgagaaatggtttgagaaatttgtagaacatgttacctataatttataaacatcatgaatgaactgaccaaaaatatgcaaaaaatgatggtaccgaaggctgcaatcatagcctacaagtatgaagacagaagaaatcttgataccaggtactttatagaattacgtccaatcagaaaaagcggacagatgggggcaggtatccccgtcacatacgaattcatgaataccctgctggaatcctatacggaagaaatgagcgggataccggcaggcagagtccctgaaaacatgctggcctgcaatccgagaaaaggacaggaagaatatatctggtacaatccgcccggaaaaagacagatgttctttcacaaggatctcaatatacaggacggcatgttcaatctgccgggaattatctaccaagtaaaaaacggaaacatggacgtgttcgctttcaaggggaaacgtccggtggagacgactccgctgttccgtgccccgttcttcaacgtgaccggatcaagtgtctgccttggcaacagttctctggaaaagccacagaacccgactttcctttccctgctggaatactgggaaaaacggttctggctgactgaattctcccatctgggaggaaatgtcaatcctaccgtttcaaatcttgtcatcgtcaccgaaaatataagaaacaatccgttcgacatgaacgaactcaagcccatgaataaaaaacttaaagacatacttccatgaaaaagatacattttaccgaccgctacctgctcaatccacgtcatccggtaacggtattcgtcatcggagctggaggtaccggctcacaagtgataaccaatctggcacgcatgagcatggcacttcaggcattaggtcatccgggactgcatgtcaccgtattcgatcccgatacggttagccaggccaatataggacgccagcttttcagtgagacggaactgggactgaacaaggccgtatcacttgtcacacgcatcaaccgtttcttcggatacgcatggactgccgaaccgaaatgtttcccaacgaagaaattttcaggatatgatacagccaacatatttatcacctgcactgacaatatacgttcacgtcttgagatttggaaatttctaaagaaaactcgtaaagagaacttcaatgactatttggttcctatatattggatggattttgggaacagccagacaaagggacaggtcatcatcgggacggtacgtgagaaagttctccaaccttcttcacaagaatatattcccatgcctaaaatgaatgtcatcaccgaggaagtggactatgcgaaaatcaaggaaaaagaatcaggaccaagctgttctctggcggaagccctggaaaaacaggatttgttcattaactccacactggcacatatcggatgtgacatattatggagaatgttcaaggaaggaaagacactgtatcgcggtgcctatgtcaatctggatacattgaaaatgaccgcaatcccggtgtaatgacagaagtgaccgtatcatctttccatcagaatacggtcacttattctatttgctacttattatttactacgttcttaccacgctggagcaggaaactctgtatctctgaggcgagatagaatgatttcccgttcttttccaccgagtaatatttaatcttgccctcttgcctgtaacgtgccaaagttctttgtgacacaccaaggagttctgccagatccacattatcaagcagtctgtctccattcatacattctttcagacgattcatctggtccagtttcttttcaatgcgggcaaatccctctaccattgttcctataagtctttcgagtatctcattatctatatatgacataattccaatgttattaagtgaataaatcgatactctcttcgtgcgcactctaagagtatgtacttatagtagtgaaaatagtatgcctgaatctaagacaaagatcaacaagcttattaggcgctgataatcaggcgtataattttttctacttaatatttagtgtaaaccaaaagtgtaaactatgtaatacagaattgggaacgggttaacacagccaccaacaatgacatctgatgctacctgacgacacctaatgacaacattttgtatcatatacatattcaaaatacatttgtacaaactcaacttttttggatatggaaatcattggaattgaaacagctacatatgaaaagacattaaaggaaattgaaaacttccttgataccattgataaattgattacagcttcttcacagaaaacaataggggaatggttggataaccaagaagtttgcctgatcctcaaaatttctccaagaacattacagaatcttagagatacagaccaaatctcttattctcaaattgggaaaaagatttattataaaaaagaagatattcagaagttcattgaaaaacacaacagaaaattatgagcaaggtaattacccaagataatgagcaagttattcagatatacaataggttaaaagatacgctaacaagactcgaagatattctgaagaataacaacccaacacttaatgggcatagatatatgaatgatgcagaattggctaattaccttaaagtatcaagacgcactttacaagaatatagaaataatggaatcttatcttattatcagattggaggtaaaattctatatcgggaatctgatatagaagaacttcttgagaaaaacagacaggaagcattccgttaaacatttcttggaattttcgttgattttcaaagcaaaaatcagtatctttgcaatactgacaaagagttgtatatcagtgcagaacaaagaagttcaatcgaggtgaaataggtggactaaatgacaaacaacaagataagtaattgattattagcgataaaaaatataaggttccgcccccaggcggatcactgaaaacaaaagagaaat